SEQUENCE LISTING (839 sequences)

SEQ ID NO: 1 - protein, 9 aa, Artificial Sequence
  Description of Artificial Sequence Synthetic peptide
  MOD_RES (1)..(1): Any amino acid
  MOD_RES (2)..(2): Ile or Leu
  MOD_RES (4)..(8): Any amino acid
XXPXXXXXL

SEQ ID NO: 2 - protein, 150 aa, Homo sapiens [sequence omitted]

SEQ ID NO: 3 - protein, 131 aa, Homo sapiens
LSTTEVAMHTSTSSSVTKSYISSQTNDTHKRDTYAATPRAHEVSEISVRTVYPPEEETGERVQLAHHFSEPEITLIIFGVMAGVIGTILLISYGIRRLIKKSPSDVKPLPSPDTDVPLSSVEIENPETSDQ

SEQ ID NO: 4 - protein, 78 aa, Homo sapiens
MQPQESHVHYSRWEDGSRDGVSLGAVSSTEEASRCRRISQRLCTGKLGIAMKVLGGVALFWIIFILGYLTGYYVHKCK

SEQ ID NO: 5 - protein, 567 aa, Anabaena variabilis
MKTLSQAQSKTSSQQFSFTGNSSANVIIGNQKLTINDVARVARNGTLVSLTNNTDILQGIQASCDYINNAVESGEPIYGVTSGFGGMANVAISREQASELQTNLVWFLKTGAGNKLPLADVRAAMLLRANSHMRGASGIRLELIKRMEIFLNAGVTPYVYEFGSIGASGDLVPLSYITGSLIGLDPSFKVDFNGKEMDAPTALRQLNLSPLTLLPKEGLAMMNGTSVMTGIAANCVYDTQILTAIAMGVHALDIQALNGTNQSFHPFIHNSKPHPGQLWAADQMISLLANSQLVRDELDGKHDYRDHELIQDRYSLRCLPQYLGPIVDGISQIAKQIEIEINSVTDNPLIDVDNQASYHGGNFLGQYVGMGMDHLRYYIGLLAKHLDVQIALLASPEFSNGLPPSLLGNRERKVNMGLKGLQICGNSIMPLLTFYGNSIADRFPTHAEQFNQNINSQGYTSATLARRSVDIFQNYVAIALMFGVQAVDLRTYKKTGHYDARACLSPATERLYSAVRHVVGQKPTSDRPYIWNDNEQGLDEHIARISADIAAGGVIVQAVQDILPCLH

SEQ ID NO: 6 - protein, 717 aa, Arabidopsis thaliana
MDQIEAMLCGGGEKTKVAVTTKTLADPLNWGLAADQMKGSHLDEVKKMVEEYRRPVVNLGGETLTIGQVAAISTVGGSVKVELAETSRAGVKASSDWVMESMNKGTDSYGVTTGFGATSHRRTKNGTALQTELIRFLNAGIFGNTKETCHTLPQSATRAAMLVRVNTLLQGYSGIRFEILEAITSLLNHNISPSLPLRGTITASGDLVPLSYIAGLLTGRPNSKATGPDGESLTAKEAFEKAGISTGFFDLQPKEGLALVNGTAVGSGMASMVLFEANVQAVLAEVLSAIFAEVMSGKPEFTDHLTHRLKHHPGQIEAAAIMEHILDGSSYMKLAQKVHEMDPLQKPKQDRYALRTSPQWLGPQIEVIRQATKSIEREINSVNDNPLIDVSRNKAIHGGNFQGTPIGVSMDNTRLAIAAIGKLMFAQFSELVNDFYNNGLPSNLTASSNPSLDYGFKGAEIAMASYCSELQYLANPVTSHVQSAEQHNQDVNSLGLISSRKTSEAVDILKLMSTTFLVGICQAVDLRHLEENLRQTVKNTVSQVAKKVLTTGINGELHPSRFCEKDLLKVVDREQVFTYVDDPCSATYPLMQRLRQVIVDHALSNGETEKNAVTSIFQKIGAFEEELKAVLPKEVEAARAAYGNGTAPIPNRIKECRSYPLYRFVREELGTKLLTGEKVVSPGEEFDKVFTAMCEGKLIDPLMDCLKEWNGAPIPIC

SEQ ID NO: 7 - protein, 469 aa, Pseudomonas putida
MRPIERLLAVVDGEVSARLDEGMRGRIDAGHALLLELIAAGAPIYGVTTGLGAAVDHAQGDAGFQQRIAAGRAVGVGRLASRREVRAIMAARLAGLALGRSGISLASAMALGDFLDHGIHPEVPLLGSLGASDLAPLAHVTLALQGQGWVEYHGERLPAAEALQRAGLAPLVPRDKDGLALVSANSASIGLGALLVSETQRLLDRQRGVLALSCEGYRAGVAPFQAAHLRPAPGLVEESTALLALLEGGDRQARRLQDPLSFRCSTVVLGAVRDALARARDIVVIELQSGADNPALVVKSREVLVTANFDSTHLALAFEGLGLALSRLAVASAERMAKLLSPGSSELPHSLSPRPGSVGLAALQRTAAALVAEIVHLANPLPALSVPVADRVEDYAGQGLAVVEKTARLVQRVEWLVRIEAVVAAQAVDLRAGITLGSEASAIYRQIRQVVAFVEDDRAIDVTGEFWGR

SEQ ID NO: 8 - protein, 348 aa, Erwinia chrysanthemi
MERWFKSLFVLVLFFVFTASAADKLPNIVILATGGTIAGSAATGTQTTGYKAGALGVDTLINAVPEVKKLANVKGEQFSNMASENMTGDVVLKLSQRVNELLARDDVDGVVITHGTDTVEESAYFLHLTVKSDKPVVFVAAMRPATAISADGPMNLLEAVRVAGDKQSRGRGVMVVLNDRIGSARYITKTNASTLDTFKANEEGYLGVIIGNRIYYQNRIDKLHTTRSVFDVRGLTSLPKVDILYGYQDDPEYLYDAAIQHGVKGIVYAGMGAGSVSVRGIAGMRKAMEKGVVVIRSTRTGNGIVPPDEELPGLVSDSLNPAHARILLMLALTRTSDPKVIQEYFHTY

SEQ ID NO: 9 - protein, 348 aa, Escherichia coli
MEFFKKTALAALVMGFSGAALALPNITILATGGTIAGGGDSATKSNYTVGKVGVENLVNAVPQLKDIANVKGEQVVNIGSQDMNDNVWLTLAKKINTDCDKTDGFVITHGTDTMEETAYFLDLTVKCDKPVVMVGAMRPSTSMSADGPFNLYNAVVTAADKASANRGVLVVMNDTVLDGRDVTKTNTTDVATFKSVNYGPLGYIHNGKIDYQRTPARKHTSDTPFDVSKLNELPKVGIVYNYANASDLPAKALVDAGYDGIVSAGVGNGNLYKSVFDTLATAAKTGTAVVRSSRVPTGATTQDAEVDDAKYGFVASGTLNPQKARVLLQLALTQTKDPQQIQQIFNQY

SEQ ID NO: 10 - protein, 338 aa, Escherichia coli
MQKKSIYVAYTGGTIGMQRSEQGYIPVSGHLQRQLALMPEFHRPEMPDFTIHEYTPLMDSSDMTPEDWQHIAEDIKAHYDDYDGFVILHGTDTMAYTASALSFMLENLGKPVIVTGSQIPLAELRSDGQINLLNALYVAANYPINEVTLFFNNRLYRGNRTTKAHADGFDAFASPNLPPLLEAGIHIRRLNTPPAPHGEGELIVHPITPQPIGVVTIYPGISADVVRNFLRQPVKALILRSYGVGNAPQNKAFLQELQEASDRGIVVVNLTQCMSGKVNMGGYATGNALAHAGVIGGADMTVEATLTKLHYLLSQELDTETIRKAMSQNLRGELTPDD

SEQ ID NO: 11 - protein, 322 aa, Staphylococcus aureus
MKHLLVIHTGGTISMSQDQSNKVVTNDINPISMHQDVINQYAQIDELNPFNVPSPHMTIQHVKQLKDIILEAVTNKYYDGFVITHGTDTLEETAFLLDLILGIEQPVVITGAMRSSNEIGSDGLYNYISAIRVASDEKARHKGVMVVFNDEIHTARNVTKTHTSNTNTFQSPNHGPLGVLTKDRVQFHHMPYRQQALENVNDKLNVPLVKAYMGMPGDIFSFYSREGIDGMVIEALGQGNIPPSALEGIQQLVSLNIPIVLVSRSFNGIVSPTYAYDGGGYQLAQQGFIFSNGLNGPKARLKLLVALSNNLDKAEIKSYFEL

SEQ ID NO: 12 - protein, 346 aa, Erwinia carotovora
MFNALFVVVFVCFSSLANAAENLPNIVILATGGTIAGSAAANTQTTGYKAGALGVETLIQAVPELKTLANIKGEQVASIGSENMTSDVLLTLSKRVNELLARSDVDGVVITHGTDTLDESPYFLNLTVKSDKPVVFVAAMRPATAISADGPMNLYGAVKVAADKNSRGRGVLVVLNDRIGSARFISKTNASTLDTFKAPEEGYLGVIIGDKIYYQTRLDKVHTTRSVFDVTNVDKLPAVDIIYGYQDDPEYMYDASIKHGVKGIVYAGMGAGSVSKRGDAGIRKAESKGIVVVRSSRTGSGIVPPDAGQPGLVADSLSPAKSRILLMLALTKTTNPAVIQDYFHAY

SEQ ID NO: 13 - protein, 330 aa, Wolinella succinogenes
MAKPQVTILATGGTIAGSGESSVKSSYSAGAVTVDKLLAAVPAINDLATIKGEQISSIGSQEMTGKVWLKLAKRVNELLAQKETEAVIITHGTDTMEETAFFLNLTVKSQKPVVLVGAMRSGSSMSADGPMNLYNAVNVAINKASTNKGVVIVMNDEIHAAREATKLNTTAVNAFASPNTGKIGTVYYGKVEYFTQSVRPHTLASEFDISKIEELPRVDILYAHPDDTDVLVNAALQAGAKGIIHAGMGNGNPFPLTQNALEKAAKSGVVVARSSRVGSGSTTQEAEVDDKKLGFVATESLNPQKARVLLMLALTKTSDREAIQKIFSTY

SEQ ID NO: 14 - protein, 328 aa, Homo sapiens
MADKLPNIVILATGGTIAGSAATGTQTTGYKAGALGVDTLINAVPEVKKLANVKGEQFSNMASENMTGDVVLKLSQRVNELLARDDVDGVVITHGTDTVEESAYFLHLTVKSDKPVVFVAAMRPATAISADGPMNLLEAVRVAGDKQSRGRGVMVVLNDRIGSARYITKTNASTLDTFKANEEGYLGVIIGNRIYYQNRIDKLHTTRSVFDVRGLTSLPKVDILYGYQDDPEYLYDAAIQHGVKGIVYAGMGAGSVSVRGIAGMRKAMEKGVVVIRSTRTGNGIVPPDEELPGLVSDSLNPAHARILLMLALTRTSDPKVIQEYFHTY

SEQ ID NO: 15 - protein, 337 aa, Pseudomonas sp.
KEVENQQKLANVVILATGGTIAGAGASAANSATYQAAKVGVDKLIAGVPELADLANVRGEQVMQIASESITNDDLLKLGKRVAELADSNDVDGIVITHGTDTLEETAYFLDLTLNTDKPIVVVGSMRPGTAMSADGMLNLYNAVAVASNKDSRGKGVLVTMNDEIQSGRDVSKSINIKTEAFKSAWGPLGMVVEGKSYWFRLPAKRHTVNSEFDIKQISSLPQVDIAYSYGNVTDTAYKALAQNGAKALIHAGTGNGSVSSRLTPALQTLRKTGTQIIRSSHVNQGGFVLRNAEQPDDKNDWVVAHDLNPEKARILVELAMVKTQDSKELQRIFWEY

SEQ ID NO: 16 - protein, 337 aa, Pseudomonas sp.
KEVENQQKLANVVILATGGTIAGAGASAANSATYQAAKVGVDKLIAGVPELADLANVRGEQVMQIASESITNDDLLKLGKRVAELADSNDVDGIVITHGTDTLEETAYFLNLVEKTDKPIVVVGSMRPGTAMSADGMLNLYNAVAVASNKDSRGKGVLVTMNDEIQSGRDVSKSINIKTEAFKSAWGPLGMVVEGKSYWFRLPAKRHTVNSEFDIKQISSLPQVDIAYSYGNVTDTAYKALAQNGAKALIHAGTGNGSVSSRVVPALQELRKNGVQIIRSSHVNQGGFVLRNAEQPDDKNDWVVAHDLNPQKARILAMVAMTKTQDSKELQRIFWEY

SEQ ID NO: 17 - protein, 331 aa, Acinetobacter glutaminasificans
KNNVVIVATGGTIAGAGASSTNSATYSAAKVPVDALIKAVPQVNDLANITGIQALQVASESITDKELLSLARQVNDLVKKPSVNGVVITHGTDTMEETAFFLNLVVHTDKPIVLVGSMRPSTALSADGPLNLYSAVALASSNEAKNKGVMVLMNDSIFAARDVTKGINIHTHAFVSQWGALGTLVEGKPYWFRSSVKKHTNNSEFNIEKIQGDALPGVQIVYGSDNMMPDAYQAFAKAGVKAIIHAGTGNGSMANYLVPEVRKLHDEQGLQIVRSSRVAQGFVLRNAEQPDDKYGWIAAHDLNPQKARLLMALALTKTNDAKEIQNMFWNY

SEQ ID NO: 18 - protein, 362 aa, Pseudomonas putida
MNAALKTFAPSALALLLILPSSASAKEAETQQKLANVVILATGGTIAGAGASAANSATYQAAKLGVDKLIAGVPELADIANVRGEQVMQIASESISNDDLLKLGKRVAELAESKDVDGIVITHGTDTLEETAFFLNLVEKTDKPIVVVGSMRPGTAMSADGMLNLYNAVAVASDKQSRGKGVLVTMNDEIQSGRDVSKAVNIKTEAFKSAWGPMGMVVEGKSYWFRLPAKRHTVNSEFDIKQISSLPQVDIAYGYGNVTDTAYKALAQNGAKALIHAGTGNGSVSSRVVPALQELRKNGVQIIRSSHVNQGGFVLRNAEQPDDKNDWVVAHDLNPQKARILAMVAMTKTQDSKELQRIFWEY

SEQ ID NO: 19 - protein, 387 aa, Aeropyrum pernix
MALADISGYLDVLDSVRGFSYLENAREVLRSGEARCLGNPRSEPEYVKALYVIGASRIPVDGCSHTLEEGVFDISVPGEMVFPSPLDFFERGKPTPLVRSRLQLPNGVRVWLKLEWYNPFSLSVKDRPAVEIISRLSRRVEKGSLVADATSSNFGVALSAVARLYGYRARVYLPGAAEEFGKLLPRLLGAQVIVDPEAPSTVHLLPRVMKDSKNEGFVHVNQFYNDANFEAHMRGTAREIFVQSRRGGLALRGVAGSLGTSGHMSAAAFYLQSVDPSIRAVLVQPAQGDSIPGIRRVETGMLWINMLDISYTLAEVTLEEAMEAVVEVARSDGLVIGPSGGAAVKALAKKAAEGDLEPGDYVVVVPDTGFKYLSLVQNALEGAGDSV

SEQ ID NO: 20 - protein, 105 aa, Artificial Sequence
  Description of Artificial Sequence Synthetic polypeptide
  MISC_FEATURE (1)..(100): This region may encompass 1-20 “Gly Gly Gly Xaa Xaa” repeating units, wherein Xaa is any amino acid
  MOD_RES (4)..(5): Any amino acid
  MOD_RES (9)..(10): Any amino acid
  MOD_RES (14)..(15): Any amino acid
  MOD_RES (19)..(20): Any amino acid
  MOD_RES (24)..(25): Any amino acid
  MOD_RES (29)..(30): Any amino acid
  MOD_RES (34)..(35): Any amino acid
  MOD_RES (39)..(40): Any amino acid
  MOD_RES (44)..(45): Any amino acid
  MOD_RES (49)..(50): Any amino acid
  MOD_RES (54)..(55): Any amino acid
  MOD_RES (59)..(60): Any amino acid
  MOD_RES (64)..(65): Any amino acid
  MOD_RES (69)..(70): Any amino acid
  MOD_RES (74)..(75): Any amino acid
  MOD_RES (79)..(80): Any amino acid
  MOD_RES (84)..(85): Any amino acid
  MOD_RES (89)..(90): Any amino acid
  MOD_RES (94)..(95): Any amino acid
  MOD_RES (99)..(100): Any amino acid
GGGXXGGGXXGGGXXGGGXXGGGXXGGGXXGGGXXGGGXXGGGXXGGGXXGGGXXGGGXXGGGXXGGGXXGGGXXGGGXXGGGXXGGGXXGGGXXGGGXXGGGGS

SEQ ID NO: 21 - protein, 105 aa, Artificial Sequence
  Description of Artificial Sequence Synthetic polypeptide
  MOD_RES (6)..(6): Any amino acid
  MISC_FEATURE (6)..(105): This region may encompass 1-20 “Xaa Gly Gly Gly Ser” repeating units, wherein Xaa is any amino acid
  MOD_RES (11)..(11): Any amino acid
  MOD_RES (16)..(16): Any amino acid
  MOD_RES (21)..(21): Any amino acid
  MOD_RES (26)..(26): Any amino acid
  MOD_RES (31)..(31): Any amino acid
  MOD_RES (36)..(36): Any amino acid
  MOD_RES (41)..(41): Any amino acid
  MOD_RES (46)..(46): Any amino acid
  MOD_RES (51)..(51): Any amino acid
  MOD_RES (56)..(56): Any amino acid
  MOD_RES (61)..(61): Any amino acid
  MOD_RES (66)..(66): Any amino acid
  MOD_RES (71)..(71): Any amino acid
  MOD_RES (76)..(76): Any amino acid
  MOD_RES (81)..(81): Any amino acid
  MOD_RES (86)..(86): Any amino acid
  MOD_RES (91)..(91): Any amino acid
  MOD_RES (96)..(96): Any amino acid
  MOD_RES (101)..(101): Any amino acid
GGGGSXGGGSXGGGSXGGGSXGGGSXGGGSXGGGSXGGGSXGGGSXGGGSXGGGSXGGGSXGGGSXGGGSXGGGSXGGGSXGGGSXGGGSXGGGSXGGGSXGGGS

SEQ ID NO: 22 - protein, 10 aa, Artificial Sequence
  Description of Artificial Sequence Synthetic peptide
GGGGSGGGGS

SEQ ID NO: 23 - protein, 10 aa, Artificial Sequence
  Description of Artificial Sequence Synthetic peptide
GSGSGSGSGS

SEQ ID NO: 24 - protein, 7 aa, Artificial Sequence
  Description of Artificial Sequence Synthetic peptide
PSTSTST

SEQ ID NO: 25 - protein, 7 aa, Artificial Sequence
  Description of Artificial Sequence Synthetic peptide
EIDKPSQ

SEQ ID NO: 26 - protein, 9 aa, Artificial Sequence
  Description of Artificial Sequence Synthetic peptide
YPYDVPDYA

SEQ ID NO: 27 - protein, 10 aa, Artificial Sequence
  Description of Artificial Sequence Synthetic peptide
EQKLISEEDL

SEQ ID NO: 28 - protein, 8 aa, Artificial Sequence
  Description of Artificial Sequence Synthetic peptide
DYKDDDDK

SEQ ID NO: 29 - DNA, 54 bp, Artificial Sequence
  Description of Artificial Sequence Synthetic oligonucleotide
gagggcagaggaagtcttctaacatgcggtgacgtggaggsgsstcccggccct

SEQ ID NO: 30 - protein, 21 aa, Artificial Sequence
  Description of Artificial Sequence Synthetic peptide
GGGGSSSGGGGSSSGGGGSSS

SEQ ID NO: 31 - protein, 4 aa, Artificial Sequence
  Description of Artificial Sequence Synthetic peptide
AGST

SEQ ID NO: 32 - protein, 5 aa, Artificial Sequence
  Description of Artificial Sequence Synthetic peptide
  MOD_RES (3)..(3): Any amino acid
LPXTG

SEQ ID NO: 33 - protein, 5 aa, Artificial Sequence
  Description of Artificial Sequence Synthetic peptide
  MOD_RES (3)..(3): Any amino acid
LPXTA

SEQ ID NO: 34 - protein, 338 aa, Homo sapiens
MVVMAPRTLFLLLSGALTLTETWAGSHSMRYFSAAVSRPGRGEPRFIAMGYVDDTQFVRFDSDSACPRMEPRAPWVEQEGPEYWEEETRNTKAHAQTDRMNLQTLRGYYNQSEASSHTLQWMIGCDLGSDGRLLRGYEQYAYDGKDYLALNEDLRSWTAADTAAQISKRKCEAANVAEQRRAYLEGTCVEWLHRYLENGKEMLQRADPPKTHVTHHPVFDYEATLRCWALGFYPAEIILTWQRDGEDQTQDVELVETRPAGDGTFQKWAAVVVPSGEEQRYTCHVQHEGLPEPLMLRWKQSSLPTIPIMGIVAGLVVLAAVVTGAAVAAVLWRKKSSD

SEQ ID NO: 35 - protein, 314 aa, Homo sapiens
GSHSMRYFSAAVSRPGRGEPRFIAMGYVDDTQFVRFDSDSACPRMEPRAPWVEQEGPEYWEEETRNTKAHAQTDRMNLQTLRGYYNQSEASSHTLQWMIGCDLGSDGRLLRGYEQYAYDGKDYLALNEDLRSWTAADTAAQISKRKCEAANVAEQRRAYLEGTCVEWLHRYLENGKEMLQRADPPKTHVTHHPVFDYEATLRCWALGFYPAEIILTWQRDGEDQTQDVELVETRPAGDGTFQKWAAVVVPSGEEQRYTCHVQHEGLPEPLMLRWKQSSLPTIPIMGIVAGLVVLAAVVTGAAVAAVLWRKKSSD

SEQ ID NO: 36 - protein, 10 aa, Artificial Sequence
  Description of Artificial Sequence Synthetic peptide
FENDAQAPKS

SEQ ID NO: 37 - protein, 9 aa, Artificial Sequence
  Description of Artificial Sequence Synthetic peptide
QNDAQAPKS

SEQ ID NO: 38 - protein, 20 aa, Homo sapiens
TVIIEQSWGSPKVTKDGVTV

SEQ ID NO: 39 - protein, 9 aa, Homo sapiens
QMRPVSRVL

SEQ ID NO: 40 - protein, 15 aa, Homo sapiens
AYVLLSEKKISSIQS

SEQ ID NO: 41 - protein, 15 aa, Homo sapiens
AYVLLSEKKISSIQS

SEQ ID NO: 42 - protein, 15 aa, Homo sapiens
GEALSTLVLNRLKVG

SEQ ID NO: 43 - protein, 15 aa, Homo sapiens
LAKLSDGVAVLKVGG

SEQ ID NO: 44 - protein, 15 aa, Homo sapiens
VMRIINEPTAAAIAY

SEQ ID NO: 45 - protein, 15 aa, Homo sapiens
  MOD_RES (13)..(13): Citrullinated
EVTFEIDVNGILRVT

SEQ ID NO: 46 - protein, 15 aa, Homo sapiens
EVTFEIDVNGILRVT

SEQ ID NO: 47 - protein, 15 aa, Homo sapiens
  MOD_RES (11)..(11): Citrullinated
TFEIDVNGILRVTAE

SEQ ID NO: 48 - protein, 15 aa, Homo sapiens
  MOD_RES (3)..(3): Citrullinated
VMRIINEPTAAAIAY

SEQ ID NO: 49 - protein, 9 aa, Homo sapiens
  MOD_RES (1)..(1): Oxidated
MLYQHLLPL

SEQ ID NO: 50 - protein, 20 aa, Homo sapiens
YVVKSFDRSTKVIDFHYPNE

SEQ ID NO: 51 - protein, 17 aa, Homo sapiens
AMMIARFKMFPEVKEKG

SEQ ID NO: 52 - protein, 12 aa, Homo sapiens
GIVEQCCTSICS

SEQ ID NO: 53 - protein, 12 aa, Homo sapiens
QCCTSICSLYQL

SEQ ID NO: 54 - protein, 14 aa, Homo sapiens
LVEALYLVCGERGF

SEQ ID NO: 55 - protein, 15 aa, Homo sapiens
FVNQHLCGSHLVEAL

SEQ ID NO: 56 - protein, 15 aa, Homo sapiens
GSHLVEALYLVCGER

SEQ ID NO: 57 - protein, 15 aa, Homo sapiens
HLCGSHLVEALYLVC

SEQ ID NO: 58 - protein, 15 aa, Homo sapiens
HLVEALYLVCGERGF

SEQ ID NO: 59 - protein, 17 aa, Homo sapiens
LCGSHLVEALYLVCGER

SEQ ID NO: 60 - protein, 15 aa, Homo sapiens
LVEALYLVCGERGFF

SEQ ID NO: 61 - protein, 15 aa, Homo sapiens
LYLVCGERGFFYTPK

SEQ ID NO: 62 - protein, 15 aa, Homo sapiens
QHLCGSHLVEALYLV

SEQ ID NO: 63 - protein, 15 aa, Homo sapiens
VEALYLVCGERGFFY

SEQ ID NO: 64 - protein, 20 aa, Homo sapiens
LVLNRLKVGLQVVAVKAPGF

SEQ ID NO: 65 - protein, 20 aa, Homo sapiens
EIIKRTLKIPAMTIAKNAGV

SEQ ID NO: 66 - protein, 20 aa, Homo sapiens
GEVIVTKDDAMLLKGKGDKA

SEQ ID NO: 67 - protein, 20 aa, Homo sapiens
IVLGGGCALLRCIPALDSLT

SEQ ID NO: 68 - protein, 20 aa, Homo sapiens
KFGADARALMLQGVDLLADA

SEQ ID NO: 69 - protein, 20 aa, Homo sapiens
LVIIAEDVDGEALSTLVLNR

SEQ ID NO: 70 - protein, 20 aa, Homo sapiens
EEIAQVATISANGDKEIGNI

SEQ ID NO: 71 - protein, 20 aa, Homo sapiens
KAPGFGDNRKNQLKDMAIAT

SEQ ID NO: 72 - protein, 20 aa, Homo sapiens
LLADAVAVTMGPKGRTVIIE

SEQ ID NO: 73 - protein, 20 aa, Homo sapiens
MLRLPTVFRQMRPVSRVLAP

SEQ ID NO: 74 - protein, 20 aa, Homo sapiens
NEEAGDGTTTATVLARSIAK

SEQ ID NO: 75 - protein, 20 aa, Homo sapiens
NPVEIRRGVMLAVDAVIAEL

SEQ ID NO: 76 - protein, 20 aa, Homo sapiens
QDAYVLLSEKKISSIQSIVP

SEQ ID NO: 77 - protein, 21 aa, Homo sapiens
QSIVPALEIANAHRKPLVIIA

SEQ ID NO: 78 - protein, 20 aa, Homo sapiens
RKGVITVKDGKTLNDELEII

SEQ ID NO: 79 - protein, 20 aa, Homo sapiens
RSIAKEGFEKISKGANPVEI

SEQ ID NO: 80 - protein, 20 aa, Homo sapiens
RVLAPHLTRAYAKDVKFGAD

SEQ ID NO: 81 - protein, 20 aa, Homo sapiens
VIAELKKQSKPVTTPEEIAQ

SEQ ID NO: 82 - protein, 20 aa, Homo sapiens
VNMVEKGIIDPTKVVRTALL

SEQ ID NO: 83 - protein, 20 aa, Homo sapiens
VTDALNATRAAVEEGIVLGG

SEQ ID NO: 84 - protein, 24 aa, Homo sapiens
VLGGGCALLRCIPALDSLTPANED

SEQ ID NO: 85 - protein, 14 aa, Homo sapiens
WSKMDQLAKELTAE

SEQ ID NO: 86 - protein, 10 aa, Homo sapiens
LLCAGQVTAL

SEQ ID NO: 87 - protein, 10 aa, Homo sapiens
TLSKPSPMPV

SEQ ID NO: 88 - protein, 13 aa, Homo sapiens
TTLLPQWRVSAFV

SEQ ID NO: 89 - protein, 9 aa, Homo sapiens
KLNWDLHTA

SEQ ID NO: 90 - protein, 9 aa, Homo sapiens
FTNHFLVEL

SEQ ID NO: 91 - protein, 33 aa, Homo sapiens
VRTYWIIIELKHKAREKPYDSKSLRTALQKEIT

SEQ ID NO: 92 - protein, 9 aa, Homo sapiens
KIIKLFFRL

SEQ ID NO: 93 - protein, 9 aa, Homo sapiens
NLAQDLATV

SEQ ID NO: 94 - protein, 9 aa, Homo sapiens
QLARQQVHV

SEQ ID NO: 95 - protein, 33 aa, Homo sapiens
MRVTLATIAWMVSFVSNYSHTANILPDIENEDF

SEQ ID NO: 96 - protein, 9 aa, Homo sapiens
TLATIAWMV

SEQ ID NO: 97 - protein, 15 aa, Homo sapiens
VTLATIAWMVSFVSN

SEQ ID NO: 98 - protein, 13 aa, Homo sapiens
EWVHIDTTPFASL

SEQ ID NO: 99 - protein, 15 aa, Homo sapiens
LYHFLQIPTHEEHLF

SEQ ID NO: 100 - protein, 20 aa, Homo sapiens
MASPGSGFWSFGSEDGSGDS

SEQ ID NO: 101 - protein, 20 aa, Homo sapiens
IPPSLRTLEDNEERMSRLSK

SEQ ID NO: 102 - protein, 20 aa, Homo sapiens
ATHQDIDFLIEEIERLGQDL

SEQ ID NO: 103 - protein, 20 aa, Homo sapiens
AALGIGTDSVILIKCDERGK

SEQ ID NO: 104 - protein, 20 aa, Homo sapiens
TNMFTYEIAPVFVLLEYVTL

SEQ ID NO: 105 - protein, 21 aa, Homo sapiens
CGRHVDVFKLWLMWRAKGTTG

SEQ ID NO: 106 - protein, 20 aa, Homo sapiens
EEILMHCQTTLKYAIKTGHP

SEQ ID NO: 107 - protein, 20 aa, Homo sapiens
ERANSVTWNPHKMMGVPLQC

SEQ ID NO: 108 - protein, 20 aa, Homo sapiens
EYGTTMVSYQPLGDKVNFFR

SEQ ID NO: 109 - protein, 20 aa, Homo sapiens
EYLYNIIKNREGYEMVFDGK

SEQ ID NO: 110 - protein, 20 aa, Homo sapiens
GGSGDGIFSPGGAISNMYAM

SEQ ID NO: 111 - protein, 22 aa, Homo sapiens
KGTTGFEAHVDKCLELAEYLYN

SEQ ID NO: 112 - protein, 20 aa, Homo sapiens
KTGHPRYFNQLSTGLDMVGL

SEQ ID NO: 113 - protein, 22 aa, Homo sapiens
LAFLQDVMNILLQYVVKSFDRS

SEQ ID NO: 114 - protein, 20 aa, Homo sapiens
LEAKQKGFVPFLVSATAGTT

SEQ ID NO: 115 - protein, 20 aa, Homo sapiens
LLYGDAEKPAESGGSQPPRA

SEQ ID NO: 116 - protein, 20 aa, Homo sapiens
QNCNQMHASYLFQQDKHYDL

SEQ ID NO: 117 - protein, 20 aa, Homo sapiens
VFDGKPQHTNVCFWYIPPSL

SEQ ID NO: 118 - protein, 20 aa, Homo sapiens
VNFFRMVISNPAATHQDIDF

SEQ ID NO: 119 - protein, 20 aa, Homo sapiens
IAPVFVLLEYVTLKKMREII

SEQ ID NO: 120 - protein, 20 aa, Homo sapiens
VAPVIKARMMEYGTTMVSYQ

SEQ ID NO: 121 - protein, 18 aa, Homo sapiens
LPRLIAFTSEHSHFSLKK

SEQ ID NO: 122 - protein, 14 aa, Homo sapiens
VNFFRMVISNPAAT

SEQ ID NO: 123 - protein, 9 aa, Homo sapiens
  MOD_RES (4)..(4): Citrullinated
ALPRLIAFT

SEQ ID NO: 124 - protein, 20 aa, Homo sapiens [sequence omitted]

SEQ ID NO: 125 - protein, 15 aa, Homo sapiens
NMFTYEIAPVFVLME

SEQ ID NO: 126 - protein, 20 aa, Homo sapiens
PTIAFLQDVMNILLQYVVKS

SEQ ID NO: 127 - protein, 9 aa, Homo sapiens
VMNILLQYW

SEQ ID NO: 128 - protein, 15 aa, Homo sapiens
CDGERPTLAFLQDVM

SEQ ID NO: 129 - protein, 13 aa, Homo sapiens
IAFTSEHSHFSLK

SEQ ID NO: 130 - protein, 20 aa, Homo sapiens
NMYAMMIARFKMFPEVKEKG

SEQ ID NO: 131 - protein, 15 aa, Homo sapiens
TYEIAPVFVLLEYVT

SEQ ID NO: 132 - protein, 13 aa, Homo sapiens
MNILLQYVVKSFD

SEQ ID NO: 133 - protein, 13 aa, Homo sapiens
NFFRMVISNPAAT

SEQ ID NO: 134 - protein, 15 aa, Homo sapiens
CFWYIPPSLRTLEDN

SEQ ID NO: 135 - protein, 15 aa, Homo sapiens
ERMSRLSKVAPVIKA

SEQ ID NO: 136 - protein, 33 aa, Homo sapiens
NMYAMMIARFKMFPEVKEKGMAALPRLIAFTSE

SEQ ID NO: 137 - protein, 20 aa, Homo sapiens
SRLSKVAPVIKARMMEYGTT

SEQ ID NO: 138 - protein, 41 aa, Homo sapiens
VSYQPLGDKVNFFRMVISNPAATHQDIDFLIEEIERLGQDL

SEQ ID NO: 139 - protein, 9 aa, Homo sapiens
FLQDVMNIL

SEQ ID NO: 140 - protein, 18 aa, Homo sapiens
KVNFFRMVISNPAATHQD

SEQ ID NO: 141 - protein, 9 aa, Homo sapiens
LLQEYNWEL

SEQ ID NO: 142 - protein, 14 aa, Homo sapiens
NILLQYVVKSFDRS

SEQ ID NO: 143 - protein, 13 aa, Homo sapiens
NPAATHQDIDFLI

SEQ ID NO: 144 - protein, 10 aa, Homo sapiens
RMMEYGTTMV

SEQ ID NO: 145 - protein, 10 aa, Homo sapiens
VMNILLQYVV

SEQ ID NO: 146 - protein, 13 aa, Homo sapiens
AKGTTGFEAHVDK

SEQ ID NO: 147 - protein, 15 aa, Homo sapiens
FDRSTKVIDFHYPNE

SEQ ID NO: 148 - protein, 20 aa, Homo sapiens
FFRMVISNPAATHQDIDFLI

SEQ ID NO: 149 - protein, 12 aa, Homo sapiens
GHPRYFNQLSTG

SEQ ID NO: 150 - protein, 15 aa, Homo sapiens
KHYDLSYDTGDKALQ

SEQ ID NO: 151 - protein, 14 aa, Homo sapiens
LPRLIAFTSEHSHF

SEQ ID NO: 152 - protein, 15 aa, Homo sapiens
LPRLIAFTSEHSHFS

SEQ ID NO: 153 - protein, 20 aa, Homo sapiens
NWELADQPQNLEEILMHCQT

SEQ ID NO: 154 - protein, 12 aa, Homo sapiens
RLIAFTSEHSHF

SEQ ID NO: 155 - protein, 15 aa, Homo sapiens
RMMEYGTTMVSYQPL

SEQ ID NO: 156 - protein, 18 aa, Homo sapiens
ANTNMFTYEIAPVFVLLE

SEQ ID NO: 157 - protein, 20 aa, Homo sapiens
EVKEKGMAALPRLIAFTSEH

SEQ ID NO: 158 - protein, 13 aa, Homo sapiens
FWYIPPSLRTLED

SEQ ID NO: 159 - protein, 21 aa, Homo sapiens
GGGLLMSRKHKWKLSGVERAN

SEQ ID NO: 160 - protein, 19 aa, Homo sapiens
GLMQNCNQMHASYLFQQDK

SEQ ID NO: 161 - protein, 20 aa, Homo sapiens
HTNVCFWYIPPSLRTLEDNE

SEQ ID NO: 162 - protein, 15 aa, Homo sapiens
MIARFKMFPEVKEKG

SEQ ID NO: 163 - protein, 20 aa, Homo sapiens
MMIARFKMFPEVKEKGMAAL

SEQ ID NO: 164 - protein, 10 aa, Homo sapiens
MYAMMIARFK

SEQ ID NO: 165 - protein, 12 aa, Homo sapiens
MYAMMIARFKMF

SEQ ID NO: 166 - protein, 12 aa, Homo sapiens
NILLQYVVKSFD

SEQ ID NO: 167 - protein, 12 aa, Homo sapiens
NYAFLHATDLLP

SEQ ID NO: 168 - protein, 20 aa, Homo sapiens
PSLRTLEDNEERMSRLSKVA

SEQ ID NO: 169 - protein, 9 aa, Homo sapiens
RFKMFPEVK

SEQ ID NO: 170 - protein, 20 aa, Homo sapiens
SCSKVDVNYAFLHATDLLPA

SEQ ID NO: 171 - protein, 9 aa, Homo sapiens
TSEHSHFSL

SEQ ID NO: 172 - protein, 9 aa, Homo sapiens
VMNILLQYV

SEQ ID NO: 173 - protein, 20 aa, Homo sapiens
YEMVFDGKPQHTNVCFWYIP

SEQ ID NO: 174 - protein, 20 aa, Homo sapiens
EYVTLKKMREIIGWPGGSGD

SEQ ID NO: 175 - protein, 20 aa, Homo sapiens
GMAALPRLIAFTSEHSHFSL

SEQ ID NO: 176 - protein, 15 aa, Homo sapiens
IKARMMEYGTTMVSY

SEQ ID NO: 177 - protein, 15 aa, Homo sapiens
MVFDGKPQHTNVCFW

SEQ ID NO: 178 - protein, 15 aa, Homo sapiens
PSLRTLEDNEERMSR

SEQ ID NO: 179 - protein, 15 aa, Homo sapiens
TGHPRYFNQLSTGLD

SEQ ID NO: 180 - protein, 9 aa, Homo sapiens
ELAEYLYNI

SEQ ID NO: 181 - protein, 9 aa, Homo sapiens
ILMHCQTTL

SEQ ID NO: 182 - protein, 20 aa, Homo sapiens
PEVKEKGMAALPRLIAFTSE

SEQ ID NO: 183 - protein, 23 aa, Homo sapiens
ARFKMFPEVKEKGMAALPRLIAF

SEQ ID NO: 184 - protein, 21 aa, Homo sapiens
DKVNFFRMVISNPAATHQDID

SEQ ID NO: 185 - protein, 20 aa, Homo sapiens
CACDQKPCSCSKVDVNYAFL

SEQ ID NO: 186 - protein, 20 aa, Homo sapiens
GGLLMSRKHKWKLSGVERAN

SEQ ID NO: 187 - protein, 20 aa, Homo sapiens
ICKKYKIWMHVDAAWGGGLL

SEQ ID NO: 188 - protein, 20 aa, Homo sapiens
REIIGWPGGSGDGIFSPGGA

SEQ ID NO: 189 - protein, 20 aa, Homo sapiens
RYFNQLSTGLDMVGLAADWL

SEQ ID NO: 190 - protein, 20 aa, Homo sapiens
YAMMIARFKMFPEVKEKGMA

SEQ ID NO: 191 - protein, 9 aa, Homo sapiens
ACDGERPTL

SEQ ID NO: 192 - protein, 9 aa, Homo sapiens
AHVDKCLEL

SEQ ID NO: 193 - protein, 9 aa, Homo sapiens
APVIKARMM

SEQ ID NO: 194 - protein, 10 aa, Homo sapiens
HPRYFNQLST

SEQ ID NO: 195 - protein, 10 aa, Homo sapiens
IPSDLERRIL

SEQ ID NO: 196 - protein, 9 aa, Homo sapiens
SPGSGFWSF

SEQ ID NO: 197 - protein, 19 aa, Homo sapiens
CKKYKIWMHVDAAWGGGLL

SEQ ID NO: 198 - protein, 18 aa, Homo sapiens
DVNYAFLHATDLLPACDG

SEQ ID NO: 199 - protein, 24 aa, Homo sapiens
EKGMAALPRLIAFTSEHSHFSLKK

SEQ ID NO: 200 - protein, 25 aa, Homo sapiens
GAISNMYAMMIARFKMFPEVKEKGM

SEQ ID NO: 201 - protein, 24 aa, Homo sapiens
GGLLMSRKHKWKLSGVERANSVTW

SEQ ID NO: 202 - protein, 16 aa, Homo sapiens
HPRYFNQLSTGLDMVG

SEQ ID NO: 203 - protein, 21 aa, Homo sapiens
LGDKVNFFRMVISNPAATHQD

SEQ ID NO: 204 - protein, 24 aa, Homo sapiens
NMFTYEIAPVFVLLEYVTLKKMRE

SEQ ID NO: 205 - protein, 20 aa, Homo sapiens
SALLVREEGLMQNCNQMHAS

SEQ ID NO: 206 - protein, 15 aa, Homo sapiens
TTVYGAFDPLLAVAD

SEQ ID NO: 207 - protein, 15 aa, Homo sapiens
VCFWYIPPSLRTLED

SEQ ID NO: 208 - protein, 21 aa, Homo sapiens
VDVFKLWLMWRAKGTTGFEAH

SEQ ID NO: 209 - protein, 17 aa, Homo sapiens
YLYNIIKNREGYEMVFD

SEQ ID NO: 210 - protein, 22 aa, Homo sapiens
VNFFRMVISNPAATHQDIDFLI

SEQ ID NO: 211 - protein, 20 aa, Homo sapiens
  MOD_RES (8)..(8): Citrullinated
KGMAALPRLIAFTSEHSHFS

SEQ ID NO: 212 - protein, 20 aa, Homo sapiens
  MOD_RES (6)..(6): Citrullinated
KVNFFRMVISNPAATHQDID

SEQ ID NO: 213 - protein, 13 aa, Homo sapiens
  MOD_RES (6)..(6): Deaminated
MNILLQYVVKSFD

SEQ ID NO: 214 - protein, 20 aa, Homo sapiens
  MOD_RES (2)..(2): Deaminated
  MOD_RES (12)..(12): Deaminated
PQNLEEILMHCQTTLKYAIK

SEQ ID NO: 215 - protein, 20 aa, Homo sapiens
  MOD_RES (17)..(17): Citrullinated
YAFLHATDLLPACDGERPTL

SEQ ID NO: 216 - protein, 9 aa, Homo sapiens
FTSEHSHFS

SEQ ID NO: 217 - protein, 9 aa, Homo sapiens
MFPEVKEKG

SEQ ID NO: 218 - protein, 20 aa, Homo sapiens
EAKQKGFVPFLVSATAGTTV

SEQ ID NO: 219 - protein, 20 aa, Homo sapiens
DERGKMIPSDLERRILEAKQ

SEQ ID NO: 220 - protein, 20 aa, Homo sapiens
DVMNILLQYVVKSFDRSTKV

SEQ ID NO: 221 - protein, 20 aa, Homo sapiens
KVNFFRMVISNPAATHQDID

SEQ ID NO: 222 - protein, 20 aa, Homo sapiens
LIAFTSEHSHFSLKKGAAAL

SEQ ID NO: 223 - protein, 20 aa, Homo sapiens
DSVILIKCDERGKMIPSDLE

SEQ ID NO: 224 - protein, 20 aa, Homo sapiens
HKWKLSGVERANSVTWNPHK

SEQ ID NO: 225 - protein, 20 aa, Homo sapiens
KCLELAEYLYNIIKNREGYE

SEQ ID NO: 226 - protein, 20 aa, Homo sapiens
KGMAALPRLIAFTSEHSHFS

SEQ ID NO: 227 - protein, 20 aa, Homo sapiens
RPTLAFLQDVMNILLQYVVK

SEQ ID NO: 228 - protein, 20 aa, Homo sapiens
YDLSYDTGDKALQCGRHVDV

SEQ ID NO: 229 - protein, 18 aa, Homo sapiens
TAGTTVYGAFDPLLAVAD

SEQ ID NO: 230 - protein, 9 aa, Homo sapiens
YMCTHRLLL

SEQ ID NO: 231 - protein, 20 aa, Homo sapiens
AEDEVQRERVSAKNALESYA

SEQ ID NO: 232 - protein, 20 aa, Homo sapiens
AEKDEFEHKRKELEQVCNPI

SEQ ID NO: 233 - protein, 20 aa, Homo sapiens
AGGVMTALIKRNSTIPTKQT

SEQ ID NO: 234 - protein, 20 aa, Homo sapiens
DTERLIGDAAKNQVALNPQN

SEQ ID NO: 235 - protein, 20 aa, Homo sapiens
GLNVLRIINEPTAAAIAYGL

SEQ ID NO: 236 - protein, 10 aa, Homo sapiens
GSGPTIEEVD

SEQ ID NO: 237 - protein, 20 aa, Homo sapiens
IAYGLDRTGKGERNVLIFDL

SEQ ID NO: 238 - protein, 20 aa, Homo sapiens
KANKITITNDKGRLSKEEIE

SEQ ID NO: 239 - protein, 20 aa, Homo sapiens
KEEIERMVQEAEKYKAEDEV

SEQ ID NO: 240 - protein, 20 aa, Homo sapiens
KRTLSSSTQASLEIDSLFEG

SEQ ID NO: 241 - protein, 20 aa, Homo sapiens
LESYAFNMKSAVEDEGLKGK

SEQ ID NO: 242 - protein, 20 aa, Homo sapiens
LLLLDVAPLSLGLETAGGVM

SEQ ID NO: 243 - protein, 20 aa, Homo sapiens
MAKAAAVGIDLGTTYSCVGV

SEQ ID NO: 244 - protein, 20 aa, Homo sapiens
NDQGNRTTPSYVAFTDTERL

SEQ ID NO: 245 - protein, 20 aa, Homo sapiens
PAPGVPQIEVTFDIDANGIL

SEQ ID NO: 246 - protein, 20 aa, Homo sapiens
PFQVINDGDKPKVQVSYKGE

SEQ ID NO: 247 - protein, 20 aa, Homo sapiens
PGPGGFGAQGPKGGSGSGPT

SEQ ID NO: 248 - protein, 20 aa, Homo sapiens
PTKQTQIFTTYSDNQPGVLI

SEQ ID NO: 249 - protein, 20 aa, Homo sapiens
RFEELCSDLFRSTLEPVEKA

SEQ ID NO: 250 - protein, 20 aa, Homo sapiens
SCVGVFQHGKVEIIANDQGN

SEQ ID NO: 251 - protein, 20 aa, Homo sapiens
THLGGEDFDNRLVNHFVEEF

SEQ ID NO: 252 - protein, 20 aa, Homo sapiens
TIDDGIFEVKATAGDTHLGG

SEQ ID NO: 253 - protein, 20 aa, Homo sapiens
VCNPIISGLYQGAGGPGPGG

SEQ ID NO: 254 - protein, 20 aa, Homo sapiens
VQKLLQDFFNGRDLNKSINP

SEQ ID NO: 255 - protein, 20 aa, Homo sapiens
  MOD_RES (9)..(9): Citrullinated
VGSNTYGKRNAVEVLKREPL

SEQ ID NO: 256 - protein, 20 aa, Homo sapiens
  MOD_RES (9)..(9): Citrullinated
  MOD_RES (17)..(17): Citrullinated
VGSNTYGKRNAVEVLKREPL

SEQ ID NO: 257 - protein, 16 aa, Homo sapiens
CARQEDTAMVYYFDYW

SEQ ID NO: 258 - protein, 10 aa, Homo sapiens
GIVEQCCTSI

SEQ ID NO: 259 - protein, 13 aa, Homo sapiens
LVEALYLVCGERG

SEQ ID NO: 260 - protein, 30 aa, Homo sapiens
AAAFVNQHLCGSHLVEALYLVCGERGFFYT

SEQ ID NO: 261 - protein, 10 aa, Homo sapiens
ALWMRLLPLL

SEQ ID NO: 262 - protein, 9 aa, Homo sapiens
LPLLALLAL

SEQ ID NO: 263 - protein, 9 aa, Homo sapiens
LWMRLLPLL

SEQ ID NO: 264 - protein, 11 aa, Homo sapiens
ALWGPDPAAAF

SEQ ID NO: 265 - protein, 10 aa, Homo sapiens
LALWGPDPAA

SEQ ID NO: 266 - protein, 11 aa, Homo sapiens
RLLPLLALLAL

SEQ ID NO: 267 - protein, 20 aa, Homo sapiens
PLLALLALWGPDPAAAFVNQ

SEQ ID NO: 268 - protein, 9 aa, Homo sapiens
GSHLVEALY

SEQ ID NO: 269 - protein, 12 aa, Homo sapiens
ALLALWGPDPAA

SEQ ID NO: 270 - protein, 12 aa, Homo sapiens
ALWGPDPAAAFV

SEQ ID NO: 271 - protein, 12 aa, Homo sapiens
PLLALLALWGPD

SEQ ID NO: 272 - protein, 13 aa, Homo sapiens
GIVEQCCTSICSL

SEQ ID NO: 273 - protein, 9 aa, Homo sapiens
GLQANPVEV

SEQ ID NO: 274 - protein, 15 aa, Homo sapiens
SHLVEALYLVCGERG

SEQ ID NO: 275 - protein, 9 aa, Homo sapiens
ALWMRLLPL

SEQ ID NO: 276 - protein, 9 aa, Homo sapiens
HLVEALYLV

SEQ ID NO: 277 - protein, 10 aa, Homo sapiens
SLQKRGIVEQ

SEQ ID NO: 278 - protein, 9 aa, Homo sapiens
SLQPLALEG

SEQ ID NO: 279 - protein, 15 aa, Homo sapiens
SLQPLALEGSLQKRG

SEQ ID NO: 280 - protein, 9 aa, Homo sapiens
SLYQLENYC

SEQ ID NO: 281 - protein, 16 aa, Homo sapiens
EDLQVGQVELGGGPGA

SEQ ID NO: 282 - protein, 16 aa, Homo sapiens
FYTPKTRREAEDLQVG

SEQ ID NO: 283 - protein, 18 aa, Homo sapiens
GAGSLQPLALEGSLQKRG

SEQ ID NO: 284 - protein, 16 aa, Homo sapiens
HLVEALYLVCGERGFF

SEQ ID NO: 285 - protein, 10 aa, Homo sapiens
VCGERGFFYT

SEQ ID NO: 286 - protein, 13 aa, Homo sapiens
VEQCCTSICSLYQ

SEQ ID NO: 287 - protein, 10 aa, Homo sapiens
ALWGPDPAAA

SEQ ID NO: 288 - protein, 13 aa, Homo sapiens
FFYTPKTRREAED

SEQ ID NO: 289 - protein, 17 aa, Homo sapiens
FYTPKTRREAEDLQVGQ

SEQ ID NO: 290 - protein, 15 aa, Homo sapiens
KRGIVEQCCTSICSL

SEQ ID NO: 291 - protein, 9 aa, Homo sapiens
LALEGSLQK

SEQ ID NO: 292 - protein, 17 aa, Homo sapiens
LVEALYLVCGERGFFYT

SEQ ID NO: 293 - protein, 16 aa, Homo sapiens
MALWMRLLPLLALLAL

SEQ ID NO: 294 - protein, 9 aa, Homo sapiens
RLLPLLALL

SEQ ID NO: 295 - protein, 8 aa, Homo sapiens
WGPDPAAA

SEQ ID NO: 296 - protein, 17 aa, Homo sapiens
AGSLQPLALEGSLQKRG

SEQ ID NO: 297 - protein, 9 aa, Homo sapiens
ALYLVCGER

SEQ ID NO: 298 - protein, 16 aa, Homo sapiens
CCTSICSLYQLENYCN

SEQ ID NO: 299 - protein, 17 aa, Homo sapiens
EDLQVGQVELGGGPGAG

SEQ ID NO: 300 - protein, 17 aa, Homo sapiens
FVNQHLCGSHLVEALYL

SEQ ID NO: 301 - protein, 17 aa, Homo sapiens
GERGFFYTPKTRREAED

SEQ ID NO: 302 - protein, 17 aa, Homo sapiens
GGGPGAGSLQPLALEGS

SEQ ID NO: 303 - protein, 15 aa, Homo sapiens
GIVEQCCTSICSLYQ

SEQ ID NO: 304 - protein, 17 aa, Homo sapiens
GQVELGGGPGAGSLQPL

SEQ ID NO: 305 - protein, 17 aa, Homo sapiens
GSLQKRGIVEQCCTSIC

SEQ ID NO: 306 - protein, 17 aa, Homo sapiens
PLALEGSLQKRGIVEQC

SEQ ID NO: 307 - protein, 17 aa, Homo sapiens
TRREAEDLQVGQVELGG

SEQ ID NO: 308 - protein, 15 aa, Homo sapiens
YLVCGERGFFYTPKT

SEQ ID NO: 309 - protein, 31 aa, Homo sapiens
EAEDLQVGQVELGGGPGAGSLQPLALEGSLQ

SEQ ID NO: 310 - protein, 18 aa, Homo sapiens
GSLQPLALEGSLQKRGIV

SEQ ID NO: 311 - protein, 16 aa, Homo sapiens
PAAAFVNQHLCGSHLV

SEQ ID NO: 312 - protein, 11 aa, Homo sapiens
EALYLVCGERG

SEQ ID NO: 313 - protein, 30 aa, Homo sapiens
VCGERGFFYTPKTRREAEDLQVGQVELGGG

SEQ ID NO: 314 - protein, 9 aa, Homo sapiens
FYTPKTRRE

SEQ ID NO: 315 - protein, 8 aa, Homo sapiens
GERGFFYT

SEQ ID NO: 316 - protein, 9 aa, Homo sapiens
ERGFFYTPK

SEQ ID NO: 317 - protein, 10 aa, Homo sapiens
LVCGERGFFY

SEQ ID NO: 318 - protein, 10 aa, Homo sapiens
LYLVCGERGF

SEQ ID NO: 319 - protein, 10 aa, Homo sapiens
AEDLQVGQVE

SEQ ID NO: 320 - protein, 9 aa, Homo sapiens
AGSLQPLAL

SEQ ID NO: 321 - protein, 10 aa, Homo sapiens
AGSLQPLALE

SEQ ID NO: 322 - protein, 10 aa, Homo sapiens
GAGSLQPLAL

SEQ ID NO: 323 - protein, 9 aa, Homo sapiens
QPLALEGSL

SEQ ID NO: 324 - protein, 10 aa, Homo sapiens
QPLALEGSLQ

SEQ ID NO: 325 - protein, 9 aa, Homo sapiens
QVELGGGPG

SEQ ID NO: 326 - protein, 10 aa, Homo sapiens
SLQPLALEGS

SEQ ID NO: 327 - protein, 9 aa, Homo sapiens
VELGGGPGA

SEQ ID NO: 328 - protein, 17 aa, Homo sapiens
GAGSLQPLALEGSLQKR

SEQ ID NO: 329 - protein, 16 aa, Homo sapiens
FVNQHLCGSHLVEALY

SEQ ID NO: 330 - protein, 14 aa, Homo sapiens
EAEDLQVGQVELGG

SEQ ID NO: 331 - protein, 7 aa, Homo sapiens
LALEGSL

SEQ ID NO: 332 - protein, 12 aa, Homo sapiens
PGAGSLQPLALE

SEQ ID NO: 333 - protein, 11 aa, Homo sapiens
QVELGGGPGAG

SEQ ID NO: 334 - protein, 11 aa, Homo sapiens
SLQPLALEGSL

SEQ ID NO: 335 - protein, 12 aa, Homo sapiens
SLQPLALEGSLQ

SEQ ID NO: 336 - protein, 8 aa, Homo sapiens
VELGGGPG

SEQ ID NO: 337 - protein, 15 aa, Homo sapiens
PGAGSLQPLALEGSL

SEQ ID NO: 338 - protein, 16 aa, Homo sapiens
GIVEQCCTSICSLYQL

SEQ ID NO: 339 - protein, 16 aa, Homo sapiens
LLALWGPDPAAAFVNQ

SEQ ID NO: 340 - protein, 16 aa, Homo sapiens
MRLLPLLALLALWGPD

SEQ ID NO: 341 - protein, 16 aa, Homo sapiens
PLLALLALWGPDPAAA

SEQ ID NO: 342 - protein, 16 aa, Homo sapiens
WGPDPAAAFVNQHLCG

SEQ ID NO: 343 - protein, 17 aa, Homo sapiens
YLVCGERGFFYTPKTRR

SEQ ID NO: 344 - protein, 17 aa, Homo sapiens
ALYLVCGERGFFYTPKT

SEQ ID NO: 345 - protein, 17 aa, Homo sapiens
CGSHLVEALYLVCGERG

SEQ ID NO: 346 - protein, 17 aa, Homo sapiens
ERGFFYTPKTRREAEDL

SEQ ID NO: 347 - protein, 17 aa, Homo sapiens
LALEGSLQKRGIVEQCC

SEQ ID NO: 348 - protein, 17 aa, Homo sapiens
PKTRREAEDLQVGQVEL

SEQ ID NO: 349 - protein, 17 aa, Homo sapiens
QKRGIVEQCCTSICSLY

SEQ ID NO: 350 - protein, 17 aa, Homo sapiens
VELGGGPGAGSLQPLAL

SEQ ID NO: 351 - protein, 13 aa, Homo sapiens
GQVELGGGPGAGS

SEQ ID NO: 352 - protein, 9 aa, Homo sapiens
FLIVLSVAL

SEQ ID NO: 353 - protein, 9 aa, Homo sapiens
KLQVFLIVL

SEQ ID NO: 354 - protein, 20 aa, Homo sapiens
  MOD_RES (9)..(9): Citrullinated
  MOD_RES (17)..(17): Citrullinated
VGSNTYGKRNAVEVLKREPL

SEQ ID NO: 355 - protein, 9 aa, Homo sapiens
VALKLQVFL

SEQ ID NO: 356 - protein, 14 aa, Homo sapiens
AFIEFKADEKKEDE

SEQ ID NO: 357 - protein, 12 aa, Homo sapiens
AFIKATGKKEDE

SEQ ID NO: 358 - protein, 15 aa, Homo sapiens
QEPSQLISLEEENQR

SEQ ID NO: 359 - protein, 9 aa, Homo sapiens
FLWSVFMLI

SEQ ID NO: 360 - protein, 9 aa, Homo sapiens
FLFAVGFYL

SEQ ID NO: 361 - protein, 9 aa, Homo sapiens
RLLCALTSL

SEQ ID NO: 362 - protein, 9 aa, Homo sapiens
LNIDLLWSV

SEQ ID NO: 363 - protein, 9 aa, Homo sapiens
VLFGLGFAI

SEQ ID NO: 364 - protein, 9 aa, Homo sapiens
FLWSVFWLI

SEQ ID NO: 365 - protein, 9 aa, Homo sapiens
NLFLFLFAV

SEQ ID NO: 366 - protein, 9 aa, Homo sapiens
YLLLRVLNI

SEQ ID NO: 367 - protein, 13 aa, Homo sapiens
DWIHIDTTPFAGL

SEQ ID NO: 368 - protein, 13 aa, Homo sapiens
QHLQKDYRAYYTF

SEQ ID NO: 369 - protein, 13 aa, Homo sapiens
RVLNIDLLWSVPI

SEQ ID NO: 370 - protein, 13 aa, Homo sapiens
YTFLNFMSNVGDP

SEQ ID NO: 371 - protein, 20 aa, Homo sapiens
KDYRAYYTFLNFMSNVGDPR

SEQ ID NO: 372 - protein, 20 aa, Homo sapiens
KWCANPDWIHIDTTPFAGLV

SEQ ID NO: 373 - protein, 9 aa, Homo sapiens
  MOD_RES (4)..(4): Citrullinated
GLVRNLGVL

SEQ ID NO: 374 - protein, 20 aa, Homo sapiens
HQVILGVIGGMLVAEAFEHT

SEQ ID NO: 375 - protein, 20 aa, Homo sapiens
QLYHFLQIPTHEEHLFYVLS

SEQ ID NO: 376 - protein, 9 aa, Homo sapiens
VMAPRTVLL

SEQ ID NO: 377 - protein, 20 aa, Homo sapiens
RLQQLVLDPGFLGLEPLLDL

SEQ ID NO: 378 - protein, 9 aa, Homo sapiens
LLLLLPPRV

SEQ ID NO: 379 - protein, 15 aa, Homo sapiens
GLSGLELDGMAELMA

SEQ ID NO: 380 - protein, 10 aa, Homo sapiens
HLCGSHLVEA

SEQ ID NO: 381 - protein, 10 aa, Homo sapiens
SHLVEALYLV

SEQ ID NO: 382 - protein, 10 aa, Homo sapiens
WMRLLPLLAL

SEQ ID NO: 383 - protein, 10 aa, Homo sapiens
LCGSHLVEAL

SEQ ID NO: 384 - protein, 20 aa, Homo sapiens
GGGPGAGSLQPLALEGSLQK

SEQ ID NO: 385 - protein, 20 aa, Homo sapiens
GAGSLQPLALEGSLQKRGIV

SEQ ID NO: 386 - protein, 10 aa, Homo sapiens
PLALEGSLQK

SEQ ID NO: 387 - protein, 10 aa, Homo sapiens
PLLALLALWG

SEQ ID NO: 388 - protein, 16 aa, Homo sapiens
TRREAEDLQVGQVELG

SEQ ID NO: 389 - protein, 16 aa, Homo sapiens
  MOD_RES (9)..(9): Deaminated
  MOD_RES (12)..(12): Deaminated
TRREAEDLQVGQVELG

SEQ ID NO: 390 - protein, 16 aa, Homo sapiens
  MOD_RES (9)..(9): Deaminated
  MOD_RES (12)..(12): Deaminated
TRREAEDLQVGQVELG

SEQ ID NO: 391 - protein, 9 aa, Homo sapiens
AMSNLVPPV

SEQ ID NO: 392 - protein, 9 aa, Homo sapiens
ALTAVAEEV

SEQ ID NO: 393 - protein, 10 aa, Homo sapiens
SLYHVYEVNL

SEQ ID NO: 394 - protein, 9 aa, Homo sapiens
TIADFWQMV

SEQ ID NO: 395 - protein, 9 aa, Homo sapiens
VIVMLTPLV

SEQ ID NO: 396 - protein, 13 aa, Homo sapiens
CAYQAEPNTCATA

SEQ ID NO: 397 - protein, 13 aa, Homo sapiens
CTVIVMLTPLVED

SEQ ID NO: 398 - protein, 13 aa, Homo sapiens
DQFEFALTAVAEE

SEQ ID NO: 399 - protein, 19 aa, Homo sapiens
FYLKNVQTQETRTLTQFHF

SEQ ID NO: 400 - protein, 13 aa, Homo sapiens
GSFINISVVGPAL

SEQ ID NO: 401 - protein, 20 aa, Homo sapiens
IKLKVESSPSRSDYINASPI

SEQ ID NO: 402 - protein, 13 aa, Homo sapiens
LEILAEHVHMSSG

SEQ ID NO: 403 - protein, 20 aa, Homo sapiens
LYHVYEVNLVSEHIWCEDFL

SEQ ID NO: 404 - protein, 21 aa, Homo sapiens
MVWESGCTVIVMLTPLVEDGV

SEQ ID NO: 405 - protein, 13 aa, Homo sapiens
PAYIATQGPLSHT

SEQ ID NO: 406 - protein, 13 aa, Homo sapiens
PSLSYEPALLQPY

SEQ ID NO: 407 - protein, 13 aa, Homo sapiens
RSVLLTLVALAGV

SEQ ID NO: 408 - protein, 20 aa, Homo sapiens
SEHIWCEDFLVRSFYLKNVQ

SEQ ID NO: 409 - protein, 21 aa, Homo sapiens
SKDQFEFALTAVAEEVNAILK

SEQ ID NO: 410 - protein, 13 aa, Homo sapiens
SLYHVYEVNLVSE

SEQ ID NO: 411 - protein, 13 aa, Homo sapiens
TYILIDMVLNRMA

SEQ ID NO: 412 - protein, 24 aa, Homo sapiens
DRGEKPASPAVQPDAALQRLAAVL

SEQ ID NO: 413 - protein, 21 aa, Homo sapiens
LPGPSPAQLFQDSGLLYLAQE

SEQ ID NO: 414 - protein, 40 aa, Homo sapiens
SPLGQSQPTVAGQPSARPAAEEYGYIVTDQKPLSLAAGVK

SEQ ID NO: 415 - protein, 25 aa, Homo sapiens
LAKEWQALCAYQAEPNTCATAQGEG

SEQ ID NO: 416 - protein, 21 aa, Homo sapiens
VSSVSSQFSDAAQASPSSHSS

SEQ ID NO: 417 - protein, 16 aa, Homo sapiens
DQFEFALTAVAEEVNA

SEQ ID NO: 418 - protein, 15 aa, Homo sapiens
FQDSGLLYLAQELPA

SEQ ID NO: 419 - protein, 15 aa, Homo sapiens
GASSSLSPLQAELLP

SEQ ID NO: 420 - protein, 17 aa, Homo sapiens
RSDYINASPIIEHDPRM

SEQ ID NO: 421 - protein, 9 aa, Homo sapiens
LLPPLLEHL

SEQ ID NO: 422 - protein, 9 aa, Homo sapiens
SLAAGVKLL

SEQ ID NO: 423 - protein, 9 aa, Homo sapiens
SLSPLQAEL

SEQ ID NO: 424 - protein, 23 aa, Homo sapiens
LAKEWQALCAYQAEPNTCATAQG

SEQ ID NO: 425 - protein, 18 aa, Homo sapiens
VSSQFSDAAQASPSSHSS

SEQ ID NO: 426 - protein, 24 aa, Homo sapiens
KLKVESSPSRSDYINASPIIEHDP

SEQ ID NO: 427 - protein, 28 aa, Homo sapiens
LAKEWQALCAYQAEPNTCATAQGEGNIK

SEQ ID NO: 428 - protein, 20 aa, Homo sapiens
SFYLKNVQTQETRTLTQFHF

SEQ ID NO: 429 - protein, 22 aa, Homo sapiens
SKDQFEFALTAVAEEVNAILKA

SEQ ID NO: 430 - protein, 29 aa, Homo sapiens
SRVSSVSSQFSDAAQASPSSHSSTPSWCE

SEQ ID NO: 431 - protein, 9 aa, Homo sapiens
MVWESGCTV

SEQ ID NO: 432 - protein, 25 aa, Homo sapiens
DFWQMVWESGCTVIVMLTPLVEDGV

SEQ ID NO: 433 - protein, 25 aa, Homo sapiens
ECDFQEFMAFVAMVTTACHEFFEHE

SEQ ID NO: 434 - protein, 20 aa, Homo sapiens
KAMVALIDVFHQYSGREGDK

SEQ ID NO: 435 - protein, 22 aa, Homo sapiens
KHKLKKSELKELINNELSHFLE

SEQ ID NO: 436 - protein, 16 aa, Homo sapiens
REGDKHKLKKSELKEL

SEQ ID NO: 437 - protein, 9 aa, Homo sapiens
ALIDVFHQY

SEQ ID NO: 438 - protein, 9 aa, Homo sapiens
GREGDKHKL

SEQ ID NO: 439 - protein, 10 aa, Homo sapiens
YLQGQRLDNV

SEQ ID NO: 440 - protein, 10 aa, Homo sapiens
AKSKNHTTHN

SEQ ID NO: 441 - protein, 27 aa, Homo sapiens
ACERLLYPDYQIQATVMIIVSSCAVAA

SEQ ID NO: 442 - protein, 27 aa, Homo sapiens
AKMHAFTLESVELQQKPVNKDQCPRER

SEQ ID NO: 443 - protein, 27 aa, Homo sapiens
ANEYAYAKWKLCSASAICFIFMIAEVV

SEQ ID NO: 444 - protein, 34 aa, Homo sapiens
ASRDSQVVRREIAKALSKSFTMHSLTIQMESPVD

SEQ ID NO: 445 - protein, 27 aa, Homo sapiens
DGVLSVHSLHIWSLTMNQVILSAHVAT

SEQ ID NO: 446 - protein, 27 aa, Homo sapiens
EELESGGMYHCHSGSKPTEKGANEYAY

SEQ ID NO: 447 - protein, 41 aa, Homo sapiens
FGWHRAEILGALLSILCIWVVTGVLVYLACERLLYPDYQIQ

SEQ ID NO: 448 - protein, 27 aa, Homo sapiens
FIFSILVLASTITILKDFSILLMEGVP

SEQ ID NO: 449 - protein, 27 aa, Homo sapiens
GHIAGSLAVVTDAAHLLIDLTSFLLSL

SEQ ID NO: 450 - protein, 27 aa, Homo sapiens
HLLIDLTSFLLSLFSLWLSSKPPSKRL

SEQ ID NO: 451 - protein, 27 aa, Homo sapiens
HQRCLGHNHKEVQANASVRAAFVHALG

SEQ ID NO: 452 - protein, 27 aa, Homo sapiens
LFQSISVLISALIIYFKPEYKIADPIC

SEQ ID NO: 453 - protein, 27 aa, Homo sapiens
LKDFSILLMEGVPKSLNYSGVKELILA

SEQ ID NO: 454 - protein, 27 aa, Homo sapiens
MEFLERTYLVNDKAAKMHAFTLESVEL

SEQ ID NO: 455 - protein, 26 aa, Homo sapiens
MHSLTIQMESPVDQDPDCLFCEDPCD

SEQ ID NO: 456 - protein, 27 aa, Homo sapiens
NASVRAAFVHALGDLFQSISVLISALI

SEQ ID NO: 457 - protein, 27 aa, Homo sapiens
QKPVNKDQCPRERPEELESGGMYHCHS

SEQ ID NO: 458 - protein, 27 aa, Homo sapiens
SAICFIFMIAEVVGGHIAGSLAVVTDA

SEQ ID NO: 459 - protein, 27 aa, Homo sapiens
SCAVAANIVLTVVLHQRCLGHNHKEVQ

SEQ ID NO: 460 - protein, 27 aa, Homo sapiens
SLNYSGVKELILAVDGVLSVHSLHIWS

SEQ ID NO: 461 - protein, 27 aa, Homo sapiens
SLWLSSKPPSKRLTFGWHRAEILGALL

SEQ ID NO: 462 - protein, 27 aa, Homo sapiens
TMNQVILSAHVATAASRDSQVVRREIA

SEQ ID NO: 463 - protein, 27 aa, Homo sapiens
YFKPEYKIADPICTFIFSILVLASTIT

SEQ ID NO: 464 - protein, 15 aa, Homo sapiens
ERTYLVNDKAAKMHA

SEQ ID NO: 465 - protein, 15 aa, Homo sapiens
IFSILVLASTITILK

SEQ ID NO: 466 - protein, 14 aa, Homo sapiens
YAYAKWKLCSASAI

SEQ ID NO: 467 - protein, 15 aa, Homo sapiens
YKIADPICTFIFSIL

SEQ ID NO: 468 - protein, 13 aa, Homo sapiens
PIILPLWHVGMND

SEQ ID NO: 469 - protein, 13 aa, Homo sapiens
PIILPLWHVGEPG

SEQ ID NO: 470 - protein, 9 aa, Homo sapiens
  MOD_RES (4)..(4): Citrullinated
VLNRMAKGV

SEQ ID NO: 471 - protein, 16 aa, Homo sapiens
GDRGEKPASPAVQPDA

SEQ ID NO: 472 - protein, 19 aa, Homo sapiens
VPRLPEQGSSSRAEDSPEG

SEQ ID NO: 473 - protein, 18 aa, Homo sapiens
  MOD_RES (4)..(4): Deaminated
  MOD_RES (7)..(7): Deaminated
  MOD_RES (12)..(12): Deaminated
TGLQILQTGVGQREEAAA

SEQ ID NO: 474 - protein, 13 aa, Homo sapiens
DKERLAALGPEGA

SEQ ID NO: 475 - protein, 13 aa, Homo sapiens
FYLKNVQTQETRT

SEQ ID NO: 476 - protein, 13 aa, Homo sapiens
MVWESGCTVIVML

SEQ ID NO: 477 - protein, 13 aa, Homo sapiens
TVIVMLTPLVEDG

SEQ ID NO: 478 - protein, 13 aa, Homo sapiens
VKEIDIAATLEHV

SEQ ID NO: 479 - protein, 9 aa, Homo sapiens
FLSGAVNRL

SEQ ID NO: 480 - protein, 10 aa, Homo sapiens
VLSRNILLEL

SEQ ID NO: 481 - protein, 9 aa, Homo sapiens
MLMPVHFLL

SEQ ID NO: 482 - protein, 9 aa, Homo sapiens
LLTTLSNRV

SEQ ID NO: 483 - protein, 9 aa, Homo sapiens
NLIKLAQKV

SEQ ID NO: 484 - protein, 10 aa, Homo sapiens
VMIIVSSLAV

SEQ ID NO: 485 - protein, 9 aa, Homo sapiens
ALGDLFQSI

SEQ ID NO: 486 - protein, 10 aa, Homo sapiens
AVAANIVLTV

SEQ ID NO: 487 - protein, 9 aa, Homo sapiens
DLTSFLLSL

SEQ ID NO: 488 - protein, 9 aa, Homo sapiens
EILGALLSI

SEQ ID NO: 489 - protein, 10 aa, Homo sapiens
FLLSLFSLWL

SEQ ID NO: 490 - protein, 9 aa, Homo sapiens
HIAGSLAVV

SEQ ID NO: 491 - protein, 10 aa, Homo sapiens
ILAVDGVLSV

SEQ ID NO: 492 - protein, 9 aa, Homo sapiens
ILGALLSIL

SEQ ID NO: 493 - protein, 9 aa, Homo sapiens
ILKDFSILL

SEQ ID NO: 494 - protein, 9 aa, Homo sapiens
ILSAHVATA

SEQ ID NO: 495 - protein, 10 aa, Homo sapiens
ILVLASTITI

SEQ ID NO: 496 - protein, 9 aa, Homo sapiens
LLIDLTSFL

SEQ ID NO: 497 - protein, 10 aa, Homo sapiens
LLMEGVPKSL

SEQ ID NO: 498 - protein, 9 aa, Homo sapiens
SISVLISAL

SEQ ID NO: 499 - protein, 10 aa, Homo sapiens
SLNYSGVKEL

SEQ ID NO: 500 - protein, 10 aa, Homo sapiens
SVHSLHIWSL

SEQ ID NO: 501 - protein, 9 aa, Homo sapiens
VVTGVLVYL

SEQ ID NO: 502 - protein, 9 aa, Homo sapiens
FIFSILVLA

SEQ ID NO: 503 - protein, 9 aa, Homo sapiens
IQATVMIIV

SEQ ID NO: 504 - protein, 9 aa, Homo sapiens
KMYAFTLES

SEQ ID NO: 505 - protein, 9 aa, Homo sapiens
KSLNYSGVK

SEQ ID NO: 506 - protein, 9 aa, Homo sapiens
LAVDGVLSV

SEQ ID NO: 507 - protein, 9 aa, Homo sapiens
LLSLFSLWL

SEQ ID NO: 508 - protein, 9 aa, Homo sapiens
RLLYPDYQI

SEQ ID NO: 509 - protein, 9 aa, Homo sapiens
TMHSLTIQM

SEQ ID NO: 510 - protein, 9 aa, Homo sapiens
VAANIVLTV

SEQ ID NO: 511 - protein, 9 aa, Homo sapiens
  MOD_RES (4)..(4): Phosphorylated
FIFSILVLA

SEQ ID NO: 512 - protein, 9 aa, Homo sapiens
CLGHNHKEV

SEQ ID NO: 513 - protein, 10 aa, Homo sapiens
KIADPICTFI

SEQ ID NO: 514 - protein, 10 aa, Homo sapiens
KMYAFTLESV

SEQ ID NO: 515 - protein, 10 aa, Homo sapiens
LLIDLTSFLL

SEQ ID NO: 516 - protein, 20 aa, Homo sapiens
ILKDFSILLMEGVPKSLNYS

SEQ ID NO: 517 - protein, 20 aa, Homo sapiens
VRREIAKALSKSFTMHSLTI

SEQ ID NO: 518 - protein, 15 aa, Homo sapiens
AKMYAFTLESVELQQ

SEQ ID NO: 519 - protein, 20 aa, Homo sapiens
SHFSLKKGAAALGIGTDSVI

SEQ ID NO: 520 - protein, 20 aa, Homo sapiens
AAALGIGTDSVILIKCDERG

SEQ ID NO: 521 - protein, 20 aa, Homo sapiens
VSYQPLGDKVNFFRMVISNP

SEQ ID NO: 522 - protein, 20 aa, Homo sapiens
MEFLERTYLVNDKAAKMYAF

SEQ ID NO: 523 - protein, 20 aa, Homo sapiens
LVNDKAAKMYAFTLESVELQ

SEQ ID NO: 524 - protein, 20 aa, Homo sapiens
MYAFTLESVELQQKPVNKDQ

SEQ ID NO: 525 - protein, 20 aa, Homo sapiens
GHNHKEVQANASVRAAFVHA

SEQ ID NO: 526 - protein, 110 aa, Homo sapiens
MALWMRLLPLLALLALWGPDPAAAFVNQHLCGSHLVEALYLVCGERGFFYTPKTRREAEDLQVGQVELGGGPGAGSLQPLALEGSLQKRGIVEQCCTSICSLYQLENYCN

SEQ ID NO: 527 - protein, 9 aa, Homo sapiens
LVCGERGFF

SEQ ID NO: 528 - protein, 11 aa, Homo sapiens
TPKTRREAEDL

SEQ ID NO: 529 - protein, 9 aa, Homo sapiens
ALEGSLQKR

SEQ ID NO: 530 - protein, 9 aa, Homo sapiens
IVEQCCTSI

SEQ ID NO: 531 - protein, 4 aa, Artificial Sequence
  Description of Artificial Sequence Synthetic peptide
LRMK

SEQ ID NO: 532 - protein, 5 aa, Artificial Sequence
  Description of Artificial Sequence Synthetic peptide
GPGPG

SEQ ID NO: 533 - protein, 661 aa, Homo sapiens
AAAAARPAGGSARRWGRPGRCGLLAAGPKRVRSEPGGRLPERSLGPAHPAPAAMAGTLDLDKGCTVEELLRGCIEAFDDSGKVRDPQLVRMFLMMHPWYIPSSQLAAKLLHIYQQSRKDNSNSLQVKTCHLVRYWISAFPAEFDLNPELAEQIKELKALLDQEGNRRHSSLIDIDSVPTYKWKRQVTQRNPVGQKKRKMSLLFDHLEPMELAEHLTYLEYRSFCKILFQDYHSFVTHGCTVDNPVLERFISLFNSVSQWVQLMILSKPTAPQRALVITHFVHVAEKLLQLQNFNTLMAVVGGLSHSSISRLKETHSHVSPETIKLWEGLTELVTATGNYGNYRRRLAACVGFRFPILGVHLKDLVALQLALPDWLDPARTRLNGAKMKQLFSILEELAMVTSLRPPVQANPDLLSLLTVSLDQYQTEDELYQLSLQREPRSKSSPTSPTSCTPPPRPPVLEEWTSAAKPKLDQALWEHIEKMVESVFRNFDVDGDGHISQEEFQIIRGNFPYLSAFGDLDQNQDGCISREEMVSYFLRSSSVLGGRMGFVHNFQESNSLRPVACRHCKALILGIYKQGLKCRACGVNCHKQCKDRLSVECRRRAQSVSLEGSAPSPSPMHSHHHRAFSFSLPRPGRRGSRPPEIREEEVQTVEDGVFDIHL

SEQ ID NO: 534 - protein, 609 aa, Homo sapiens
MAGTLDLDKGCTVEELLRGCIEAFDDSGKVRDPQLVRMFLMMHPWYIPSSQLAAKLLHIYQQSRKDNSNSLQVKTCHLVRYWISAFPAEFDLNPELAEQIKELKALLDQEGNRRHSSLIDIDSVPTYKWKRQVTQRNPVGQKKRKMSLLFDHLEPMELAEHLTYLEYRSFCKILFQDYHSFVTHGCTVDNPVLERFISLFNSVSQWVQLMILSKPTAPQRALVITHFVHVAEKLLQLQNFNTLMAVVGGLSHSSISRLKETHSHVSPETIKLWEGLTELVTATGNYGNYRRRLAACVGFRFPILGVHLKDLVALQLALPDWLDPARTRLNGAKMKQLFSILEELAMVTSLRPPVQANPDLLSLLTVSLDQYQTEDELYQLSLQREPRSKSSPTSPTSCTPPPRPPVLEEWTSAAKPKLDQALVVEHIEKMVESVFRNFDVDGDGHISQEEFQIIRGNFPYLSAFGDLDQNQDGCISREEMVSYFLRSSSVLGGRMGFVHNFQESNSLRPVACRHCKALILGIYKQGLKCRACGVNCHKQCKDRLSVECRRRAQSVSLEGSAPSPSPMHSHHHRAFSFSLPRPGRRGSRPPEIREEEVQTVEDGVFDIHL

SEQ ID NO: 535 - protein, 670 aa, Homo sapiens
MGTQRLCGRGTQGWPGSSEQHVQEATSSAGLHSGVDELGVRSEPGGRLPERSLGPAHPAPAAMAGTLDLDKGCTVEELLRGCIEAFDDSGKVRDPQLVRMFLMMHPWYIPSSQLAAKLLHIYQQSRKDNSNSLQVKTCHLVRYWISAFPAEFDLNPELAEQIKELKALLDQEGNRRHSSLIDIDSVPTYKWKRQVTQRNPVGQKKRKMSLLFDHLEPMELAEHLTYLEYRSFCKILFQDYHSFVTHGCTVDNPVLERFISLFNSVSQWVQLMILSKPTAPQRALVITHFVHVAEKLLQLQNFNTLMAVVGGLSHSSISRLKETHSHVSPETIKLWEGLTELVTATGNYGNYRRRLAACVGFRFPILGVHLKDLVALQLALPDWLDPARTRLNGAKMKQLFSILEELAMVTSLRPPVQANPDLLSLLTVSLDQYQTEDELYQLSLQREPRSKSSPTSPTSCTPPPRPPVLEEWTSAAKPKLDQALWEHIEKMVESVFRNFDVDGDGHISQEEFQIIRGNFPYLSAFGDLDQNQDGCISREEMVSYFLRSSSVLGGRMGFVHNFQESNSLRPVACRHCKALILGIYKQGLKCRACGVNCHKQCKDRLSVECRRRAQSVSLEGSAPSPSPMHSHHHRAFSFSLPRPGRRGSRPPEIREEEVQTVEDGVFDIHL

SEQ ID NO: 536 - protein, 579 aa, Homo sapiens
MAGTLDLDKGCTVEELLRGCIEAFDDSGKVRDPQLVRMFLMMHPWYIPSSQLAAKLLHIYQQSRKDNSNSLQVKTCHLVRYWISAFPAEFDLNPELAEQIKELKALLDQEGNRRHSSLIDIDSVPTYKWKRQVTQRNPVGQKKRKMSLLFDHLEPMELAEHLTYLEYRSFCKILFQDYHSFVTHGCTVDNPVLERFISLFNSVSQWVQLMILSKPTAPQRALVITHFVHVAEKLLQLQNFNTLMAVVGGLSHSSISRLKETHSHVSPETIKLWEGLTELVTATGNYGNYRRRLAACVGFRFPILGVHLKDLVALQLALPDWLDPARTRLNGAKMKQLFSILEELAMVTSLRPPVQANPDLLSLLTVSLDQYQTEDELYQLSLQREPRSKSSPTSPTSCTPPPRPPVLEEWTSAAKPKLDQALVVEHIEKMVESVFRNFDVDGDGHISQEEFQIIRGNFPYLSAFGDLDQNQDGCISREEMVSYFLRSSSVLGGRMGFVHNFQESNSLRPVACRHCKALILGIYKQGLKCRACGVNCHKQCKDRLSVECRRRAQSVSLEGSAPSPSPMHSHHHRAFSFSL

SEQ ID NO: 537 - protein, 10 aa, Homo sapiens
LVRYWISAFP

SEQ ID NO: 538 - protein, 23 aa, Homo sapiens
LLFDHLEPMELAEHLTYLEYRSF

SEQ ID NO: 539 - protein, 28 aa, Homo sapiens
NFNTLMAVVGGLSHSSISRLKETHSHVS

SEQ ID NO: 540 - protein, 15 aa, Homo sapiens
PAAMAGTLDLDKGCT

SEQ ID NO: 541 - protein, 15 aa, Homo sapiens
DKGCTVEELLRGCIE

SEQ ID NO: 542 - protein, 15 aa, Homo sapiens
RGCIEAFDDSGKVRD

SEQ ID NO: 543 - protein, 15 aa, Homo sapiens
GKVRDPQLVRMFLMM

SEQ ID NO: 544 - protein, 15 aa, Homo sapiens
MFLMMHPWYIPSSQL

SEQ ID NO: 545 - protein, 15 aa, Homo sapiens
PSSQLAAKLLHIYQQ

SEQ ID NO: 546 - protein, 15 aa, Homo sapiens
HIYQQSRKDNSNSLQ

SEQ ID NO: 547 - protein, 15 aa, Homo sapiens
SNSLQVKTCHLVRYW

SEQ ID NO: 548 - protein, 15 aa, Homo sapiens
LVRYWISAFPAEFDL

SEQ ID NO: 549 - protein, 15 aa, Homo sapiens
AEFDLNPELAEQIKE

SEQ ID NO: 550 - protein, 15 aa, Homo sapiens
EQIKELKALLDQEGN

SEQ ID NO: 551 - protein, 15 aa, Homo sapiens
DQEGNRRHSSLIDID

SEQ ID NO: 552 - protein, 15 aa, Homo sapiens
LIDIDSVPTYKWKRQ

SEQ ID NO: 553 - protein, 15 aa, Homo sapiens
KWKRQVTQRNPVGQK

SEQ ID NO: 554 - protein, 15 aa, Homo sapiens
PVGQKKRKMSLLFDH

SEQ ID NO: 555 - protein, 15 aa, Homo sapiens
LLFDHLEPMELAEHL

SEQ ID NO: 556 - protein, 15 aa, Homo sapiens
LAEHLTYLEYRSFCK

SEQ ID NO: 557 - protein, 15 aa, Homo sapiens
RSFCKILFQDYHSFV

SEQ ID NO: 558 - protein, 15 aa, Homo sapiens
YHSFVTHGCTVDNPV

SEQ ID NO: 559 - protein, 15 aa, Homo sapiens
VDNPVLERFISLFNS

SEQ ID NO: 560 - protein, 15 aa, Homo sapiens
SLFNSVSQWVQLMIL

SEQ ID NO: 561 - protein, 15 aa, Homo sapiens
QLMILSKPTAPQRAL

SEQ ID NO: 562 - protein, 15 aa, Homo sapiens
PQRALVITHFVHVAE

SEQ ID NO: 563 - protein, 15 aa, Homo sapiens
VHVAEKLLQLQNFNT

SEQ ID NO: 564 - protein, 15 aa, Homo sapiens
QNFNTLMAVVGGLSH

SEQ ID NO: 565 - protein, 15 aa, Homo sapiens
GGLSHSSISRLKETH

SEQ ID NO: 566 - protein, 15 aa, Homo sapiens
LKETHSHVSPETIKL

SEQ ID NO: 567 - protein, 15 aa, Homo sapiens
ETIKLWEGLTELVTA

SEQ ID NO: 568 - protein, 15 aa, Homo sapiens
ELVTATGNYGNYRRR

SEQ ID NO: 569 - protein, 15 aa, Homo sapiens
NYRRRLAACVGFRFP

SEQ ID NO: 570 - protein, 15 aa, Homo sapiens
GFRFPILGVHLKDLV

SEQ ID NO: 571 - protein, 15 aa, Homo sapiens
LKDLVALQLALPDWL

SEQ ID NO: 572 - protein, 15 aa, Homo sapiens
LPDWLDPARTRLNGA

SEQ ID NO: 573 - protein, 15 aa, Homo sapiens
RLNGAKMKQLFSILE

SEQ ID NO: 574 - protein, 15 aa, Homo sapiens
FSILEELAMVTSLRP

SEQ ID NO: 575 - protein, 15 aa, Homo sapiens
TSLRPPVQANPDLLS

SEQ ID NO: 576 - protein, 15 aa, Homo sapiens
PDLLSLLTVSLDQYQ

SEQ ID NO: 577 - protein, 15 aa, Homo sapiens
LDQYQTEDELYQLSL

SEQ ID NO: 578 - protein, 15 aa, Homo sapiens
YQLSLQREPRSKSSP

SEQ ID NO: 579 - protein, 15 aa, Homo sapiens
SKSSPTSPTSCTPPP

SEQ ID NO: 580 - protein, 15 aa, Homo sapiens
CTPPPRPPVLEEWTS

SEQ ID NO: 581 - protein, 15 aa, Homo sapiens
EEWTSAAKPKLDQAL

SEQ ID NO: 582 - protein, 14 aa, Homo sapiens
LDQAVVEHIEKMVE

SEQ ID NO: 583 - protein, 15 aa, Homo sapiens
EKMVESVFRNFDVDG

SEQ ID NO: 584 - protein, 15 aa, Homo sapiens
FDVDGDGHISQEEFQ

SEQ ID NO: 585 - protein, 15 aa, Homo sapiens
QEEFQIIRGNFPYLS

SEQ ID NO: 586 - protein, 15 aa, Homo sapiens
FPYLSAFGDLDQNQD

SEQ ID NO: 587 - protein, 15 aa, Homo sapiens
DQNQDGCISREEMVS

SEQ ID NO: 588 - protein, 15 aa, Homo sapiens
EEMVSYFLRSSSVLG

SEQ ID NO: 589 - protein, 15 aa, Homo sapiens
SSVLGGRMGFVHNFQ

SEQ ID NO: 590 - protein, 15 aa, Homo sapiens
VHNFQESNSLRPVAC

SEQ ID NO: 591 - protein, 15 aa, Homo sapiens
RPVACRHCKALILGI

SEQ ID NO: 592 - protein, 15 aa, Homo sapiens
LILGIYKQGLKCRAC

SEQ ID NO: 593 - protein, 15 aa, Homo sapiens
KCRACGVNCHKQCKD

SEQ ID NO: 594 - protein, 15 aa, Homo sapiens
KQCKDRLSVECRRRA

SEQ ID NO: 595 - protein, 15 aa, Homo sapiens
CRRRAQSVSLEGSAP

SEQ ID NO: 596 - protein, 15 aa, Homo sapiens
EGSAPSPSPMHSHHH

SEQ ID NO: 597 - protein, 15 aa, Homo sapiens
HSHHHRAFSFSLPRP

SEQ ID NO: 598 - protein, 15 aa, Homo sapiens
SLPRPGRRGSRPPEI

SEQ ID NO: 599 - protein, 15 aa, Homo sapiens
RPPEIREEEVQTVED

SEQ ID NO: 600 - protein, 15 aa, Homo sapiens
EEVQTVEDGVFDIHL

SEQ ID NO: 601 - protein, 11 aa, Homo sapiens
AFSFSLPRPGR

SEQ ID NO: 602 - protein, 8 aa, Homo sapiens
ALILGIYK

SEQ ID NO: 603 - protein, 10 aa, Homo sapiens
ALLDQEGNRR

SEQ ID NO: 604 - protein, 13 aa, Homo sapiens
ALVITHFVHVAEK

SEQ ID NO: 605 - protein, 17 aa, Homo sapiens
DLVALQLALPDWLDPAR

SEQ ID NO: 606 - protein, 9 aa, Homo sapiens
DNSNSLQVK

SEQ ID NO: 607 - protein, 14 aa, Homo sapiens
HSSLIDIDSVPTYK

SEQ ID NO: 608 - protein, 10 aa, Homo sapiens
KDNSNSLQVK

SEQ ID NO: 609 - protein, 12 aa, Homo sapiens
LDQALVVEHIEK

SEQ ID NO: 610 - protein, 9 aa, Homo sapiens
LLHIYQQSR

SEQ ID NO: 611 - protein, 24 aa, Homo sapiens
LLQLQNFNTLMAVVGGLSHSSISR

SEQ ID NO: 612 - protein, 18 aa, Homo sapiens
MFLMMHPWYIPSSQLAAK

SEQ ID NO: 613 - protein, 8 aa, Homo sapiens
VRDPQLVR

SEQ ID NO: 614 - protein, 21 aa, Homo sapiens
YWISAFPAEFDLNPELAEQIK

SEQ ID NO: 615 - protein, 15 aa, Homo sapiens
PAAMAGTLDLDKGCT

SEQ ID NO: 616 - protein, 15 aa, Homo sapiens
DKGCTVEELLRGCIE

SEQ ID NO: 617 - protein, 15 aa, Homo sapiens
RGCIEAFDDSGKVRD

SEQ ID NO: 618 - protein, 15 aa, Homo sapiens
GKVRDPQLVRMFLMM

SEQ ID NO: 619 - protein, 15 aa, Homo sapiens
PSSQLAAKLLHIYQQ

SEQ ID NO: 620 - protein, 15 aa, Homo sapiens
HIYQQSRKDNSNSLQ

SEQ ID NO: 621 - protein, 15 aa, Homo sapiens
SNSLQVKTCHLVRYW

SEQ ID NO: 622 - protein, 15 aa, Homo sapiens
LVRYWISAFPAEFDL

SEQ ID NO: 623 - protein, 15 aa, Homo sapiens
AEFDLNPELAEQIKE

SEQ ID NO: 624 - protein, 15 aa, Homo sapiens
EQIKELKALLDQEGN

SEQ ID NO: 625 - protein, 15 aa, Homo sapiens
DQEGNRRHSSLIDID

SEQ ID NO: 626 - protein, 15 aa, Homo sapiens
LIDIDSVPTYKWKRQ

SEQ ID NO: 627 - protein, 15 aa, Homo sapiens
KWKRQVTQRNPVGQK

SEQ ID NO: 628 - protein, 15 aa, Homo sapiens
PVGQKKRKMSLLFDH

SEQ ID NO: 629 - protein, 15 aa, Homo sapiens
LLFDHLEPMELAEHL

SEQ ID NO: 630 - protein, 15 aa, Homo sapiens
LAEHLTYLEYRSFCK

SEQ ID NO: 631 - protein, 15 aa, Homo sapiens
RSFCKILFQDYHSFV

SEQ ID NO: 632 - protein, 15 aa, Homo sapiens
YHSFVTHGCTVDNPV

SEQ ID NO: 633 - protein, 15 aa, Homo sapiens
VDNPVLERFISLFNS

SEQ ID NO: 634 - protein, 15 aa, Homo sapiens
SLFNSVSQWVQLMIL

SEQ ID NO: 635 - protein, 15 aa, Homo sapiens
QLMILSKPTAPQRAL

SEQ ID NO: 636 - protein, 15 aa, Homo sapiens
PQRALVITHFVHVAE

SEQ ID NO: 637 - protein, 15 aa, Homo sapiens
VHVAEKLLQLQNFNT

SEQ ID NO: 638 - protein, 15 aa, Homo sapiens
QNFNTLMAVVGGLSH

SEQ ID NO: 639 - protein, 15 aa, Homo sapiens
GGLSHSSISRLKETH

SEQ ID NO: 640 - protein, 15 aa, Homo sapiens
LKETHSHVSPETIKL

SEQ ID NO: 641 - protein, 15 aa, Homo sapiens
ELVTATGNYGNYRRR

SEQ ID NO: 642 - protein, 15 aa, Homo sapiens
NYRRRLAACVGFRFP

SEQ ID NO: 643 - protein, 15 aa, Homo sapiens
GFRFPILGVHLKDLV

SEQ ID NO: 644 - protein, 15 aa, Homo sapiens
LKDLVALQLALPDWL

SEQ ID NO: 645 - protein, 15 aa, Homo sapiens
LPDWLDPARTRLNGA

SEQ ID NO: 646 - protein, 15 aa, Homo sapiens
RLNGAKMKQLFSILE

SEQ ID NO: 647 - protein, 15 aa, Homo sapiens
FSILEELAMVTSLRP

SEQ ID NO: 648 - protein, 15 aa, Homo sapiens
TSLRPPVQANPDLLS

SEQ ID NO: 649 - protein, 15 aa, Homo sapiens
PDLLSLLTVSLDQYQ

SEQ ID NO: 650 - protein, 15 aa, Homo sapiens
LDQYQTEDELYQLSL

SEQ ID NO: 651 - protein, 15 aa, Homo sapiens
YQLSLQREPRSKSSP

SEQ ID NO: 652 - protein, 15 aa, Homo sapiens
SKSSPTSPTSCTPPP

SEQ ID NO: 653 - protein, 15 aa, Homo sapiens
CTPPPRPPVLEEWTS

SEQ ID NO: 654 - protein, 15 aa, Homo sapiens
EEWTSAAKPKLDQAL

SEQ ID NO: 655 - protein, 15 aa, Homo sapiens
LDQALVVEHIEKMVE

SEQ ID NO: 656 - protein, 15 aa, Homo sapiens
FDVDGDGHISQEEFQ

SEQ ID NO: 657 - protein, 15 aa, Homo sapiens
QEEFQIIRGNFPYLS

SEQ ID NO: 658 - protein, 15 aa, Homo sapiens
FPYLSAFGDLDQNQD

SEQ ID NO: 659 - protein, 15 aa, Homo sapiens
DQNQDGCISREEMVS

SEQ ID NO: 660 - protein, 15 aa, Homo sapiens
EEMVSYFLRSSSVLG

SEQ ID NO: 661 - protein, 15 aa, Homo sapiens
SSVLGGRMGFVHNFQ

SEQ ID NO: 662 - protein, 15 aa, Homo sapiens
VHNFQESNSLRPVAC

SEQ ID NO: 663 - protein, 15 aa, Homo sapiens
RPVACRHCKALILGI

SEQ ID NO: 664 - protein, 15 aa, Homo sapiens
LILGIYKQGLKCRAC

SEQ ID NO: 665 - protein, 15 aa, Homo sapiens
KCRACGVNCHKQCKD

SEQ ID NO: 666 - protein, 15 aa, Homo sapiens
KQCKDRLSVECRRRA

SEQ ID NO: 667 - protein, 15 aa, Homo sapiens
CRRRAQSVSLEGSAP

SEQ ID NO: 668 - protein, 15 aa, Homo sapiens
EGSAPSPSPMHSHHH

SEQ ID NO: 669 - protein, 15 aa, Homo sapiens
HSHHHRAFSFSLPRP

SEQ ID NO: 670 - protein, 15 aa, Homo sapiens
SLPRPGRRGSRPPEI

SEQ ID NO: 671 - protein, 15 aa, Homo sapiens
RPPEIREEEVQTVED

SEQ ID NO: 672 - protein, 15 aa, Homo sapiens
EEVQTVEDGVFDIHL

SEQ ID NO: 673 - protein, 15 aa, Homo sapiens
MGEPQGSMRILVTGG

SEQ ID NO: 674 - protein, 15 aa, Homo sapiens
VVADGAGLPGEDWVF

SEQ ID NO: 675 - protein, 15 aa, Homo sapiens
TAQTRALFEKVQPTH

SEQ ID NO: 676 - protein, 15 aa, Homo sapiens
LFRNIKYNLDFWRKN

SEQ ID NO: 677 - protein, 15 aa, Homo sapiens
VHMNDNVLHSAFEVG

SEQ ID NO: 678 - protein, 10 aa, Homo sapiens
DNVLHSAFEV

SEQ ID NO: 679 - protein, 10 aa, Homo sapiens
NVLHSAFEVG

SEQ ID NO: 680 - protein, 15 aa, Homo sapiens
NVLHSAFEVGARKVV

SEQ ID NO: 681 - protein, 10 aa, Homo sapiens
VLHSAFEVGA

SEQ ID NO: 682 - protein, 15 aa, Homo sapiens
KTTYPIDETMIHNGP

SEQ ID NO: 683 - protein, 15 aa, Homo sapiens
IHNGPPHNSNFGYSY

SEQ ID NO: 684 - protein, 15 aa, Homo sapiens
PHNSNFGYSYAKRMI

SEQ ID NO: 685 - protein, 15 aa, Homo sapiens
AYFQQYGCTFTAVIP

SEQ ID NO: 686 - protein, 15 aa, Homo sapiens
YGCTFTAVIPTNVFG

SEQ ID NO: 687 - protein, 15 aa, Homo sapiens
LFIWVLREYNEVEPI

SEQ ID NO: 688 - protein, 15 aa, Homo sapiens
LREYNEVEPIILSVG

SEQ ID NO: 689 - protein, 15 aa, Homo sapiens
EVEPIILSVGEEDEV

SEQ ID NO: 690 - protein, 15 aa, Homo sapiens
ILSVGEEDEVSIKEA

SEQ ID NO: 691 - protein, 15 aa, Homo sapiens
EEDEVSIKEAAEAVV

SEQ ID NO: 692 - protein, 15 aa, Homo sapiens
SIKEAAEAVVEAMDF

SEQ ID NO: 693 - protein, 15 aa, Homo sapiens
AEAVVEAMDFHGEVT

SEQ ID NO: 694 - protein, 15 aa, Homo sapiens
FDTTKSDGQFKKTAS

SEQ ID NO: 695 - protein, 15 aa, Homo sapiens
FRFTPFKQAVKETCA

SEQ ID NO: 696 - protein, 10 aa, Homo sapiens
KLLLHSGVEN

SEQ ID NO: 697 - protein, 15 aa, Homo sapiens
GSMRILVTGGSGLVG

SEQ ID NO: 698 - protein, 15 aa, Homo sapiens
LVTGGSGLVGKAIQK

SEQ ID NO: 699 - protein, 15 aa, Homo sapiens
SGLVGKAIQKVVADG

SEQ ID NO: 700 - protein, 15 aa, Homo sapiens
KAIQKVVADGAGLPG

SEQ ID NO: 701 - protein, 15 aa, Homo sapiens
VVADGAGLPGEDWVF

SEQ ID NO: 702 - protein, 15 aa, Homo sapiens
AGLPGEDWVFVSSKD

SEQ ID NO: 703 - protein, 15 aa, Homo sapiens
EDWVFVSSKDADLTD

SEQ ID NO: 704 - protein, 15 aa, Homo sapiens
VSSKDADLTDTAQTR

SEQ ID NO: 705 - protein, 15 aa, Homo sapiens
ADLTDTAQTRALFEK

SEQ ID NO: 706 - protein, 15 aa, Homo sapiens
ALFEKVQPTHVIHLA

SEQ ID NO: 707 - protein, 15 aa, Homo sapiens
VQPTHVIHLAAMVGG

SEQ ID NO: 708 - protein, 15 aa, Homo sapiens
VIHLAAMVGGLFRNI

SEQ ID NO: 709 - protein, 15 aa, Homo sapiens
AMVGGLFRNIKYNLD

SEQ ID NO: 710 - protein, 15 aa, Homo sapiens
KYNLDFWRKNVHMND

SEQ ID NO: 711 - protein, 15 aa, Homo sapiens
FWRKNVHMNDNVLHS

SEQ ID NO: 712 - protein, 15 aa, Homo sapiens
NVLHSAFEVGARKVV

SEQ ID NO: 713 - protein, 15 aa, Homo sapiens
AFEVGARKVVSCLST

SEQ ID NO: 714 - protein, 15 aa, Homo sapiens
ARKVVSCLSTCIFPD

SEQ ID NO: 715 - protein, 15 aa, Homo sapiens
SCLSTCIFPDKTTYP

SEQ ID NO: 716 - protein, 15 aa, Homo sapiens
CIFPDKTTYPIDETM

SEQ ID NO: 717 - protein, 15 aa, Homo sapiens
IDETMIHNGPPHNSN

SEQ ID NO: 718 - protein, 15 aa, Homo sapiens
FGYSYAKRMIDVQNR

SEQ ID NO: 719 - protein, 15 aa, Homo sapiens
AKRMIDVQNRAYFQQ

SEQ ID NO: 720 - protein, 15 aa, Homo sapiens
DVQNRAYFQQYGCTF

SEQ ID NO: 721 - protein, 15 aa, Homo sapiens
TAVIPTNVFGPHDNF

SEQ ID NO: 722 - protein, 15 aa, Homo sapiens
TNVFGPHDNFNIEDG

SEQ ID NO: 723 - protein, 15 aa, Homo sapiens
PHDNFNIEDGHVLPG

SEQ ID NO: 724 - protein, 15 aa, Homo sapiens
NIEDGHVLPGLIHKV

SEQ ID NO: 725 - protein, 15 aa, Homo sapiens
HVLPGLIHKVHLAKS

SEQ ID NO: 726 - protein, 15 aa, Homo sapiens
LIHKVHLAKSSGSAL

SEQ ID NO: 727 - protein, 15 aa, Homo sapiens
HLAKSSGSALTVWGT

SEQ ID NO: 728 - protein, 15 aa, Homo sapiens
SGSALTVWGTGNPRR

SEQ ID NO: 729 - protein, 15 aa, Homo sapiens
TVWGTGNPRRQFIYS

SEQ ID NO: 730 - protein, 15 aa, Homo sapiens
GNPRRQFIYSLDLAQ

SEQ ID NO: 731 - protein, 15 aa, Homo sapiens
QFIYSLDLAQLFIWV

SEQ ID NO: 732 - protein, 15 aa, Homo sapiens
LDLAQLFIWVLREYN

SEQ ID NO: 733 - protein, 15 aa, Homo sapiens
EEDEVSIKEAAEAVV

SEQ ID NO: 734 - protein, 15 aa, Homo sapiens
EAMDFHGEVTFDTTK

SEQ ID NO: 735 - protein, 15 aa, Homo sapiens
HGEVTFDTTKSDGQF

SEQ ID NO: 736 - protein, 15 aa, Homo sapiens
SDGQFKKTASNSKLR

SEQ ID NO: 737 - protein, 15 aa, Homo sapiens
KKTASNSKLRTYLPD

SEQ ID NO: 738 - protein, 15 aa, Homo sapiens
NSKLRTYLPDFRFTP

SEQ ID NO: 739 - protein, 15 aa, Homo sapiens
TYLPDFRFTPFKQAV

SEQ ID NO: 740 - protein, 15 aa, Homo sapiens
FKQAVKETCAWFTDN

SEQ ID NO: 741 - protein, 16 aa, Homo sapiens
KETCAWFTDNYEQARK

SEQ ID NO: 742 - protein, 19 aa, Homo sapiens
LWEGLTELVTATGNYGNYR

SEQ ID NO: 743 - protein, 12 aa, Homo sapiens
ILVTGGSGLVGK

SEQ ID NO: 744 - protein, 19 aa, Homo sapiens
VVADGAGLPGEDWVFVSSK

SEQ ID NO: 745 - protein, 11 aa, Homo sapiens
DADLTDTAQTR

SEQ ID NO: 746 - protein, 18 aa, Homo sapiens
VQPTHVIHLAAMVGGLFR

SEQ ID NO: 747 - protein, 7 aa, Homo sapiens
YNLDFWR

SEQ ID NO: 748 - protein, 8 aa, Homo sapiens
YNLDFWRK

SEQ ID NO: 749 - protein, 18 aa, Homo sapiens
NVHMNDNVLHSAFEVGAR

SEQ ID NO: 750 - protein, 19 aa, Homo sapiens
NVHMNDNVLHSAFEVGARK

SEQ ID NO: 751 - protein, 13 aa, Homo sapiens
VVSCLSTCIFPDK

SEQ ID NO: 752 - protein, 7 aa, Homo sapiens
MIDVQNR

SEQ ID NO: 753 - protein, 8 aa, Homo sapiens
RMIDVQNR

SEQ ID NO: 754 - protein, 15 aa, Homo sapiens
SSGSALTVWGTGNPR

SEQ ID NO: 755 - protein, 16 aa, Homo sapiens
SSGSALTVWGTGNPRR

SEQ ID NO: 756 - protein, 26 aa, Homo sapiens
TTYPIDETMIHNGPPHNSNFGYSYAK

SEQ ID NO: 757 - protein, 21 aa, Homo sapiens
EYNEVEPIILSVGEEDEVSIK

SEQ ID NO: 758 - protein, 7 aa, Homo sapiens
TYLPDFR

SEQ ID NO: 759 - protein, 9 aa, Homo sapiens
LRTYLPDFR

SEQ ID NO: 760 - protein, 160 aa, Homo sapiens
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 761 - protein, 160 aa, Homo sapiens
SPGQGTQSENSCTHFPGNLPNMLRDLRDAFSRVKTFFQMKDQLDNLLLKESLLEDFKGYLGCQALSEMIQFYLEEVMPQAENQDPDAKAHVNSLGENLKTLRLRLRRCHRFLPCENKSKAVEQVKNAFNKLQEKGIYKAMSEFDIFINYIEAYMTMKIRN

SEQ ID NO: 762 - protein, 166 aa, Homo sapiens
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENGGGSGGKSKAVEQVKNAFNKLQEKGIYKAMSEFDIFINYIEAYMTMKIRN

SEQ ID NO: 763 - protein, 166 aa, Homo sapiens
SPGQGTQSENSCTHFPGNLPNMLRDLRDAFSRVKTFFQMKDQLDNLLLKESLLEDFKGYLGCQALSEMIQFYLEEVMPQAENQDPDAKAHVNSLGENLKTLRLRLRRCHRFLPCENGGGSGGKSKAVEQVKNAFNKLQEKGIYKAMSEFDIFINYIEAYMTMKIRN

SEQ ID NO: 764 - protein, 220 aa, Homo sapiens
FTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNER

SEQ ID NO: 765 - protein, 16 aa, Homo sapiens
LRMKLPKPPKPVSKMR

SEQ ID NO: 766 - protein, 16 aa, Homo sapiens
YRMKLPKPPKPVSKMR

SEQ ID NO: 767 - protein, 4 aa, Homo sapiens
LRMK

SEQ ID NO: 768 - protein, 4 aa, Homo sapiens
YRMK

SEQ ID NO: 769 - protein, 7 aa, Homo sapiens
LRMKLPK

SEQ ID NO: 770 - protein, 7 aa, Homo sapiens
YRMKLPK

SEQ ID NO: 771 - protein, 8 aa, Homo sapiens
YRMKLPKP

SEQ ID NO: 772 - protein, 8 aa, Homo sapiens
LRMKLPKP

SEQ ID NO: 773 - protein, 8 aa, Homo sapiens
LRMKLPKS

SEQ ID NO: 774 - protein, 8 aa, Homo sapiens
YRMKLPKS

SEQ ID NO: 775 - protein, 11 aa, Homo sapiens
LRMKLPKSAKP

SEQ ID NO: 776 - protein, 14 aa, Homo sapiens
LRMKLPKSAKPVSK

SEQ ID NO: 777 - protein, 175 aa, Artificial Sequence
  Description of Artificial Sequence Synthetic polypeptide
LSTTEVAMHTSTSSSVTKSYISSQTNDTHKRDTYAATPRAHEVSEISVRTVYPPEEETGERVQLAHHFSEPEITLIIFGVMAGVIGTILLISYGIRRLIKKSPSDVKPLPSPDTDVPLSSVEIENPETSDQGSGSGSGSGSGSGSGSGSLRMKGAGSLQPLALEGSLQKRGIVEQ

SEQ ID NO: 778 - protein, 331 aa, Artificial Sequence
  Description of Artificial Sequence Synthetic polypeptide
LSTTEVAMHTSTSSSVTKSYISSQTNDTHKRDTYAATPRAHEVSEISVRTVYPPEEETGERVQLAHHFSEPEITLIIFGVMAGVIGTILLISYGIRRLIKKSPSDVKPLPSPDTDVPLSSVEIENPETSDQGSGSGSGSGSGSGSGSGSQQQGRLDKLTVTSQNLQLENLRMKLPKPPKPVSKMRMATPLLMQALPMGALPQGPMQNATKYGNMTEDHVMHLLQNADPLKVYPPLKGSFPENLRHLKNTMETIDWKVFESWMHHWLLFEMSRHSLEQKPTDAPPKESLELEDPSSGLGVTKQDLGPVPMGAGSLQPLALEGSLQKRGIVEQ

SEQ ID NO: 779 - protein, 307 aa, Artificial Sequence
  Description of Artificial Sequence Synthetic polypeptide
LSTTEVAMHTSTSSSVTKSYISSQTNDTHKRDTYAATPRAHEVSEISVRTVYPPEEETGERVQLAHHFSEPEITLIIFGVMAGVIGTILLISYGIRRLIKKSPSDVKPLPSPDTDVPLSSVEIENPETSDQGSGSGSGSGSGSGSGSGSQQQGRLDKLTVTSQNLQLENLRMKGAGSLQPLALEGSLQKRGIVEQGALPQGPMQNATKYGNMTEDHVMHLLQNADPLKVYPPLKGSFPENLRHLKNTMETIDWKVFESWMHHWLLFEMSRHSLEQKPTDAPPKESLELEDPSSGLGVTKQDLGPVPM

SEQ ID NO: 780 - protein, 124 aa, Artificial Sequence
  Description of Artificial Sequence Synthetic polypeptide
MQPQESHVHYSRWEDGSRDGVSLGAVSSTEEASRCRRISQRLCTGKLGIAMKVLGGVALFWIIFILGYLTGYYVHKCKGGGGSGGGGSGGGGSGGGGSLRMKGAGSLQPLALEGSLQKRGIVEQ

SEQ ID NO: 781 - protein, 279 aa, Artificial Sequence
  Description of Artificial Sequence Synthetic polypeptide
MQPQESHVHYSRWEDGSRDGVSLGAVSSTEEASRCRRISQRLCTGKLGIAMVLGGVALFWIIFILGYLTGYYVHKCKGGGGSGGGGSGGGGSGGGGSQQQGRLDKLTVTSQNLQLENLRMKLPKPPKPVSKMRMATPLLMQALPMGALPQGPMQNATKYGNMTEDHVMHLLQNADPLKVYPPLKGSFPENLRHLKNTMETIDWKVFESWMHHWLLFEMSRHSLEQKPTDAPPKESLELEDPSSGLGVTKQDLGPVPMGAGSLQPLALEGSLQKRGIVEQ

SEQ ID NO: 782 - protein, 256 aa, Artificial Sequence
  Description of Artificial Sequence Synthetic polypeptide
MQPQESHVHYSRWEDGSRDGVSLGAVSSTEEASRCRRISQRLCTGKLGIAMKVLGGVALFWIIFILGYLTGYYVHKCKGGGGSGGGGSGGGGSGGGGSQQQGRLDKLTVTSQNLQLENLRMKGAGSLQPLALEGSLQKRGIVEQGALPQGPMQNATKYGNMTEDHVMHLLQNADPLKVYPPLKGSFPENLRHLKNTMETIDWKVFESWMHHWLLFEMSRHSLEQKPTDAPPKESLELEDPSSGLGVTKQDLGPVPM

SEQ ID NO: 783 - protein, 184 aa, Artificial Sequence
  Description of Artificial Sequence Synthetic polypeptide
MAGWNAYIDNLMADGTCQDAAIVGYKDSPSVWAAVPGKTFVNITPAEVGVLVGKDRSSFYVNGLTLGGQKCSVIRDSLLQDGEFSMDLRTKSTGGAPTFNVTVTKTDKTLVLLMGKEGVHGGLINKKCYEMASHLRRSQYGSGSGSGSGSGSGSGSGSLRMKGAGSLQPLALEGSLQKRGIVEQ

SEQ ID NO: 784 - protein, 340 aa, Artificial Sequence
  Description of Artificial Sequence Synthetic polypeptide
MAGWNAYIDNLMADGTCQDAAIVGYKDSPSVWAAVPGKTFVNITPAEVGVLVGKDRSSFYVNGLTLGGQKCSVIRDSLLQDGEFSMDLRTKSTGGAPTFNVTVTKTDKTLVLLMGKEGVHGGLINKKCYEMASHLRRSQYGSGSGSGSGSGSGSGSGSQQQGRLDKLTVTSQNLQLENLRMKLPKPPKPVSKMRMATPLLMQALPMGALPQGPMQNATKYGNMTEDHVMHLLQNADPLKVYPPLKGSFPENLRHLKNTMETIDWKVFESWMHHWLLFEMSRHSLEQKPTDAPPKESLELEDPSSGLGVTKQDLGPVPMGAGSLQPLALEGSLQKRGIVEQ

SEQ ID NO: 785 - protein, 316 aa, Artificial Sequence
  Description of Artificial Sequence Synthetic polypeptide
MAGWNAYIDNLMADGTCQDAAIVGYKDSPSVWAAVPGKTFVNITPAEVGVLVGKDRSSFYVNGLTLGGQKCSVIRDSLLQDGEFSMDLRTKSTGGAPTFNVTVTKTDKTLVLLMGKEGVHGGLINKKCYEMASHLRRSQYGSGSGSGSGSGSGSGSGSQQQGRLDKLTVTSQNLQLENLRMKGAGSLQPLALEGSLQKRGIVEQGALPQGPMQNATKYGNMTEDHVMHLLQNADPLKVYPPLKGSFPENLRHLKNTMETIDWKVFESWMHHWLLFEMSRHSLEQKPTDAPPKESLELEDPSSGLGVTKQDLGPVPM

SEQ ID NO: 786 - protein, 179 aa, Artificial Sequence
  Description of Artificial Sequence Synthetic polypeptide
LSTTEVAMHTSTSSSVTKSYISSQTNDTHKRDTYAATPRAHEVSEISVRTVYPPEEETGERVQLAHHFSEPEITLIIFGVMAGVIGTILLISYGIRRLIKKSPSDVKPLPSPDTDVPLSSVEIENPETSDQGSGSGSGSGSGSGSGSGSLRMKLPKPGAGSLQPLALEGSLQKRGIVEQ

SEQ ID NO: 787 - protein, 146 aa, Artificial Sequence
  Description of Artificial Sequence Synthetic polypeptide
MQPQESHVHYSRWEDGSRDGVSLGAVSSTEEASRCRRISQRLCTGKLGIAMKVLGGVALFWIIFILGYLTGYYVHKCKGGGGSGGGGSGGGGSGGGGSLRMKLPKPGAGSLQPLALEGSLQKRGIVEQGGGGSGGGGSDYKDDDDK

SEQ ID NO: 788 - protein, 205 aa, Artificial Sequence
  Description of Artificial Sequence Synthetic polypeptide
MAGWNAYIDNLMADGTCQDAAIVGYKDSPSVWAAVPGKTFVNITPAEVGVLVGKDRSSFYVNGLTLGGQKCSVIRDSLLQDGEFSMDLRTKSTGGAPTFNVTVTKTDKTLVLLMGKEGVHGGLINKKCYEMASHLRRSQYGGSGGSGGYPYDVPDYAGSGSGSGSGSGSGSGSGSLRMKLPKPGAGSLQPLALEGSLQKRGIVEQ

SEQ ID NO: 789 - protein, 245 aa, Artificial Sequence
  Description of Artificial Sequence Synthetic polypeptide
LSTTEVAMHTSTSSSVTKSYISSQTNDTHKRDTYAATPRAHEVSEISVRTVYPPEEETGERVQLAHHFSEPEITLIIFGVMAGVIGTILLISYGIRRLIKKSPSDVKPLPSPDTDVPLSSVEIENPETSDQGSGSGSGSGSGSGSGSGSLRMKLPKPGAGSLQPLALEGSLQKRGIVEQGPGPGLRMKLPKPKVNFFRMVISNPAATHQDIDGPGPGLRMKLPKPDVMNILLQYVVKSFDRSTKV

SEQ ID NO: 790 - protein, 245 aa, Artificial Sequence
  Description of Artificial Sequence Synthetic polypeptide
LSTTEVAMHTSTSSSVTKSYISSQTNDTHKRDTYAATPRAHEVSEISVRTVYPPEEETGERVQLAHHFSEPEITLIIFGVMAGVIGTILLISYGIRRLIKKSPSDVKPLPSPDTDVPLSSVEIENPETSDQGSGSGSGSGSGSGSGSGSLRMKLPKPKVNFFRMVISNPAATHQDIDGPGPGLRMKLPKPGAGSLQPLALEGSLQKRGIVEQGPGPGLRMKLPKPDVMNILLQYVVKSFDRSTKV

SEQ ID NO: 791 - protein, 245 aa, Artificial Sequence
  Description of Artificial Sequence Synthetic polypeptide
LSTTEVAMHTSTSSSVTKSYISSQTNDTHKRDTYAATPRAHEVSEISVRTVYPPEEETGERVQLAHHFSEPEITLIIFGVMAGVIGTILLISYGIRRLIKKSPSDVKPLPSPDTDVPLSSVEIENPETSDQGSGSGSGSGSGSGSGSGSLRMKLPKPKVNFFRMVISNPAATHQDIDGPGPGLRMKLPKPDVMNILLQYVVKSFDRSTKVGPGPGLRMKLPKPGAGSLQPLALEGSLQKRGIVEQ

SEQ ID NO: 792 - protein, 232 aa, Artificial Sequence
  Description of Artificial Sequence Synthetic polypeptide
LRMKLPKPGAGSLQPLALEGSLQKRGIVEQGPGPGLRMKLPKPKVNFFRMVISNPAATHQDIDGPGPGLRMKLPKPDVMNILLQYVVKSFDRSTKVGPGPGLSTTEVAMHTSTSSSVTKSYISSQTNDTHKRDTYAATPRAHEVSEISVRTVYPPEEETGERVQLAHHFSEPEITLIIFGVMAGVIGTILLISYGIRRLIKKSPSDVKPLPSPDTDVPLSSVEIENPETSDQ

SEQ ID NO: 793 - protein, 232 aa, Artificial Sequence
  Description of Artificial Sequence Synthetic polypeptide
LRMKLPKPKVNFFRMVISNPAATHQDIDGPGPGLRMKLPKPGAGSLQPLALEGSLQKRGIVEQGPGPGLRMKLPKPDVMNILLQYVVKSFDRSTKVGPGPGLSTTEVAMHTSTSSSVTKSYISSQTNDTHKRDTYAATPRAHEVSEISVRTVYPPEEETGERVQLAHHFSEPEITLIIFGVMAGVIGTILLISYGIRRLIKKSPSDVKPLPSPDTDVPLSSVEIENPETSDQ

SEQ ID NO: 794 - protein, 232 aa, Artificial Sequence
  Description of Artificial Sequence Synthetic polypeptide
LRMKLPKPKVNFFRMVISNPAATHQDIDGPGPGLRMKLPKPDVMNILLQYVVKSFDRSTKVGPGPGLRMKLPKPGAGSLQPLALEGSLQKRGIVEQGPGPGLSTTEVAMHTSTSSSVTKSYISSQTNDTHKRDTYAATPRAHEVSEISVRTVYPPEEETGERVQLAHHFSEPEITLIIFGVMAGVIGTILLISYGIRRLIKKSPSDVKPLPSPDTDVPLSSVEIENPETSDQ

SEQ ID NO: 795 - protein, 254 aa, Artificial Sequence
  Description of Artificial Sequence Synthetic polypeptide
MAGWNAYIDNLMADGTCQDAAIVGYKDSPSVWAAVPGKTFVNITPAEVGVLVGKDRSSFYVNGLTLGGQKCSVIRDSLLQDGEFSMDLRTKSTGGAPTFNVTVTKTDKTLVLLMGKEGVHGGLINKKCYEMASHLRRSQYGSGSGSGSGSGSGSGSGSLRMKLPKPGAGSLQPLALEGSLQKRGIVEQGPGPGLRMKLPKPKVNFFRMVISNPAATHQDIDGPGPGLRMKLPKPDVMNILLQYVVKSFDRSTKV

SEQ ID NO: 796 - protein, 267 aa, Artificial Sequence
  Description of Artificial Sequence Synthetic polypeptide
MAGWNAYIDNLMADGTCQDAAIVGYKDSPSVWAAVPGKTFVNITPAEVGVLVGKDRSSFYVNGLTLGGQKCSVIRDSLLQDGEFSMDLRTKSTGGAPTFNVTVTKTDKTLVLLMGKEGVHGGLINKKCYEMASHLRRSQYGSGSGSGSGSGSGSGSGSLRMKLPKPKVNFFRMVISNPAATHQDIDGPGPGLRMKLPKPGAGSLQPLALEGSLQKRGIVEQGPGPGLRMKLPKPDVMNILLQYVVKSFDRSTKVGPGPGDYKDDDDK

SEQ ID NO: 797 - protein, 254 aa, Artificial Sequence
  Description of Artificial Sequence Synthetic polypeptide
MAGWNAYIDNLMADGTCQDAAIVGYKDSPSVWAAVPGKTFVNITPAEVGVLVGKDRSSFYVNGLTLGGQKCSVIRDSLLQDGEFSMDLRTKSTGGAPTFNVTVTKTDKTLVLLMGKEGVHGGLINKKCYEMASHLRRSQYGSGSGSGSGSGSGSGSGSLRMKLPKPKVNFFRMVISNPAATHQDIDGPGPGLRMKLPKPDVMNILLQYVVKSFDRSTKVGPGPGLRMKLPKPGAGSLQPLALEGSLQKRGIVEQ

SEQ ID NO: 798 - protein, 164 aa, Artificial Sequence
  Description of Artificial Sequence Synthetic polypeptide
LSTTEVAMHTSTSSSVTKSYISSQTNDTHKRDTYAATPRAHEVSEISVRTVYPPEEETGERVQLAHHFSEPEITLIIFGVMAGVIGTILLISYGIRRLIKKSPSDVKPLPSPDTDVPLSSVEIENPETSDQGSGSGSGSGSGSGSGSGSSLQPLALEGSLQKRG

SEQ ID NO: 799 - protein, 187 aa, Artificial Sequence
  Description of Artificial Sequence Synthetic polypeptide
LSTTEVAMHTSTSSSVTKSYISSQTNDTHKRDTYAATPRAHEVSEISVRTVYPPEEETGERVQLAHHFSEPEITLIIFGVMAGVIGTILLISYGIRRLIKKSPSDVKPLPSPDTDVPLSSVEIENPETSDQGSGSGSGSGSGSGSGSGSEAEDLQVGQVELGGGPGAGSLQPLALEGSLQKRGIVEQ

SEQ ID NO: 800 - protein, 235 aa, Artificial Sequence
  Description of Artificial Sequence Synthetic polypeptide
LSTTEVAMHTSTSSSVTKSYISSQTNDTHKRDTYAATPRAHEVSEISVRTVYPPEEETGERVQLAHHFSEPEITLIIFGVMAGVIGTILLISYGIRRLIKKSPSDVKPLPSPDTDVPLSSVEIENPETSDQGSGSGSGSGSGSGSGSGSFVNQHLCGSHLVEALYLVCGERGFFYTPKTRREAEDLQVGQVELGGGPGAGSLQPLALEGSLQKRGIVEQCCTSICSLYQLENYCN

SEQ ID NO: 801 - protein, 260 aa, Artificial Sequence
  Description of Artificial Sequence Synthetic polypeptide
LSTTEVAMHTSTSSSVTKSYISSQTNDTHKRDTYAATPRAHEVSEISVRTVYPPEEETGERVQLAHHFSEPEITLIIFGVMAGVIGTILLISYGIRRLIKKSPSDVKPLPSPDTDVPLSSVEIENPETSDQGSGSGSGSGSGSGSGSGSSLQPLALEGSLQKRGGGGGSGGGGSNFFRMVISNPAATGGGGSGGGGSVMNILLQYVVGGGGSGGGGSSRLSKVAPVIKARMMEYGTTGGGGSGGGGSIAFTSEHSHFSLK

SEQ ID NO: 802 - protein, 269 aa, Artificial Sequence
  Description of Artificial Sequence Synthetic polypeptide
LSTTEVAMHTSTSSSVTKSYISSQTNDTHKRDTYAATPRAHEVSEISVRTVYPPEEETGERVQLAHHFSEPEITLIIFGVMAGVIGTILLISYGIRRLIKKSPSDVKPLPSPDTDVPLSSVEIENPETSDQGSGSGSGSGSGSGSGSGSEAEDLQVGQVELGGGPGAGSLQPLALEGSLQKRGIVEQSRLSKVAPVIKARMMEYGTTMVSYQPLGDKVNFFRMVISNPAATHQDIDDVMNILLQYVVKSFDRSTKVIAFTSEHSHFSLK

SEQ ID NO: 803 - protein, 317 aa, Artificial Sequence
  Description of Artificial Sequence Synthetic polypeptide
LSTTEVAMHTSTSSSVTKSYISSQTNDTHKRDTYAATPRAHEVSEISVRTVYPPEEETGERVQLAHHFSEPEITLIIFGVMAGVIGTILLISYGIRRLIKKSPSDVKPLPSPDTDVPLSSVEIENPETSDQGSGSGSGSGSGSGSGSGSFVNQHLCGSHLVEALYLVCGERGFFYTPKTRREAEDLQVGQVELGGGPGAGSLQPLALEGSLQKRGIVEQCCTSICSLYQLENYCNSRLSKVAPVIKARMMEYGTTMVSYQPLGDKVNFFRMVISNPAATHQDIDDVMNILLQYVVKSFDRSTKVIAFTSEHSHFSLK

SEQ ID NO: 804 - protein, 198 aa, Artificial Sequence
  Description of Artificial Sequence Synthetic polypeptide
LSTTEVAMHTSTSSSVTKSYISSQTNDTHKRDTYAATPRAHEVSEISVRTVYPPEEETGERVQLAHHFSEPEITLIIFGVMAGVIGTILLISYGIRRLIKKSPSDVKPLPSPDTDVPLSSVEIENPETSDQGSGSGSGSGSGSGSGSGSSRLSKVAPVIKARMMEYGTTMVSYQPLGDKVNFFRMVISNPAATHQDID

SEQ ID NO: 805 - protein, 162 aa, Artificial Sequence
  Description of Artificial Sequence Synthetic polypeptide
LSTTEVAMHTSTSSSVTKSYISSQTNDTHKRDTYAATPRAHEVSEISVRTVYPPEEETGERVQLAHHFSEPEITLIIFGVMAGVIGTILLISYGIRRLIKKSPSDVKPLPSPDTDVPLSSVEIENPETSDQGSGSGSGSGSGSGSGSGSNFFRMVISNPAAT

SEQ ID NO: 806 - protein, 169 aa, Artificial Sequence
  Description of Artificial Sequence Synthetic polypeptide
LSTTEVAMHTSTSSSVTKSYISSQTNDTHKRDTYAATPRAHEVSEISVRTVYPPEEETGERVQLAHHFSEPEITLIIFGVMAGVIGTILLISYGIRRLIKKSPSDVKPLPSPDTDVPLSSVEIENPETSDQGSGSGSGSGSGSGSGSGSDVMNILLQYVVKSFDRSTKV

SEQ ID NO: 807 - protein, 159 aa, Artificial Sequence
  Description of Artificial Sequence Synthetic polypeptide
LSTTEVAMHTSTSSSVTKSYISSQTNDTHKRDTYAATPRAHEVSEISVRTVYPPEEETGERVQLAHHFSEPEITLIIFGVMAGVIGTILLISYGIRRLIKKSPSDVKPLPSPDTDVPLSSVEIENPETSDQGSGSGSGSGSGSGSGSGSVMNILLQYVV

SEQ ID NO: 808 - protein, 169 aa, Artificial Sequence
  Description of Artificial Sequence Synthetic polypeptide
LSTTEVAMHTSTSSSVTKSYISSQTNDTHKRDTYAATPRAHEVSEISVRTVYPPEEETGERVQLAHHFSEPEITLIIFGVMAGVIGTILLISYGIRRLIKKSPSDVKPLPSPDTDVPLSSVEIENPETSDQGSGSGSGSGSGSGSGSGSSRLSKVAPVIKARMMEYGTT

SEQ ID NO: 809 - protein, 162 aa, Artificial Sequence
  Description of Artificial Sequence Synthetic polypeptide
LSTTEVAMHTSTSSSVTKSYISSQTNDTHKRDTYAATPRAHEVSEISVRTVYPPEEETGERVQLAHHFSEPEITLIIFGVMAGVIGTILLISYGIRRLIKKSPSDVKPLPSPDTDVPLSSVEIENPETSDQGSGSGSGSGSGSGSGSGSIAFTSEHSHFSLK

SEQ ID NO: 810 - protein, 198 aa, Artificial Sequence
  Description of Artificial Sequence Synthetic polypeptide
SRLSKVAPVIKARMMEYGTTMVSYQPLGDKVNFFRMVISNPAATHQDIDGSGSGSGSGSGSGSGSGSLSTTEVAMHTSTSSSVTKSYISSQTNDTHKRDTYAATPRAHEVSEISVRTVYPPEEETGERVQLAHHFSEPEITLIIFGVMAGVIGTILLISYGIRRLIKKSPSDVKPLPSPDTDVPLSSVEIENPETSDQ

SEQ ID NO: 811 - protein, 19 aa, Homo sapiens
MYGKIIFVLLLSEIVSISA

SEQ ID NO: 812 - protein, 24 aa, Artificial Sequence
  Description of Artificial Sequence Synthetic peptide
GGSGGSGGGGGSGGGSGGGSGGGS

SEQ ID NO: 813 - protein, 131 aa, Homo sapiens; identical to SEQ ID NO: 3

SEQ ID NO: 814 - protein, 78 aa, Homo sapiens; identical to SEQ ID NO: 4

SEQ ID NO: 815 - protein, 15 aa, Artificial Sequence
  Description of Artificial Sequence Synthetic peptide
GGGGSGGGGSGGGGS

SEQ ID NO: 816 - protein, 315 aa, Artificial Sequence
  Description of Artificial Sequence Synthetic polypeptide
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNGGSGGSGGGGGSGGGSGGGSGGGSLSTTEVAMHTSTSSSVTKSYISSQTNDTHKRDTYAATPRAHEVSEISVRTVYPPEEETGERVQLAHHFSEPEITLIIFGVMAGVIGTILLISYGIRRLIKKSPSDVKPLPSPDTDVPLSSVEIENPETSDQ

SEQ ID NO: 817 - protein, 253 aa, Artificial Sequence
  Description of Artificial Sequence Synthetic polypeptide
MQPQESHVHYSRWEDGSRDGVSLGAVSSTEEASRCRRISQRLCTGKLGIAMKVLGGVALFWIIFILGYLTGYYVHKCKGGGGSGGGGSGGGGSSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 818 - protein, 315 aa, Artificial Sequence
  Description of Artificial Sequence Synthetic polypeptide
SPGQGTQSENSCTHFPGNLPNMLRDLRDAFSRVKTFFQMKDQLDNLLLKESLLEDFKGYLGCQALSEMIQFYLEEVMPQAENQDPDAKAHVNSLGENLKTLRLRLRRCHRFLPCENKSKAVEQVKNAFNKLQEKGIYKAMSEFDIFINYIEAYMTMKIRNGGSGGSGGGGGSGGGSGGGSGGGSLSTTEVAMHTSTSSSVTKSYISSQTNDTHKRDTYAATPRAHEVSEISVRTVYPPEEETGERVQLAHHFSEPEITLIIFGVMAGVIGTILLISYGIRRLIKKSPSDVKPLPSPDTDVPLSSVEIENPETSDQ

SEQ ID NO: 819 - protein, 253 aa, Artificial Sequence
  Description of Artificial Sequence Synthetic polypeptide
MQPQESHVHYSRWEDGSRDGVSLGAVSSTEEASRCRRISQRLCTGKLGIAMKVLGGVALFWIIFILGYLTGYYVHKCKGGGGSGGGGSGGGGSSPGQGTQSENSCTHFPGNLPNMLRDLRDAFSRVKTFFQMKDQLDNLLLKESLLEDFKGYLGCQALSEMIQFYLEEVMPQAENQDPDAKAHVNSLGENLKTLRLRLRRCHRFLPCENKSKAVEQVKNAFNKLQEKGIYKAMSEFDIFINYIEAYMTMKIRN

SEQ ID NO: 820 - protein, 321 aa, Artificial Sequence
  Description of Artificial Sequence Synthetic polypeptide
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENGGGSGGKSKAVEQVKNAFNKLQEKGIYKAMSEFDIFINYIEAYMTMKIRNGGSGGSGGGGGSGGGSGGGSGGGSLSTTEVAMHTSTSSSVTKSYISSQTNDTHKRDTYAATPRAHEVSEISVRTVYPPEEETGERVQLAHHFSEPEITLIIFGVMAGVIGTILLISYGIRRLIKKSPSDVKPLPSPDTDVPLSSVEIENPETSDQ

SEQ ID NO: 821 - protein, 259 aa, Artificial Sequence
  Description of Artificial Sequence Synthetic polypeptide
MQPQESHVHYSRWEDGSRDGVSLGAVSSTEEASRCRRISQRLCTGKLGIAMKVLGGVALFWIIFILGYLTGYYVHKCKGGGGSGGGGSGGGGSSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENGGGSGGKSKAVEQVKNAFNKLQEKGIYKAMSEFDIFINYIEAYMTMKIRN

SEQ ID NO: 822 - protein, 375 aa, Artificial Sequence
  Description of Artificial Sequence Synthetic polypeptide
FTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERGGSGGSGGGGGSGGGSGGGSGGGSLSTTEVAMHTSTSSSVTKSYISSQTNDTHKRDTYAATPRAHEVSEISVRTVYPPEEETGERVQLAHHFSEPEITLIIFGVMAGVIGTILLISYGIRRLIKKSPSDVKPLPSPDTDVPLSSVEIENPETSDQ

SEQ ID NO: 823 - protein, 313 aa, Artificial Sequence
  Description of Artificial Sequence Synthetic polypeptide
MQPQESHVHYSRWEDGSRDGVSLGAVSSTEEASRCRRISQRLCTGKLGIAMKVLGGVALFWIIFILGYLTGYYVHKCKGGGGSGGGGSGGGGSFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNER

SEQ ID NO: 824 - protein, 162 aa, Artificial Sequence
  Description of Artificial Sequence Synthetic polypeptide
NFFRMVISNPAATGSGSGSGSGSGSGSGSGSLSTTEVAMHTSTSSSVTKSYISSQTNDTHKRDTYAATPRAHEVSEISVRTVYPPEEETGERVQLAHHFSEPEITLIIFGVMAGVIGTILLISYGIRRLIKKSPSDVKPLPSPDTDVPLSSVEIENPETSDQ

SEQ ID NO: 825 - protein, 169 aa, Artificial Sequence
  Description of Artificial Sequence Synthetic polypeptide
DVMNILLQYVVKSFDRSTKVGSGSGSGSGSGSGSGSGSLSTTEVAMHTSTSSSVTKSYISSQTNDTHKRDTYAATPRAHEVSEISVRTVYPPEEETGERVQLAHHFSEPEITLIIFGVMAGVIGTILLISYGIRRLIKKSPSDVKPLPSPDTDVPLSSVEIENPETSDQ

SEQ ID NO: 826 - protein, 159 aa, Artificial Sequence
  Description of Artificial Sequence Synthetic polypeptide
VMNILLQYVVGSGSGSGSGSGSGSGSGSLSTTEVAMHTSTSSSVTKSYISSQTNDTHKRDTYAATPRAHEVSEISVRTVYPPEEETGERVQLAHHFSEPEITLIIFGVMAGVIGTILLISYGIRRLIKKSPSDVKPLPSPDTDVPLSSVEIENPETSDQ

SEQ ID NO: 827 - protein, 169 aa, Artificial Sequence
  Description of Artificial Sequence Synthetic polypeptide
SRLSKVAPVIKARMMEYGTTGSGSGSGSGSGSGSGSGSLSTTEVAMHTSTSSSVTKSYISSQTNDTHKRDTYAATPRAHEVSEISVRTVYPPEEETGERVQLAHHFSEPEITLIIFGVMAGVIGTILLISYGIRRLIKKSPSDVKPLPSPDTDVPLSSVEIENPETSDQ

SEQ ID NO: 828 - protein, 162 aa, Artificial Sequence
  Description of Artificial Sequence Synthetic polypeptide
IAFTSEHSHFSLKGSGSGSGSGSGSGSGSGSLSTTEVAMHTSTSSSVTKSYISSQTNDTHKRDTYAATPRAHEVSEISVRTVYPPEEETGERVQLAHHFSEPEITLIIFGVMAGVIGTILLISYGIRRLIKKSPSDVKPLPSPDTDVPLSSVEIENPETSDQ

SEQ ID NO: 829 - protein, 187 aa, Artificial Sequence
  Description of Artificial Sequence Synthetic polypeptide
EAEDLQVGQVELGGGPGAGSLQPLALEGSLQKRGIVEQGSGSGSGSGSGSGSGSGSLSTTEVAMHTSTSSSVTKSYISSQTNDTHKRDTYAATPRAHEVSEISVRTVYPPEEETGERVQLAHHFSEPEITLIIFGVMAGVIGTILLISYGIRRLIKKSPSDVKPLPSPDTDVPLSSVEIENPETSDQ

SEQ ID NO: 830 - protein, 164 aa, Artificial Sequence
  Description of Artificial Sequence Synthetic polypeptide
SLQPLALEGSLQKRGGSGSGSGSGSGSGSGSGSLSTTEVAMHTSTSSSVTKSYISSQTNDTHKRDTYAATPRAHEVSEISVRTVYPPEEETGERVQLAHHFSEPEITLIIFGVMAGVIGTILLISYGIRRLIKKSPSDVKPLPSPDTDVPLSSVEIENPETSDQ

SEQ ID NO: 831 - protein, 269 aa, Artificial Sequence
  Description of Artificial Sequence Synthetic polypeptide
EAEDLQVGQVELGGGPGAGSLQPLALEGSLQKRGIVEQSRLSKVAPVIKARMMEYGTTMVSYQPLGDKVNFFRMVISNPAATHQDIDDVMNILLQYVVKSFDRSTKVIAFTSEHSHFSLKGSGSGSGSGSGSGSGSGSLSTTEVAMHTSTSSSVTKSYISSQTNDTHKRDTYAATPRAHEVSEISVRTVYPPEEETGERVQLAHHFSEPEITLIIFGVMAGVIGTILLISYGIRRLIKKSPSDVKPLPSPDTDVPLSSVEIENPETSDQ

SEQ ID NO: 832 - protein, 259 aa, Artificial Sequence
  Description of Artificial Sequence Synthetic polypeptide
SLQPLALEGSLQKRGGGGSGGGGSNFFRMVISNPAATGGGGSGGGGSVMNILLQYVVGGGGSGGGGSSRLSKVAPVIKARMMEYGTTGGGGSGGGGSIAFTSEHSHFSLKGSGSGSGSGSGSGSGSGSLSTTEVAMHTSTSSSVTKSYISSQTNDTHKRDTYAATPRAHEVSEISVRTVYPPEEETGERVQLAHHFSEPEITLIIFGVMAGVIGTILLISYGIRRLIKKSPSDVKPLPSPDTDVPLSSVEIENPETSDQ

SEQ ID NO: 833 - protein, 140 aa, Homo sapiens
MAGWNAYIDNLMADGTCQDAAIVGYKDSPSVWAAVPGKTFVNITPAEVGVLVGKDRSSFYVNGLTLGGQKCSVIRDSLLQDGEFSMDLRTKSTGGAPTFNVTVTKTDKTLVLLMGKEGVHGGLINKKCYEMASHLRRSQY

SEQ ID NO: 834 - protein, 22 aa, Homo sapiens
GAGSLQPLALEGSLQKRGIVEQ

SEQ ID NO: 835 - protein, 14 aa, Homo sapiens
  MOD_RES (6)..(6): Citrullinated
VEKAKRALSSQHQA

SEQ ID NO: 836 - protein, 160 aa, Homo sapiens
QQQGRLDKLTVTSQNLQLENLRMKLPKPPKPVSKMRMATPLLMQALPMGALPQGPMQNATKYGNMTEDHVMHLLQNADPLKVYPPLKGSFPENLRHLKNTMETIDWKVFESWMHHWLLFEMSRHSLEQKPTDAPPKESLELEDPSSGLGVTKQDLGPVPM

SEQ ID NO: 837 - protein, 112 aa, Homo sapiens
GALPQGPMQNATKYGNMTEDHVMHLLQNADPLKVYPPLKGSFPENLRHLKNTMETIDWKVFESWMHHWLLFEMSRHSLEQKPTDAPPKESLELEDPSSGLGVTKQDLGPVPM

SEQ ID NO: 838 - protein, 38 aa, Homo sapiens
EAEDLQVGQVELGGGPGAGSLQPLALEGSLQKRGIVEQ

SEQ ID NO: 839 - protein, 100 aa, Artificial Sequence
  Description of Artificial Sequence Synthetic polypeptide
  MISC_FEATURE (1)..(100): This sequence may encompass 1-20 “Gly Gly Gly Gly Ser” repeating units
GGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGS

(19) United States
(12) Patent Application Publication
(10) Pub. No.: US 2021/0246426 A1
(43) Pub. Date: Aug. 12, 2021

(54) Engineered Erythroid Cells Including HLA-G Polypeptides and Methods of Use Thereof

(71) Applicant: Rubius Therapeutics, Inc., Cambridge, MA (US)

(72) Inventors: Regina Sophia Salvat, Boston, MA (US); Christopher Lawrence Moore, Norfolk, MA (US); Abdulsalam Shaaban, Danbury, CT (US)

(73) Assignee: Rubius Therapeutics, Inc., Cambridge, MA (US)

(21) Appl. No.: 16/889,783

(22) Filed: Jun. 1, 2020

Related U.S. Application Data
(60) Provisional application No. 62/972,632, filed on Feb. 10, 2020.

(51) Int. Cl. C12N 5/078; C07K 14/74

(52) U.S. Cl. CPC C12N 5/0641; A61K 38/00; C12N 2510/02; C07K 14/70539

(57) ABSTRACT

The present disclosure relates to engineered erythroid cells and enucleated cells that include one or more of exogenous HLA-G polypeptides, exogenous immunogenic polypeptides, and exogenous coinhibitory polypeptides wherein the cells are capable of inducing immune tolerance and/or reducing immune response to the exogenous immunogenic polypeptides when administered to a subject.

### CROSS-REFERENCE TO RELATED APPLICATIONS

This application claims priority to U.S. Provisional Patent Application Ser. No. 62/972,632, filed Feb. 10, 2020; the entire contents of which is herein incorporated by reference.

### SEQUENCE LISTING

The instant application contains a Sequence Listing which has been submitted electronically in ASCII format and is hereby incorporated by reference in its entirety. Said ASCII copy, created on Jun. 1, 2020, is named 47472-0053001_SL.txt and is 364,950 bytes in size.

### TECHNICAL FIELD

The present disclosure relates generally to the field of immunology. More specifically, the present disclosure relates to the use of immunogenic polypeptides.

### BACKGROUND

Administration of immunogenic polypeptides, e.g., enzymes, can provide life-saving therapies for patients in need of them. Polypeptides used to treat a range of human diseases are often destroyed, neutralized, or otherwise rendered ineffective by immune cells that respond to them as though they were foreign antigens. This powerful alloresponse by the adaptive and/or innate immune system is often controlled by administration of immunosuppressive drugs. However, treatments with immunosuppressive drugs are associated with significant morbidities because they broadly suppress the immune system. Furthermore, the toxicity of immunosuppressive drugs raises other issues. Thus, the success of immunogenic polypeptide administration often depends on the balance between rejection and the side effects of modern immunosuppressive drugs.

The induction of immune tolerance can diminish the risk of acute and chronic rejection of immunogenic polypeptides, and ultimately, their therapeutic effectiveness. There remains a need for improved compositions and methods for administering immunogenic polypeptides to a subject for therapeutic purposes.

### SUMMARY

The present disclosure relates to engineered erythroid cells (e.g., engineered enucleated erythroid cells) or enucleated cells (e.g., modified enucleated cells), that are engineered to include an exogenous human leukocyte antigen-G (HLA-G) polypeptide and an exogenous immunogenic polypeptide, wherein both the exogenous HLA-G and the exogenous immunogenic polypeptide are on the cell surface.

Also provided are engineered erythroid cells (e.g., engineered enucleated erythroid cells) or enuclated cells (e.g., modified enucleated cells) that include an exogenous HLA-G polypeptide and an exogenous immunogenic polypeptide, wherein the exogenous HLA-G polypeptide is present on the cell surface and the exogenous immunogenic polypeptide is within the cell. In some embodiments, the exogenous immunogenic polypeptide is in the cytosol of the cell. In some embodiments, the exogenous immunogenic polypeptide is on the intracellular side of the plasma membrane. In some embodiments, the exogenous immunogenic polypeptide is secreted or released by the cell.

Also provided herein are engineered erythroid cells (e.g., engineered enucleated erythroid cells) or enucleated cells (e.g., modified enucleated cells) that include an exogenous autoantigenic polypeptide (e.g., any of the exogenous autoantigenic polypeptides described herein) and at least one exogenous coinhibitory polypeptide (e.g., any of the exogenous coinhibitory polypeptides described herein). In some embodiments, the exogenous autoantigenic polypeptide is in the cytosol of the cell. In some embodiments, the exogenous autoantigenic polypeptide is on the intracellular side of the plasma membrane. In some embodiments, the exogenous autoantigenic polypeptide is secreted or released by the cell. In some embodiments, the at least one exogenous coinhibitory polypeptide is on the intracellular side of the plasma membrane. In some embodiments, the at least one exogenous coinhibitory polypeptide is secreted or released by the cell. In some embodiments, the at least one exogenous coinhibitory polypeptide is IL-10, IL-27, IL-37, TGFβ, CD39, CD73, arginase 1 (ARG1), Annexin 1, fibrinogen-like protein 2 (FGL2), or PD-L1.

In some embodiments, the engineered erythroid cells (e.g., engineered enucleated erythroid cells) or enucleated cells (e.g., modified enucleated cells) further comprise an exogenous antigenic polypeptide on the cell surface, wherein optionally, the exogenous antigenic polypeptide is bound to the exogenous HLA-G polypeptide.

In some embodiments of any of the engineered erythroid cells described herein, the engineered erythroid cells (e.g., engineered enucleated erythroid cells) or enucleated cells (e.g., modified enucleated cells) further comprise an exogenous antigenic polypeptide within the cell. In some embodiments, the exogenous antigenic polypeptide is in the cytosol of the cell. In some embodiments, the exogenous antigenic polypeptide is on the intracellular side of the plasma membrane.

In some embodiments, the engineered erythroid cells (e.g., engineered enucleated erythroid cells) or enucleated cells (e.g., modified enucleated cells) further secrete or release an exogenous antigenic polypeptide, wherein optionally, the exogenous antigenic polypeptide is bound to the exogenous HLA-G polypeptide.

In some embodiments, the engineered erythroid cells (e.g., engineered enucleated erythroid cells) or enucleated cells (e.g., modified enucleated cells) include an exogenous HLA-G polypeptide and an exogenous immunogenic polypeptide on the cell surface, wherein the exogenous immunogenic polypeptide is not bound to the exogenous HLA-G polypeptide. In some embodiments, the engineered erythroid cells (e.g., engineered enucleated erythroid cells) or enucleated cells (e.g., modified enucleated cells) include an exogenous HLA-G polypeptide on the cell surface and an exogenous immunogenic polypeptide within the cell, wherein the exogenous immunogenic polypeptide is not bound to the exogenous HLA-G polypeptide.

In some embodiments, the engineered erythroid cells are engineered enucleated erythroid cells, e.g., reticulocytes or erythrocytes. In some embodiments, the enucleated cell (e.g., modified enucleated cell) is a reticulocyte, an erythrocyte or a platelet. In some embodiments, the engineered erythroid cells are nucleated engineered erythroid cells.

In one aspect, the disclosure provides engineered enucleated erythroid cells comprising an exogenous HLA-G polypeptide and an exogenous immunogenic polypeptide, wherein both the exogenous HLA-G polypeptide and the exogenous immunogenic polypeptide are on the cell surface. In some embodiments, the exogenous immunogenic polypeptide is not bound by the exogenous HLA-G polypeptide.

In another aspect, the disclosure provides engineered enucleated erythroid cells comprising an exogenous HLA-G polypeptide and an exogenous immunogenic polypeptide, wherein the exogenous HLA-G polypeptide is on the cell surface and the exogenous immunogenic polypeptide is within the cell (i.e., intracellular), and optionally, the exogenous immunogenic polypeptide is not bound by the exogenous HLA-G polypeptide. In some embodiments, the exogenous immunogenic polypeptide is in the cytosol of the cell, and optionally, is not bound by the exogenous HLA-G polypeptide. In some embodiments, the exogenous immunogenic polypeptide is on the intracellular side of the plasma membrane, and optionally, is not bound by the exogenous HLA-G polypeptide. In some embodiments, the exogenous immunogenic polypeptide comprises a transmembrane domain that positions the exogenous immunogenic polypeptide on the intracellular side of the plasma membrane, and optionally, is not bound by the exogenous HLA-G polypeptide. In some embodiments, the exogenous immunogenic polypeptide is secreted or released by the cell, and optionally, is not bound by the exogenous HLA-G polypeptide.

In some embodiments, the exogenous HLA-G polypeptide comprises any one of a HLA-G1 isoform polypeptide, a HLA-G2 isoform polypeptide, a HLA-G3 isoform polypeptide, a HLA-G4 isoform polypeptide, a HLA-G5 isoform polypeptide, a HLA-G6 isoform polypeptide, and a HLA-G7 isoform polypeptide. In some embodiments, the exogenous HLA-G polypeptide comprises any one of a HLA-G1 isoform polypeptide, a HLA-G2 isoform polypeptide, a HLA-G5 isoform polypeptide, and a HLA-G6 isoform polypeptide.

In some embodiments, the exogenous immunogenic polypeptide comprises a human polypeptide. In some embodiments, the exogenous immunogenic polypeptide comprises a non-human polypeptide (e.g., a polypeptide derived from a bacterium, a plant, a yeast, a fungus, a virus, a prion, or a protozoan). In some embodiments, the exogenous immunogenic polypeptide comprises a polypeptide listed in Table 1 or Table 2. In some embodiments, the exogenous immunogenic polypeptide comprises an amino acid-degrading polypeptide, a uric acid-degrading polypeptide, or oxalate oxidase (OxOx).

In some embodiments, the exogenous immunogenic polypeptide comprises an amino acid-degrading polypeptide, and wherein the amino acid-degrading polypeptide is an asparaginase, a phenylalanine ammonium lyase (PAL), or a phenylalanine hydroxylase (PAH). In some embodiments, the exogenous immunogenic polypeptide comprises a d-aminolevulinate dehydrogenase (ALA-D).

In some embodiments, the exogenous immunogenic polypeptide comprises an amino acid-degrading polypeptide, and wherein the amino acid-degrading polypeptide is a homocysteine-reducing polypeptide or a homocysteine-degrading polypeptide. In some embodiments, the amino acid-degrading polypeptide is the homocysteine-reducing polypeptide, and wherein the homocysteine-reducing polypeptide is a methionine adenosyltransferase, an alanine transaminase, an L-alanine-L-anticapsin ligase, an L-cysteine desulfidase, a methylenetetrahydrofolate reductase, a 5-methyltetrahydrofolate-homocysteine methyltransferase reductase, a methylmalonic aciduria or a homocystinuria, cblD type, or a variant thereof. In some embodiments, the amino acid-degrading polypeptide is the homocysteine-degrading polypeptide, and wherein the homocysteine-degrading polypeptide is a cystathionine-β-synthase (CBS), a methionine gamma-lyase, a sulfide:quinone reductase, a methionine synthase, a 5-methyltetrahydropteroyltriglutamate-homocysteine S-methyltransferase, an adenosylhomocysteinase, a cystathionine gamma-lyase, a methionine gamma-lyase, an L-amino-acid oxidase, a thetin-homocysteine S-methyltransferase, a betaine-homocysteine S-methyltransferase, a homocysteine S-methyltransferase, a 5-methyltetrahydropteroyltriglutamate-homocysteine S-methyltransferase, a selenocysteine Se-methyltransferase, a cystathionine gamma-synthase, an O-acetylhomoserine aminocarboxypropyltransferase, an asparagine-oxo-acid transaminase, a glutamine-phenylpyruvate transaminase, a 3-mercaptopyruvate sulfurtransferase, a homocysteine desulfhydrase, a cystathionine beta-lyase, an amino-acid racemase, a methionine-tRNA ligase, a glutamate-cysteine ligase, an N-(5-amino-5-carboxypentanoyl)-L-cysteinyl-D-valine synthase, an L-isoleucine 4-hydroxylase, an L-lysine N6-monooxygenase (NADPH), a methionine decarboxylase, 2,2-dialkylglycine decarboxylase (pyruvate), and a cysteine synthase (CysO), or a variant thereof.

In some embodiments, the exogenous immunogenic polypeptide comprises the uric acid-degrading polypeptide, and the uric acid-degrading polypeptide is a uricase, a HIU hydrolase, an OHCU decarboxylase, an allantoinase, an allantoicase, a myeloperoxidase, a FAD-dependent urate hydroxylase, a xanthine dehydrogenase, an nucleoside deoxyribosyltransferase, a dioxotetrahydropyrimidine phosphoribosyltransferase, a dihydropyrimidinase, or a guanine deaminase, or a variant thereof.

In some embodiments, the exogenous HLA-G polypeptide is capable of inducing immune tolerance (e.g., short-term immune tolerance or long-term immune tolerance) to the exogenous immunogenic polypeptide upon administration of the cell to a subject.

In some embodiments, the exogenous HLA-G polypeptide is capable of inducing short-term immune tolerance, and the short-term immune tolerance comprises inducing apoptosis or inhibiting the activation, differentiation, and/or proliferation of an immune cell that is contacted by the engineered enucleated erythroid cell, and optionally, wherein the immune cell is a T cell, a natural killer (NK) cell, or a B cell. In some embodiments, the short-term immune tolerance comprises inhibiting the cytotoxicity of a T cell or of an NK cell that is contacted by the engineered enucleated erythroid cell. In some embodiments, the short-term immune tolerance comprises inhibiting antibody secretion by a B cell that is contacted by the engineered enucleated erythroid cell.

In some embodiment, the exogenous HLA-G polypeptide is capable of inducing long-term immune tolerance, wherein the long-term immune tolerance comprises inhibiting the maturation of a dendritic cell (DC) that is contacted by the engineered enucleated erythroid cell. In some embodiments, the long-term immune tolerance comprises inducing anergy of a DC that is contacted by the engineered enucleated erythroid cell. In some embodiments, the long-term immune tolerance comprises: inducing the differentiation of CD4⁺ T cell that is contacted by the engineered enucleated erythroid cell into a regulatory T cell (Treg); and/or inducing the differentiation of CD8⁺ T cell that is contacted by the engineered enucleated erythroid cell into a Treg.

In some embodiments, the exogenous HLA-G polypeptide is bound (e.g., covalently or non-covalently bound) to an exogenous antigenic polypeptide (e.g., an exogenous antigenic polypeptide comprises the motif XI/LPXXXXXL (SEQ ID NO:1)). In some embodiments, the exogenous antigenic polypeptide comprises or consists of an amino acid sequence selected from RIIPRHLQL (SEQ ID NO: 842), KLPAQFYIL (SEQ ID NO: 843), or KGPPAALTL (SEQ ID NO: 844). In some embodiments, the exogenous antigenic polypeptide is between about 8 amino acids in length and about 24 amino acids in length.

In some embodiments, the exogenous HLA-G polypeptide comprises one or more alpha domains of an HLA-G alpha chain, or a fragment thereof, and a β2M polypeptide, or a fragment thereof. In some embodiments, the exogenous HLA-G polypeptide is linked to a membrane anchor. In some embodiments, the exogenous HLA-G polypeptide is a single chain fusion protein comprising an exogenous antigenic polypeptide linked to the exogenous HLA-G polypeptide via a linker (e.g., a cleavable linker), and optionally comprises a membrane anchor. In some embodiments, the membrane anchor comprises a glycophorin A (GPA) protein, or a transmembrane domain thereof; a small integral membrane protein 1 (SMIM1), or a transmembrane domain thereof; or a transferrin receptor or a transmembrane domain thereof.

In some embodiments, the exogenous immunogenic polypeptide is not bound to the exogenous HLA-G polypeptide.

In some embodiments of any of the engineered enucleated erythroid cells described herein, the engineered enucleated erythroid cell further comprises an exogenous autoantigenic polypeptide. In some embodiments, the exogenous autoantigenic polypeptide is on the cell surface. In some embodiments, the exogenous autoantigenic polypeptide further comprises a membrane anchor or is tethered to the plasma membrane of the cell via attachment to a lipid moiety. In some embodiments, the exogenous antigenic polypeptide comprises Formula I in an N-terminal to a C-terminal direction: X₁-X₂-X₃ (Formula I), where: X₁ comprises a type II membrane protein or a transmembrane domain thereof; X₂ comprises a Ii key peptide; and X₃ comprises an autoantigen. In some embodiments, the exogenous autoantigenic polypeptide comprises Formula II in an N-terminal to C-terminal direction: X₁-X₂-X₃-X₄ Formula II), where: X₁ comprises a type II membrane protein or a transmembrane domain thereof; X₂ comprises a linker; X₃ comprises a Ii key peptide; and X₄ comprises an autoantigen. In some embodiments, the linker is a polyGS linker. In some embodiments, the linker comprises GSGSGSGSGSGSGSGSGS (SEQ ID NO: 840) or GPGPG (SEQ ID NO: 841). In some embodiments, the Ii key peptide comprises a sequence selected from the group of: LRMKLPKPPKPVSKMR (SEQ ID NO: 765); YRMKLPKPPKPVSKMR (SEQ ID NO: 766); LRMK (SEQ ID NO: 767); YRMK (SEQ ID NO: 768); LRMKLPK (SEQ ID NO: 769); YRMKLPK (SEQ ID NO: 770); YRMKLPKP (SEQ ID NO: 771); LRMKLPKP (SEQ ID NO: 772); LRMKLPKS (SEQ ID NO: 773); YRMKLPKS (SEQ ID NO: 774); LRMKLPKSAKP (SEQ ID NO: 775); and LRMKLPKSAKPVSK (SEQ ID NO: 776). In some embodiments, the exogenous autoantigenic polypeptide further comprises, at its C-terminus, one or more additional autoantigens. In some embodiments, any two autoantigens are separated by a linker. In some embodiments, the linker is a polyGS linker. In some embodiments, the linker comprises

(SEQ ID NO: 840)
GSGSGSGSGSGSGSGSGS
or
(SEQ ID NO: 841)
GPGPG.

In some embodiments, the exogenous autoantigenic polypeptide is within the cell. In some embodiments, the exogenous autoantigenic polypeptide is on the intracellular side of the plasma membrane. In some embodiments, the exogenous autoantigenic polypeptide further comprises a membrane anchor or is tethered to the plasma membrane of the cell via attachment to a lipid moiety. In some embodiments, the exogenous autoantigenic polypeptide comprises Formula III in an N-terminal to a C-terminal direction: X₁-X₂-X₃ (Formula III), where: X₁ comprises a type I membrane protein or a transmembrane domain thereof; X₂ comprises a Ii key peptide; and X₃ comprises an autoantigen. In some embodiments, the exogenous autoantigenic polypeptide comprises Formula IV: X₁-X₂-X₃-X₄ (Formula IV), where: X₁ comprises a type I membrane protein or a transmembrane domain thereof; X₂ comprises a linker; X₃ comprises a Ii key peptide; and X₄ comprises an autoantigen. In some embodiments, the linker is a polyGS linker. In some embodiments, the polyGS linker comprises GSGSGSGSGSGSGSGSGS (SEQ ID NO: 840) or GPGPG (SEQ ID NO: 841). In some embodiments, the exogenous autoantigenic polypeptide further comprises, at its N-terminus, a signal peptide. In some embodiments, the signal peptide is a GPA signal peptide. In some embodiments, the Ii key peptide is selected from the group of: LRMKLPKPPKPVSKMR (SEQ ID NO: 765); YRMKLPKPPKPVSKMR (SEQ ID NO: 766); LRMK (SEQ ID NO: 767); YRMK (SEQ ID NO: 768); LRMKLPK (SEQ ID NO: 769); YRMKLPK (SEQ ID NO: 770); YRMKLPKP (SEQ ID NO: 771); LRMKLPKP (SEQ ID NO: 772); LRMKLPKS (SEQ ID NO: 773); YRMKLPKS (SEQ ID NO: 774); LRMKLPKSAKP (SEQ ID NO: 775); and LRMKLPKSAKPVSK (SEQ ID NO: 776). In some embodiments, the exogenous autoantigenic polypeptide further comprises, at its C-terminus, one or more additional autoantigens. In some embodiments, any two autoantigens are separated by a linker. In some embodiments, the linker is a polyGS linker. In some embodiments, the linker comprises GSGSGSGSGSGSGSGSGS (SEQ ID NO: 840) or GPGPG (SEQ ID NO: 841).

In some embodiments, the exogenous autoantigenic polypeptide comprises Formula VII in an N-terminal to C-terminal direction: X₁-X₂-X₃-X₄ (Formula VII), where: X₁ comprises a type I membrane protein or a transmembrane domain thereof; X₂ comprises a linker; X₃ comprises a cytoplasmic portion of CD74 or a fragment thereof; and X₄ comprises an autoantigen. In some embodiments, the linker comprises GSGSGSGSGSGSGSGSGS (SEQ ID NO: 840). In some embodiments, the cytoplasmic portion of CD74 comprises

(SEQ ID NO: 845)
QQQGRLDKLTVTSQNLQLENLRMKLPKPPKPVSKMRMATPLLMQALPMGA
LPQGPMQNATKYGNMTEDHVMHLLQNADPLKVYPPLKGSFPENLRHLKNT
METIDWKVFESWMHHWLLFEMSRHSLEQKPTDAPPKESLELEDPSSGLGV
TKQDLGPVPM.

In some embodiments, the N-terminus of the exogenous autoantigenic polypeptide further comprises a signal peptide.

In some embodiments, the exogenous autoantigenic polypeptide comprises Formula VIII in an N-terminal to C-terminal direction: X₁-X₂-X₃-X₄-X₅ (Formula VIII), where: X₁ comprises a type I membrane protein or a transmembrane domain thereof; X₂ comprises a linker; X₃ comprises a N-terminal cytoplasmic portion of CD74 or a fragment thereof; X₄ comprises an autoantigen; and X₅ comprises a C-terminal cytoplasmic portion of CD74. In some embodiments, the linker comprises GSGSGSGSGSGSGSGSGS (SEQ ID NO: 840). In some embodiments, the N-terminal cytoplasmic portion of CD74 comprises QQQGRLDKLTVTSQNLQLENLRMK (SEQ ID NO: 847). In some embodiments, the C-terminal cytoplasmic portion of CD74 comprises GALPQGPMQNATKYGNMTEDHVMHLLQNADPLKVYPPLKGSFPENLRHLKNTMETID WKVFESWMHHWLLFEMSRHSLEQKPTDAPPKESLELEDPSSGLGVTKQDLGPVPM (SEQ ID NO: 849). In some embodiments, the N-terminus of the exogenous autoantigenic polypeptide further comprises a signal peptide.

In some embodiments, the exogenous autoantigenic polypeptide comprises Formula XI in an N-terminal to C-terminal direction: X₁-X₂-X₃-X₄ (Formula XI), where: X₁ comprises a cytosolic protein or a fragment thereof; X₂ comprises a linker; X₃ comprises a cytoplasmic portion of CD74 or a fragment thereof; and X₄ comprises an autoantigen. In some embodiments, the cytosolic protein comprises MAGWNAYIDNLMADGTCQDAAIVGYKDSPSVWAAVPGKTFVNITPAEVGVLVGKDRS SFYVNGLTLGGQKCSVIRDSLLQDGEF SMDLRTKSTGGAPTFNVTVTKTDKTLVLLMG KEGVHGGLINKKCYEMASHLRRSQY (SEQ ID NO: 846). In some embodiments, the linker comprises GSGSGSGSGSGSGSGSGS (SEQ ID NO: 840). In some embodiments, the cytoplasmic portion of CD74 comprises

(SEQ ID NO: 845)
QQQGRLDKLTVTSQNLQLENLRMKLPKPPKPVSKMRMATPLLMQALPMGA
LPQGPMQNATKYGNMTEDHVMHLLQNADPLKVYPPLKGSFPENLRHLKNT
METIDWKVFESWMHHWLLFEMSRHSLEQKPTDAPPKESLELEDPSSGLGV
TKQDLGPVPM.

In some embodiments, the N-terminus of the exogenous autoantigenic polypeptide further comprises a signal peptide.

In some embodiments, the exogenous autoantigenic polypeptide comprises Formula XII in an N-terminal to C-terminal direction: X₁-X₂-X₃-X₄-X₅ (Formula XII), where: X₁ comprises a cytoplasmice protein or a fragment thereof; X₂ comprises a linker; X₃ comprises a N-terminal cytoplasmic portion of CD74 or a fragment thereof; X₄ comprises an autoantigen; and X₅ comprises a C-terminal cytoplasmic portion of CD74. In some embodiments, the cytoplasmic protein comprises MAGWNAYIDNLMADGTCQDAAIVGYKDSPSVWAAVPGKTFVNITPAEVGVLVGKDRS SFYVNGLTLGGQKCSVIRDSLLQDGEFSMDLRTKSTGGAPTFNVTVTKTDKTLVLLMG KEGVHGGLINKKCYEMASHLRRSQY (SEQ ID NO: 846). In some embodiments, the linker comprises GSGSGSGSGSGSGSGSGS (SEQ ID NO: 840). In some embodiments, the N-terminal cytoplasmic portion of CD74 comprises: QQQGRLDKLTVTSQNLQLENLRMK (SEQ ID NO: 847). In some embodiments, the C-terminal cytoplasmic portion of CD74 comprises: GALPQGPMQNATKYGNMTEDHVMHLLQNADPLKVYPPLKGSFPENLRHLKNTMETID WKVFESWMHHWLLFEMSRHSLEQKPTDAPPKESLELEDPSSGLGVTKQDLGPVPM (SEQ ID NO: 849). In some embodiments, the N-terminus of the exogenous autoantigenic polypeptide further comprises a signal peptide.

In some embodiments, the exogenous autoantigenic polypeptide is present on the cell surface. In some embodiments, the exogenous autoantigenic polypeptide comprises Formula IX in an N-terminal to C-terminal direction: X₁-X₂-X₃ (Formula IX), where: X₁ comprises a type II membrane protein or a transmembrane domain thereof; X₂ comprises a cytoplasmic portion of CD74 or a fragment thereof; and X₃ comprises an autoantigen. In some embodiments, the cytoplasmic portion of CD74 comprises

(SEQ ID NO: 845)
QQQGRLDKLTVTSQNLQLENLRMKLPKPPKPVSKMRMATPLLMQALPMGA
LPQGPMQNATKYGNMTEDHVMHLLQNADPLKVYPPLKGSFPENLRHLKNT
METIDWKVFESWMHHWLLFEMSRHSLEQKPTDAPPKESLELEDPSSGLGV
TKQDLGPVPM.

In some embodiments, the N-terminus of the exogenous autoantigenic polypeptide further comprises a signal peptide.

In some embodiments, the exogenous autoantigenic polypeptide comprises Formula X in an N-terminal to C-terminal direction: X₁-X₂-X₃-X₄-X₅ (Formula X), where: X₁ comprises a type II membrane protein or a transmembrane domain thereof; X₂ comprises a linker; X₃ comprises a N-terminal cytoplasmic portion of CD74 or a fragment thereof; X₄ comprises an autoantigen; and X₅ comprises a C-terminal cytoplasmic portion of CD74. In some embodiments, the linker comprises GGGGSGGGGSGGGGSGGGGS (SEQ ID NO: 850). In some embodiments, the N-terminal cytoplasmic portion of CD74 comprises QQQGRLDKLTVTSQNLQLENLRMK (SEQ ID NO: 847). In some embodiments, the C-terminal cytoplasmic portion of CD74 comprises ALPQGPMQNATKYGNMTEDHVMHLLQNADPLKVYPPLKGSFPENLRHLKNTMETIDW KVFESWMHHWLLFEMSRHSLEQKPTDAPPKESLELEDPSSGLGVTKQDLGPVPM (SEQ ID NO: 848). In some embodiments, the N-terminus of the exogenous autoantigenic polypeptide further comprises a signal peptide.

In some embodiments, the exogenous autoantigenic polypeptide comprises Formula XIII in an N-terminal to C-terminal direction: X₁-X₂-X₃-X₄ (Formula XIII), where: X₁ comprises an Ii key peptide; X₂ comprises an autoantigen; X₃ comprises a linker; and X₄ comprises a Type I membrane protein or a transmembrane domain thereof. In some embodiments, the linker comprises GPGPG (SEQ ID NO: 841). In some embodiments, X₁ comprises two or more (e.g., three, four, five, or six) Ii key peptides. In some embodiments, the N-terminus of the exogenous autoantigenic polypeptide further comprises a signal peptide.

In some embodiments, the exogenous autoantigenic polypeptide is in the cytosol of the cell. In some embodiments, the exogenous autoantigenic polypeptide comprises Formula V in an N-terminal to a C-terminal direction: X₁-X₂-X₃ (Formula V), where: X₁ comprises a cytosolic polypeptide or a fragment thereof; X₂ comprises a Ii key peptide; and X₃ comprises an autoantigen. In some embodiments, the exogenous autoantigenic polypeptide comprises Formula VI in an N-terminal to a C-terminal direction: X₁-X₂-X₃-X₄ (Formula VI), where: X₁ comprises a cytosolic polypeptide or a fragment thereof; X₂ comprises a linker; X₃ comprises a Ii key peptide; and X₄ comprises an autoantigen. In some embodiments, the linker is a polyGS linker. In some embodiments, the linker comprises GSGSGSGSGSGSGSGSGS (SEQ ID NO: 840) or GPGPG (SEQ ID NO: 841). In some embodiments, the cytosolic polypeptide comprises profilin or a fragment thereof. In some embodiments, the cytosolic polypeptide comprises ferritin or a fragment thereof. In some embodiments, the Ii key peptide is selected from the group of: LRMKLPKPPKPVSKMR (SEQ ID NO: 765); YRMKLPKPPKPVSKMR (SEQ ID NO: 766); LRMK (SEQ ID NO: 767); YRMK (SEQ ID NO: 768); LRMKLPK (SEQ ID NO: 769); YRMKLPK (SEQ ID NO: 770); YRMKLPKP (SEQ ID NO: 771); LRMKLPKP (SEQ ID NO: 772); LRMKLPKS (SEQ ID NO: 773); YRMKLPKS (SEQ ID NO: 774); LRMKLPKSAKP (SEQ ID NO: 775); and LRMKLPKSAKPVSK (SEQ ID NO: 776). In some embodiments, the exogenous autoantigenic polypeptide further comprises, at its C-terminus, one or more additional autoantigens. In some embodiments, any two autoantigens are separated by a linker. In some embodiments, the linker is a polyGS linker. In some embodiments, the linker comprises GSGSGSGSGSGSGSGSGS (SEQ ID NO: 840) or GPGPG (SEQ ID NO: 841).

In some embodiments of any of the engineered enucleated erythroid cells described herein, the engineered enucleated erythroid cell further comprises at least one exogenous coinhibitory polypeptide. In some embodiments, one of the at least one exogenous coinhibitory polypeptide(s) is on the cell surface. In some embodiments, one of the least one exogenous coinhibitory polypeptide(s) further comprises a transmembrane domain. In some embodiments, the transmembrane domain is a glycophorin A (GPA) transmembrane domain, a small integral membrane protein 1 (SMIM1) transmembrane domain, or a transferrin receptor transmembrane domain. In some embodiments, one of the at least one exogenous coinhibitory polypeptide(s) is within the cell. In some embodiments, one of the at least one exogenous coinhibitory polypeptide(s) is secreted/released by the cell.

In some embodiments, the at least one exogenous coinhibitory polypeptide is/are selected from the group consisting of: IL-10, IL-27, IL-37, TGFβ, CD39, CD73, arginase 1 (ARG1), annexin 1, fibrinogen-like protein 2 (FGL2), and PD-L1. In some embodiments, the at least one exogenous coinhibitory polypeptide is IL-10, and comprises an amino acid sequence that is at least 90%, 92%, 94%, 95%, 96%, 97%, 98%, or 99% identical to SEQ ID NO: 760, 761, 762, or 763. In some embodiments, the at least one exogenous coinhibitory polypeptide is PD-L1, and comprises an amino acid sequence that is at least 90%, 92%, 94%, 95%, 96%, 97%, 98%, or 99% identical to SEQ ID NO: 764.

In some embodiments of any of the engineered erythroid cells described herein, the engineered erythroid cell has been treated to increased presence of phosphatidylserine on the outer leaflet of the plasma membrane. In some embodiments, the engineered erythroid cell has been treated with a calcium ionophore. In some embodiments, the engineered erythroid cell has been treated with one or more of ionomycin, A23187, and BS3.

In some embodiments of any of the engineered enucleated erythroid cells described herein, one of the at least one the exogenous coinhibitory polypeptide comprises a sequence that is at least 90% identical to any one of SEQ ID NOs: 760-764 and 816-823.

Also provided herein are engineered enucleated erythroid cells that include an exogenous autoantigenic polypeptide and at least one exogenous coinhibitory polypeptide. In some embodiments, the exogenous autoantigenic polypeptide is on the cell surface. In some embodiments, the exogenous autoantigenic polypeptide further comprises a membrane anchor or is tethered to the plasma membrane of the cell via attachment to a lipid moiety. In some embodiments, the exogenous autoantigenic polypeptide comprises Formula I in an N-terminal to a C-terminal direction: X₁-X₂-X₃ (Formula I), where: X₁ comprises a type II membrane protein or a transmembrane domain thereof; X₂ comprises a Ii key peptide; and X₃ comprises an autoantigen. In some embodiments, the exogenous autoantigenic polypeptide comprises Formula II in an N-terminal to a C-terminal direction: X₁-X₂-X₃-X₄ (Formula II), where: X₁ comprises a type II membrane protein or a transmembrane domain thereof; X₂ comprises a linker; X₃ comprises a Ii key peptide; and X₄ comprises an autoantigen. In some embodiments, the linker is a polyGS linker. In some embodiments, the linker comprises GSGSGSGSGSGSGSGSGS (SEQ ID NO: 840) or GPGPG (SEQ ID NO: 841). In some embodiments, the Ii key peptide comprises a sequence selected from the group of: LRMKLPKPPKPVSKMR (SEQ ID NO: 765); YRMKLPKPPKPVSKMR (SEQ ID NO: 766); LRMK (SEQ ID NO: 767); YRMK (SEQ ID NO: 768); LRMKLPK (SEQ ID NO: 769); YRMKLPK (SEQ ID NO: 770); YRMKLPKP (SEQ ID NO: 771); LRMKLPKP (SEQ ID NO: 772); LRMKLPKS (SEQ ID NO: 773); YRMKLPKS (SEQ ID NO: 774); LRMKLPKSAKP (SEQ ID NO: 775); and LRMKLPKSAKPVSK (SEQ ID NO: 776). In some embodiments, the exogenous autoantigenic polypeptide further comprises, at its C-terminus, one or more additional autoantigens. In some embodiments, any two autoantigens are separated by a linker. In some embodiments, the linker is a polyGS linker. In some embodiments, the linker comprises

(SEQ ID NO: 840)
GSGSGSGSGSGSGSGSGS
or
(SEQ ID NO: 841)
GPGPG.

In some embodiments, the exogenous autoantigenic polypeptide is within the cell. In some embodiments, the exogenous autoantigenic polypeptide is on the intracellular side of the plasma membrane. In some embodiments, the exogenous antigenic polypeptide further comprises a membrane anchor or is tethered to the plasma membrane of the cell via attachment to a lipid moiety. In some embodiments, the exogenous autoantigenic polypeptide comprises Formula III in an N-terminal to a C-terminal direction: X₁-X₂-X₃ (Formula III), where: X₁ comprises a type I membrane protein or a transmembrane domain thereof; X₂ comprises a Ii key peptide; and X₃ comprises an autoantigen. In some embodiments, the exogenous autoantigenic polypeptide comprises Formula IV in an N-terminal to C-terminal direction: X₁-X₂-X₃-X₄ (Formula IV), where: X₁ comprises a type I membrane protein or a transmembrane domain thereof; X₂ comprises a linker; X₃ comprises a Ii key peptide; and X₄ comprises an autoantigen. In some embodiments, the linker is a polyGS linker. In some embodiments, the linker comprises GSGSGSGSGSGSGSGSGS (SEQ ID NO: 840) or GPGPG (SEQ ID NO: 841). In some embodiments, the exogenous autoantigenic polypeptide further comprises, at its N-terminus, a signal peptide. In some embodiments, the signal peptide is a GPA signal peptide. In some embodiments, the Ii key peptide is selected from the group of: LRMKLPKPPKPVSKMR (SEQ ID NO: 765); YRMKLPKPPKPVSKMR (SEQ ID NO: 766); LRMK (SEQ ID NO: 767); YRMK (SEQ ID NO: 768); LRMKLPK (SEQ ID NO: 769); YRMKLPK (SEQ ID NO: 770); YRMKLPKP (SEQ ID NO: 771); LRMKLPKP (SEQ ID NO: 772); LRMKLPKS (SEQ ID NO: 773); YRMKLPKS (SEQ ID NO: 774); LRMKLPKSAKP (SEQ ID NO: 775); and LRMKLPKSAKPVSK (SEQ ID NO: 776). In some embodiments, the exogenous autoantigenic polypeptide further comprises, at its C-terminus, one or more additional autoantigens. In some embodiments, any two autoantigens are separated by a linker. In some embodiments, the linker is a polyGS linker. In some embodiments, the linker comprises

(SEQ ID NO: 840)
GSGSGSGSGSGSGSGSGS
or
(SEQ ID NO: 841)
GPGPG.

In some embodiments, the exogenous autoantigenic polypeptide comprises Formula VII in an N-terminal to C-terminal direction: X₁-X₂-X₃-X₄ (Formula VII), where: X₁ comprises a type I membrane protein or a transmembrane domain thereof; X₂ comprises a linker; X₃ comprises a cytoplasmic portion of CD74 or a fragment thereof; and X₄ comprises an autoantigen. In some embodiments, the linker comprises GSGSGSGSGSGSGSGSGS (SEQ ID NO: 840). In some embodiments, the cytoplasmic portion of CD74 comprises

(SEQ ID NO: 845)
QQQGRLDKLTVTSQNLQLENLRMKLPKPPKPVSKMRMATPLLMQALPMGA
LPQGPMQNATKYGNMTEDHVMHLLQNADPLKVYPPLKGSFPENLRHLKNT
METIDWKVFESWMHHWLLFEMSRHSLEQKPTDAPPKESLELEDPSSGLGV
TKQDLGPVPM.

In some embodiments, the N-terminus of the exogenous autoantigenic polypeptide further comprises a signal peptide.

In some embodiments, the exogenous autoantigenic polypeptide comprises Formula VIII in an N-terminal to C-terminal direction: X₁-X₂-X₃-X₄-X₅ (Formula VIII), where: X₁ comprises a type I membrane protein or a transmembrane domain thereof; X₂ comprises a linker; X₃ comprises a N-terminal cytoplasmic portion of CD74 or a fragment thereof; X₄ comprises an autoantigen; and X₅ comprises a C-terminal cytoplasmic portion of CD74. In some embodiments, the linker comprises GSGSGSGSGSGSGSGSGS (SEQ ID NO: 840). In some embodiments, the N-terminal cytoplasmic portion of CD74 comprises QQQGRLDKLTVTSQNLQLENLRMK (SEQ ID NO: 847). In some embodiments, the C-terminal cytoplasmic portion of CD74 comprises GALPQGPMQNATKYGNMTEDHVMHLLQNADPLKVYPPLKGSFPENLRHLKNTMETID WKVFESWMHHWLLFEMSRHSLEQKPTDAPPKESLELEDPSSGLGVTKQDLGPVPM (SEQ ID NO: 849). In some embodiments, the N-terminus of the exogenous autoantigenic polypeptide further comprises a signal peptide.

In some embodiments, the exogenous autoantigenic polypeptide comprises Formula XI in an N-terminal to C-terminal direction: X₁-X₂-X₃-X₄ (Formula XI), where: X₁ comprises a cytosolic protein or a fragment thereof; X₂ comprises a linker; X₃ comprises a cytoplasmic portion of CD74 or a fragment thereof; and X₄ comprises an autoantigen. In some embodiments, the cytosolic protein comprises MAGWNAYIDNLMADGTCQDAAIVGYKDSPSVWAAVPGKTFVNITPAEVGVLVGKDRS SFYVNGLTLGGQKCSVIRDSLLQDGEFSMDLRTKSTGGAPTFNVTVTKTDKTLVLLMG KEGVHGGLINKKCYEMASHLRRSQY (SEQ ID NO: 846). In some embodiments, the linker comprises GSGSGSGSGSGSGSGSGS (SEQ ID NO: 840). In some embodiments, the cytoplasmic portion of CD74 comprises

(SEQ ID NO: 845)
QQQGRLDKLTVTSQNLQLENLRMKLPKPPKPVSKMRMATPLLMQALPMGA
LPQGPMQNATKYGNMTEDHVMHLLQNADPLKVYPPLKGSFPENLRHLKNT
METIDWKVFESWMHHWLLFEMSRHSLEQKPTDAPPKESLELEDPSSGLGV
TKQDLGPVPM.

In some embodiments, the N-terminus of the exogenous autoantigenic polypeptide further comprises a signal peptide.

In some embodiments, the exogenous autoantigenic polypeptide comprises Formula XII in an N-terminal to C-terminal direction: X₁-X₂-X₃-X₄-X₅ (Formula XII), where: X₁ comprises a cytoplasmice protein or a fragment thereof; X₂ comprises a linker; X₃ comprises a N-terminal cytoplasmic portion of CD74 or a fragment thereof; X₄ comprises an autoantigen; and X₅ comprises a C-terminal cytoplasmic portion of CD74. In some embodiments, the cytoplasmic protein comprises MAGWNAYIDNLMADGTCQDAAIVGYKDSPSVWAAVPGKTFVNITPAEVGVLVGKDRS SFYVNGLTLGGQKCSVIRDSLLQDGEFSMDLRTKSTGGAPTFNVTVTKTDKTLVLLMG KEGVHGGLINKKCYEMASHLRRSQY (SEQ ID NO: 846). In some embodiments, the linker comprises GSGSGSGSGSGSGSGSGS (SEQ ID NO: 840). In some embodiments, the N-terminal cytoplasmic portion of CD74 comprises: QQQGRLDKLTVTSQNLQLENLRMK (SEQ ID NO: 847). In some embodiments, the C-terminal cytoplasmic portion of CD74 comprises: GALPQGPMQNATKYGNMTEDHVMHLLQNADPLKVYPPLKGSFPENLRHLKNTMETID WKVFESWMHHWLLFEMSRHSLEQKPTDAPPKESLELEDPSSGLGVTKQDLGPVPM (SEQ ID NO: 849). In some embodiments, the N-terminus of the exogenous autoantigenic polypeptide further comprises a signal peptide.

In some embodiments, the exogenous autoantigenic polypeptide is present on the cell surface. In some embodiments, the exogenous autoantigenic polypeptide comprises Formula IX in an N-terminal to C-terminal direction: X₁-X₂-X₃ (Formula IX), where: X₁ comprises a type II membrane protein or a transmembrane domain thereof; X₂ comprises a cytoplasmic portion of CD74 or a fragment thereof; and X₃ comprises an autoantigen. In some embodiments, the cytoplasmic portion of CD74 comprises

(SEQ ID NO: 845)
QQQGRLDKLTVTSQNLQLENLRMKLPKPPKPVSKMRMATPLLMQALPMGA
LPQGPMQNATKYGNMTEDHVMHLLQNADPLKVYPPLKGSFPENLRHLKNT
METIDWKVFESWMHHWLLFEMSRHSLEQKPTDAPPKESLELEDPSSGLGV
TKQDLGPVPM.

In some embodiments, the N-terminus of the exogenous autoantigenic polypeptide further comprises a signal peptide.

In some embodiments, the exogenous autoantigenic polypeptide comprises Formula X in an N-terminal to C-terminal direction: X₁-X₂-X₃-X₄-X₅ (Formula X), where: X₁ comprises a type II membrane protein or a transmembrane domain thereof; X₂ comprises a linker; X₃ comprises a N-terminal cytoplasmic portion of CD74 or a fragment thereof; X₄ comprises an autoantigen; and X₅ comprises a C-terminal cytoplasmic portion of CD74. In some embodiments, the linker comprises GGGGSGGGGSGGGGSGGGGS (SEQ ID NO: 850). In some embodiments, the N-terminal cytoplasmic portion of CD74 comprises QQQGRLDKLTVTSQNLQLENLRMK (SEQ ID NO: 847). In some embodiments, the C-terminal cytoplasmic portion of CD74 comprises ALPQGPMQNATKYGNMTEDHVMHLLQNADPLKVYPPLKGSFPENLRHLKNTMETIDW KVFESWMHHWLLFEMSRHSLEQKPTDAPPKESLELEDPSSGLGVTKQDLGPVPM (SEQ ID NO: 848). In some embodiments, the N-terminus of the exogenous autoantigenic polypeptide further comprises a signal peptide.

In some embodiments, the exogenous autoantigenic polypeptide comprises Formula XIII in an N-terminal to C-terminal direction: X₁-X₂-X₃-X₄ (Formula XIII), where: X₁ comprises an Ii key peptide; X₂ comprises an autoantigen; X₃ comprises a linker; and X₄ comprises a Type I membrane protein or a transmembrane domain thereof. In some embodiments, the linker comprises GPGPG (SEQ ID NO: 841). In some embodiments, X₁ comprises two or more (e.g., three, four, five, or six) Ii key peptides. In some embodiments, the N-terminus of the exogenous autoantigenic polypeptide further comprises a signal peptide.

In some embodiments, the exogenous autoantigenic polypeptide is in the cytosol of the cell. In some embodiments, the exogenous autoantigenic polypeptide comprises Formula V in an N-terminal to a C-terminal direction: X₁-X₂-X₃ (Formula V), where: X₁ comprises a cytosolic polypeptide or a fragment thereof; X₂ comprises a Ii key peptide; and X₃ comprises an autoantigen. In some embodiments, the exogenous autoantigenic polypeptide comprises Formula VI: X₁-X₂-X₃-X₄ (Formula VI), where: X₁ comprises a cytosolic polypeptide or a fragment thereof; X₂ comprises a linker; X₃ comprises a Ii key peptide; and X₄ comprises an autoantigen. In some embodiments, the linker is a polyGS linker. In some embodiments, the linker comprises GSGSGSGSGSGSGSGSGS (SEQ ID NO: 840) or GPGPG (SEQ ID NO: 841). In some embodiments, the cytosolic polypeptide comprises profilin or a fragment thereof. In some embodiments, the cytosolic polypeptide comprises ferritin or a fragment thereof. In some embodiments, the Ii key peptide is selected from the group of: LRMKLPKPPKPVSKMR (SEQ ID NO: 765); YRMKLPKPPKPVSKMR (SEQ ID NO: 766); LRMK (SEQ ID NO: 767); YRMK (SEQ ID NO: 768); LRMKLPK (SEQ ID NO: 769); YRMKLPK (SEQ ID NO: 770); YRMKLPKP (SEQ ID NO: 771); LRMKLPKP (SEQ ID NO: 772); LRMKLPKS (SEQ ID NO: 773); YRMKLPKS (SEQ ID NO: 774); LRMKLPKSAKP (SEQ ID NO: 775); and LRMKLPKSAKPVSK (SEQ ID NO: 776). In some embodiments, the exogenous autoantigenic polypeptide further comprises, at its C-terminus, one or more additional autoantigens. In some embodiments, any two autoantigens are separated by a linker. In some embodiments, the linker is a polyGS linker. In some embodiments, the linker comprises

(SEQ ID NO: 840)
GSGSGSGSGSGSGSGSGS
or
(SEQ ID NO: 841)
GPGPG.

In some embodiments of any of the engineered enucleated cells described herein, the engineered enucleated erythroid cell further comprises at least one exogenous coinhibitory polypeptide. In some embodiments, one of the at least one exogenous coinhibitory polypeptide(s) is on the cell surface. In some embodiments, one of the least one exogenous coinhibitory polypeptide(s) further comprises a transmembrane domain. In some embodiments, the transmembrane domain is a glycophorin A (GPA) transmembrane domain, a small integral membrane protein 1 (SMIM1) transmembrane domain, or a transferrin receptor transmembrane domain.

In some embodiments, one of the at least one exogenous coinhibitory polypeptide(s) is within the cell. In some embodiments, one of the at least one exogenous coinhibitory polypeptide(s) is secreted/released by the cell.

In some embodiments, the at least one exogenous coinhibitory polypeptide is/are selected from the group consisting of: IL-10, IL-27, IL-37, TGFβ, CD39, CD73, arginase 1 (ARG1), annexin 1, fibrinogen-like protein 2 (FGL2), and PD-L1. In some embodiments, one of the at least one exogenous coinhibitory polypeptide is IL-10, and comprises an amino acid sequence that is at least 90%, 92%, 94%, 95%, 96%, 97%, 98%, or 99% identical to SEQ ID NO: 760, 761, 762, or 763. In some embodiments, one of the at least one exogenous coinhibitory polypeptide is PD-L1, and comprises an amino acid sequence that is at least 90%, 92%, 94%, 95%, 96%, 97%, 98%, or 99% identical to SEQ ID NO: 764.

In some embodiments of any of the engineered enucleated erythroid cells described herein, the engineered erythroid cell has been treated to increased presence of phosphatidylserine on the outer leaflet of the plasma membrane. In some embodiments, the engineered erythroid cell has been treated with a calcium ionophore. In some embodiments, the engineered erythroid cell has been treated with one or more of ionomycin, A23187, and BS3.

In some embodiments, one of the at least one the exogenous coinhibitory polypeptide comprises a sequence that is at least 90% identical to any one of SEQ ID NOs: 760-764 and 816-832.

In some embodiments, the engineered enucleated erythroid cell is a reticulocyte or an erythrocyte.

In some embodiments, the engineered enucleated erythroid cell is a human cell.

In another aspect, the disclosure provides pharmaceutical compositions comprising a plurality of the engineered enucleated erythroid cells described herein, and a pharmaceutically acceptable carrier.

In another aspect, the disclosure provides methods of inducing immune tolerance in a subject to an exogenous immunogenic polypeptide, the methods comprising administering to the subject a plurality of the engineered enucleated erythroid cells described herein, or the pharmaceutical compositions described herein, thereby inducing immune tolerance to the exogenous immunogenic polypeptide.

In some embodiments, the immune tolerance comprises short-term immune tolerance. In some embodiments, the short-term immune tolerance comprises inducing apoptosis or inhibiting the activation, differentiation, and/or proliferation of an immune cell that is contacted by the engineered enucleated erythroid cell, and optionally, wherein the immune cell is a T cell, a NK cell, or a B cell. In some embodiments, the short-term immune tolerance comprises inhibiting the cytotoxicity of a T cell or of a NK cell that is contacted by the engineered enucleated erythroid cell. In some embodiments, the short-term immune tolerance comprises inhibiting antibody secretion by a B cell that is contacted by the engineered enucleated erythroid cell.

In some embodiments, the immune tolerance comprises long-term immune tolerance. In some embodiments, the long-term immune tolerance comprises inhibiting the maturation of a DC that is contacted by the engineered enucleated erythroid cell. In some embodiments, the long-term immune tolerance comprises inducing anergy of a DC that is contacted by the engineered enucleated erythroid cell. In some embodiments, the long-term immune tolerance comprises: inducing the differentiation of CD4⁺ T cell that is contacted by the engineered enucleated erythroid cell into a Treg; and/or inducing the differentiation of CD8⁺ T cell that is contacted by the engineered enucleated erythroid cell into a Treg.

In other aspects, the disclosure provides methods of treating a disease in a subject in need thereof, the method comprising administering to the subject (e.g., intravenously) a plurality of the engineered enucleated erythroid cells described herein, or the pharmaceutical compositions described, thereby treating the disease in the subject.

In some embodiments, an immune response in the subject to the exogenous immunogenic polypeptide is reduced as compared to the immune response in the subject to the exogenous immunogenic polypeptide when the exogenous immunogenic polypeptide is administered to the subject alone; and/or an immune response in the subject to the exogenous immunogenic polypeptide is reduced as compared to the immune response in the subject to the exogenous immunogenic polypeptide when the exogenous immunogenic polypeptide is administered to the subject when present on the surface of a plurality of engineered enucleated erythroid cells lacking the exogenous HLA-G polypeptide.

In some embodiments, the disease is a cancer (e.g., a leukemia). In some embodiments, the disease is a cancer selected from acute lymphoblastic leukemia (ALL), anal cancer, bile duct cancer, bladder cancer, bone cancer, bowel cancer, brain cancer, breast cancer, liver cancer, lung cancer, cancer of unknown primary, cervical cancer, choriocarcinoma, chronic lymphocytic leukemia (CLL), chronic myeloid leukemia (CML), colon cancer, colorectal cancer, endometrial cancer, eye cancer, gallbladder cancer, gastric cancer, gestational trophoblastic tumors (GTT), hairy cell leukemia, head and neck cancer, Hodgkin lymphoma, kidney cancer, laryngeal cancer, leukemia, lymphoma, skin cancer, mesothelioma, mouth and oropharyngeal cancer, myeloma, nasal and sinus cancer, nasopharyngeal cancer, non-Hodgkin lymphoma (NEIL), esophageal cancer, ovarian cancer, pancreatic cancer, penile cancer, prostate cancer, rectal cancer, salivary gland cancer, soft tissue sarcoma, stomach cancer, testicular cancer, thyroid cancer, uterine cancer, vaginal cancer, or vulvar cancer.

In some embodiments, the disease is a homocysteine-related disease (e.g., homocystinuria). In some embodiments, the disease is a uric acid-related disease (e.g., hyperuricemia, gout, rheumatoid arthritis, osteoarthritis, cerebral stroke, ischemic heart disease, arrhythmia, or chronic renal disease). In some embodiments, the disease is hyperoxaluria. In some embodiments, the disease is phenylketonuria.

In some embodiments, the disease is an autoimmune disease. In some embodiments, the autoimmune disease is type 1 diabetes, multiple sclerosis, connective tissue disorder, Celiac disease, bullous pemphigoid, membranous glomerulonephritis, neuromyelitis optica, pemphigus vulgaris, autoimmune encephalitis, autoimmune hepatitis, chronic inflammatory demyelinating polyneuropathy (CIPD), polymyositis and dermatomyositis (PM/DM), mixed connective tissue disease (MCTD), myasthenia gravis, rheumatoid arthritis, autoimmune liver disease, uveitis, autoimmune myocarditis, vitiligo, alopecis areata, or scleroderma. In some embodiments, the autoimmune disease is type 1 diabetes, multiple sclerosis, connective tissue disorder, or Celiac disease. In some embodiments, the autoimmune disease is type 1 diabetes. In some embodiments, the autoimmune disease is bullous pemphigoid, membranous glomerulonephritis, neuromyelitis optica, or pemphigus vulgaris. In some embodiments, the autoimmune disease is autoimmune encephalitis, autoimmune hepatitis, chronic inflammatory demyelinating polyneuropathy (CIPD), polymyositis and dermatomyositis (PM/DM), mixed connective tissue disease (MCTD), myasthenia gravis, rheumatoid arthritis, autoimmune liver disease, uveitis, autoimmune myocarditis, vitiligo, alopecis areata, or scleroderma.

### BRIEF DESCRIPTION OF THE DRAWINGS

The figures are meant to be illustrative of one or more features, aspects, or embodiments of the present disclosure and are not intended to be limiting.

FIGS. 1A-1D are schematic diagrams showing exemplary constructs including an HLA-G polypeptide.

FIG. 1A depicts an erythroid cell comprising exemplary single chain fusion polypeptide comprising an HLA-G polypeptide, as well as two exemplary single chain fusion polypeptides comprising an exogenous β2M polypeptide linked to one or more alpha domains of an HLA-G alpha chain linked to a membrane anchor (e.g., a GPA polypeptide or a transmembrane domain thereof), optionally linked to an exogenous antigenic polypeptide.

FIG. 1B depicts an HLA-G construct which comprises an exogenous antigenic peptide linked to a β2M polypeptide, which is linked to one or more alpha domains of an HLA-G alpha chain (e.g., alpha1, alpha2, and alpha3 domains), which is linked to a membrane anchor, such as GPA, SMIM1, or transferrin receptor.

FIG. 1C depicts an open conformation (OC) construct (e.g., not fused to an exogenous antigenic polypeptide), which comprises a β2M polypeptide linked to one or more alpha domains of an HLA-G alpha chain (e.g., one or more of alpha1, alpha2, and alpha3 domains), which is linked to a membrane anchor, such as GPA, SMIM1, or transferrin receptor, wherein the HLA-G open conformation is capable of binding an exogenous antigenic polypeptide. The construct further includes a β2M leader sequence.

FIG. 1D depicts an HLA-G2 construct, which comprises HLA-G2 alpha1 and alpha2 domains linked to a membrane anchor, such as GPA, SMIM1, or transferrin receptor. The construct further includes a β2M or alpha leader sequence.

### DETAILED DESCRIPTION

The present disclosure describes engineered erythroid cells (e.g., engineered enucleated erythroid cells) or enucleated cells (e.g., modified enucleated cells) that include, on their surface (e.g., on the outer leaflet of the cell plasma membrane), an exogenous HLA-G polypeptide and an exogenous immunogenic polypeptide (e.g., on the cell surface or within the cell (e.g., in the cytosol of the cell or on the intracellular side of the plasma membrane). In some embodiments, the exogenous immunogenic polypeptide is secreted or released by the cell. In some embodiments, the exogenous immunogenic polypeptide is not bound to the exogenous HLA-G polypeptide.

In some embodiments, the engineered erythroid cells (e.g., engineered enucleated erythroid cells) or enucleated cells (e.g., modified enucleated cells) further include an exogenous antigenic polypeptide. In some embodiments, the exogenous HLA-G polypeptide is bound to the exogenous antigenic polypeptide. In some embodiments, the exogenous antigenic polypeptide is not bound to the exogenous HLA-G polypeptide. In some embodiments, the engineered erythroid cells or enucleated cells include an exogenous HLA-G polypeptide and an exogenous immunogenic polypeptide on the cell surface, wherein the exogenous immunogenic polypeptide is not bound to the exogenous HLA-G polypeptide (e.g., is not bound to the antigen-binding cleft of the HLA-G polypeptide). In some embodiments, the engineered erythroid cells or enucleated cells include an exogenous HLA-G polypeptide and an exogenous immunogenic polypeptide within the cell, wherein the exogenous immunogenic polypeptide is not bound to the exogenous HLA-G polypeptide (e.g., is not bound to the antigen-binding cleft of the HLA-G polypeptide).

In some embodiments, the exogenous HLA-G polypeptide is a single chain fusion polypeptide comprising or consisting of the ectodomain of an HLA-G polypeptide (e.g., alpha1, alpha2, and alpha3 domains), a beta-2 microglobulin (β2M) polypeptide, and a membrane anchor (e.g., comprising a GPA transmembrane domain, SMIM1 transmembrane domain, or a transferrin receptor transmembrane domain), wherein the single chain fusion polypeptide is optionally linked to an exogenous antigenic polypeptide. In other embodiments, the exogenous HLA-G polypeptide is a single chain fusion polypeptide comprising an HLA-G polypeptide linked to an exogenous antigenic polypeptide, e.g., comprising the motif XI/LPXXXXXL, wherein X is any amino acid residue (SEQ ID NO: 1). In some embodiments, the exogenous antigenic polypeptide comprises or consists of an amino acid sequence selected from RIIPRHLQL (SEQ ID NO: 842), KLPAQFYIL (SEQ ID NO: 843), and KGPPAALTL (SEQ ID NO: 844). In some embodiments, the exogenous HLA-G polypeptide is a single chain fusion polypeptide comprising or consisting of the ectodomain of an HLA-G polypeptide (e.g., alpha1, alpha2, and alpha3 domains of an HLA-G1 or an HLA-G5 isoform polypeptide; alpha1 and alpha3 domains of an HLA-G2 or an HLA-G6 isoform polypeptide; alpha1 and alpha2 domains of an HLA-G4 isoform polypeptide; alpha1 and alpha2 domains of an HLA-G4 isoform polypeptide; or alpha1 domain of an HLA-G3 or an HLA-G7 polypeptide), a β2M polypeptide, and a membrane anchor (e.g., comprising a GPA transmembrane domain), wherein the single chain fusion polypeptide is optionally linked to an exogenous antigenic polypeptide. In some embodiments, the exogenous HLA-G polypeptide is a single chain fusion polypeptide comprising or consisting of one or more alpha domains of an HLA-G alpha chain (e.g., alpha1, alpha2, and/or alpha3 domains of an HLA-G1 or an HLA-G5 isoform polypeptide; alpha1 and alpha3 domains of an HLA-G2 or an HLA-G6 isoform polypeptide; alpha1 and alpha 2 domains of an HLA-G4 isoform polypeptide; alpha1 and alpha2 domains of an HLA-G4 isoform polypeptide; or alpha1 domain of an HLA-G3 or an HLA-G7 polypeptide), a β2M polypeptide, and a membrane anchor (e.g., a GPA transmembrane domain), wherein the single chain fusion polypeptide is optionally linked to an exogenous antigenic polypeptide. In some embodiments, the exogenous HLA-G polypeptide is a single chain fusion polypeptide comprising or consisting of one or more alpha domains of an HLA-G alpha chain (e.g., alpha1, alpha2, and/or alpha3 domains of an HLA-G1 or an HLA-G5 isoform polypeptide; alpha1 and alpha3 domains of an HLA-G2 or an HLA-G6 isoform polypeptide; alpha1 and alpha 2 domains of an HLA-G4 isoform polypeptide; alpha1 and alpha2 domains of an HLA-G4 isoform polypeptide; or alpha1 domain of an HLA-G3 or an HLA-G7 polypeptide), and a membrane anchor (e.g., a GPA transmembrane domain), wherein the single chain fusion polypeptide is optionally linked to an exogenous antigenic polypeptide.

The engineered erythroid cells (e.g., engineered enucleated erythroid cells) or enucleated cells (e.g., modified enucleated cells) that include an exogenous HLA-G polypeptide and an exogenous immunogenic polypeptide on their surface, can, inter alia, induce immune tolerance in a subject to the exogenous immunogenic polypeptide on the cell surface.

In some embodiments, the engineered erythroid cells (e.g., engineered enucleated erythroid cells) or enucleated cells (e.g., modified enucleated cells) that include an exogenous HLA-G polypeptide on the cell surface and an exogenous immunogenic polypeptide within in the cell, can, inter alia, induce immune tolerance in a subject to the exogenous immunogenic polypeptide.

For example, the engineered erythroid cells or enucleated cells described herein may mask the exogenous immunogenic polypeptide from a potential immune response in a subject to whom the cells are administered. Thus, the engineered erythroid cells and enucleated cells can be advantageously used for the treatment of diseases treatable by the administration of the exogenous immunogenic polypeptide without inducing an undesirable immune response, or inducing a reduced immune response, against the exogenous immunogenic polypeptide, in the subject(s) to whom the cells are administered.

In some embodiments, the engineered enucleated erythroid cells or enucleated cells comprising the exogenous immunogenic polypeptide and an exogenous HLA-G polypeptide, or a pharmaceutical composition comprising the cells, can be administered to a subject to treat a disease, resulting in a reduced immune response in the subject to the exogenous immunogenic polypeptide as compared to an immune response in the subject to the exogenous immunogenic polypeptide when the exogenous immunogenic polypeptide is administered alone. In other embodiments, the engineered enucleated erythroid cells or enucleated cells comprising the exogenous immunogenic polypeptide and an exogenous HLA-G polypeptide, or a pharmaceutical composition comprising the cells, can be administered to a subject to treat a disease, resulting in a reduced immune response in the subject to the exogenous immunogenic polypeptide as compared to the immune response in the subject to the exogenous immunogenic polypeptide when the exogenous immunogenic polypeptide is administered to the subject when present on the surface of a plurality of engineered enucleated erythroid cells lacking the exogenous HLA-G polypeptide.

In some embodiments, the engineered erythroid cells or enucleated cells comprising an exogenous HLA-G polypeptide and an exogenous immunogenic polypeptide on the cell surface, and optionally an exogenous antigenic polypeptide, as described herein, induce long-term immune tolerance to the exogenous immunogenic polypeptide in a subject to whom the cells are administered. For example, the engineered erythroid cells or enucleated cells described herein can inhibit the maturation of a dendritic cell (DC), induce anergy of a dendritic cell (DC), induce the differentiation into a regulatory T cell (Treg) of a CD4⁺ T cell that is contacted by an engineered enucleated erythroid cell or an enucleated cell described herein, and/or induce the differentiation into a regulatory T cell (Treg) of a CD8⁺ T cell that is contacted by an engineered enucleated erythroid cell or an enucleated cell described herein.

In other embodiments, the engineered erythroid cells or enucleated cells comprising an exogenous HLA-G polypeptide and an exogenous immunogenic polypeptide on the cell surface, and optionally an exogenous antigenic polypeptide, as described herein, induce short-term immune tolerance to the exogenous immunogenic polypeptide in a subject to whom the cells are administered. For example, the engineered erythroid cells or enucleated cells described herein can: induce apoptosis of an immune cell (e.g., a T cell, a natural killer (NK) cell, or a B cell), and/or inhibit the activation, differentiation, and/or proliferation of an immune cell (e.g., a T cell, a NK cell, or a B cell), inhibit the cytotoxicity of a T cell or an NK cell, and/or inhibit antibody secretion by a B cell.

In some embodiments, the engineered erythroid cells or enucleated cells comprise an exogenous HLA-G polypeptide on the cell surface and an exogenous immunogenic polypeptide in the cell, and optionally one or more exogenous antigenic polypeptide(s) and/or one or more exogenous coinhibitory polypeptide(s), as described herein, induce long-term immune tolerance to the exogenous immunogenic polypeptide in a subject to whom the cells are administered. For example, the engineered erythroid cells or enucleated cells described herein can inhibit the maturation of a dendritic cell (DC), induce anergy of a dendritic cell (DC), induce the differentiation into a regulatory T cell (Treg) of a CD4⁺ T cell that is contacted by an engineered enucleated erythroid cell or an enucleated cell described herein, and/or induce the differentiation into a regulatory T cell (Treg) of a CD8⁺ T cell that is contacted by an engineered enucleated erythroid cell or an enucleated cell described herein.

In other embodiments, the engineered erythroid cells or enucleated cells comprise an exogenous HLA-G polypeptide on the cell surface and an exogenous immunogenic polypeptide within the cell, and optionally one or more exogenous antigenic polypeptide(s) and/or one or more exogenous coinhibitory polypeptides, as described herein, induce short-term immune tolerance to the exogenous immunogenic polypeptide in a subject to whom the cells are administered. For example, the engineered erythroid cells or enucleated cells described herein can: induce apoptosis of an immune cell (e.g., a T cell, a natural killer (NK) cell, or a B cell), and/or inhibit the activation, differentiation, and/or proliferation of an immune cell (e.g., a T cell, a NK cell, or a B cell), inhibit the cytotoxicity of a T cell or an NK cell, and/or inhibit antibody secretion by a B cell.

Also provided herein are engineered enucleated erythroid cells that include at least one exogenous autoantigenic polypeptide (e.g., any of the exemplary exogenous autoantigenic polypeptides described herein or known in the art) and at least one exogenous coinhibitory polypeptide (e.g., any of the exemplary exogenous coinhibitory polypeptides described herein or known in the art).

Additional non-limiting aspects of exogenous autoantigenic polypeptides and exogenous coinhibitory polypeptides that can present in any of the engineered enucleated erythroid cells are described herein (and can be used in any combination).

In some embodiments of the present disclosure, the engineered erythroid cells are engineered enucleated erythroid cells, e.g., reticulocytes or erythrocytes. In some embodiments, the enucleated cell (e.g., modified enucleated cell) is a reticulocyte, an erythrocyte or a platelet.

Many modifications and other embodiments of the engineered erythroid cells (e.g., engineered enucleated erythroid cells) or enucleated cells (e.g., modified enucleated cells) and methods set forth herein will easily come to mind to one skilled in the art to which this disclosure pertains having the benefit of the teachings presented in the foregoing descriptions and the associated drawings. Therefore, it is to be understood that the disclosure herein is not to be limited to the specific embodiments disclosed and that modifications and other embodiments are intended to be included within the scope of the appended claims. Although specific terms are employed herein, they are used in a generic and descriptive sense only and not for purposes of limitation.

### Definitions

As used in this specification and the appended claims, the singular forms “a”, “an” and “the” include plural references unless the content clearly dictates otherwise.

The use of the alternative (e.g., “or”) should be understood to mean either one, both, or any combination thereof of the alternatives.

As used herein, the term “about,” when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

As used herein, any concentration range, percentage range, ratio range, or integer range is to be understood to include the value of any integer within the recited range and, when appropriate, fractions thereof (such as one tenth and one hundredth of an integer), unless otherwise indicated.

As used herein, “comprise,” “comprising,” and “comprises” and “comprised of” are meant to be synonymous with “include”, “including”, “includes” or “contain”, “containing”, “contains” and are inclusive or open-ended terms that specifies the presence of what follows, e.g., component and do not exclude or preclude the presence of additional, non-recited components, features, element, members, steps, known in the art or disclosed therein.

As used herein, the terms “such as,” “for example,” and the like are intended to refer to exemplary embodiments and not to limit the scope of the present disclosure.

Unless defined otherwise, all technical and scientific terms used herein have the same meaning as commonly understood by one of ordinary skill in the art to which the disclosure pertains. Although any methods and materials similar or equivalent to those described herein can be used in the practice for testing of the present disclosure, preferred materials and methods are described herein.

As used herein, the term “codon-optimized” refers to the modification of codons in the gene or coding regions of a nucleic acid molecule to reflect the typical codon usage of the host organism (e.g., a human erythroid cell) without altering the polypeptide encoded by the nucleic acid molecule. Such optimization includes replacing at least one, or more than one, or a significant number, of codons with one or more codons that are more frequently used in the genes of the host organism. Codon optimization may improve translation in an expression host cell or organism of a transcript RNA molecule transcribed from the coding sequence, or to improve transcription of a coding sequence.

As used herein, “dose” and “dosage” are used interchangeably herein to refer to a specific quantity of a pharmacologically active material for administration to a subject for a given time. Unless otherwise specified, the doses recited refer to a plurality of engineered erythroid cells or enucleated cells comprising at least one exogenous polypeptide and at least one exogenous immunogenic polypeptide, as described herein.

As used herein, the term “click chemistry” refers to a range of reactions used to covalently link a first and a second moiety, for convenient production of linked products. It typically has one or more of the following characteristics: it is fast, is specific, is high-yield, is efficient, is spontaneous, does not significantly alter biocompatibility of the linked entities, has a high reaction rate, produces a stable product, favors production of a single reaction product, has high atom economy, is chemoselective, is modular, is stereoselective, is insensitive to oxygen, is insensitive to water, is high purity, generates only inoffensive or relatively non-toxic by-products that can be removed by nonchromatographic methods (e.g., crystallization or distillation), needs no solvent or can be performed in a solvent that is benign or physiologically compatible, e.g., water, stable under physiological conditions. Examples include an alkyne/azide reaction, a diene/dienophile reaction, or a thiol/alkene reaction. Other reactions can be used. In some embodiments, the click chemistry reaction is fast, specific, and high-yield.

As used herein, the term “click chemistry handle” refers to a chemical moiety that is capable of reacting with a second click chemistry handle in a click reaction to produce a click signature. In some embodiments, a click chemistry handle is comprised by a coupling reagent, and the coupling reagent may further comprise a substrate reactive moiety.

As used herein, the term “endogenous” is meant to refer to a native form of compound (e.g., a small molecule) or process. For example, in some embodiments, the term “endogenous” refers to the native form of a nucleic acid or polypeptide in its natural location in an organism or a cell or in the genome of an organism or a cell.

As used herein, the term an “engineered cell” refers to a genetically-modified cell or progeny thereof.

As used herein, the term “enucleated cell” refers to a cell that lacks a nucleus (e.g., due to a differentiation process such as erythropoiesis). In some embodiments, an enucleated cell is incapable of expressing a polypeptide. In some embodiments, an enucleated cell is an erythrocyte, a reticulocyte, or a platelet.

As used herein, “engineered enucleated cell” refers to a cell that originated from a genetically-modified nucleated cell or progeny thereof, and lacks a nucleus (e.g., due to differentiation). In some embodiments, the engineered enucleated cell includes an exogenous polypeptide that was produced by the genetically-modified nucleated cell or progeny thereof (e.g., prior to enucleation) from which the engineered enucleated cell originated.

As used herein, “engineered erythroid cell” refers to a genetically-modified erythroid cell or progeny thereof. Engineered erythroid cells include engineered nucleated erythroid cells (e.g., genetically-modified erythroid precursor cells) and engineered enucleated erythroid cells (e.g., reticulocytes and erythrocytes that originated from a genetically modified erythroid precursor cell).

As used herein, “engineered enucleated erythroid cell” refers to an erythroid cell that originated from a genetically-modified nucleated erythroid cell or progeny thereof, and lacks a nucleus (e.g., due to differentiation). In some embodiments, an engineered enucleated erythroid cell comprises an erythrocyte or a reticulocyte that originated from a genetically-modified nucleated erythroid cell or progeny thereof. In some embodiments, the engineered enucleated erythroid cell did not originate from an immortalized nucleated erythroid cell or progeny thereof.

An “erythroid precursor cell”, as used herein, refers to a cell capable of differentiating into a reticulocyte or erythrocyte. Generally, erythroid precursor cells are nucleated. Erythroid precursor cells include a cord blood stem cell, a CD34⁺ cell, a hematopoietic stem cell (HSC), a spleen colony forming (CFU-S) cell, a common myeloid progenitor (CMP) cell, a blastocyte colony-forming cell, a burst forming unit-erythroid (BFU-E), a megakaryocyte-erythroid progenitor (MEP) cell, an erythroid colony-forming unit (CFU-E), an induced pluripotent stem cell (iPSC), a mesenchymal stem cell (MSC), a polychromatic normoblast, and an orthochromatic normoblast. In some embodiments, an erythroid precursor cell is an immortal or immortalized cell. For example, immortalized erythroblast cells can be generated by retroviral transduction of CD34⁺ hematopoietic progenitor cells to express Oct4, Sox2, Klf4, cMyc, and suppress TP53 (e.g., as described in Huang et al. (2014) Mol. Ther. 22(2): 451-63, the entire contents of which are incorporated by reference herein).

As used herein, the term “exogenous nucleic acid” refers to a nucleic acid (e.g., a gene) which is not native to a cell, but which is introduced into the cell or a progenitor of the cell. An exogenous nucleic acid may include a region or open reading frame (e.g., a gene) that is homologous to, or identical to, an endogenous nucleic acid native to the cell. In some embodiments, the exogenous nucleic acid comprises RNA. In some embodiments, the exogenous nucleic acid comprises DNA. In some embodiments, the exogenous nucleic acid is integrated into the genome of the cell. In some embodiments, the exogenous nucleic acid is processed by the cellular machinery to produce an exogenous polypeptide. In some embodiments, the exogenous nucleic acid is not retained by the cell or by a cell that is the progeny of the cell into which the exogenous nucleic acid was introduced.

As used herein, the term “exogenous” in reference to a polypeptide refers to a polypeptide that is introduced into or onto a cell, or is caused to be expressed by the cell by introducing an exogenous nucleic acid encoding the exogenous polypeptide into the cell or into a progenitor of the cell. In some embodiments, an exogenous polypeptide is a polypeptide encoded by an exogenous nucleic acid that was introduced into the cell or a progenitor of the cell, which nucleic acid is optionally not retained by the cell. In some embodiments, an exogenous polypeptide is a polypeptide conjugated to the surface of the cell by chemical or enzymatic means.

As used herein, the term “express” or “expression” refers to processes by which a cell produces a polypeptide, including transcription and translation. The expression of a particular polypeptide in a cell can be increased using several different approaches, including, but not limited to, increasing the copy number of genes encoding the polypeptide, increasing the transcription of a gene, and increasing the translation of an mRNA encoding the polypeptide.

As used herein, the terms “first”, “second”, and “third”, etc., with respect to exogenous polypeptides or nucleic acids are used for convenience of distinguishing when there is more than one type of exogenous polypeptide or nucleic acid. Use of these terms is not intended to confer a specific order or orientation of the exogenous polypeptides or nucleic acid unless explicitly so stated.

As used herein the term “nucleic acid molecule” refers to a single or double-stranded polymer of deoxyribonucleotide and/or ribonucleotide bases. It includes, but is not limited to, chromosomal DNA, plasmids, vectors, mRNA, tRNA, siRNA, etc. which can be recombinant and from which exogenous polypeptides can be expressed when the nucleic acid is introduced into a cell.

As used herein, the term “pharmaceutically acceptable carrier” includes any of the standard pharmaceutical excipients, carrier or stabilizer which are not toxic or deleterious to a mammal being exposed thereto at the dosage and/or concentration employed.

As used herein, the terms “polypeptide”, “peptide” and “protein” are used interchangeably herein to refer to a polymer of amino acid residues. The terms “polypeptide”, “peptide” and “protein” also are inclusive of modifications including, but not limited to, glycosylation, phosphorylation, lipid attachment, sulfation, gamma-carboxylation of glutamic acid residues, hydroxylation, and ADP-ribosylation. It will be appreciated, as is well known and as noted above, that polypeptides may not be entirely linear. For instance, polypeptides can be branched as a result of ubiquitination, and they can be circular, with or without branching, generally as a result of posttranslational events, including natural processing event and events brought about by human manipulation which do not occur naturally.

As used herein, polypeptides referred to herein as “recombinant” refers to polypeptides which have been produced by recombinant DNA methodology, including those that are generated by procedures which rely upon a method of artificial recombination, such as the polymerase chain reaction (PCR) and/or cloning into a vector using restriction enzymes.

As used herein, the terms “subject”, “individual” and “patient” are used interchangeably herein and refer to any mammalian subject for whom diagnosis, treatment, or therapy is desired, particularly humans. The methods described herein are applicable to both human therapy and veterinary applications. In some embodiments, the subject is a mammal (e.g., a human subject). In some embodiments, the subject is a non-human mammal (e.g., mouse, rat, guinea pig, dog, cat, horse, cow, pig, rabbit, sheep, or non-human primate, such as a monkey, chimpanzee, or baboon).

As used herein, the terms “therapeutically effective amount” and “effective amount” are used interchangeably to refer to an amount of an active agent (e.g. an engineered erythroid cell or an enucleated cell described herein) that is sufficient to provide the intended benefit (e.g. prevention, prophylaxis, delay of onset of symptoms, or amelioration of symptoms of a disease). In prophylactic or preventative applications, an effective amount can be administered to a subject susceptible to, or otherwise at risk of developing a disease, disorder or condition to eliminate or reduce the risk, lessen the severity, or delay the onset of the disease, disorder or condition, including a biochemical, histologic and/or behavioral symptoms of the disease, disorder or condition, its complications, and intermediate pathological phenotypes.

As used herein the term “therapeutic effect” refers to a consequence of treatment, the results of which are judged to be desirable and beneficial. A therapeutic effect can include, directly or indirectly, the arrest, reduction, or elimination of a disease manifestation. A therapeutic effect can also include, directly or indirectly, the arrest reduction or elimination of the progression of a disease manifestation. As used herein, the terms “treat,” “treating,” and/or “treatment” include abrogating, substantially inhibiting, slowing or reversing the progression of a disorder, disease or condition, substantially ameliorating clinical symptoms of a disorder, disease or condition, or substantially preventing the appearance of clinical symptoms of a disorder, disease or condition, obtaining beneficial or desired clinical results. Treating further refers to accomplishing one or more of the following: (a) reducing the severity of the disorder, disease or condition); (b) limiting development of symptoms characteristic of the disorder, disease or condition(s) being treated; (c) limiting worsening of symptoms characteristic of the disorder, disease or condition(s) being treated; (d) limiting recurrence of the disorder, disease or condition(s) in subjects that have previously had the disorder, disease or condition(s); and (e) limiting recurrence of symptoms in subjects that were previously asymptomatic for the disorder, disease or condition(s). Beneficial or desired clinical results, such as pharmacologic and/or physiologic effects include, but are not limited to, preventing the disease, disorder or condition from occurring in a subject predisposed to the disease, disorder or condition but does not yet experience or exhibit symptoms of the disease (prophylactic treatment), alleviation of symptoms of the disease, disorder or condition, diminishment of extent of the disease, disorder or condition, stabilization (i.e., not worsening) of the disease, disorder or condition, preventing spread of the disease, disorder or condition, delaying or slowing of the disease, disorder or condition progression, amelioration or palliation of the disease, disorder or condition, and combinations thereof, as well as prolonging survival as compared to expected survival if not receiving treatment.

As used herein, the term “variant” of a polypeptide refers to a polypeptide having at least one amino acid residue difference as compared to a reference polypeptide, e.g., one or more substitutions, insertions, or deletions. In some embodiments, a variant has at least about 70%, 75%, 80%, 85%, 90%, 91%, 92%, 93%, 94% 95%, 96%, 97%, 98%, or 99% identity to that polypeptide. A variant may include a fragment (e.g., an enzymatically active fragment of an immunogenic polypeptide (e.g., an enzyme)). In some embodiments, a fragment may lack up to about 1, 2, 3, 4, 5, 10, 20, 30, 40, 50, or 100 amino acid residues on the N-terminus, C-terminus, or both ends (each independently) of a polypeptide, as compared to the full-length polypeptide. Variants may occur naturally or be non-naturally occurring. Non-naturally occurring variants can be generated using mutagenesis methods known in the art. Variant polypeptides may comprise conservative or non-conservative amino acid substitutions, deletions or additions.

As used herein, the term “sequence identity” or “identity,” in reference to nucleic acid and amino acid sequences refers to the percentage of amino acid residues or nucleotides in a candidate sequence that are identical with the amino acid residues or nucleotides in the reference sequences after aligning the sequences and introducing gaps, if necessary, to achieve the maximum percent sequence identity, and not considering any conservative substitutions as part of the sequence identity. Optimal alignment of the sequences for comparison can be produced, besides manually, by means of the local homology algorithm of Smith and Waterman, 1981, Ads App. Math. 2, 482; by means of the local homology algorithm of Neddleman and Wunsch, 1970, J. Mol. Biol. 48, 443; by means of the similarity search method of Pearson and Lipman, 1988, Proc. Natl. Acad. Sci. USA 85, 2444; or by means of computer programs which use these algorithms (GAP, BESTFIT, FASTA, BLAST P, BLAST N and TFASTA in Wisconsin Genetics Software Package, Genetics Computer Group, 575 Science Drive, Madison, Wis.).

The term “exogenous immunogenic polypeptide,” as used herein, refers to an exogenous polypeptide that elicits a cellular and/or humoral immune response when administered to a subject, either alone or in or on a carrier (e.g., an engineered erythroid cell (e.g., engineered enucleated erythroid cell) or enucleated cell (e.g., modified enucleated cell)). An exogenous immunogenic polypeptide can be derived from any source. In some embodiments, an exogenous immunogenic polypeptide comprises a human polypeptide. In some embodiments, the exogenous immunogenic polypeptide comprises an alloreactive polypeptide (e.g., a human alloreactive polypeptide). In other embodiments, an exogenous immunogenic polypeptide comprises a non-human polypeptide. In some embodiments, an exogenous immunogenic polypeptide comprises a non-human polypeptide derived from a bacterium, a plant, a yeast, a fungus, a virus, a prion, or a protozoan.

The term “exogenous autoantigenic polypeptide” as used herein, referns to an exogenous polypeptide that is capable of eliciting or inducing immune tolerance to an autoantigen (e.g., an autoantigen associated with an autoimmune disorder) in a mammal.

The term “exogenous autoantigenic polypeptide” as used herein, referns to an exogenous polypeptide that is capable of eliciting or inducing immune tolerance to an autoantigen (e.g., an autoantigen associated with an autoimmune disorder) in a mammal.

An “amino acid-degrading polypeptide,” as used herein, refers to a polypeptide (e.g., an enzyme) that utilizes an amino acid as a substrate and catalyzes the conversion of the amino acid to a metabolite or degradation product. In some embodiments, the amino acid-degrading polypeptide hydrolyzes a bond in an amino acid residue. Amino acid-degrading polypeptides may include both wild-type or modified polypeptides. In some embodiments, an amino acid-degrading polypeptide is an asparaginase polypeptide, a phenylalanine ammonium lyase (PAL) polypeptide, a phenylalanine hydroxylase (PAH) polypeptide, a homocysteine-reducing polypeptide or a homocysteine-degrading polypeptide.

As used herein, the term “asparaginase polypeptide” refers to any polypeptide that degrades L-asparagine, e.g., to aspartic acid and ammonia (also referred to herein as asparagine-degrading activity). In some embodiments, the asparaginase polypeptide has both asparagine-degrading activity and glutamine-degrading activity (i.e., glutaminase activity). “Glutamine-degrading activity”, as used herein, refers to the ability of an enzyme to catalyze the hydrolysis of glutamine to glutamate and ammonia. Thus, in some embodiments, the asparaginase polypeptide catalyzes the hydrolysis of asparagine and glutamine to aspartic acid and glutamic acid, respectively, and ammonia. In some embodiments, the asparaginase polypeptide lacks glutamine-degrading activity. Methods for assaying the asparagine-degrading or glutamine-degrading activity of asparaginase polypeptides are described for example, in Gervais and Foote (2014) Mol. Biotechnol. 45(10): 865-877, which is herein incorporated by reference in its entirety). Asparaginase polypeptides may include both wild-type or modified polypeptides.

As used herein, a “homocysteine-reducing polypeptide” refers to any polypeptide that, when administered to a subject (e.g., on or in an engineered erythroid cell (e.g., engineered enucleated erythroid cell) or enucleated cell (e.g., modified enucleated cell), as described herein) has the effect of reducing the level of homocysteine, or any one or more of its metabolites in the subject, e.g., in the plasma or serum of the subject. As used herein, a homocysteine-reducing polypeptide does not utilize homocysteine as a substrate, i.e., does not include a homocysteine-degrading polypeptide as used herein. In some embodiments, homocysteine metabolites include, e.g., disulfide homocysteine (Hcy-S—S-Hcy), mixed disulfide of Hcy and Cys (Hcy-S—S-Cys), mixed disulfide of Hcy with plasma protein (S-Hcy-protein), Hcy-thiolactone, N-Hcy-protein, Nε-Hcy-Lys, AdoHcy, cystathionine, homocysteine sulfinic acid, homocysteic acid, and methionine. Homocysteine-reducing polypeptides may include both wild-type or modified polypeptides. Erythroid cells and enucleated cells including an exogenous polypeptide comprising a homocysteine-reducing polypeptide can be used to treat a homocysteine-related disease, or to reduce homocysteine levels and/or methionine levels, in a subject.

As used herein, a “homocysteine-degrading polypeptide” refers to any polypeptide that utilizes homocysteine as substrate and converts homocysteine to a metabolite or degradation product of homocysteine. Homocysteine-degrading polypeptides include both wild-type or modified polypeptides. Erythroid cells and enucleated cells including an exogenous polypeptide comprising a homocysteine-degrading polypeptide, can be used to treat a homocysteine-related disease, or to reduce homocysteine levels and/or methionine levels, in a subject.

As used herein, a “uric acid-degrading polypeptide” refers to any polypeptide that catabolizes or degrades uric acid. Examples of uric acid-degrading polypeptides include urate oxidase (also known as uricase), allantoinase and allantoicase. Other examples of uric acid-degrading polypeptides are described herein and are not intended to be limiting. In some embodiments, a uric acid-degrading polypeptide catalyzes the hydrolysis of uric acid.

As used herein, the term “cancer” includes any cancer including leukemia, acute lymphoblastic leukemia (ALL), an acute myeloid leukemia (AML), an anal cancer, a bile duct cancer, a bladder cancer, a bone cancer, a bowel cancer, a brain tumor, a breast cancer, a carcinoid, a cervical cancer, a choriocarcinoma, a chronic lymphocytic leukemia (CLL), a chronic myeloid leukemia (CML), a colon cancer, a colorectal cancer, an endometrial cancer, an eye cancer, a gallbladder cancer, a gastric cancer, a gestational trophoblastic tumor (GTT), a hairy cell leukemia, a head and neck cancer, a Hodgkin lymphoma, a kidney cancer, a laryngeal cancer, a liver cancer, a lung cancer, a lymphoma, a melanoma, a skin cancer, a mesothelioma, a mouth or oropharyngeal cancer, a myeloma, a nasal or sinus cancer, a nasopharyngeal cancer, a non-Hodgkin lymphoma (NHL), an esophageal cancer, an ovarian cancer, a pancreatic cancer, a penile cancer, a prostate cancer, a rectal cancer, a salivary gland cancer, a non-melanoma skin cancer, a soft tissue sarcoma, a stomach cancer, a testicular cancer, a thyroid cancer, a uterine cancer, a vaginal cancer, and a vulvar cancer.

As used herein, the term “uric acid-related disease” refers to a disease associated with excess uric acid in a subject (e.g., a human subject”). In some embodiments, the uric acid-related disease is selected from hyperuricemia, asymptomatic hyperuricemia, hyperuricosuria, gout (e.g., chronic refractory gout), lesch-nyhan syndrome, uric acid nephrolothiasis, vascular conditions, diabetes, metabolic syndrome, inflammatory responses, cognitive impairment, rheumatoid arthritis, osteoarthritis, cerebral stroke, ischemic heart disease, arrhythmia, and chronic renal disease.

As used herein, the term “homocysteine-related disease,” refers to a disease associated with excess homocysteine in a subject (e.g., a human subject”) and/or involving abnormal (e.g., increased) levels of homocysteine or molecules directly upstream, such as glyoxylate. In some embodiments, the homocysteine-related disease is homocystinuria. In some embodiments, the homocystinuria is symptomatic homocystinuria. In other embodiments, the homocystinuria is asymptomatic homocystinuria.

The term “exogenous antigenic polypeptide” as used herein, refers to an exogenous polypeptide that is capable of binding to the antigen-binding cleft of an exogenous HLA-G polypeptide. As used herein, an exogenous antigenic polypeptide is distinct from an exogenous immunogenic polypeptide.

The terms “HLA-G polypeptide” and “HLA-G” are used interchangeably herein to refer to a polypeptide comprising one or more alpha domains (e.g., alpha1, alpha2, and alpha3 domains) of a heavy a chain of a human HLA class I histocompatibility antigen, alpha chain G polypeptide. The full length a heavy chain of HLA-G is approximately 45 kDa and its gene contains 8 exons. Exon one encodes the leader peptide, exons 2 and 3 encode the alpha1 and alpha2 domain, which both bind the peptide, exon 4 encodes the alpha3 domain, exon 5 encodes the transmembrane region, and exon 6 encodes the cytoplasmic tail. As described herein, in some embodiments, an HLA-G polypeptide can comprise less than all three of the endogenous alpha domains (i.e., the HLA-G polypeptide can comprise one, two or three of the alpha domains). In some embodiments, an HLA-G polypeptide comprises the ectodomain of a naturally-occurring HLA-G polypeptide (e.g., one or more of alpha1, alpha2, and alpha 3 domains) and excludes the transmembrane domain and the cytoplasmic tail of the naturally-occurring HLA-G polypeptide. In some embodiments, an HLA-G polypeptide comprises alpha1, alpha2 and alpha 3 domains of an HLA-G1 or an HLA-G5 isoform polypeptide. In some embodiments, an HLA-G polypeptide comprises alpha1, alpha2 and alpha 3 domains of an HLA-G1 isoform polypeptide (e.g., HLA-G1*01:01 allele or HLA-G1*01:04 allele). In some embodiments, an HLA-G polypeptide comprises alpha1 and alpha3 domains of an HLA-G2 or an HLA-G6 isoform polypeptide. In some embodiments, an HLA-G polypeptide comprises alpha1 and alpha 2 domains of an HLA-G4 isoform polypeptide. In some embodiments, an HLA-G2 polypeptide comprises alpha1 and alpha2 domains of an HLA-G4 isoform polypeptide. In some embodiments, an HLA-G polypeptide comprises an alpha1 domain of an HLA-G3 or an HLA-G7 polypeptide. In some embodiments, an HLA-G polypeptide comprises the ectodomain of a naturally occurring HLA-G polypeptide (e.g., one or more of alpha1, alpha2, and alpha3 domains) and is fused to a membrane anchor (e.g., a glycophorin A (GPA) transmembrane domain). In some embodiments, an HLA-G polypeptide comprises the ectodomain of a naturally-occurring HLA-G polypeptide (e.g., one or more of alpha1, alpha2, and alpha3 domains), and is fused to a membrane anchor (e.g., a GPA transmembrane domain) which comprises an HLA-G cytoplasmic domain). As described herein, in some embodiments, an HLA-G polypeptide also includes an HLA-G heavy chain that is bound or linked to a light chain (i.e., beta-2 microglobulin or β2M polypeptide), to form a heterodimer (e.g., as a single chain fusion polypeptide). In some embodiments, an HLA-G polypeptide is not bound or linked to a light chain (i.e., a β2M polypeptide). In some embodiments, an HLA-G polypeptide binds or is bound to an exogenous antigenic polypeptide, and/or is linked to a membrane anchor. In some embodiments, an HLA-G polypeptide comprises an “HLA-G single chain fusion polypeptide,” wherein the HLA-G polypeptide comprises one or more alpha domains of an HLA-G heavy chain (e.g., one or more of alpha1, alpha2, and alpha3 domains) linked to β2M polypeptide, and optionally the β2M polypeptide is linked to an exogenous antigenic polypeptide. In some embodiments, the single chain fusion polypeptide includes a membrane anchor, e.g., a GPA polypeptide, or a transmembrane domain thereof; a SMIM1 polypeptide, or a transmembrane domain thereof or a transferrin receptor, or a transmembrane domain thereof.

The term “immune tolerance,” as used herein, refers to any mechanism resulting in the inhibition, reduction, or prevention of immune activation, or the suppression or inhibition of an immune response in a subject. Immune tolerance includes central tolerance and peripheral tolerance. In some embodiments, central tolerance refers to the antigen-specific deletion of autoreactive T cells and B cells during development in the primary lymphoid organs, e.g. thymus and bone marrow. In some embodiments, peripheral tolerance refers to the deletion or inactivation of mature T and B lymphocytes outside of the primary lymphoid organs. In some embodiments, peripheral tolerance includes the suppression of autoreactive lymphocytes by regulatory T cells (Tregs) or the induction of anergy or non-responsiveness in antigen-specific effector lymphocytes by exposure to continuous low doses of antigen in the absence of costimulatory danger signals. Both Treg activation and lymphocyte anergy can be induced by the secretion of inhibitory factors such as, for example, TGF-beta, IL-10, and IL-4. The inhibitory effects of tolerance can be induced over a long- or a short-term (i.e., long-term immune tolerance or short-term immune tolerance).

The term “long-term immune tolerance,” as used herein, refers to the long-term inhibitory effects on an immune response (e.g., to an exogenous immunogenic polypeptide) related to, for example, the induction of regulatory or suppressor T cells that contribute to the development of tolerance. In some embodiments, the interaction of an exogenous HLA-G polypeptide with an ILT4 receptor favors the induction of Tregs, which can initiate such long-term effects (see, e.g., Rebmann et al., J Immunol Res. 2014; 2014:297073, incorporated herein by reference). In some embodiments, long-term immune tolerance comprises inhibiting the maturation of a DC that is contacted by an engineered erythroid cell or enucleated cell described herein. In other embodiments, long-term immune tolerance comprises inducing anergy of a DC that is contacted by an engineered erythroid cell or enucleated cell. In other embodiments, long-term immune tolerance comprises inducing the differentiation into a Treg of a CD4⁺ T cell that is contacted by an engineered erythroid cell or enucleated cell described herein; or inducing the differentiation into a Treg of a CD8⁺ T cell that is contacted by an engineered erythroid cell or enucleated cell described herein.

The term “short-term immune tolerance,” as used herein, refers to the short-term inhibitory effects on an immune response (e.g., to an exogenous immunogenic polypeptide) related to, for example, inhibition of T and NK cell cytotoxicity, inhibition of T, NK and B cell proliferation, and/or the inhibition of antibody production. Short-term immune tolerance can be induced by the interaction of an exogenous HLA-G polypeptide (e.g., bound to an exogenous antigenic polypeptide), with the ILT2 receptor on T, NK and B cells, and with the cognate inhibitory receptor heterodimer CD94 and NKG2A on T and NK cells (see, e.g., Rebmann et al., J Immunol Res. 2014; 2014:297073, incorporated herein by reference). In some embodiments, short-term immune tolerance comprises inducing apoptosis or inhibiting the activation, differentiation, and/or proliferation of an immune cell (e.g., a T cell, a NK cell, or a B cell, or populations thereof) that is contacted by an engineered erythroid cell or an enucleated cell provided herein. In some embodiments, short-term immune tolerance comprises inhibiting the cytotoxicity of a T cell or an NK cell that is contacted by an engineered erythroid cell or an enucleated cell provided herein. In some embodiments, short-term immune tolerance comprises inhibiting antibody secretion by a B cell that is contacted by an engineered erythroid cell or an enucleated cell provided herein.

As used herein, the term “induce” in reference to an immune tolerance refers to increasing, stimulating or enhancing either directly or indirectly, immune tolerance, e.g., long-term immune tolerance or short-term immune tolerance, in a subject.

As used herein, the terms “suppressing” or “inhibiting” in reference to immune cells refer to a process (e.g., a signaling event) causing or resulting in the inhibition or suppression of one or more cellular responses or activities of an immune cell, selected from: proliferation, differentiation, cytokine secretion, cytotoxic effector molecule release, cytotoxic activity, and expression of activation markers, or resulting in anergizing of an immune cell or induction of apoptosis of an immune cell. Suitable assays to measure immune cell inhibition or suppression are known in the art and are described herein.

As used herein, the term “reduce” in reference to an immune response refers to decreasing, inhibiting, or suppressing the form or character of the immune response, e.g., as measured by ELISPOT assay (cellular immune response), ICS (intracellular cytokine staining assay) and major histocompatibility complex (MHC) tetramer assay to detect and quantify antigen-specific T cells, quantifying the blood population of antigen-specific CD4⁺ T cells, or quantifying the blood population of antigen-specific CD8⁺ T cells by a measurable amount, or where the reduction is by at least 10%, at least 20%, at least 30%, at least 40%, at least 50%, at least 60%, at least 70%, at least 80%, at least 90%, at least 95%, at least 96%, at least 97%, at least 98%, at least 99%, or 100%, when compared to a suitable control.

The term “coinhibitory polypeptide” as used herein refers to any polypeptide that suppresses an immune cell, including inhibition of immune cell activity, inhibition of immune cell proliferation, anergizing of an immune cell, or induction of apoptosis of an immune cell. In some embodiments, an exogenous coinhibitory polypeptide is capable of specifically binding to a cognate coinhibitory polypeptide on an immune cell.

The term “polyGS linker” means a peptide sequence comprising one or more (e.g., two, three, four, five, six, seven, eight, nine, or ten) consecutive copies of the dipeptide of glycine and serine (GS). Non-limiting examples of polyGS linkers are described herein.

The term “Ii key peptide” is a peptide that, when positioned N-terminally relative to an exogenous autoantigenic polypeptide, facilitates the binding of the exogenous autoantigenic polypeptide to the antigen-binding cleft of an MEW class II molecule. Non-limiting examples of Ii key peptides are described herein. Additional examples of Ii key peptides are known in the art.

The term “specifically binds,” as used herein refers to the binding of a ligand to a polypeptide of interest (as opposed to non-specific binding of the ligand to other, non-specific polypeptides). In some embodiments, the binding is covalent. In other embodiments, the binding is non-covalent. For example, an exogenous antigenic polypeptide may be specifically bound either covalently or non-covalently to an exogenous HLA-G polypeptide, as described herein.

### I. Engineered Erythroid Cells and Enucleated Cells

The present disclosure features engineered erythroid cells (e.g., engineered enucleated erythroid cells) or enucleated cells (e.g., modified enucleated cells) that are engineered to include an exogenous HLA-G polypeptide and an exogenous immunogenic polypeptide on the surface of the cells, whereby when the cells are administered to a subject, immune tolerance (e.g., short-term immune tolerance or long-term immune tolerance) to the exogenous immunogenic polypeptide is induced and/or a reduced immune response to the exogenous immunogenic polypeptide is induced.

In some embodiments, the disclosure provides engineered erythroid cells or enucleated cells that include, on the cell surface, one or more exogenous HLA-G polypeptides and one or more exogenous immunogenic polypeptide(s), e.g., an amino acid-degrading polypeptide (e.g., an asparaginase polypeptide, a phenylalanine ammonium lyase (PAL) polypeptide, a phenylalanine hydroxylase (PAH) polypeptide, a homocysteine-reducing polypeptide or a homocysteine-degrading polypeptide), a uric acid-degrading polypeptide, oxalate oxidase, a d-aminolevulinate dehydrogenase (ALA-D), or any one or more of the polypeptides set forth in Tables 1 or 2 herein.

In some embodiments, the disclosure provides engineered erythroid cells (e.g., engineered enucleated erythroid cells) or enucleated cells (e.g., modified enucleated cells) that include, one or more exogenous HLA-G polypeptides on the cell surface and one or more exogenous immunogenic polypeptide(s) within the cell, e.g., any one or more of the polypeptides set forth in Table 1 or 2 herein).

Some embodiments of any of the engineered erythroid cells (e.g., engineered enucleated erythroid cells) or enucleated cells (e.g., modified enucleated cells) further include one or more exogenous antigenic polypeptides (e.g., any of the exemplary exogenous antigenic polypeptides described herein or known in the art) and/or one or more exogenous coinhibitory polypeptides (e.g., any of the exemplary coinhibitory polypeptides described herein or known in the art). In some embodiments, the one or more exogenous antigenic polypeptides can be present on the cell surface, in the cytoplasm of the cell, on the intracellular surface of the plasma membrane, or secreted or released by the cell. In some embodiments, the one or more exogenous inhibitory polypeptides can be present on the cell surface, in the cytoplasm of the cell, on the intracellular surface of the plasma membrane, or secreted/released by the cell. In some embodiments, the one or more exogenous antigenic polypeptides are not bound by the exogenous HLA-G polypeptide. In some embodiments, the one or more exogenous antigenic polypeptides are bound by an exogenous HLA-G polypeptide. In some embodiments, the one or more exogenous coinhibitory polypeptides are not bound by an exogenous HLA-G.

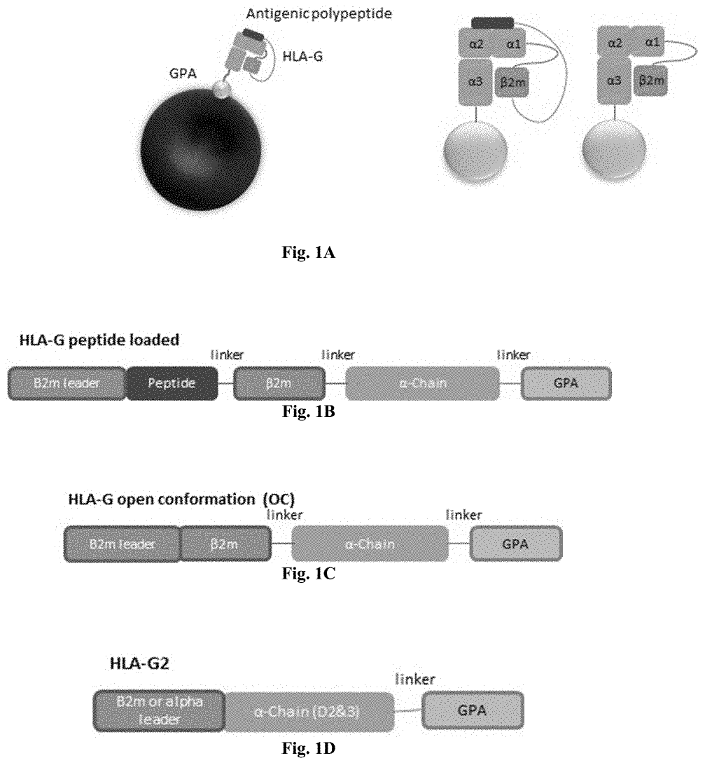

In some embodiments, the present disclosure provides an engineered erythroid cell (e.g., engineered enucleated erythroid cell) or an enucleated cell (e.g., a modified enucleated cell) comprising an immunogenic polypeptide, an exogenous HLA-G polypeptide, and an exogenous antigenic polypeptide, whereby the exogenous HLA-G polypeptide is bound (e.g., specifically bound) to the exogenous antigenic polypeptide. In some embodiments, the disclosure also provides an engineered erythroid cell or an enucleated cell comprising at least one immunogenic polypeptide, an exogenous HLA-G polypeptide, and an exogenous antigenic polypeptide, whereby the exogenous HLA-G polypeptide is linked to the exogenous antigenic polypeptide as part of a single chain fusion polypeptide (see, e.g., FIG. 1A)). In some embodiments, also provided is an engineered erythroid cell or an enucleated cell comprising at least one immunogenic polypeptide, an exogenous HLA-G polypeptide, and an exogenous antigenic polypeptide, whereby the exogenous HLA-G polypeptide is not linked to the exogenous antigenic polypeptide (e.g., the exogenous HLA-G polypeptide and the exogenous antigenic polypeptide are two distinct polypeptides).

In some embodiments, any of the engineered erythroid cells and enucleated cells described herein may also comprise one or more additional exogenous polypeptides including, but not limited to, an exogenous coinhibitory polypeptide, as described below.

Also provided herein are engineered erythroid cells (e.g., engineered enucleated erythroid cells) or enucleated cells (e.g., modified enucleated cells) including at least one exogenous autoantigenic polypeptide (e.g., one or more of any of the exemplary autoantigenic polypeptides described herein or known in the art) and at least one exogenous coinhibitory polypeptide (e.g., one or more of any of the exemplary coinhibitory polypeptides described herein). In some embodiments of these engineered erythroid cells or enucleated cells, the cell does not comprise a HLA-G polypeptide or a functional fragment thereof. In some embodiments of these engineered erythroid cells or enucleated cells, the cell does not include a MHC polypeptide or a functional fragment thereof.

### Exogenous HLA-G Polypeptides

The present disclosure includes engineered erythroid cells (e.g., engineered enucleated erythroid cells) or enucleated cells (e.g., modified enucleated cells) including one or more (e.g., one, two, three, four, five or more) exogenous HLA-G polypeptides. In some embodiments, the exogenous HLA-G polypeptide is an exogenous antigen-presenting HLA-G polypeptide. In some embodiments, the exogenous HLA-G polypeptide includes an exogenous antigenic polypeptide loaded onto (bound to) the exogenous HLA-G polypeptide's antigen-binding cleft. In some embodiments, the exogenous antigenic polypeptide may be bound either covalently or non-covalently to the exogenous HLA-G polypeptide. In some embodiments, the exogenous HLA-G polypeptide includes an endogenous antigenic polypeptide loaded onto (bound to) the exogenous HLA-G polypeptide's antigen-binding cleft. In some embodiments, the endogenous or exogenous antigenic polypeptide comprises an amino acid sequence having the motif XI/LPXXXXXL, wherein X is any amino acid (SEQ ID NO: 1). In some embodiments, the exogenous or endogenous antigenic polypeptide comprises or consists of an amino acid sequence selected from RIIPRHLQL (SEQ ID NO: 842), KLPAQFYIL (SEQ ID NO: 843), and KGPPAALTL (SEQ ID NO: 844).

In some embodiments, the exogenous HLA-G polypeptide comprises a functional HLA-G polypeptide. In some embodiments, the exogenous HLA-G polypeptide comprises one or more of alpha domains (alpha1, alpha2, and alpha3 domains) of an HLA-G alpha chain, or fragments or variants thereof. In some embodiments, the exogenous HLA-G polypeptide includes a beta-2 microglobulin (β2M) polypeptide, or a fragment or variant thereof. In some embodiments, the exogenous HLA-G polypeptide comprises one or more alpha domains of an HLA-G chain bound, e.g., covalently bound or non-covalently bound, to a β2M polypeptide (or a fragment or variant thereof). In some embodiments, the exogenous HLA-G polypeptide does not include a β2M polypeptide (or a fragment or variant thereof).

In some embodiments, the exogenous HLA-G polypeptide comprises or consists of a HLA-G1 isoform polypeptide, a HLA-G2 isoform polypeptide, a HLA-G3 isoform polypeptide, a HLA-G4 isoform polypeptide, a HLA-G5 isoform polypeptide, a HLA-G6 isoform polypeptide, or a HLA-G7 isoform polypeptide, or a fragment thereof (e.g., one or more alpha domains thereof). In some embodiments, the exogenous HLA-G polypeptide is capable of oligomerizing (e.g., of forming a dimer). In some embodiments, the HLA-G polypeptide is of the HLA-G1*01:01 allele. In some embodiments, the HLA-G polypeptide is of the HLA-G1*01:04 allele.

In some embodiments, an exogenous antigenic polypeptide is linked to the exogenous HLA-G polypeptide as part of a fusion polypeptide, e.g., a single chain fusion polypeptide. In some embodiments, the exogenous antigenic polypeptide comprises an amino acid sequence having the motif XI/LPXXXXXL, wherein X is any amino acid (SEQ ID NO: 1). In some embodiments, the exogenous antigenic polypeptide comprises or consists of an amino acid sequence selected from RIIPRHLQL (SEQ ID NO: 842), KLPAQFYIL (SEQ ID NO: 843), and KGPPAALTL (SEQ ID NO: 844). For example, in some embodiments, the exogenous HLA-G polypeptide linked to the exogenous antigenic polypeptide has the structure set forth in FIG. 1B. In other embodiments, the exogenous HLA-G polypeptide has the structure set forth in FIG. 1C. In other embodiments, the exogenous HLA-G polypeptide has the structure set forth in FIG. 1D.

In some embodiments, the exogenous HLA-G polypeptide comprises one or more alpha domains 1-3 (e.g., alpha1, alpha2, and alpha3 domains) of an HLA-G polypeptide and does not include a β2M polypeptide. In some embodiments, the exogenous HLA-G polypeptide comprises one or more alpha domains 1-3 (e.g., alpha1, alpha2, and alpha3 domains) of an HLA-G polypeptide and a β2M polypeptide, or a fragment or variant thereof. In some embodiments, the exogenous HLA-G polypeptide comprises one or more alpha domains 1-3 (e.g., alpha1, alpha2, and alpha3 domains) of an HLA-G polypeptide, and a membrane anchor (e.g., GPA or a transmembrane domain thereof). In some embodiments, the exogenous HLA-G polypeptide comprises one or more alpha domains 1-3 (e.g., alpha1, alpha2, and alpha3 domains) of an HLA-G polypeptide, a β2M polypeptide (or a fragment or variant thereof), and a membrane anchor. In some embodiments, the exogenous HLA-G polypeptide comprises one or more alpha domains 1-3 (e.g., alpha1, alpha2, and alpha3 domains) of an HLA-G polypeptide, a membrane anchor, and one or more linkers (e.g., a flexible linker). In some embodiments, the exogenous HLA-G polypeptide comprises one or more alpha domains 1-3 (e.g., alpha1, alpha2, and alpha3 domains) of an HLA-G polypeptide, a β2M polypeptide (or a fragment or variant thereof), a membrane anchor, and one or more linkers (e.g., a flexible linker). In some embodiments, the exogenous HLA-G polypeptide comprises one or more alpha domains 1-3 (e.g., alpha1, alpha2, and alpha3 domains) of an HLA-G polypeptide, a membrane anchor, and one or more linkers (e.g., a flexible linker), and is linked to an exogenous antigenic polypeptide (e.g., via a linker). In some embodiments, the exogenous HLA-G polypeptide comprises one or more alpha domains 1-3 (e.g., alpha1, alpha2, and alpha3 domains) of an HLA-G polypeptide, a β2M polypeptide (or a fragment or variant thereof), a membrane anchor, and one or more linkers (e.g., a flexible linker), and is linked to an exogenous antigenic polypeptide (e.g., via a linker).

In some embodiments, the exogenous HLA-G polypeptide comprises alpha1, alpha2, and alpha3 domains of an HLA-G1 isoform polypeptide (e.g., HLA-G1*01:01 allele or HLA-G1*01:04 allele) and does not include a β2M polypeptide. In some embodiments, the exogenous HLA-G polypeptide comprises alpha1, alpha2, and alpha3 domains of an HLA-G1 isoform polypeptide (e.g., HLA-G1*01:01 allele or HLA-G1*01:04 allele) and a β2M polypeptide, or a fragment or variant thereof. In some embodiments, the exogenous HLA-G polypeptide comprises alpha1, alpha2, and alpha3 domains of an HLA-G1 isoform polypeptide (e.g., HLA-G1*01:01 allele or HLA-G1*01:04 allele), and a membrane anchor. In some embodiments, the exogenous HLA-G polypeptide comprises alpha1, alpha2, and alpha3 domains of an HLA-G1 isoform polypeptide, a β2M polypeptide (or a fragment or variant thereof), and a membrane anchor. In some embodiments, the exogenous HLA-G polypeptide comprises alpha1, alpha2, and alpha3 domains of an HLA-G1 isoform polypeptide (e.g., HLA-G1*01:01 allele or HLA-G1*01:04 allele), a membrane anchor, and one or more linkers (e.g., a flexible linker). In some embodiments, the exogenous HLA-G polypeptide comprises alpha1, alpha2, and alpha3 domains of an HLA-G1 isoform polypeptide (e.g., HLA-G1*01:01 allele or HLA-G1*01:04 allele), a β2M polypeptide (or a fragment or variant thereof), a membrane anchor, and one or more linkers. In some embodiments, the exogenous HLA-G polypeptide comprises alpha1, alpha2, and alpha3 domains of an HLA-G1 isoform polypeptide (e.g., HLA-G1*01:01 allele or HLA-G1*01:04 allele), a membrane anchor, and one or more linkers, and is linked to an exogenous antigenic polypeptide (e.g., via a linker). In some embodiments, the exogenous HLA-G polypeptide comprises alpha1, alpha2, and alpha3 domains of an HLA-G1 isoform polypeptide (e.g., HLA-G1*01:01 allele or HLA-G1*01:04 allele), a β2M polypeptide (or a fragment or variant thereof), a membrane anchor, and one or more linkers, and is linked to an exogenous antigenic polypeptide (e.g., via a linker).

In some embodiments, the exogenous HLA-G polypeptide comprises alpha1 and alpha3 domains of an HLA-G2 isoform polypeptide and does not include a β2M polypeptide. In some embodiments, the exogenous HLA-G polypeptide comprises alpha1 and alpha3 domains of an HLA-G2 isoform polypeptide and a β2M polypeptide, or a fragment or variant thereof. In some embodiments, the exogenous HLA-G polypeptide comprises alpha1 and alpha3 domains of an HLA-G2 isoform polypeptide, and a membrane anchor. In some embodiments, the exogenous HLA-G polypeptide comprises alpha1 and alpha3 domains of an HLA-G2 isoform polypeptide, a β2M polypeptide (or a fragment or variant thereof), and a membrane anchor. In some embodiments, the exogenous HLA-G polypeptide comprises alpha1 and alpha3 domains of an HLA-G2 isoform polypeptide, a membrane anchor, and one or more linkers. In some embodiments, the exogenous HLA-G polypeptide comprises alpha1 and alpha3 domains of an HLA-G2 isoform polypeptide, a β2M polypeptide (or a fragment or variant thereof), a membrane anchor, and one or more linkers. In some embodiments, the exogenous HLA-G polypeptide comprises alpha1 and alpha3 domains of an HLA-G2 isoform polypeptide, a membrane anchor, and one or more linkers, and is linked to an exogenous antigenic polypeptide (e.g., via a linker). In some embodiments, the exogenous HLA-G polypeptide comprises alpha1 and alpha3 domains of an HLA-G2 isoform polypeptide, a β2M polypeptide (or a fragment or variant thereof), a membrane anchor, and one or more linkers, and is linked to an exogenous antigenic polypeptide (e.g., via a linker). In some embodiments, the exogenous HLA-G polypeptide comprises or consists of the amino acid sequence:

(SEQ ID NO: 34)
MVVMAPRTLFLLLSGALTLTETWAGSHSMRYFSAAVSRPGRGEPRFIAMG
YVDDTQFVRFDSDSACPRMEPRAPWVEQEGPEYWEEETRNTKAHAQTDRM
NLQTLRGYYNQSEASSHTLQWMIGCDLGSDGRLLRGYEQYAYDGKDYLAL
NEDLRSWTAADTAAQISKRKCEAANVAEQRRAYLEGTCVEWLHRYLENGK
EMLQRADPPKTHVTHHPVFDYEATLRCWALGFYPAEIILTWQRDGEDQTQ
DVELVETRPAGDGTFQKWAAVVVPSGEEQRYTCHVQHEGLPEPLMLRWKQ
SSLPTIPIMGIVAGLVVLAAVVTGAAVAAVLWRKKSSD
(signal peptide underlined).

In some embodiments, the HLA-G polypeptide comprises or consists of the amino acid sequence:

(SEQ ID NO: 35)
GSHSMRYFSAAVSRPGRGEPRFIAMGYVDDTQFVRFDSDSACPRMEPRAP
WVEQEGPEYWEEETRNTKAHAQTDRMNLQTLRGYYNQSEASSHTLQWMIG
CDLGSDGRLLRGYEQYAYDGKDYLALNEDLRSWTAADTAAQISKRKCEAA
NVAEQRRAYLEGTCVEWLHRYLENGKEMLQRADPPKTHVTHHPVFDYEAT
LRCWALGFYPAEIILTWQRDGEDQTQDVELVETRPAGDGTFQKWAAVVVP
SGEEQRYTCHVQHEGLPEPLMLRWKQSSLPTIPIMGIVAGLVVLAAVVTG
AAVAAVLWRKKSSD

In some embodiments, the exogenous HLA-G polypeptide or consists of the amino acid sequence of SEQ ID NO: 35, and comprises an unpaired cysteine at residue 42 of SEQ ID NO: 35.

In some embodiments, the HLA-G polypeptide comprises an amino acid sequence having at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 8′7%, at least 88%, at least 89%, at least 90%, at least 91%, at least 92%, at least 93%, at least 94%, at least 95%, at least 96%, at least 97%, at least 98%, or at least 99% sequence identity to the amino acid sequence of a corresponding wild-type HLA-G polypeptide, e.g., SEQ ID NO: 34 or SEQ ID NO: 35.

In some embodiments, the exogenous HLA-G polypeptide comprises alpha1 and alpha3 domains of an HLA-G2 isoform polypeptide comprising the amino acid sequence of SEQ ID NO: 34 or 35 and does not include a β2M polypeptide. In some embodiments, the exogenous HLA-G polypeptide comprises alpha1 and alpha3 domains of an HLA-G2 isoform polypeptide comprising the amino acid sequence of SEQ ID NO: 34 or 35 and a β2M polypeptide, or a fragment or variant thereof. In some embodiments, the exogenous HLA-G polypeptide comprises alpha1 and alpha3 domains of an HLA-G2 isoform polypeptide comprising the amino acid sequence of SEQ ID NO: 34 or 35, and a membrane anchor. In some embodiments, the exogenous HLA-G polypeptide comprises alpha1 and alpha3 domains of an HLA-G2 isoform polypeptide comprising the amino acid sequence of SEQ ID NO: 34 or 35, a β2M polypeptide (or a fragment or variant thereof), and a membrane anchor. In some embodiments, the exogenous HLA-G polypeptide comprises alpha1 and alpha3 domains of an HLA-G2 isoform polypeptide comprising the amino acid sequence of SEQ ID NO: 34 or 35, a membrane anchor, and one or more linkers. In some embodiments, the exogenous HLA-G polypeptide comprises alpha1 and alpha3 domains of an HLA-G2 isoform polypeptide comprising the amino acid sequence of SEQ ID NO: 34 or 35, a β2M polypeptide (or a fragment or variant thereof), a membrane anchor, and one or more linkers. In some embodiments, the exogenous HLA-G polypeptide comprises alpha1 and alpha3 domains of an HLA-G2 isoform polypeptide comprising the amino acid sequence of SEQ ID NO: 34 or 35, a membrane anchor, and one or more linkers, and is linked to an exogenous antigenic polypeptide (e.g., via a linker). In some embodiments, the exogenous HLA-G polypeptide comprises alpha1 and alpha3 domains of an HLA-G2 isoform polypeptide comprising the amino acid sequence of SEQ ID NO: 34 or 35, a β2M polypeptide (or a fragment or variant thereof), a membrane anchor, and one or more linkers (e.g., a flexible linker), and is linked to an exogenous antigenic polypeptide (e.g., via a linker).

In some embodiments, the exogenous HLA-G polypeptide comprises an alpha1 domain of an HLA-G3 isoform polypeptide and does not include a β2M polypeptide. In some embodiments, the exogenous HLA-G polypeptide comprises an alpha1 domain of an HLA-G3 isoform polypeptide and a β2M polypeptide, or a fragment or variant thereof. In some embodiments, the exogenous HLA-G polypeptide comprises an alpha1 domain of an HLA-G3 isoform polypeptide, and a membrane anchor. In some embodiments, the exogenous HLA-G polypeptide comprises an alpha1 domain of an HLA-G3 isoform polypeptide, a β2M polypeptide (or a fragment or variant thereof), and a membrane anchor. In some embodiments, the exogenous HLA-G polypeptide comprises an alpha1 domain of an HLA-G3 isoform polypeptide, a membrane anchor, and one or more linkers (e.g., a flexible linker). In some embodiments, the exogenous HLA-G polypeptide comprises an alpha1 domain of an HLA-G3 isoform polypeptide, a β2M polypeptide (or a fragment or variant thereof), a membrane anchor, and one or more linkers. In some embodiments, the exogenous HLA-G polypeptide comprises an alpha1 domain of an HLA-G3 isoform polypeptide, a membrane anchor, and one or more linkers, and is linked to an exogenous antigenic polypeptide (e.g., via a linker). In some embodiments, the exogenous HLA-G polypeptide comprises an alpha1 domain of an HLA-G3 isoform polypeptide, a β2M polypeptide (or a fragment or variant thereof), a membrane anchor, and one or more linkers, and is linked to an exogenous antigenic polypeptide (e.g., via a linker).

In some embodiments, the exogenous HLA-G polypeptide comprises alpha1 and alpha2 domains of an HLA-G4 isoform polypeptide and does not include a β2M polypeptide. In some embodiments, the exogenous HLA-G polypeptide comprises alpha1 and alpha2 domains of an HLA-G4 isoform polypeptide and a β2M polypeptide, or a fragment or variant thereof. In some embodiments, the exogenous HLA-G polypeptide comprises alpha1 and alpha2 domains of an HLA-G4 isoform polypeptide, and a membrane anchor. In some embodiments, the exogenous HLA-G polypeptide comprises alpha1 and alpha2 domains of an HLA-G4 isoform polypeptide, a β2M polypeptide (or a fragment or variant thereof), and a membrane anchor. In some embodiments, the exogenous HLA-G polypeptide comprises alpha1 and alpha2 domains of an HLA-G4 isoform polypeptide, a membrane anchor, and one or more linkers. In some embodiments, the exogenous HLA-G polypeptide comprises alpha1 and alpha2 domains of an HLA-G4 isoform polypeptide, a β2M polypeptide (or a fragment or variant thereof), a membrane anchor, and one or more linkers. In some embodiments, the exogenous HLA-G polypeptide comprises alpha1 and alpha2 domains of an HLA-G4 isoform polypeptide, a membrane anchor, and one or more linkers, and is linked to an exogenous antigenic polypeptide (e.g., via a linker). In some embodiments, the exogenous HLA-G polypeptide comprises alpha1 and alpha2 domains of an HLA-G4 isoform polypeptide, a β2M polypeptide (or a fragment or variant thereof), a membrane anchor, and one or more linkers, and is linked to an exogenous antigenic polypeptide (e.g., via a linker).

In some embodiments, the exogenous HLA-G polypeptide comprises alpha1, alpha2, and alpha3 domains of an HLA-G5 isoform polypeptide and does not include a β2M polypeptide. In some embodiments, the exogenous HLA-G polypeptide comprises alpha1, alpha2, and alpha3 domains of an HLA-G5 isoform polypeptide and a β2M polypeptide, or a fragment or variant thereof. In some embodiments, the exogenous HLA-G polypeptide comprises alpha1, alpha2, and alpha3 domains of an HLA-G5 isoform polypeptide, and a membrane anchor. In some embodiments, the exogenous HLA-G polypeptide comprises alpha1, alpha2, and alpha3 domains of an HLA-G5 isoform polypeptide, a β2M polypeptide (or a fragment or variant thereof), and a membrane anchor. In some embodiments, the exogenous HLA-G polypeptide comprises alpha1, alpha2, and alpha3 domains of an HLA-G5 isoform polypeptide, a membrane anchor, and one or more linkers. In some embodiments, the exogenous HLA-G polypeptide comprises alpha1, alpha2, and alpha3 domains of an HLA-G5 isoform polypeptide, a β2M polypeptide (or a fragment or variant thereof), a membrane anchor, and one or more linkers. In some embodiments, the exogenous HLA-G polypeptide comprises alpha1, alpha2, and alpha3 domains of an HLA-G5 isoform polypeptide, a membrane anchor, and one or more linkers, and is linked to an exogenous antigenic polypeptide (e.g., via a linker). In some embodiments, the exogenous HLA-G polypeptide comprises alpha1, alpha2, and alpha3 domains of an HLA-G5 isoform polypeptide, a β2M polypeptide (or a fragment or variant thereof), a membrane anchor, and one or more linkers, and is linked to an exogenous antigenic polypeptide (e.g., via a linker).

In some embodiments, the exogenous HLA-G polypeptide comprises alpha1 and alpha3 domains of an HLA-G6 isoform polypeptide and does not include a β2M polypeptide. In some embodiments, the exogenous HLA-G polypeptide comprises alpha1 and alpha3 domains of an HLA-G6 isoform polypeptide and a β2M polypeptide, or a fragment or variant thereof. In some embodiments, the exogenous HLA-G polypeptide comprises alpha1 and alpha3 domains of an HLA-G6 isoform polypeptide, and a membrane anchor. In some embodiments, the exogenous HLA-G polypeptide comprises alpha1 and alpha3 domains of an HLA-G6 isoform polypeptide, a β2M polypeptide (or a fragment or variant thereof), and a membrane anchor. In some embodiments, the exogenous HLA-G polypeptide comprises alpha1 and alpha3 domains of an HLA-G6 isoform polypeptide, a membrane anchor, and one or more linkers. In some embodiments, the exogenous HLA-G polypeptide comprises alpha1 and alpha3 domains of an HLA-G6 isoform polypeptide, a β2M polypeptide (or a fragment or variant thereof), a membrane anchor, and one or more linkers (e.g., a flexible linker). In some embodiments, the exogenous HLA-G polypeptide comprises alpha1 and alpha3 domains of an HLA-G6 isoform polypeptide, a membrane anchor, and one or more linkers, and is linked to an exogenous antigenic polypeptide (e.g., via a linker). In some embodiments, the exogenous HLA-G polypeptide comprises alpha1 and alpha3 domains of an HLA-G6 isoform polypeptide, a β2M polypeptide (or a fragment or variant thereof), a membrane anchor, and one or more linkers, and is linked to an exogenous antigenic polypeptide (e.g., via a linker).

In some embodiments, the exogenous HLA-G polypeptide comprises an alpha1 domain of an HLA-G7 isoform polypeptide and does not include a β2M polypeptide. In some embodiments, the exogenous HLA-G polypeptide comprises an alpha1 domain of an HLA-G7 isoform polypeptide and a β2M polypeptide (or a fragment or variant thereof). In some embodiments, the exogenous HLA-G polypeptide comprises an alpha1 domain of an HLA-G7 isoform polypeptide, and a membrane anchor. In some embodiments, the exogenous HLA-G polypeptide comprises an alpha1 domain of an HLA-G7 isoform polypeptide, a β2M polypeptide (or a fragment or variant thereof), and a membrane anchor. In some embodiments, the exogenous HLA-G polypeptide comprises an alpha1 domain of an HLA-G7 isoform polypeptide, a membrane anchor, and one or more linkers. In some embodiments, the exogenous HLA-G polypeptide comprises an alpha1 domain of an HLA-G7 isoform polypeptide, a β2M polypeptide (or a fragment or variant thereof), a membrane anchor, and one or more linkers. In some embodiments, the exogenous HLA-G polypeptide comprises an alpha1 domain of an HLA-G7 isoform polypeptide, a membrane anchor, and one or more linkers, and is linked to an exogenous antigenic polypeptide (e.g., via a linker). In some embodiments, the exogenous HLA-G polypeptide comprises an alpha1 domain of an HLA-G7 isoform polypeptide, a β2M polypeptide (or a fragment or variant thereof), a membrane anchor, and one or more linkers, and is linked to an exogenous antigenic polypeptide (e.g., via a linker).

In some embodiments, the alpha1 domain of an HLA-G isoform polypeptide (e.g., any of the HLA-G isoform polypeptides described herein) corresponds to the amino acid residues at positions 25 to 114 of SEQ ID NO: 34, or the amino acid residues at positions 1 to 90 of SEQ ID NO: 35. In some embodiments, the exogenous HLA-G polypeptide is encoded by a nucleic acid comprising or consisting of a nucleic acid sequence that is at least 40%, at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to a nucleic acid sequence encoding the amino acid residues at positions 1 to 90 of SEQ ID NO: 35.

In some embodiments, the alpha2 domain of an HLA-G isoform polypeptide (e.g., any of the HLA-G isoform polypeptides described herein) corresponds to the amino acid residues at positions 115 to 206 of SEQ ID NO: 34, or the amino acid residues at positions 91 to 182 of SEQ ID NO: 35. In some embodiments, the exogenous HLA-G polypeptide is encoded by a nucleic acid comprising or consisting of a nucleic acid sequence that is at least 40%, at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to a nucleic acid sequence encoding the amino acid residues at positions 91 to 182 of SEQ ID NO: 35.

In some embodiments, the alpha3 domain of an HLA-G isoform polypeptide (e.g., any of the HLA-G isoform polypeptides described herein) corresponds to the amino acid residues at positions 207 to 298 of SEQ ID NO: 34, or the amino acid residues at positions 183 to 274 of SEQ ID NO: 35. See, e.g., Geraghty et al., PNAS 84(24):9145-9149, 1987. In some embodiments, the exogenous HLA-G polypeptide is encoded by a nucleic acid comprising or consisting of a nucleic acid sequence that is at least 40%, at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to a nucleic acid sequence encoding the amino acid residues at positions 183 to 274 of SEQ ID NO: 35.

Nucleic acids comprising or consisting of a nucleic acid sequence encoding an exogenous HLA-G polypeptide described herein are also provided. In some embodiments, the nucleic acid comprises at least one promoter (e.g., a constitutive or an inducible promoter) operably-linked to the open reading frame or gene encoding the exogenous HLA-G polypeptide. In some embodiments, the exogenous HLA-G polypeptide is encoded by a nucleic acid comprising or consisting of a nucleic acid sequence that is at least 40%, at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to a nucleic acid sequence encoding a wild-type HLA-G polypeptide. In some embodiments, the exogenous HLA-G polypeptide is encoded by a nucleic acid comprising or consisting of a nucleic acid sequence that is at least 40%, at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to a nucleic acid sequence encoding a wild-type HLA-G polypeptide, wherein the exogenous HLA-G polypeptide does not include a signal sequence. In some embodiments, the nucleic acid is codon-optimized (e.g., for expression in a human cell). In some embodiments, the nucleic acid is not codon-optimized.

Exogenous HLA-G polypeptides may include full-length HLA-G polypeptides and functional fragments thereof, as well as homologs, isoforms, and variants of a wild-type naturally occurring HLA-G polypeptides. For example, in some embodiments, the amino acid sequence of an exogenous HLA-G polypeptide may differ from the amino acid sequence of a wild-type exogenous HLA-G polypeptide from which it was derived at one or more amino acid residues. For example, in some embodiments, the exogenous HLA-G polypeptide may be modified from the wild-type amino acid sequence, to include, for example, one or more amino acid deletions, insertions, and/or substitutions. In some embodiments, the amino acid sequence of an exogenous HLA-G polypeptide is modified as compared to the amino acid sequence of a wild-type HLA-G polypeptide to include a conservative (e.g., structurally-similar) amino acid substitution. For instance, structurally similar amino acids include: (isoleucine (I), leucine (L) and valine (V)); (phenylalanine (F) and tyrosine (Y)); (lysine (K) and arginine (R)); (glutamine (Q) and asparagine (N)); (aspartic acid (D) and glutamic acid (E)); and (glycine (G) and alanine (A)). In some embodiments, the amino acid sequence of an exogenous HLA-G polypeptide is modified as compared to the amino acid sequence of a wild-type exogenous HLA-G polypeptide to include a non-conservative amino acid substitution. In some embodiments, the exogenous HLA-G polypeptide comprises an amino acid sequence that differs from a wild-type HLA-G polypeptide amino acid sequence (e.g., by truncation, deletion, substitution, or addition) by no more than 1, 2, 3, 4, 5, 8, 10, 20, or 50 residues, and retains a function of the wild-type HLA-G polypeptide from which it was derived.

In some embodiments, an exogenous HLA-G polypeptide may include an additional amino acid sequence not present in a wild-type amino acid sequence, such as a regulatory peptide sequence, a linker, a epitope tag (e.g., a His-tag, a FLAG-tag or a myc tag), a membrane anchor, e.g., a glycophorin A (GPA) protein, a transmembrane domain of GPA, a transmembrane domain of small integral membrane protein 1 (SMIM1), or a transmembrane domain of transferrin receptor, and other peptide sequence. The additional amino acid sequence may be present at the N-terminus or C-terminus of the exogenous HLA-G polypeptide or may be disposed within the polypeptide's amino acid sequence. In some embodiments, an exogenous HLA-G polypeptide comprises a post-translational modification (e.g., glycosylation). In some embodiments, an exogenous HLA-G polypeptide oligomerizes within or on the surface of a cell described herein. In some embodiments, the exogenous HLA-G polypeptide comprises a leader sequence (e.g., a naturally-occurring leader sequence or a leader sequence of a different polypeptide). In some embodiments, the exogenous HLA-G polypeptide lacks a leader sequence (e.g., is genetically modified to remove a naturally-occurring leader sequence). In some embodiments, the exogenous HLA-G polypeptide has an N-terminal methionine residue. In some embodiments, the exogenous HLA-G polypeptide lacks an N-terminal methionine residue.

In some embodiments, an engineered erythroid cell or enucleated cell described herein comprises a non-transmembrane polypeptide on the cell surface, e.g., an exogenous antigenic polypeptide, an exogenous immunogenic polypeptide, or an exogenous (32 microglobulin polypeptide. Thenon-transmembrane polypeptide may be either: assembled with another agent within the cell prior to trafficking to the cell surface; secreted by the cell and then captured on the cell surface by a membrane-tethered polypeptide on the cell surface (e.g., an exogenous HLA-G polypeptide); or has been contacted with the cell (e.g., in purified form) and is then captured on the cell surface by a membrane-tethered polypeptide on the cell surface.

In some embodiments, the exogenous HLA-G polypeptide can be tethered to the plasma membrane of the cell via attachment to a lipid moiety (e.g., N-myristoylation, S-palmitoylation, farnesylation, geranylgeranylation, or glycosylphosphatidyl inositol (GPI) anchor).

In some embodiments, the exogenous HLA-G polypeptide comprises one or more alpha domains 1-3 (e.g., alpha1, alpha2, and alpha3 domains) of an HLA-G polypeptide, a membrane anchor, and a β2M polypeptide (or a fragment or variant thereof). In some embodiments, the exogenous HLA-G polypeptide further comprises an antigenic polypeptide linked via a linker (e.g., a linker provided herein (e.g., a cleavable linker or a flexible linker)). In some embodiments, the membrane anchor is a glycophorin anchor, and in particular glycophorin A (GPA), or the membrane anchor is small integral membrane protein 1 (SMIM1). In some embodiments, the membrane anchor comprises full-length GPA. In some embodiments, the membrane anchor comprises full-length SMIM1. In some embodiments, the membrane anchor comprises the transmembrane domain of SMIM1 or the transmembrane domain of GPA. In some embodiments, the membrane anchor comprises full-length transferrin receptor or a fragment thereof (e.g., a fragment comprising the transferrin receptor transmembrane domain). In some embodiments, the membrane anchor comprises or consists of an amino acid sequence set forth in the Table A.

TABLE A

| SEQ | Membrane | | |
|---|---|---|---|
| ID | anchor | | |
| NO: | name | Description | Amino acid sequence |
| SEQ | GPA | Full length GPA | MYGKIIFVLLLSAIVSISALSTTEVAMHTSTSSS |
| ID | | | VTKSYISSQTNDTHKRDTYAATPRAHEVSEISV |
| NO: 2 | | | RTVYPPEEETGERVQLAHHFSEPEITLIIFGVMA |
| | | | GVIGTILLISYGIRRLIKKSPSDVKPLPSPDTDVP |
| | | | LSSVEIENPETSDQ |
| SEQ | GPA | Fragment of | LSTTEVAMHTSTSSSVTKSYISSQTNDTHKRDT |
| ID | | GPA comprising | YAATPRAHEVSEISVRTVYPPEEETGERVQLA |
| NO: 3 | | a transmembrane | HHFSEPEITLIIFGVMAGVIGTILLISYGIRRLIKK |
| | | domain | SPSDVKPLPSPDTDVPLSSVEIENPETSDQ |
| SEQ | SMIM1 | SMIM1 | MQPQESHVHYSRWEDGSRDGVSLGAVSSTEE |
| ID | | | ASRCRRISQRLCTGKLGIAMKVLGGVALFWIIF |
| NO: 4 | | | ILGYLTGYYVHKCK |

In some embodiments, the exogenous HLA-G polypeptide or fusion protein comprises the structure set forth in FIG. 1A, 1B, 1C or 1D.

In some embodiments, an exogenous HLA-G polypeptide present on an engineered enucleated erythroid cell or enucleated cell described herein is capable of binding to one or more HLA-G receptors, such as ILT4, ILT2, and/or KIR2DL4 (e.g., present on the surface of a NK cell, a CD8⁺ T cell, a CD4⁺ T cell, a B cell, a monocyte, and/or a dendritic cell).

### Exogenous Immunogenic Polypeptides

In some embodiments, the engineered erythroid cells (e.g., engineered enucleated erythroid cells) or enucleated cells (e.g., modified enucleated cells) described herein include an exogenous HLA-G polypeptide and an exogenous immunogenic polypeptide, wherein both the exogenous HLA-G polypeptide and the exogenous immunogenic polypeptide are on the cell surface. In some embodiments, the exogenous immunogenic polypeptide is not bound to the exogenous HLA-G polypeptide.

In other embodiments, the engineered erythroid cells (e.g., engineered enucleated erythroid cells) or enucleated cells (e.g., modified enucleated cells) described herein include an exogenous HLA-G polypeptide and an exogenous immunogenic polypeptide, wherein the exogenous HLA-G polypeptide is on the cell surface and the exogenous immunogenic polypeptide is within the cell (i.e., intracellular) (e.g., an exogenous immunogenic enzyme, e.g., IDO or CD39). In some embodiments, the exogenous immunogenic polypeptide is in the cytoplasm of the cell. In some embodiments, the exogenous immunogenic polypeptide is on the intracellular side of the plasma membrane (e.g., positioned at the intracellular side of the plasma membrane using any of the exemplary membrane anchors described herein). In some embodiments, the exogenous immunogenic polypeptide is secreted or released by the cell. In some embodiments, the intracellular exogenous immunogenic polypeptide is not bound to the exogenous HLA-G polypeptide.

Also provided herein are engineered erythroid cells (e.g., engineered enucleated erythroid cells) or enucleated cells (e.g., modified enucleated cells) that include an exogenous immunogenic polypeptide (e.g., any of the exogenous immunogenic polypeptides described herein) and at least one exogenous coinhibitory polypeptide (e.g., any of the exogenous coinhibitory polypeptides described herein). In some embodiments, the exogenous immunogenic polypeptide is in the cytosol of the cell. In some embodiments, the exogenous immunogenic polypeptide is on the intracellular side of the plasma membrane. In some embodiments, the exogenous immunogenic polypeptide is secreted or released by the cell. In some embodiments, the at least one exogenous coinhibitory polypeptide is on the intracellular side of the plasma membrane. In some embodiments, the at least one exogenous coinhibitory polypeptide is secreted or released by the cell.

In some embodiments, the exogenous immunogenic polypeptide can be tethered to the plasma membrane of the cell via attachment to a lipid moiety (e.g., N-myristoylation, S-palmitoylation, farnesylation, geranylgeranylation, and glycosylphosphatidyl inositol (GPI) anchor).

In some embodiments, the exogenous immunogenic polypeptide can include a membrane anchor. In some embodiments, the membrane anchor is on the N-terminus of the exogenous immunogenic polypeptide. In other embodiments, the membrane anchor is on the C-terminus of the exogenous immunogenic polypeptide.

An exogenous immunogenic polypeptide for use as described herein may be derived from any source. In some embodiments, the exogenous immunogenic polypeptide comprises a human polypeptide. In some embodiments, the exogenous immunogenic polypeptide comprises an alloreactive polypeptide (e.g., a human alloreactive polypeptide). In some embodiments, the exogenous immunogenic polypeptide comprises a non-human polypeptide. In some embodiments, the exogenous immunogenic polypeptide comprises a non-human polypeptide derived from a bacterium, a plant, a yeast, a fungus, a virus, a prion, or a protozoan. In some embodiments, the exogenous immunogenic polypeptide comprises any one of the polypeptide set forth in Tables 1 or 2 herein.

In some embodiments, the exogenous immunogenic polypeptide for use as described herein comprises an amino acid-degrading polypeptide (e.g., an asparaginase polypeptide, a phenylalanine ammonium lyase (PAL) polypeptide, a phenylalanine hydroxylase (PAH) polypeptide, a homocysteine-reducing polypeptide or a homocysteine-degrading polypeptide), a uric acid-degrading polypeptide, or an oxalate oxidase). In some embodiments, the exogenous immunogenic polypeptide comprises a d-aminolevulinate dehydrogenase (ALA-D), also known as porphobilinogen synthase or delta-aminolevulinic acid dehydratase.

In some embodiments, an engineered erythroid cell (e.g., engineered enucleated erythroid cell) or enucleated cell (e.g., modified enucleated cell) described herein comprises two or more, (e.g., at least 2, 3, 4, 5, 6, 7, 8, 9, 10, 11, 12, 13, 14, 15, 16, 17, 18, 19, 20, or more) exogenous immunogenic polypeptides. In some embodiments, a population of engineered erythroid cells or enucleated cells described herein comprises two or more (e.g., at least 2, 3, 4, 5, 6, 7, 8, 9, 10, 11, 12, 13, 14, 15, 16, 17, 18, 19, 20, or more) exogenous immunogenic polypeptides, wherein different engineered erythroid cells or enucleated cells in the population comprise different types of exogenous immunogenic polypeptides or wherein different erythroid cells in the population comprise different pluralities of types of exogenous immunogenic polypeptides.

In some embodiments, the exogenous immunogenic polypeptide comprises an amino acid sequence having at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the amino acid sequence of a corresponding wild-type immunogenic polypeptide.

Nucleic acids comprising or consisting of a nucleic acid sequence encoding an exogenous immunogenic polypeptide described herein are also provided. In some embodiments, the nucleic acid comprises at least one promoter (e.g., a constitutive or an inducible promoter) operably-linked to the open reading frame or gene encoding the exogenous immunogenic polypeptide. In some embodiments, the exogenous immunogenic polypeptide is encoded by a nucleic acid (e.g., an exogenous nucleic acid) comprising or consisting of a nucleic acid sequence that is at least 40%, at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to a nucleic acid sequence encoding a wild-type immunogenic polypeptide. In some embodiments, the exogenous immunogenic polypeptide is encoded by a nucleic acid comprising or consisting of a nucleic acid sequence that is at least 40%, at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identical to a nucleic acid sequence encoding a wild-type exogenous immunogenic polypeptide, wherein the exogenous immunogenic polypeptide does not include a signal sequence. In some embodiments, the nucleic acid is codon-optimized (e.g., for expression in a human cell). In some embodiments, the nucleic acid is not codon-optimized.

Exogenous immunogenic polypeptide include full-length polypeptides and functional fragments thereof (e.g., enzymatically-active fragments thereof), as well as homologs, isoforms, and variants of a wild-type naturally occurring exogenous immunogenic polypeptides which may retain activity, e.g., enzymatic activity. For example, in some embodiments, the amino acid sequence of an exogenous immunogenic polypeptide may differ from the amino acid sequence of a wild-type exogenous immunogenic polypeptide from which it was derived at one or more amino acid residues. For example, in some embodiments, the exogenous immunogenic polypeptide may be modified from the wild-type amino acid sequence, to include, for example, one or more amino acid deletions, insertions, and/or substitutions. In some embodiments, the amino acid sequence of an exogenous immunogenic polypeptide is modified as compared to the amino acid sequence of a wild-type exogenous immunogenic polypeptide to include a conservative (e.g., structurally-similar) amino acid substitution or a non-conservative amino acid substitution. In some embodiments, the exogenous immunogenic polypeptide amino acid sequence differs from a wild-type immunogenic polypeptide amino acid sequence (e.g., by truncation, deletion, substitution, or addition) by no more than 1, 2, 3, 4, 5, 8, 10, 20, or 50 residues, and retains a function of the wild-type exogenous immunogenic polypeptide from which it was derived.

In some embodiments, fragments or variants of an exogenous immunogenic polypeptide comprise at least 25%, at least 30%, at least 40%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100% of the exogenous immunogenic polypeptide activity of the wild-type exogenous immunogenic polypeptide from which the fragment or variant was derived.

In some embodiments, an exogenous immunogenic polypeptide may include an additional amino acid sequence not present in a wild-type amino acid sequence, such as a regulatory peptide sequence, a linker, an epitope tag (e.g., a His-tag, a FLAG-tag or a myc tag), a membrane anchor, e.g., transmembrane protein (e.g., GPA, SMIM1 or Kell, or transferrin receptor) or transmembrane domain thereof. The additional amino acid sequence may be present at the N-terminus or C-terminus of the exogenous immunogenic polypeptide or may be disposed within the polypeptide's amino acid sequence. In some embodiments, the exogenous immunogenic polypeptide comprises a membrane anchor (e.g., a Type I or Type II membrane polypeptide or a transmembrane domain thereof) disposed such that a portion or all of the exogenous immunogenic polypeptide (except for the membrane anchor) locates to the cytosol of the cell (e.g., proximate to the inner leaflet of the plasma membrane). In some embodiments, the exogenous immunogenic polypeptide comprises a membrane domain (e.g., a transmembrane domain or a transmembrane polypeptide) disposed such that a portion or all of the exogenous immunogenic polypeptide (except for the membrane anchor) locates in the outer surface of the cell (e.g., facing the extracellular milieu of the cell). In some embodiments, the exogenous immunogenic polypeptide does not include a membrane anchor (e.g., a transmembrane domain or a transmembrane polypeptide).

In some embodiments, the exogenous immunogenic polypeptide comprises a post-translational modification (e.g., glycosylation). In some embodiments, the exogenous immunogenic polypeptide oligomerizes within or on the cell surface of a cell described herein. In some embodiments, the exogenous immunogenic polypeptide comprises a leader sequence (e.g., a naturally-occurring leader sequence or a leader sequence of a different polypeptide). In some embodiments, the exogenous immunogenic polypeptide lacks a leader sequence (e.g., is genetically modified to remove a naturally-occurring leader sequence). In some embodiments, the exogenous immunogenic polypeptide has an N-terminal methionine residue. In some embodiments, the exogenous immunogenic polypeptide lacks an N-terminal methionine residue.

In some embodiments, the exogenous immunogenic polypeptide may include a linker (e.g., disposed between the membrane anchor and the remaining amino acid sequence of the exogenous immunogenic polypeptide). Any linker provided herein may be included in the exogenous immunogenic polypeptide. In some embodiments, the linker is a poly-glycine poly-serine linker. For example, in some embodiments, the linker comprises or consists of the amino acid sequence (Gly₄Ser)ₙ, wherein (n=1-20) (SEQ ID NO: 839). In some embodiments, the poly-glycine poly-serine linker exclusively includes glycine and/or serine amino acid residues. In some embodiments, the linker comprises or consists of a poly-glycine poly-serine linker with one or more amino acid substitutions, deletions, and/or additions and which lacks the amino acid sequence GSG. In some embodiments, a linker comprises or consists of the amino acid sequence (GGGXX)ₙGGGGS (SEQ ID NO:20) or GGGGS(XGGGS)ₙ (SEQ ID NO:21), where n is greater than or equal to one. In some embodiments, n is between 1 and 20, inclusive (e.g., n may be 1, 2, 3, 4, 5, 6, 7, 8, 9, 10, 11, 12, 13, 14, 15, 16, 17, 18, 19, or 20). Additional linkers include, but are not limited to, GGGGSGGGGS (SEQ ID NO: 22), GSGSGSGSGS (SEQ ID NO:23), PSTSTST (SEQ ID NO:24), and EIDKPSQ (SEQ ID NO:25), and multimers thereof. In some embodiments, the linker is GSGSGSGSGSGSGSGSGS (SEQ ID NO: 840) or GPGPG (SEQ ID NO: 841).

In some embodiments, an engineered erythroid cell or enucleated cell described herein is contacted with, comprises, or expresses a nucleic acid (e.g., DNA or RNA) encoding an exogenous immunogenic polypeptide described herein.

In some embodiments, an exogenous immunogenic polypeptide comprises a polypeptide described in Table 1, below. In some embodiments, an exogenous immunogenic polypeptide comprises a polypeptide disclosed in U.S. Pat. No. 9,644,180, the contents of which are incorporated by reference herein in their entirety.

TABLE 1
Exemplary immunogenic polypeptides

| | | | |
|---|---|---|---|
| triacylglycerol lipase | bile-acid-CoA hydrolase | feruloyl esterase | phosphatidate |
| | | | phosphatase |
| (S)-methylmalonyl-CoA | bis(2-ethylhexyl)phthalate | formyl-CoA hydrolase | phosphatidylglyceropho |
| hydrolase | esterase | | sphatase |
| [acyl-carrier-protein] | bisphosphoglycerate | fructose- | phosphatidylinositol |
| phosphodiesterase | phosphatase | bisphosphatase | deacylase |
| [phosphorylase] | carboxylic-Ester Hydrolases | fumarylacetoacetase | phosphodiesterase I |
| phosphatase | | | |
| 1,4-lactonase | Carboxymethylenebutenolidase | fusarinine-C | phosphoglycerate |
| | | ornithinesterase | phosphatase |
| 11-cis-retinyl-palmitate | cellulose-polysulfatase | galactolipase | phosphoglycolate |
| hydrolase | | | phosphatase |
| 1-alkyl-2- | cephalosporin-C | gluconolactonase | phosphoinositide |
| acetylglycerophosphocholine | deacetylase | | phospholipase C |
| esterase | | | |
| 2′-hydroxybiphenyl- | cerebroside-sulfatase | glucose-1-phosphatase | phospholipase A1 |
| 2-sulfinate desulfinase | | | |
| 2-pyrone-4,6- | cetraxate benzylesterase | glucose-6-phosphatase | phospholipase A2 |
| dicarboxylate lactonase | | | |
| 3′,5′-bisphosphate | chlorogenate hydrolase | glutathione thiolesterase | phospholipase C |
| nucleotidase | | | |
| 3-hydroxyisobutyryl- | chlorophyllase | glycerol-1-phosphatase | phospholipase D |
| CoA hydrolase | | | |
| 3′-nucleotidase | Cholinesterase | glycerol-2-phosphatase | phosphonoacetaldehyde |
| | | | hydrolase |
| 3-oxoadipate enol- | choline-sulfatase | glycerophosphocholine | phosphonoacetate |
| lactonase | | phosphodiesterase | hydrolase |
| 3-phytase | choloyl-CoA hydrolase | glycosidases (i.e. | phosphonopyruvate |
| | | enzymes that hydrolyse | hydrolase |
| | | O- and S-glycosyl | |
| | | compounds) | |
| 4-hydroxybenzoyl- | chondro-4-sulfatase | glycosulfatase | phosphoprotein |
| CoA thioesterase | | | phosphatase |
| 4-methyloxaloacetate | chondro-6-sulfatase | glycosylases | phosphoric-diester |
| esterase | | | hydrolases |
| 4-phytase | citrate-lyase | histidinol-phosphatase | phosphoric-monoester |
| | deacetylase | | hydrolases |
| 4-pyridoxolactonase | cocaine esterase | hormone-sensitive lipase | phosphoric-triester |
| | | | hydrolases |
| 5′-nucleotidase | Cutinase | hydrolysing N-glycosyl | phosphoserine phosphatase |
| | | compounds | |
| 6-acetylglucose | cyclamate | hydrolysing S-glycosyl | poly(3-hydroxybutyrate) |
| deacetylase | sulfohydrolase | compounds | depolymerase |
| 6- | cysteine | hydroxyacylglutathione | poly(3-hydroxyoctanoate) |
| phosphogluconolactonase | endopeptidases | hydrolase | depolymerase |
| a-amino-acid esterase | cysteine-type | hydroxybutyrate-dimer | polyneuridine-aldehyde |
| | carboxypeptidases | hydrolase | esterase |
| a-amino-acyl-peptide | D-arabinonolactonase | hydroxymethylglutaryl- | protein-glutamate |
| hydrolases | | CoA hydrolase | methylesterase |
| acetoacetyl-CoA | deoxylimonate A- | iduronate-2-sulfatase | quorum-quenching N-acyl- |
| hydrolase | ring-lactonase | | homoserine lactonase |
| acetoxybutynylbithiophene | dGTPase | inositol-phosphate | retinyl-palmitate esterase |
| deacetylase | | phosphatase | |
| acetylajmaline esterase | dihydrocoumarin | juvenile-hormone | serine dehydratase |
| | hydrolase | esterase | |
| acetylalkylglycerol | Dipeptidases | kynureninase | serine endopeptidases |
| acetylhydrolase | | | |
| acetylcholinesterase | dipeptide hydrolases | L-arabinonolactonase | serine- |
| | | | ethanolaminephosphate |
| | | | phosphodiesterase |
| acetyl-CoA hydrolase | dipeptidyl-peptidases | limonin-D-ring- | serine-type |
| | and tripeptidyl- | lactonase | carboxypeptidases |
| | peptidases | | |
| acetylesterase | diphosphoric- | lipoprotein lipase | S-formylglutathione |
| | monoester hydrolases | | hydrolase |
| acetylpyruvate hydrolase | disulfoglucosamine- | L-rhamnono-1,4- | sialate O-acetylesterase |
| | 6-sulfatase | lactonase | |
| acetylsalicylate | dodecanoyl-[acyl- | lysophospholipase | sinapine esterase |
| deacetylase | carrier-protein] | | |
| | hydrolase | | |
| acetylxylan esterase | Endodeoxyribonucleases | mannitol-1-phosphatase | endodeoxyribonucleases |
| acid phosphatase | endopeptidases | metallocarboxypeptidases | sphingomyelin |
| | | | phosphodiesterase |
| actinomycin lactonase | endoribonucleases | metalloendopeptidases | S-succinylglutathione |
| | | | hydrolase |
| acylcamitine hydrolase | enzymes acting on | methylphosphothioglycerate | steroid-lactonase |
| | carbon-nitrogen | phosphatase | |
| | bonds, other than | | |
| | peptide bonds | | |
| acyl-CoA hydrolase | enzymes acting on | methylumbelliferyl- | sterol esterase |
| | carbon-phosphorus bonds | acetate deacetylase | |
| acylglycerol lipase | enzymes acting on | monoterpene e-lactone | steryl-sulfatase |
| | carbon-sulfur bonds | hydrolase | |
| acyloxyacyl hydrolase | enzymes acting | N-acetylgalactosamine-4- | succinyl-CoA |
| | on ether bonds | sulfatase | hydrolase |
| acylpyruvate hydrolase | enzymes acting | N-acetylgalactosamine-6- | sucrose-phosphate |
| | on halide bonds | sulfatase | phosphatase |
| ADAMTS13 | enzymes acting | N- | sugar-phosphatase |
| | on peptide bonds | acetylgalactosaminoglycan | |
| | (peptidases) | deacetylase | |
| adenosine deaminase | enzymes acting on | N-acetylglucosamine-6- | sulfuric-ester |
| | phosphorus-nitrogen | sulfatase | hydrolases |
| | bonds | | |
| adenylyl-[glutamate- | enzymes acting on | N-sulfoglucosamine | tannase |
| ammonia ligase] hydrolase | sulfur-nitrogen bonds | sulfohydrolase | |
| ADP-dependent medium- | enzymes acting on | oleoyl-[acyl-carrier- | thioester hydrolases |
| chain-acyl-CoA hydrolase | sulfur-sulfur bonds | protein] hydrolase | |
| ADP-dependent short- | ether hydrolases | omega peptidases | thioether and |
| chain-acyl-CoA hydrolase | | | trialkylsulfonium |
| | | | hydrolases |
| ADP-phosphoglycerate | exodeoxyribonucleases | orsellinate-depside | threonine endopeptidases |
| phosphatase | producing 5′- | hydrolase | |
| | phosphomonoesters | | |
| alkaline phosphatase | Exonucleases | oxaloacetase | thymidine phosphorylase |
| all-trans-retinyl- | exoribonucleases | palmitoyl[protein] | trehalose-phosphatase |
| palmitate hydrolase | | hydrolase | |
| aminoacyl-tRNA | Factor IX | palmitoyl-CoA | triacetate-lactonase |
| hydrolase | | hydrolase | |
| aminopeptidases | Factor VIII | pectinesterase | triphosphoric- |
| | | | monoester hydrolases |
| arylesterase | fatty-acyl-ethyl- | peptidyl peptide | trithionate hydrolase |
| | ester synthase | hydrolases | |
| arylsulfatase | phorbol-diester | peptidyl-amino-acid | tropinesterase |
| | hydrolase | hydrolases | |
| asparaginase | phloretin hydrolase | peptidylamino-acid | ubiquitin thiolesterase |
| | | hydrolases | |
| aspartic endopeptidases | acylamino-acid | peptidyl-dipeptidases | UDP-sulfoquinovose |
| | hydrolases | | synthase |
| uronolactonase | serine hydroxymethyl | phenylacetyl-CoA | uricase |
| | transferase | hydrolase | |
| phenylalanine ammonia | pheophorbidase | phenylalanine | wax-ester hydrolase |
| lyase (PAL) | | hydroxylase (PAH) | |

a. Amino Acid-Degrading Polypeptides

In some embodiments, an exogenous immunogenic polypeptide provided herein comprises or consists of an amino acid-degrading polypeptide. U.S. Patent Publication No. 2019/0160102 (which is incorporated herein by reference in its entirety) describes amino acid-degrading polypeptides that can be included in an exogenous immunogenic polypeptide on the cell surface of the engineered erythroid cells (e.g., engineered enucleated erythroid cells) or enucleated cells (e.g., modified enucleated cells) described herein. Exemplary amino acid-degrading polypeptides include, for example, an asparaginase, a phenylalanine ammonium lyase (PAL), a phenylalanine hydroxylase (PAH), a homocysteine-reducing polypeptide, and a homocysteine-degrading polypeptide.

In some embodiments, the amino acid-degrading polypeptide comprises an asparaginase, a serine dehydratase, a serine hydroxymethyltransferase polypeptide, a NAD-dependent L-serine dehydrogenase, an arginase, an arginine deiminase, a methionine gamma-lyase, a L-amino acid oxidase, a S-adenosylmethionine synthase, a cystathionine gamma-lyase, an indoleamine 2,3-dioxygenase, or a phenylalanine ammonia lyase. In some embodiments, the amino acid-degrading polypeptide comprises a glutaminase, a glutamine-pyruvate transaminase, a branched-chain-amino-acid transaminase, an amidase, an arginine decarboxylase, an aromatic-L-amino-acid decarboxylase, a cysteine lyase, or an argininosuccinate lyase. In some embodiments, the amino acid-degrading polypeptide comprises an enzymatically-active polypeptide.

1. Asparaginases

In some embodiments, the exogenous immunogenic polypeptide provided herein comprises or consists of an amino acid-degrading polypeptide comprising an asparaginase, or a fragment or variant thereof. In some embodiments, the asparaginase is an asparaginase described in Covini et al. (2012) Recent Pat. Anticancer Drug Discov. 7(1):4-13 (which is herein incorporated by reference in its entirety, including Table 1 therein), or an asparaginase having an amino acid sequence having at least 70%, 80%, 85%, 90%, 95%, 96%, 97%, 98%, or 99% sequence identity thereto. In some embodiments, the asparaginase is an asparaginase from either Arabidopsis thaliana, Homo sapiens, Erwinia chrysanthemi, or Helicobacter pylori, or a fragment or variant thereof. In some embodiments, the exogenous immunogenic polypeptide comprising an asparaginase can metabolize asparagine with a k_{cat} at least 90%, 80%, 70%, 60%, or 50% of that of a wild-type asparaginase from which it was derived, or a Kₘ less than 150%, 125%, 100%, 75%, or 50% of the Kₘ of a wild-type asparaginase, or a combination thereof. Additional asparaginases are described, e.g., in Gervais and Foote, (supra), Nguyen et al. (2016) J. Biol Chem. 291(34): 17664-76, and Moola et al. (1994) Biochem. J. 302(3): 921-7, each of which is herein incorporated by reference in their entireties.

Numerous asparaginases have been identified in bacteria, plants, yeast, algae, fungi and mammals, and may be used as described herein. For example, in some embodiments, the exogenous immunogenic polypeptide comprises an asparaginase from Escherichia coli (see, e.g., UnitProt Accession No. P00805), Erwinia carotovora (also known as Pectobacterium atrosepticum; see, e.g., GenBank Accession No. AAS67027), Erwinia chrysanthemi (also known as Dickeya chrysanthemi; see, e.g., UniProt Accession Nos. P06608, and AAS67028; and GenBank Accession No. CAA31239); Erwinia carotovora (also known as Pectobacterium atrosepticum; see, e.g., GenBank Accession Nos. AAS67027, AAP92666 and Q6Q4F4), Pseudomonas stutzeri (see, e.g., GenBank Accession No. AVX11435), Delftia acidovoras (also known as Pseudomonas acidovorans; see, e.g., GenBank Accession No. ABX36200), Pectobacterium carotovorum (also known as Erwinia aroideae; see, e.g., NCBI Reference No. WP_015842013), Thermus thermophilus (see, e.g., GenBank Accession Nos. BAD69890 and BAW01549), Thermus aquaticus (see, e.g., GenBank Accession Nos. KOX89292 and EED09821), Staphylococcus aureus (see, e.g., GenBank Accession Nos KII20890, ARI73732, and P1195560), Wolinella succinogenes (also known as Vibrio succinogenes; see, e.g., GenBank Accession No. CAA61503), Citrobacter freundi (see, e.g., GenBank Accession No. EXF30424), Proteus vulgaris (see, e.g., GenBank Accession No. KGA60073), Zymomonas mobilis (see, e.g., GenBank Accession Nos. AHB10760, ART93886, AAV90307, AEH63277, and ACV76074), Bacillus subtilis (see, e.g., UniProt Accession No. 03448), Bacillus licheniformis (see, e.g., GenBank Accession Nos. ARW56273, ARW54537, ARW44915, and AOP17372), Bacillus circulans (see, e.g., GenBank Accession Nos. KLV25750, PAE13094, PAD89980, PAD81349, PAD90008, and PAE13121), Enterobacter aerogenes (see, e.g., NCBI Reference No. YP_004594521, and GenBank Accession No. SFX86538), Serratia marcescens (see, e.g., GenBank Accession Nos. ALD46588, ALE95248, OSX81952, and PHI53192), Wolinella succinogenes (see, e.g., UniProt Accession No. P50286), Helicobacter pylori (see, e.g., UniProt Accession No. 025424), and Cavia porcellus (guinea pig) (see, e.g., UniProt Accession No. H0W0T5), Aspergillus nomius (see, e.g., NCBI Reference No. XP_015407819), Aspergillus terreus (see, e.g., GenBank Accession Nos. EAU36905 and KT728852), Aspergillus fischeri (NCBI Reference No. XP_001265372), Aspergillus fumigatus (NCBI Reference No. XP_750028), Glarea lozoyensis (see, e.g., NCBI Reference No. XP_008086736), Saccharomyces cerevisae (see, e.g., NCBI Reference No. NP_010607), Cyberlindnera jadinii (also known as Candida utilis; see, e.g., GenBank Accession No. CEP24033); Meyerozyma guilliermondii (also known as Candida guilliermondii; see, e.g., NCBI Reference No. XP_001485067; and GenBank Accession No. EDK36913), and Rhodotorula toruloides (see, e.g., NCBI Reference Nos. XP_016274149.1 and XP_016272508), of a fragment or variant of any of the foregoing.

In some embodiments, the exogenous immunogenic polypeptide comprising an asparagine (and cells comprising the exogenous immunogenic polypeptide) has asparaginase activity. Asparaginase activity can be measured, e.g., using an assay of Gervais and Foote (2014) Mol. Biotechnol. 45(10): 865-77, which is herein incorporated by reference in its entirety.

Engineered erythroid cells and enucleated cells comprising an exogenous immunogenic polypeptide comprising an asparaginase can be used in the treatment of a cancer described herein, including e.g., a leukemia (e.g., acute myeloid leukemia (AML), acute lymphoblastic leukemia (ALL), lymphoblastic lymphoma), a lymphoma (e.g., NK/T cell lymphoma or non-Hodgkin lymphoma), pancreatic cancer, ovarian cancer, fallopian cancer, and peritoneal cancer.

2. Phenylalanine Ammonia Lyases (PALs)

In some embodiments, the exogenous immunogenic polypeptide comprises or consists of an amino acid-degrading polypeptide comprising a phenylalanine ammonia lyase (PAL), or a fragment or variant thereof. Engineered erythroid cells or enucleated cells comprising an exogenous immunogenic polypeptide comprising a PAL, or a fragment or variant thereof, may be used to treat subjects having phenylketonuria (PKU). In some embodiments, the exogenous immunogenic polypeptide comprises a PAL from Anabaena variabilis Arabidopsis thaliana, Pseudomonas putida, or a fragment or variant thereof.

In some embodiments, an exogenous immunogenic polypeptide comprising a PAL provided herein (and cells comprising the exogenous immunogenic polypeptide) is capable of degrading phenylalanine to produce trans-cinnamate and ammonia. PAL activity can be measured using an assay described by Moffitt et al. (2007) Biochemistry 46:1004-12, which is herein incorporated by reference in its entirety.

3. Glutaminases

In some embodiments, the exogenous immunogenic polypeptide comprises or consists of an amino acid-degrading polypeptide comprising a glutaminase, or a fragment or variant thereof. In some embodiments, the exogenous immunogenic polypeptide comprising a glutaminase, or a fragment or variant thereof, has both glutamine-degrading activity and asparaginase activity.

Numerous glutaminases have been identified, and may be used as described herein. For example, in some embodiments, the exogenous immunogenic polypeptide comprises a glutaminase from Pseudomonas, Acinetobacter glutaminasificans, or Pseudomonas putida, or a fragment or variant thereof.

Engineered erythroid cells comprising an exogenous immunogenic polypeptide comprising an glutaminase can be used in the treatment of a cancer described herein, including e.g., a leukemia (e.g., AML, ALL, lymphoblastic lymphoma), a lymphoma (e.g., NK/T cell lymphoma or non-Hodgkin lymphoma), pancreatic cancer, ovarian cancer, fallopian cancer, and peritoneal cancer.

Exemplary amino acid sequences of asparaginases, phenylalanine ammonia lyases, and glutaminases that can be included in an exogenous immunogenic polypeptide of the engineered erythroid cells or enucleated cells described herein are set forth in Table 2. In some embodiments, the exogenous immunogenic polypeptide comprises or consists of a PAL comprising the amino acid sequence of any one of SEQ ID NOs: 5, 6 and 7, or a fragment or variant thereof. In some embodiments, the exogenous immunogenic polypeptide comprises or consists of an asparaginase comprising the amino acid sequence of any one of SEQ ID NOs: 8, 9, 10, 11, 12, 13, and 14, or a fragment or variant thereof. In some embodiments, the exogenous immunogenic polypeptide comprises or consists of a glutaminase comprising the amino acid sequence of any one of SEQ ID NOs: 15, 16, 17, and 18, or a fragment or variant thereof. In some embodiments, the exogenous immunogenic polypeptide comprises or consists of an amino acid sequence having at least 70%, 80%, 85%, 90%, 95%, 96%, 97%, 98%, or 99% sequence identity to an amino acid sequence of any one of SEQ ID NOs: 8, 9, 10, 11, 12, 13, 14, 15, 16, 17, and 18, or a fragment thereof (e.g., an enzymatically-active fragment thereof).

TABLE 2
Exemplary exogenous immunogenic polypeptides

| Sequence Name | |
|---|---|
| (SEQ ID NO) | Amino acid sequence |
| Anabaena | MKTLSQAQSKTSSQQFSFTGNSSANVIIGNQKLTINDVARVARNGTLVSLT |
| variabilis | NNTDILQGIQASCDYINNAVESGEPIYGVTSGFGGMANVAISREQASELQT |
| phenylalanine | NLVWFLKTGAGNKLPLADVRAAMLLRANSHMRGASGIRLELIKRMEIFL |
| ammonia lyase | NAGVTPYVYEFGSIGASGDLVPLSYITGSLIGLDPSFKVDFNGKEMDAPTA |
| UniProt Accession | LRQLNLSPLTLLPKEGLAMMNGTSVMTGIAANCVYDTQILTAIAMGVHA |
| No. Q3M5Z3 | LDIQALNGTNQSFHPFIHNSKPHPGQLWAADQMISLLANSQLVRDELDGK |
| (SEQ ID NO: 5) | HDYRDHELIQDRYSLRCLPQYLGPIVDGISQIAKQIEIEINSVTDNPLIDVDN |
| | QASYHGGNFLGQYVGMGMDHLRYYIGLLAKHLDVQIALLASPEFSNGLP |
| | PSLLGNRERKVNMGLKGLQICGNSIMPLLTFYGNSIADRFPTHAEQFNQNI |
| | NSQGYTSATLARRSVDIFQNYVAIALMFGVQAVDLRTYKKTGHYDARAC |
| | LSPATERLYSAVRHVVGQKPTSDRPYIWNDNEQGLDEHIARISADIAAGG |
| | VIVQAVQDILPCLH |
| Arabidopsis | MDQIEAMLCGGGEKTKVAVTTKTLADPLNWGLAADQMKGSHLDEVKK |
| thaliana | MVEEYRRPVVNLGGETLTIGQVAAISTVGGSVKVELAETSRAGVKASSD |
| phenylalanine | WVMESMNKGTDSYGVTTGFGATSHRRTKNGTALQTELIRFLNAGIFGNT |
| ammonia lyase 2 | KETCHTLPQSATRAAMLVRVNTLLQGYSGIRFEILEAITSLLNHNISPSLPL |
| NCBI Accession | RGTITASGDLVPLSYIAGLLTGRPNSKATGPDGESLTAKEAFEKAGISTGFF |
| No. NP_190894.1 | DLQPKEGLALVNGTAVGSGMASMVLFEANVQAVLAEVLSAIFAEVMSG |
| (SEQ ID NO: 6) | KPEFTDHLTHRLKHHPGQIEAAAIMEHILDGSSYMKLAQKVHEMDPLQK |
| | PKQDRYALRTSPQWLGPQIEVIRQATKSIEREINSVNDNPLIDVSRNKAIHG |
| | GNFQGTPIGVSMDNTRLAIAAIGKLMFAQFSELVNDFYNNGLPSNLTASS |
| | NPSLDYGFKGAEIAMASYCSELQYLANPVTSHVQSAEQHNQDVNSLGLIS |
| | SRKTSEAVDILKLMSTTFLVGICQAVDLRHLEENLRQTVKNTVSQVAKKV |
| | LTTGINGELHPSRFCEKDLLKVVDREQVFTYVDDPCSATYPLMQRLRQVI |
| | VDHALSNGETEKNAVTSIFQKIGAFEEELKAVLPKEVEAARAAYGNGTAP |
| | IPNRIKECRSYPLYRFVREELGTKLLTGEKVVSPGEEFDKVFTAMCEGKLI |
| | DPLMDCLKEWNGAPIPIC |
| Pseudomonas | MRPIERLLAVVDGEVSARLDEGMRGRIDAGHALLLELIAAGAPIYGVTTG |
| putida | LGAAVDHAQGDAGFQQRIAAGRAVGVGRLASRREVRAIMAARLAGLAL |
| phenylalanine | GRSGISLASAMALGDFLDHGIHPEVPLLGSLGASDLAPLAHVTLALQGQG |
| ammonia lyase | WVEYHGERLPAAEALQRAGLAPLVPRDKDGLALVSANSASIGLGALLVS |
| NCBI Accession | ETQRLLDRQRGVLALSCEGYRAGVAPFQAAHLRPAPGLVEESTALLALLE |
| No. | GGDRQARRLQDPLSFRCSTVVLGAVRDALARARDIVVIELQSGADNPAL |
| WP_064302405.1 | VVKSREVLVTANFDSTHLALAFEGLGLALSRLAVASAERMAKLLSPGSSE |
| (SEQ ID NO: 7) | LPHSLSPRPGSVGLAALQRTAAALVAEIVHLANPLPALSVPVADRVEDYA |
| | GQGLAVVEKTARLVQRVEWLVRIEAVVAAQAVDLRAGITLGSEASAIYR |
| | QIRQVVAFVEDDRAIDVTGEFWGR |
| Erwinia | MERWFKSLFVLVLFFVFTASAADKLPNIVILATGGTIAGSAATGTQTTGY |
| chrysanthemi L- | KAGALGVDTLINAVPEVKKLANVKGEQFSNMASENMTGDVVLKLSQRV |
| asparaginase | NELLARDDVDGVVITHGTDTVEESAYFLHLTVKSDKPVVFVAAMRPATA |
| UniProt Accession | ISADGPMNLLEAVRVAGDKQSRGRGVMVVLNDRIGSARYITKTNASTLD |
| No. P06608 | TFKANEEGYLGVIIGNRIYYQNRIDKLHTTRSVFDVRGLTSLPKVDILYGY |
| (SEQ ID NO: 8) | QDDPEYLYDAAIQHGVKGIVYAGMGAGSVSVRGIAGMRKAMEKGVVVI |
| | RSTRTGNGIVPPDEELPGLVSDSLNPAHARILLMLALTRTSDPKVIQEYFH |
| | TY |
| Escherichia coli L- | MEFFKKTALAALVMGFSGAALALPNITILATGGTIAGGGDSATKSNYTVG |
| asparaginase 2 | KVGVENLVNAVPQLKDIANVKGEQVVNIGSQDMNDNVWLTLAKKINTD |
| UniProt Accession | CDKTDGFVITHGTDTMEETAYFLDLTVKCDKPVVMVGAMRPSTSMSAD |
| No. P00805 | GPFNLYNAVVTAADKASANRGVLVVMNDTVLDGRDVTKTNTTDVATFK |
| (SEQ ID NO: 9) | SVNYGPLGYIHNGKIDYQRTPARKHTSDTPFDVSKLNELPKVGIVYNYAN |
| | ASDLPAKALVDAGYDGIVSAGVGNGNLYKSVFDTLATAAKTGTAVVRSS |
| | RVPTGATTQDAEVDDAKYGFVASGTLNPQKARVLLQLALTQTKDPQQIQ |
| | QIFNQY |
| E. coli L- | MQKKSIYVAYTGGTIGMQRSEQGYIPVSGHLQRQLALMPEFHRPEMPDFT |
| asparaginase 1 | IHEYTPLMDSSDMTPEDWQHIAEDIKAHYDDYDGFVILHGTDTMAYTAS |
| NCBI Accession | ALSFMLENLGKPVIVTGSQIPLAELRSDGQINLLNALYVAANYPINEVTLF |
| No. NP_416281.1 | FNNRLYRGNRTTKAHADGFDAFASPNLPPLLEAGIHIRRLNTPPAPHGEGE |
| (SEQ ID NO: 10) | LIVHPITPQPIGVVTIYPGISADVVRNFLRQPVKALILRSYGVGNAPQNKAF |
| | LQELQEASDRGIVVVNLTQCMSGKVNMGGYATGNALAHAGVIGGADMT |
| | VEATLTKLHYLLSQELDTETIRKAMSQNLRGELTPDD |
| Staphylococcus | MKHLLVIHTGGTISMSQDQSNKVVTNDINPISMHQDVINQYAQIDELNPF |
| aureus L- | NVPSPHMTIQHVKQLKDIILEAVTNKYYDGFVITHGTDTLEETAFLLDLIL |
| asparaginase | GIEQPVVITGAMRSSNEIGSDGLYNYISAIRVASDEKARHKGVMVVFNDEI |
| NCBI Accession | HTARNVTKTHTSNTNTFQSPNHGPLGVLTKDRVQFHHMPYRQQALENV |
| No. YP_500016.1 | NDKLNVPLVKAYMGMPGDIFSFYSREGIDGMVIEALGQGNIPPSALEGIQ |
| (SEQ ID NO: 11) | QLVSLNIPIVLVSRSFNGIVSPTYAYDGGGYQLAQQGFIFSNGLNGPKARL |
| | KLLVALSNNLDKAEIKSYFEL |
| Erwinia carotovora | MFNALFVVVFVCFSSLANAAENLPNIVILATGGTIAGSAAANTQTTGYKA |
| L-asparaginase | GALGVETLIQAVPELKTLANIKGEQVASIGSENMTSDVLLTLSKRVNELLA |
| UniProt Accession | RSDVDGVVITHGTDTLDESPYFLNLTVKSDKPVVFVAAMRPATAISADGP |
| No. I1SBD9 | MNLYGAVKVAADKNSRGRGVLVVLNDRIGSARFISKTNASTLDTFKAPE |
| (SEQ ID NO: 12) | EGYLGVIIGDKIYYQTRLDKVHTTRSVFDVTNVDKLPAVDITYGYQDDPE |
| | YMYDASIKHGVKGIVYAGMGAGSVSKRGDAGIRKAESKGIVVVRSSRTG |
| | SGIVPPDAGQPGLVADSLSPAKSRILLMLALTKTTNPAVIQDYFHAY |
| Wolinella | MAKPQVTILATGGTIAGSGESSVKSSYSAGAVTVDKLLAAVPAINDLATI |
| succinogenes L- | KGEQISSIGSQEMTGKVWLKLAKRVNELLAQKETEAVIITHGTDTMEETA |
| asparaginase | FFLNLTVKSQKPVVLVGAMRSGSSMSADGPMNLYNAVNVAINKASTNK |
| UniProt Accession | GVVIVMNDEIHAAREATKLNTTAVNAFASPNTGKIGTVYYGKVEYFTQS |
| No. P50286 | VRPHTLASEFDISKIEELPRVDILYAHPDDTDVLVNAALQAGAKGIIHAGM |
| (SEQ ID NO: 13) | GNGNPFPLTQNALEKAAKSGVVVARSSRVGSGSTTQEAEVDDKKLGFVA |
| | TESLNPQKARVLLMLALTKTSDREAIQKIFSTY |
| Asparaginase | MADKLPNIVILATGGTIAGSAATGTQTTGYKAGALGVDTLINAVPEVKKL |
| (SEQ ID NO: 14) | ANVKGEQFSNMASENMTGDVVLKLSQRVNELLARDDVDGVVITHGTDT |
| | VEESAYFLHLTVKSDKPVVFVAAMRPATAISADGPMNLLEAVRVAGDKQ |
| | SRGRGVMVVLNDRIGSARYITKTNASTLDTFKANEEGYLGVIIGNRIYYQ |
| | NRIDKLHTTRSVFDVRGLTSLPKVDILYGYQDDPEYLYDAAIQHGVKGIV |
| | YAGMGAGSVSVRGIAGMRKAMEKGVVVIRSTRTGNGIVPPDEELPGLVS |
| | DSLNPAHARILLMLALTRTSDPKVIQEYFHTY |
| Glutaminase- | KEVENQQKLANVVILATGGTIAGAGASAANSATYQAAKVGVDKLIAGVP |
| asparaginase | ELADLANVRGEQVMQIASESITNDDLLKLGKRVAELADSNDVDGIVITHG |
| UniProt Accession | TDTLEETAYFLDLTLNTDKPIVVVGSMRPGTAMSADGMLNLYNAVAVAS |
| No. P10182 | NKDSRGKGVLVTMNDEIQSGRDVSKSINIKTEAFKSAWGPLGMVVEGKS |
| (SEQ ID NO: 15) | YWFRLPAKRHTVNSEFDIKQISSLPQVDIAYSYGNVTDTAYKALAQNGAK |
| | ALIHAGTGNGSVSSRLTPALQTLRKTGTQIIRSSHVNQGGFVLRNAEQPDD |
| | KNDWVVAHDLNPEKARILVELAMVKTQDSKELQRIFWEY |
| Pseudomonas 7A | KEVENQQKLANVVILATGGTIAGAGASAANSATYQAAKVGVDKLIAGVP |
| glutaminase- | ELADLANVRGEQVMQIASESITNDDLLKLGKRVAELADSNDVDGIVITHG |
| asparaginase | TDTLEETAYFLNLVEKTDKPIVVVGSMRPGTAMSADGMLNLYNAVAVA |
| (SEQ ID NO: 16) | SNKDSRGKGVLVTMNDEIQSGRDVSKSINIKTEAFKSAWGPLGMVVEGK |
| | SYWFRLPAKRHTVNSEFDIKQISSLPQVDIAYSYGNVTDTAYKALAQNGA |
| | KALIHAGTGNGSVSSRVVPALQELRKNGVQIIRSSHVNQGGFVLRNAEQP |
| | DDKNDWVVAHDLNPQKARILAMVAMTKTQDSKELQRIFWEY |
| Acinetobacter | KNNVVIVATGGTIAGAGASSTNSATYSAAKVPVDALIKAVPQVNDLANIT |
| glutaminasificans | GIQALQVASESITDKELLSLARQVNDLVKKPSVNGVVITHGTDTMEETAF |
| glutaminase- | FLNLVVHTDKPIVLVGSMRPSTALSADGPLNLYSAVALASSNEAKNKGV |
| asparaginase | MVLMNDSIFAARDVTKGINIHTHAFVSQWGALGTLVEGKPYWFRSSVKK |
| UniProt Accession | HTNNSEFNIEKIQGDALPGVQIVYGSDNMMPDAYQAFAKAGVKAIIHAGT |
| No. P10172 | GNGSMANYLVPEVRKLHDEQGLQIVRSSRVAQGFVLRNAEQPDDKYGW |
| (SEQ ID NO: 17) | IAAHDLNPQKARLLMALALTKTNDAKEIQNMFWNY |
| Pseudomonas | MNAALKTFAPSALALLLILPSSASAKEAETQQKLANVVILATGGTIAGAG |
| putida | ASAANSATYQAAKLGVDKLIAGVPELADIANVRGEQVMQIASESISNDDL |
| glutaminase- | LKLGKRVAELAESKDVDGIVITHGTDTLEETAFFLNLVEKTDKPIVVVGS |
| asparaginase | MRPGTAMSADGMLNLYNAVAVASDKQSRGKGVLVTMNDEIQSGRDVS |
| UniProt Accession | KAVNIKTEAFKSAWGPMGMVVEGKSYWFRLPAKRHTVNSEFDIKQISSL |
| No. Q88K39 | PQVDIAYGYGNVTDTAYKALAQNGAKALIHAGTGNGSVSSRVVPALQEL |
| (SEQ ID NO: 18) | RKNGVQIIRSSHVNQGGFVLRNAEQPDDKNDWVVAHDLNPQKARILAM |
| | VAMTKTQDSKELQRIFWEY |

4. Phenylalanine Hydroxylases (PAHs)

In some embodiments, the exogenous immunogenic polypeptide comprises or consists of an amino acid-degrading polypeptide comprising a phenylalanine hydroxylase (PAH), or a fragment or variant thereof. Engineered erythroid cells or erythroid cells comprising an exogenous immunogenic polypeptide comprising a PAH, or a fragment or variant thereof, may be used to treat subjects having PKU. Phenylalanine hydroxylases may be derived from any source, e.g., mammalian, fungal, plant or bacterial sources. For example, in some embodiments, the PAH is from Chromobacterium violaceum (see, e.g., Yew et al. (2013) Mol. Gen. Metab. 109: 339-44, incorporated herein by reference).

5. Homocysteine-Reducing and Homocysteine-Degrading Polypeptides

In some embodiments, the exogenous immunogenic polypeptide comprises or consists of an amino acid-degrading polypeptide comprising a homocysteine-reducing polypeptide or a homocysteine-degrading polypeptide, or a fragment or variant thereof. U.S. Patent Publication No. 2019/0309271 (which is incorporated herein by reference in its entirety) describes multiple homocysteine-reducing polypeptides and homocysteine-degrading polypeptides that can be included in an exogenous immunogenic polypeptide on the cell surface of the engineered erythroid cells (e.g., engineered enucleated erythroid cells) or enucleated cells (e.g., modified enucleated cells) described herein. Engineered erythroid cells comprising an exogenous immunogenic polypeptide comprising a homocysteine-reducing polypeptide (or a fragment or variant thereof) or a homocysteine-degrading polypeptide (or a fragment of variant thereof) may be used to reduce homocysteine levels in a subject in need thereof, and/or to treat subjects having a homocysteine-related disease.

In some embodiments, an engineered erythroid cell or enucleated cell provided herein comprises a first exogenous immunogenic polypeptide comprising or consisting of a homocysteine-degrading polypeptide, such as a cystathionine beta-synthase or a methioning gamma-lyase, or a fragment or variant thereof, and a second exogenous immunogenic polypeptide comprising or consisting of a homocysteine-reducing polypeptide, or a variant thereof. In some embodiments, an engineered erythroid cell or enucleated cell provided herein comprises a first exogenous immunogenic polypeptide comprising or consisting of a homocysteine-degrading polypeptide, or a fragment or variant thereof, and a second exogenous immunogenic polypeptide comprising or consisting of a homocysteine-degrading polypeptide, or a fragment or variant thereof. In some embodiments, an engineered erythroid cell or enucleated cell provided herein comprises a first exogenous immunogenic polypeptide comprising or consisting of a homocysteine-reducing polypeptide, or a fragment or variant thereof, and a second exogenous immunogenic polypeptide comprising or consisting of a homocysteine-reducing polypeptide, or a fragment or variant thereof.

Homocysteine-reducing polypeptides, and homocysteine-degrading polypeptides, as well as fragments and variant thereof, can be derived from any source or species, e.g., mammalian, fungal (including yeast), plant or bacterial sources. In some embodiments, a homocysteine-reducing polypeptide for use as described herein is a chimeric homocysteine-reducing polypeptide or a chimeric homocysteine-degrading polypeptide (e.g., derived from two different polypeptides, e.g., from two different organism species).

In some embodiments, an exogenous immunogenic polypeptide provided herein comprises or consists of a homocysteine-reducing polypeptide. In some embodiments, the exogenous immunogenic polypeptide comprises a homocysteine-reducing polypeptide comprising a methionine adenosyltransferase (e.g., enzyme commission number (E.C.) 2.5.1.6), an alanine transaminase (e.g., E.C. 2.6.1.2), a L-alanine-L-anticapsin ligase (e.g., E.C. 6.3.2.49), a L-cysteine desulfidase (e.g., E.C. 4.4.1.28), a methylenetetrahydrofolate reductase (MTHFR) (e.g., E.C. 1.5.1.20), a 5-methyltetrahydrofolate-homocysteine methyltransferase reductase (MTRR) (e.g., E.C. 1.16.1.8), or a methylmalonic aciduria and homocystinuria, cblD type (MMADHC), or a fragment or variant of any of the foregoing.

In some embodiments, the exogenous immunogenic polypeptide provided herein comprises or consists of a homocysteine-degrading polypeptide. In some embodiments, the exogenous immunogenic polypeptide comprises or consists of a homocysteine-degrading polypeptide comprising a cystathionine beta-synthase, a methionine gamma-lyase (e.g., E.C. 4.4.1.11), a sulfide:quinone reductase (e.g., E.C. 1.8.5.4), a methionine synthase (e.g., E.C. 2.1.1.13), a 5-methyl-tetrahydropteroyltriglutamate-homocysteine S-methyltransferase (e.g., E.C. 2.1.1.14), an adenosylhomocysteinase (e.g., E.C. 3.3.1.1), a cystathionine gamma-lyase (e.g., E.C. 4.4.1.1), a L-amino-acid oxidase (e.g., E.C. 1.4.3.2), a thetin-homocysteine S-methyltransferase polypeptide (e.g., E.C. 2.1.1.3), a betaine-homocysteine S-methyltransferase (e.g., E.C. 2.1.1.5), a homocysteine S-methyltransferase (e.g., E.C. 2.1.1.10), a selenocysteine Se-methyltransferase (e.g., E.C. 2.1.1.280), a cystathionine gamma-synthase (e.g., E.C. 2.5.1.48), a O-acetylhomoserine aminocarboxypropyltransferase (e.g., E.C. 2.5.1.49), an asparagine-oxo-acid transaminase (e.g., E.C. 2.6.1.14), a glutamine-phenylpyruvate transaminase (e.g., E.C. 2.6.1.64), a 3-mercaptopyruvate sulfurtransferase (e.g., E.C. 2.8.1.2), a homocysteine desulfhydrase (e.g., E.C. 4.4.1.2), a cystathionine beta-lyase (e.g., E.C. 4.4.1.8), an amino-acid racemase (e.g., E.C. 5.1.1.10), a methionine-tRNA ligase (e.g., E.C. 6.1.1.10), a glutamate-cysteine ligase (e.g., E.C. 6.3.2.2), a N-(5-amino-5-carboxypentanoyl)-L-cysteinyl-D-valine synthase (e.g., E.C. 6.3.2.26), a L-isoleucine 4-hydroxylase (e.g., E.C. 1.14.11.45), a L-lysine N6-monooxygenase (NADPH) (e.g., E.C. 1.14.13.59), a methionine decarboxylase (e.g., E.C. 4.1.1.57), a 2,2-dialkylglycine decarboxylase (pyruvate) (e.g., E.C. 4.1.1.64), or a cysteine synthase (CysO) (e.g., E.C. 2.5.1.47, e.g., a Aeropyrum pernix CysO polypeptide), or a fragment or variant of any of the foregoing.

### Uric Acid-Degrading Polypeptides

In some embodiments, the exogenous immunogenic polypeptide comprises or consists of a uric acid-degrading polypeptide, or a fragment or variant thereof. U.S. Patent Publication No. 2019/0309269 (which is incorporated herein by reference in its entirety) describes multiple uric acid-degrading polypeptides that can be included in an exogenous immunogenic polypeptide on the cell surface of the engineered erythroid cells (e.g., engineered enucleated erythroid cells) or enucleated cells (e.g., modified enucleated cells) described herein. Engineered erythroid cells comprising an exogenous immunogenic polypeptide comprising a uric acid-degrading polypeptide (or a fragment or variant thereof) may be used to treat subjects having a uric acid-related disease (e.g., gout).

Uric acid-degrading polypeptides, as well as fragments and variant thereof, can be derived from any source or species, e.g., mammalian, fungal (including yeast), plant or bacterial sources, or can be recombinantly engineered. In some embodiments, the exogenous immunogenic polypeptide comprises or consists of a chimeric uric acid-degrading polypeptide (e.g., derived from two different polypeptides, e.g., from two different organism species).

In some embodiments, the exogenous immunogenic polypeptide comprises or consists of a uric acid-degrading polypeptide comprising a uricase, an HIU hydrolase, an OHCU decarboxylase, an allantoinase, an allantoicase, a myeloperoxidase, a FAD-dependent urate hydroxylase, a xanthine dehydrogenase, a nucleoside deoxyribosyltransferase, a dioxotetrahydropyrimidine phosphoribosyltransferase, a dihydropyrimidinase, or a guanine deaminase, or a fragment or variant of any of the foregoing.

### Oxalate Oxidases

In some embodiments, the exogenous immunogenic polypeptide comprises or consists of an oxalate oxidase (OxOx), or a fragment or variant thereof. Engineered erythroid cells and enucleated cells comprising an exogenous immunogenic polypeptide comprising an OxOx, or a fragment or variant thereof, may be used to treat subjects having hyperoxaluria, e.g., primary hyperoxaluria.

Oxalate oxidases, as well as fragments and variant thereof, can be derived from any source or species, e.g., mammalian, fungal (including yeast), plant or bacterial sources, or can be recombinantly engineered. In some embodiments, the exogenous immunogenic polypeptide comprises or consists of a chimeric oxalate oxidase (e.g., derived from two different polypeptides, e.g., from two different organism species).

### Exogenous Antigenic Polypeptides

In some embodiments, the engineered erythroid cells (e.g., engineered enucleated erythroid cells) or enucleated cells (e.g., modified enucleated cells) described herein include at least one (e.g., one, two, three, or more) exogenous immunogenic polypeptide, at least one (e.g., one, two, three, or more) exogenous HLA-G polypeptide, and at least one (e.g., one, two, three, or more) exogenous antigenic polypeptide. In some embodiments, the engineered erythroid cells or enucleated cells described herein include an exogenous immunogenic polypeptide and an exogenous HLA-G polypeptide bound (e.g., specifically bound) to an exogenous antigenic polypeptide. In some embodiments, the exogenous antigenic polypeptide is bound to the exogenous HLA-G polypeptide, e.g., either covalently or non-covalently, and both polypeptides are not fused to each other (e.g., as a single fusion polypeptide). In other embodiments, the exogenous antigenic polypeptide is linked to a portion of the exogenous HLA-G polypeptide as a fusion polypeptide. In some embodiments, the exogenous antigenic polypeptide is a tolerogenic polypeptide. In some embodiments, the exogenous antigenic polypeptide comprises or consists of the motif XI/LPXXXXXL, wherein X is any amino acid residue (SEQ ID NO: 1). In some embodiments, the exogenous antigenic polypeptide comprises or consists of an amino acid sequence selected from RIIPRHLQL (SEQ ID NO: 842), KLPAQFYIL (SEQ ID NO: 843), and KGPPAALTL (SEQ ID NO: 844).

In some embodiments, a portion of the exogenous antigenic polypeptide is capable of binding (e.g., specifically binding) to the antigen-binding cleft of the exogenous HLA-G polypeptide. One of ordinary skill the art can readily identify exogenous antigenic polypeptides (or fragments thereof) that are capable of binding to (e.g., specifically binding to) an exogenous HLA-G polypeptide provided herein, and which may be included in the engineered erythroid cells or enucleated cells described herein. For example, search tools and algorithms known in the art may be used, including, but not limited to, T cell epitope prediction tools and algorithms described in Kessler and Melief (2007) Leukemia 21: 1859-74 (the entire contents of which are incorporated herein by reference; see, e.g., Table 1). Additional search tools include BIMAS (available on the world wide web at bimas.dcrt.nih.gov/molbio/hla bind), SYFPEITHI (available on the world wide web at syfpeithi.de), NetMHC (available on the world wide web at cbs.dtu.dk/services/NetMHC), PREDEP (available on the world wide web at margalit.huji.ac.il), ProPred-1 (available on the world wide web at imtech.res.in/raghava/propredl/index.html), nHLAPred (available on the world wide web at imtech.res.in/raghava/nhlapred/), and IEDB (available on the world wide web at tools.immuneepitope.org/analyze/html/mhc binding.html). International Patent Publication No. WO 2018/005559, the contents of which are hereby incorporated herein by reference, also describes methods of identifying exogenous antigenic polypeptides, or fragments thereof, that are capable of binding to exogenous HLA-G polypeptides. Multiple assays for assessing binding affinity and/or determining whether an exogenous antigenic polypeptide, or fragment thereof, specifically binds to an exogenous HLA-G polypeptide are known in the art. For example, surface plasmon resonance (Biacore®) can be used to determine the binding constant of a complex between two polypeptides. Other suitable assays include, for example, immunoassays such as enzyme linked immunosorbent assays (ELISA) and radioimmunoassays (MA), or determination of binding by monitoring the change in the spectroscopic or optical properties of the proteins using fluorescence, UV absorption, circular dichroism, nuclear magnetic resonance (NMR), Western blot, analytical ultracentrifugation, and spectroscopy (see, e.g., Scatchard et al. (1949) Ann. N.Y. Acad. Sci. 51:660-72; Wilson (2002) Science 295: 2103-5; U.S. Pat. Nos. 5,283,173, and 5,468,614; and International Patent Publication No. WO 2018/005559). Alternatively, binding of an exogenous antigenic polypeptide, or a fragment thereof, to an exogenous HLA-G polypeptide may be determined using a predictive algorithm (see, e.g., Kessler and Melief (2007), supra).

In some embodiments, the exogenous antigenic polypeptide is derived from a human polypeptide. In some embodiments, the exogenous antigenic polypeptide is derived from an infectious disease agent (e.g., a virus, a parasite (e.g., an intracellular parasite), a prion, a bacterium (e.g., an intracellular pathogenic bacterium)). For example, in some embodiments, the exogenous antigenic polypeptide comprises a fragment of an infectious disease agent polypeptide capable of binding to the antigen-binding cleft of an exogenous HLA-G polypeptide. In some embodiments, the exogenous antigenic polypeptide is derived from a virus (e.g., an Epstein Barr virus or HIV). In some embodiments, the exogenous antigenic polypeptide is derived from a bacterium (e.g., Mycobacterium tuberculosis).

In some embodiments, the exogenous antigenic polypeptide is between about 8 and about 24 amino acid residues in length. In some embodiments, the exogenous antigenic polypeptide is between about 8 amino acid residues in length to 24 amino acid residues in length, e.g., 8, 9, 10, 11, 12, 13, 14, 15, 16, 17, 18, 19, 20, 21, 22, 23, or 24 amino acid residues in length. In some embodiments, the exogenous antigenic polypeptide is between about 10 and about 150 amino acid residues (e.g., 10, 15, 20, 25, 30, 35, 40, 45, 50, 60, 70, 80, 90, 100, 110, 120, 130, 140, or 150 amino acid residues in length).

In some embodiments, the exogenous antigenic polypeptide comprises a cleavable site. In some embodiments, the cleavable site is adjacent to an amino acid sequence of the exogenous antigenic polypeptide which binds to an antigen-binding cleft of an exogenous HLA-G polypeptide. In some embodiments, the cleavable site is present within a linker of the exogenous antigenic polypeptide. In some embodiments, the cleavable site is present within an amino acid sequence of the exogenous antigenic polypeptide which binds to an antigen-binding cleft of an exogenous HLA-G polypeptide.

In some embodiments, the exogenous antigenic polypeptide comprises a membrane anchor (e.g., a transmembrane domain, such as a Type I membrane protein transmembrane domain (e.g., a glycophorin A (GPA) transmembrane domain), or a Type II membrane protein transmembrane domain (e.g., a Kell transmembrane domain or a small integral membrane protein 1 (SMIM1) transmembrane domain)), as either an N-terminal or C-terminal fusion, e.g., such that the portion of the exogenous antigenic polypeptide that is capable of binding to an exogenous HLA-G polypeptide described herein is present on the outer side of the surface of the engineered erythroid cell or enucleated cell. In some embodiments, the exogenous antigenic polypeptide comprises a membrane anchor (e.g., a transmembrane domain), a linker, and an amino acid sequence (e.g., an antigen) that is capable of binding to the antigen-binding cleft of an exogenous HLA-G polypeptide. Any of the linkers provided herein may be disposed between the membrane anchor and the amino acid sequence that is capable of binding to the antigen-binding cleft of an exogenous HLA-G polypeptide. For example, in some embodiments, the linker is a flexible linker (e.g., a GlySer linker). In some embodiments, the linker is from about 30 to about 100 amino acid residues in length. In other embodiments, the linker is between about 40 amino acid residues in length and 70 amino acids in length. In some embodiments, the linker is a cleavable linker (e.g., comprising a cleavable site).

In some embodiments, the exogenous antigenic polypeptide can be tethered to the plasma membrane via attachment to a lipid moiety (e.g., N-myristoylation, S-palmitoylation, farnesylation, geranylgeranylation, and glycosylphosphatidyl inositol (GPI) anchor).

Nucleic acids (e.g., an exogenous nucleic acid) comprising or consisting of a nucleic acid sequence encoding an exogenous antigenic polypeptide described herein are also provided. In some embodiments, the nucleic acid comprises at least one promoter (e.g., a constitutive or an inducible promoter) operably-linked to the open reading frame or gene encoding the exogenous antigenic polypeptide. In some embodiments, the nucleic acid is codon-optimized (e.g., for expression in a human cell). In some embodiments, the nucleic acid is not codon-optimized.

Non-limiting examples of exogenous antigenic polypeptides are listed in Table 3.

### Exogenous Autoantigenic Polypeptides

Non-limiting examples of exogenous autoantigenic polypeptides include preproinsulin, proinsulin, and insulin peptides (e.g., optionally fused to any of the membrane anchors described herein or attached to the plasma membrane via attachment to a lipid moiety (e.g., N-myristoylation, S-palmitoylation, farnesylation, geranylgeranylation, and glycosylphosphatidyl inositol (GPI) anchor)). Additional examples of exogenous autoantigenic polypeptides include RAS guanyl-releasing protein 2 (RasGRP2), CDP L-fucose synthase, or a fragment thereof. Additional non-limiting examples of exogenous antigenic polypeptides, exogenous autoantigenic polypeptides, and autoantigens are shown in Table 3.

TABLE 3
Exemplary Exogenous Antigenic Polypeptides, Exogenous Autoantigenic
Polypeptides, and Autoantigens

| Sequence Name (SEQ ID NO:) | Sequence |
|---|---|
| 60 kDa Heat Shock Protein | TVIIEQSWGSPKVTKDGVTV |
| (SEQ ID NO: 38) | |
| 60 kDa Heat Shock Protein | QMRPVSRVL |
| (SEQ ID NO: 39) | |
| 60 kDa Heat Shock Protein | AYVLLSEKKISSIQS |
| (SEQ ID NO: 40) | |
| 60 kDa Heat Shock Protein | GEALSTLVLNRLKVG |
| (SEQ ID NO: 42) | |
| 60 kDa Heat Shock Protein | LAKLSDGVAVLKVGG |
| (SEQ ID NO: 43) | |
| 78 kDa glucose-regulated protein | VMRIINEPTAAAIAY |
| (SEQ ID NO: 44) | |
| 78 kDa glucose-regulated protein | EVTFEIDVNGILRVT + CITR(R13) |
| (SEQ ID NO: 45) | |
| 78 kDa glucose-regulated protein | EVTFEIDVNGILRVT |
| (SEQ ID NO: 46) | |
| 78 kDa glucose-regulated protein | TFEIDVNGILRVTAE + CITR(R11) |
| (SEQ ID NO: 47) | |
| 78 kDa glucose-regulated protein | VMRIINEPTAAAIAY + CITR(R3) |
| (SEQ ID NO: 48) | |
| 78 kDa glucose-regulated protein | VEKAKRALSSQHQA + CITR(R6) |
| (SEQ ID NO: 835) | |
| Alternatively spliced insulin (SEQ ID NO: 49) | MLYQHLLPL + OX(M1) |
| Chain A, Glutamate Decarboxylase 2 | YVVKSFDRSTKVIDFHYPNE |
| (SEQ ID NO: 50) | |
| Chain A, Glutamate Decarboxylase 2 | AMMIARFKMFPEVKEKG |
| (SEQ ID NO: 51) | |
| Chain A, Insulin, Monoclinic Crystal Form | GIVEQCCTSICS |
| (SEQ ID NO: 52) | |
| Chain A, Insulin, Monoclinic Crystal Form | QCCTSICSLYQL |
| (SEQ ID NO: 53) | |
| Chain B, Insulin B Chain (SEQ ID NO: 54) | LVEALYLVCGERGF |
| Chain B, Structure Of Insulin (SEQ ID NO: 55) | FVNQHLCGSHLVEAL |
| Chain B, Structure Of Insulin (SEQ ID NO: 56) | GSHLVEALYLVCGER |
| Chain B, Structure Of Insulin (SEQ ID NO: 57) | HLCGSHLVEALYLVC |
| Chain B, Structure Of Insulin (SEQ ID NO: 58) | HLVEALYLVCGERGF |
| Chain B, Structure Of Insulin (SEQ ID NO: 59) | LCGSHLVEALYLVCGER |
| Chain B, Structure Of Insulin (SEQ ID NO: 60) | LVEALYLVCGERGFF |
| Chain B, Structure Of Insulin (SEQ ID NO: 61) | LYLVCGERGFFYTPK |
| Chain B, Structure Of Insulin (SEQ ID NO: 62) | QHLCGSHLVEALYLV |
| Chain B, Structure Of Insulin (SEQ ID NO: 63) | VEALYLVCGERGFFY |
| chaperonin (HSP60) (SEQ ID NO: 64) | LVLNRLKVGLQVVAVKAPGF |
| chaperonin (HSP60) (SEQ ID NO: 65) | EIIKRTLKIPAMTIAKNAGV |
| chaperonin (HSP60) (SEQ ID NO: 66) | GEVIVTKDDAMLLKGKGDKA |
| chaperonin (HSP60) (SEQ ID NO: 67) | IVLGGGCALLRCIPALDSLT |
| chaperonin (HSP60) (SEQ ID NO: 68) | KFGADARALMLQGVDLLADA |
| chaperonin (HSP60) (SEQ ID NO: 69) | LVIIAEDVDGEALSTLVLNR |
| chaperonin (HSP60) (SEQ ID NO: 70) | EEIAQVATISANGDKEIGNI |
| chaperonin (HSP60) (SEQ ID NO: 71) | KAPGFGDNRKNQLKDMAIAT |
| chaperonin (HSP60) (SEQ ID NO: 72) | LLADAVAVTMGPKGRTVIIE |
| chaperonin (HSP60) (SEQ ID NO: 73) | MLRLPTVFRQMRPVSRVLAP |
| chaperonin (HSP60) (SEQ ID NO: 74) | NEEAGDGTTTATVLARSIAK |
| chaperonin (HSP60) (SEQ ID NO: 75) | NPVEIRRGVMLAVDAVIAEL |
| chaperonin (HSP60) (SEQ ID NO: 76) | QDAYVLLSEKKISSIQSIVP |
| chaperonin (HSP60) (SEQ ID NO: 77) | QSIVPALEIANAHRKPLVIIA |
| chaperonin (HSP60) (SEQ ID NO: 78) | RKGVITVKDGKTLNDELEII |
| chaperonin (HSP60) (SEQ ID NO: 79) | RSIAKEGFEKISKGANPVEI |
| chaperonin (HSP60) (SEQ ID NO: 80) | RVLAPHLTRAYAKDVKFGAD |
| chaperonin (HSP60) (SEQ ID NO: 81) | VIAELKKQSKPVTTPEEIAQ |
| chaperonin (HSP60) (SEQ ID NO: 82) | VNMVEKGIIDPTKVVRTALL |
| chaperonin (HSP60) (SEQ ID NO: 83) | VTDALNATRAAVEEGIVLGG |
| chaperonin (HSP60) (SEQ ID NO: 84) | VLGGGCALLRCIPALDSLTPANED |
| chromogranin A (SEQ ID NO: 85) | WSKMDQLAKELTAE |
| chromogranin A (SEQ ID NO: 86) | LLCAGQVTAL |
| chromogranin A (SEQ ID NO: 87) | TLSKPSPMPV |
| claudin-17 (SEQ ID NO: 88) | TTLLPQWRVSAFV |
| cyclin-I isoform b (SEQ ID NO: 89) | KLNWDLHTA |
| endoprotease (SEQ ID NO: 90) | FTNHFLVEL |
| Epithelial cell adhesion molecule | VRTYWIIIELKHKAREKPYDSKSLRTALQKEIT |
| (SEQ ID NO: 91) | |
| GAD2 protein, partial (SEQ ID NO: 92) | KIIKLFFRL |
| glial fibrillary acidic protein isoform 2 | NLAQDLATV |
| (SEQ ID NO: 93) | |
| glial fibrillary acidic protein isoform 2 | QLARQQVHV |
| (SEQ ID NO: 94) | |
| Glioma pathogenesis-related protein 1 | MRVTLATIAWMVSFVSNYSHTANILPDIENEDF |
| (SEQ ID NO: 95) | |
| Glioma pathogenesis-related protein 1 | TLATIAWMV |
| (SEQ ID NO: 96) | |
| Glioma pathogenesis-related protein 1 | VTLATIAWMVSFVSN |
| (SEQ ID NO: 97) | |
| Glucose-6-phosphatase (SEQ ID NO: 98) | EWVHIDTTPFASL |
| glucose-6-phosphatase 2 isoform X2 | LYHFLQIPTHEEHLF |
| (SEQ ID NO: 99) | |
| glutamate decarboxylase (SEQ ID NO: 100) | MASPGSGFWSFGSEDGSGDS |
| glutamate decarboxylase (SEQ ID NO: 101) | IPPSLRTLEDNEERMSRLSK |
| glutamate decarboxylase (SEQ ID NO: 102) | ATHQDIDFLIEEIERLGQDL |
| glutamate decarboxylase (SEQ ID NO: 103) | AALGIGTDSVILIKCDERGK |
| glutamate decarboxylase (SEQ ID NO: 104) | TNMFTYEIAPVFVLLEYVTL |
| glutamate decarboxylase (SEQ ID NO: 105) | CGRHVDVFKLWLMWRAKGTTG |
| glutamate decarboxylase (SEQ ID NO: 106) | EEILMHCQTTLKYAIKTGHP |
| glutamate decarboxylase (SEQ ID NO: 107) | ERANSVTWNPHKMMGVPLQC |
| glutamate decarboxylase (SEQ ID NO: 108) | EYGTTMVSYQPLGDKVNFFR |
| glutamate decarboxylase (SEQ ID NO: 109) | EYLYNIIKNREGYEMVFDGK |
| glutamate decarboxylase (SEQ ID NO: 110) | GGSGDGIFSPGGAISNMYAM |
| glutamate decarboxylase (SEQ ID NO: 111) | KGTTGFEAHVDKCLELAEYLYN |
| glutamate decarboxylase (SEQ ID NO: 112) | KTGHPRYFNQLSTGLDMVGL |
| glutamate decarboxylase (SEQ ID NO: 113) | LAFLQDVMNILLQYVVKSFDRS |
| glutamate decarboxylase (SEQ ID NO: 114) | LEAKQKGFVPFLVSATAGTT |
| glutamate decarboxylase (SEQ ID NO: 115) | LLYGDAEKPAESGGSQPPRA |
| glutamate decarboxylase (SEQ ID NO: 116) | QNCNQMHASYLFQQDKHYDL |
| glutamate decarboxylase (SEQ ID NO: 117) | VFDGKPQHTNVCFWYIPPSL |
| glutamate decarboxylase (SEQ ID NO: 118) | VNFFRMVISNPAATHQDIDF |
| glutamate decarboxylase (SEQ ID NO: 119) | IAPVFVLLEYVTLKKMREII |
| glutamate decarboxylase (SEQ ID NO: 120) | VAPVIKARMMEYGTTMVSYQ |
| glutamate decarboxylase (SEQ ID NO: 121) | LPRLIAFTSEHSHFSLKK |
| glutamate decarboxylase (SEQ ID NO: 122) | VNFFRMVISNPAAT |
| glutamate decarboxylase (SEQ ID NO: 123) | ALPRLIAFT + CITR(R4) |
| Glutamate decarboxylase 1 (SEQ ID NO: 124) | TYEIAPVFVLLFYVTLKKMR |
| Glutamate decarboxylase 1 (SEQ ID NO: 125) | NMFTYEIAPVFVLME |
| Glutamate decarboxylase 1 (SEQ ID NO: 126) | PTIAFLQDVMNILLQYVVKS |
| Glutamate decarboxylase 1 (SEQ ID NO: 127) | VMNILLQYW |
| Glutamate decarboxylase 2 (SEQ ID NO: 128) | CDGERPTLAFLQDVM |
| Glutamate decarboxylase 2 (SEQ ID NO: 129) | IAFTSEHSHFSLK |
| Glutamate decarboxylase 2 (SEQ ID NO: 130) | NMYAMMIARFKMFPEVKEKG |
| Glutamate decarboxylase 2 (SEQ ID NO: 131) | TYEIAPVFVLLEYVT |
| Glutamate decarboxylase 2 (SEQ ID NO: 132) | MNILLQYVVKSFD |
| Glutamate decarboxylase 2 (SEQ ID NO: 133) | NFFRMVISNPAAT |
| Glutamate decarboxylase 2 (SEQ ID NO: 134) | CFWYIPPSLRTLEDN |
| Glutamate decarboxylase 2 (SEQ ID NO: 135) | ERMSRLSKVAPVIKA |
| Glutamate decarboxylase 2 (SEQ ID NO: 136) | NMYAMMIARFKMFPEVKEKGMAALPRLIAFTSE |
| Glutamate decarboxylase 2 (SEQ ID NO: 137) | SRLSKVAPVIKARMMEYGTT |
| Glutamate decarboxylase 2 (SEQ ID NO: 138) | VSYQPLGDKVNFFRMVISNPAATHQDIDFLIEE |
| | IERLGQDL |
| Glutamate decarboxylase 2 (SEQ ID NO: 139) | FLQDVMNIL |
| Glutamate decarboxylase 2 (SEQ ID NO: 140) | KVNFFRMVISNPAATHQD |
| Glutamate decarboxylase 2 (SEQ ID NO: 141) | LLQEYNWEL |
| Glutamate decarboxylase 2 (SEQ ID NO: 142) | NILLQYVVKSFDRS |
| Glutamate decarboxylase 2 (SEQ ID NO: 143) | NPAATHQDIDFLI |
| Glutamate decarboxylase 2 (SEQ ID NO: 144) | RMMEYGTTMV |
| Glutamate decarboxylase 2 (SEQ ID NO: 145) | VMNILLQYVV |
| Glutamate decarboxylase 2 (SEQ ID NO: 146) | AKGTTGFEAHVDK |
| Glutamate decarboxylase 2 (SEQ ID NO: 147) | FDRSTKVIDFHYPNE |
| Glutamate decarboxylase 2 (SEQ ID NO: 148) | FFRMVISNPAATHQDIDFLI |
| Glutamate decarboxylase 2 (SEQ ID NO: 149) | GHPRYFNQLSTG |
| Glutamate decarboxylase 2 (SEQ ID NO: 150) | KHYDLSYDTGDKALQ |
| Glutamate decarboxylase 2 (SEQ ID NO: 151) | LPRLIAFTSEHSHF |
| Glutamate decarboxylase 2 (SEQ ID NO: 152) | LPRLIAFTSEHSHFS |
| Glutamate decarboxylase 2 (SEQ ID NO: 153) | NWELADQPQNLEEILMHCQT |
| Glutamate decarboxylase 2 (SEQ ID NO: 154) | RLIAFTSEHSHF |
| Glutamate decarboxylase 2 (SEQ ID NO: 155) | RMMEYGTTMVSYQPL |
| Glutamate decarboxylase 2 (SEQ ID NO: 156) | ANTNMFTYEIAPVFVLLE |
| Glutamate decarboxylase 2 (SEQ ID NO: 157) | EVKEKGMAALPRLIAFTSEH |
| Glutamate decarboxylase 2 (SEQ ID NO: 158) | FWYIPPSLRTLED |
| Glutamate decarboxylase 2 (SEQ ID NO: 159) | GGGLLMSRKHKWKLSGVERAN |
| Glutamate decarboxylase 2 (SEQ ID NO: 160) | GLMQNCNQMHASYLFQQDK |
| Glutamate decarboxylase 2 (SEQ ID NO: 161) | HTNVCFWYIPPSLRTLEDNE |
| Glutamate decarboxylase 2 (SEQ ID NO: 162) | MIARFKMFPEVKEKG |
| Glutamate decarboxylase 2 (SEQ ID NO: 163) | MMIARFKMFPEVKEKGMAAL |
| Glutamate decarboxylase 2 (SEQ ID NO: 164) | MYAMMIARFK |
| Glutamate decarboxylase 2 (SEQ ID NO: 165) | MYAMMIARFKMF |
| Glutamate decarboxylase 2 (SEQ ID NO: 166) | NILLQYVVKSFD |
| Glutamate decarboxylase 2 (SEQ ID NO: 167) | NYAFLHATDLLP |
| Glutamate decarboxylase 2 (SEQ ID NO: 168) | PSLRTLEDNEERMSRLSKVA |
| Glutamate decarboxylase 2 (SEQ ID NO: 169) | RFKMFPEVK |
| Glutamate decarboxylase 2 (SEQ ID NO: 170) | SCSKVDVNYAFLHATDLLPA |
| Glutamate decarboxylase 2 (SEQ ID NO: 171) | TSEHSHFSL |
| Glutamate decarboxylase 2 (SEQ ID NO: 172) | VMNILLQYV |
| Glutamate decarboxylase 2 (SEQ ID NO: 173) | YEMVFDGKPQHTNVCFWYIP |
| Glutamate decarboxylase 2 (SEQ ID NO: 174) | EYVTLKKMREIIGWPGGSGD |
| Glutamate decarboxylase 2 (SEQ ID NO: 175) | GMAALPRLIAFTSEHSHFSL |
| Glutamate decarboxylase 2 (SEQ ID NO: 176) | IKARMMEYGTTMVSY |
| Glutamate decarboxylase 2 (SEQ ID NO: 177) | MVFDGKPQHTNVCFW |
| Glutamate decarboxylase 2 (SEQ ID NO: 178) | PSLRTLEDNEERMSR |
| Glutamate decarboxylase 2 (SEQ ID NO: 179) | TGHPRYFNQLSTGLD |
| Glutamate decarboxylase 2 (SEQ ID NO: 180) | ELAEYLYNI |
| Glutamate decarboxylase 2 (SEQ ID NO: 181) | ILMHCQTTL |
| Glutamate decarboxylase 2 (SEQ ID NO: 182) | PEVKEKGMAALPRLIAFTSE |
| Glutamate decarboxylase 2 (SEQ ID NO: 183) | ARFKMFPEVKEKGMAALPRLIAF |
| Glutamate decarboxylase 2 (SEQ ID NO: 184) | DKVNFFRMVISNPAATHQDID |
| Glutamate decarboxylase 2 (SEQ ID NO: 185) | CACDQKPCSCSKVDVNYAFL |
| Glutamate decarboxylase 2 (SEQ ID NO: 186) | GGLLMSRKHKWKLSGVERAN |
| Glutamate decarboxylase 2 (SEQ ID NO: 187) | ICKKYKIWMHVDAAWGGGLL |
| Glutamate decarboxylase 2 (SEQ ID NO: 188) | REIIGWPGGSGDGIFSPGGA |
| Glutamate decarboxylase 2 (SEQ ID NO: 189) | RYFNQLSTGLDMVGLAADWL |
| Glutamate decarboxylase 2 (SEQ ID NO: 190) | YAMMIARFKMFPEVKEKGMA |
| Glutamate decarboxylase 2 (SEQ ID NO: 191) | ACDGERPTL |
| Glutamate decarboxylase 2 (SEQ ID NO: 192) | AHVDKCLEL |
| Glutamate decarboxylase 2 (SEQ ID NO: 193) | APVIKARMM |
| Glutamate decarboxylase 2 (SEQ ID NO: 194) | HPRYFNQLST |
| Glutamate decarboxylase 2 (SEQ ID NO: 195) | IPSDLERRIL |
| Glutamate decarboxylase 2 (SEQ ID NO: 196) | SPGSGFWSF |
| Glutamate decarboxylase 2 (SEQ ID NO: 197) | CKKYKIWMHVDAAWGGGLL |
| Glutamate decarboxylase 2 (SEQ ID NO: 198) | DVNYAFLHATDLLPACDG |
| Glutamate decarboxylase 2 (SEQ ID NO: 199) | EKGMAALPRLIAFTSEHSHFSLKK |
| Glutamate decarboxylase 2 (SEQ ID NO: 200) | GAISNMYAMMIARFKMFPEVKEKGM |
| Glutamate decarboxylase 2 (SEQ ID NO: 201) | GGLLMSRKHKWKLSGVERANSVTW |
| Glutamate decarboxylase 2 (SEQ ID NO: 202) | HPRYFNQLSTGLDMVG |
| Glutamate decarboxylase 2 (SEQ ID NO: 203) | LGDKVNFFRMVISNPAATHQD |
| Glutamate decarboxylase 2 (SEQ ID NO: 204) | NMFTYEIAPVFVLLEYVTLKKMRE |
| Glutamate decarboxylase 2 (SEQ ID NO: 205) | SALLVREEGLMQNCNQMHAS |
| Glutamate decarboxylase 2 (SEQ ID NO: 206) | TTVYGAFDPLLAVAD |
| Glutamate decarboxylase 2 (SEQ ID NO: 207) | VCFWYIPPSLRTLED |
| Glutamate decarboxylase 2 (SEQ ID NO: 208) | VDVFKLWLMWRAKGTTGFEAH |
| Glutamate decarboxylase 2 (SEQ ID NO: 209) | YLYNIIKNREGYEMVFD |
| Glutamate decarboxylase 2 (SEQ ID NO: 210) | VNFFRMVISNPAATHQDIDFLI |
| Glutamate decarboxylase 2 (SEQ ID NO: 211) | KGMAALPRLIAFTSEHSHFS + CITR(R8) |
| Glutamate decarboxylase 2 (SEQ ID NO: 212) | KVNFFRMVISNPAATHQDID + CITR(R6) |
| Glutamate decarboxylase 2 (SEQ ID NO: 213) | MNILLQYVVKSFD + DEAM(Q6) |
| Glutamate decarboxylase 2 (SEQ ID NO: 214) | PQNLEEILMHCQTTLKYAIK + DEAM(Q2, Q12) |
| Glutamate decarboxylase 2 (SEQ ID NO: 215) | YAFLHATDLLPACDGERPTL + CITR(R17) |
| Glutamate decarboxylase 2 (SEQ ID NO: 216) | FTSEHSHFS |
| Glutamate decarboxylase 2 (SEQ ID NO: 217) | MFPEVKEKG |
| glutamate decarboxylase 2 (pancreatic islets and | EAKQKGFVPFLVSATAGTTV |
| brain, 65 kDa) (SEQ ID NO: 218) | |
| glutamate decarboxylase 2 (pancreatic islets and | DERGKMIPSDLERRILEAKQ |
| brain, 65 kDa) (SEQ ID NO: 219) | |
| glutamate decarboxylase 2 (pancreatic islets and | DVMNILLQYVVKSFDRSTKV |
| brain, 65 kDa) (SEQ ID NO: 220) | |
| glutamate decarboxylase 2 (pancreatic islets and | KVNFFRMVISNPAATHQDID |
| brain, 65 kDa) (SEQ ID NO: 221) | |
| glutamate decarboxylase 2 (pancreatic islets and | LIAFTSEHSHFSLKKGAAAL |
| brain, 65 kDa) (SEQ ID NO: 222) | |
| glutamate decarboxylase 2 (pancreatic islets and | DSVILIKCDERGKMIPSDLE |
| brain, 65 kDa) (SEQ ID NO: 223) | |
| glutamate decarboxylase 2 (pancreatic islets and | HKWKLSGVERANSVTWNPHK |
| brain, 65 kDa) (SEQ ID NO: 224) | |
| glutamate decarboxylase 2 (pancreatic islets and | KCLELAEYLYNIIKNREGYE |
| brain, 65 kDa) (SEQ ID NO: 225) | |
| glutamate decarboxylase 2 (pancreatic islets and | KGMAALPRLIAFTSEHSHFS |
| brain, 65 kDa) (SEQ ID NO: 226) | |
| glutamate decarboxylase 2 (pancreatic islets and | RPTLAFLQDVMNILLQYVVK |
| brain, 65 kDa) (SEQ ID NO: 227) | |
| glutamate decarboxylase 2 (pancreatic islets and | YDLSYDTGDKALQCGRHVDV |
| brain, 65 kDa) (SEQ ID NO: 228) | |
| glutamate decarboxylase 2 (pancreatic islets and | TAGTTVYGAFDPLLAVAD |
| brain, 65 kDa) (SEQ ID NO: 229) | |
| GNAS1, partial (SEQ ID NO: 230) | YMCTHRLLL |
| heat shock protein (SEQ ID NO: 231) | AEDEVQRERVSAKNALESYA |
| heat shock protein (SEQ ID NO: 232) | AEKDEFEHKRKELEQVCNPI |
| heat shock protein (SEQ ID NO: 233) | AGGVMTALIKRNSTIPTKQT |
| heat shock protein (SEQ ID NO: 234) | DTERLIGDAAKNQVALNPQN |
| heat shock protein (SEQ ID NO: 235) | GLNVLRIINEPTAAAIAYGL |
| heat shock protein (SEQ ID NO: 236) | GSGPTIEEVD |
| heat shock protein (SEQ ID NO: 237) | IAYGLDRTGKGERNVLIFDL |
| heat shock protein (SEQ ID NO: 238) | KANKITITNDKGRLSKEEIE |
| heat shock protein (SEQ ID NO: 239) | KEEIERMVQEAEKYKAEDEV |
| heat shock protein (SEQ ID NO: 240) | KRTLSSSTQASLEIDSLFEG |
| heat shock protein (SEQ ID NO: 241) | LESYAFNMKSAVEDEGLKGK |
| heat shock protein (SEQ ID NO: 242) | LLLLDVAPLSLGLETAGGVM |
| heat shock protein (SEQ ID NO: 243) | MAKAAAVGIDLGTTYSCVGV |
| heat shock protein (SEQ ID NO: 244) | NDQGNRTTPSYVAFTDTERL |
| heat shock protein (SEQ ID NO: 245) | PAPGVPQIEVTFDIDANGIL |
| heat shock protein (SEQ ID NO: 246) | PFQVINDGDKPKVQVSYKGE |
| heat shock protein (SEQ ID NO: 247) | PGPGGFGAQGPKGGSGSGPT |
| heat shock protein (SEQ ID NO: 248) | PTKQTQIFTTYSDNQPGVLI |
| heat shock protein (SEQ ID NO: 249) | RFEELCSDLFRSTLEPVEKA |
| heat shock protein (SEQ ID NO: 250) | SCVGVFQHGKVEIIANDQGN |
| heat shock protein (SEQ ID NO: 251) | THLGGEDFDNRLVNHFVEEF |
| heat shock protein (SEQ ID NO: 252) | TIDDGIFEVKATAGDTHLGG |
| heat shock protein (SEQ ID NO: 253) | VCNPIISGLYQGAGGPGPGG |
| heat shock protein (SEQ ID NO: 254) | VQKLLQDFFNGRDLNKSINP |
| IAPP (SEQ ID NO: 255) | VGSNTYGKRNAVEVLKREPL + CITR(R73) |
| IAPP (SEQ ID NO: 256) | VGSNTYGKRNAVEVLKREPL + CITR(R73, R81) |
| Immunoglobulin heavy chain (SEQ ID NO: 257) | CARQEDTAMVYYFDYW |
| Insulin (SEQ ID NO: 258) | GIVEQCCTSI |
| Insulin (SEQ ID NO: 259) | LVEALYLVCGERG |
| Insulin (SEQ ID NO: 260) | AAAFVNQHLCGSHLVEALYLVCGERGFFYT |
| Insulin (SEQ ID NO: 261) | ALWMRLLPLL |
| Insulin (SEQ ID NO: 262) | LPLLALLAL |
| Insulin (SEQ ID NO: 263) | LWMRLLPLL |
| Insulin (SEQ ID NO: 264) | ALWGPDPAAAF |
| Insulin (SEQ ID NO: 265) | LALWGPDPAA |
| Insulin (SEQ ID NO: 266) | RLLPLLALLAL |
| Insulin (SEQ ID NO: 267) | PLLALLALWGPDPAAAFVNQ |
| Insulin (SEQ ID NO: 268) | GSHLVEALY |
| Insulin (SEQ ID NO: 269) | ALLALWGPDPAA |
| Insulin (SEQ ID NO: 270) | ALWGPDPAAAFV |
| Insulin (SEQ ID NO: 271) | PLLALLALWGPD |
| Insulin (SEQ ID NO: 272) | GIVEQCCTSICSL |
| Insulin (SEQ ID NO: 838) | EAEDLQVGQVELGGGPGAGSLQPLALEGSLQ |
| | KRGIVEQ |
| insulin gene enhancer protein ISL-1 | GLQANPVEV |
| (SEQ ID NO: 273) | |
| Insulin precursor (SEQ ID NO: 274) | SHLVEALYLVCGERG |
| Insulin precursor (SEQ ID NO: 275) | ALWMRLLPL |
| Insulin precursor (SEQ ID NO: 276) | HLVEALYLV |
| Insulin precursor (SEQ ID NO: 277) | SLQKRGIVEQ |
| Insulin precursor (SEQ ID NO: 278) | SLQPLALEG |
| Insulin precursor (SEQ ID NO: 279) | SLQPLALEGSLQKRG |
| Insulin precursor (SEQ ID NO: 280) | SLYQLENYC |
| Insulin precursor (SEQ ID NO: 281) | EDLQVGQVELGGGPGA |
| Insulin precursor (SEQ ID NO: 282) | FYTPKTRREAEDLQVG |
| Insulin precursor (SEQ ID NO: 283) | GAGSLQPLALEGSLQKRG |
| Insulin precursor (SEQ ID NO: 284) | HLVEALYLVCGERGFF |
| Insulin precursor (SEQ ID NO: 285) | VCGERGFFYT |
| Insulin precursor (SEQ ID NO: 286) | VEQCCTSICSLYQ |
| Insulin precursor (SEQ ID NO: 287) | ALWGPDPAAA |
| Insulin precursor (SEQ ID NO: 288) | FFYTPKTRREAED |
| Insulin precursor (SEQ ID NO: 289) | FYTPKTRREAEDLQVGQ |
| Insulin precursor (SEQ ID NO: 290) | KRGIVEQCCTSICSL |
| Insulin precursor (SEQ ID NO: 291) | LALEGSLQK |
| Insulin precursor (SEQ ID NO: 292) | LVEALYLVCGERGFFYT |
| Insulin precursor (SEQ ID NO: 293) | MALWMRLLPLLALLAL |
| Insulin precursor (SEQ ID NO: 294) | RLLPLLALL |
| Insulin precursor (SEQ ID NO: 295) | WGPDPAAA |
| Insulin precursor (SEQ ID NO: 296) | AGSLQPLALEGSLQKRG |
| Insulin precursor (SEQ ID NO: 297) | ALYLVCGER |
| Insulin precursor (SEQ ID NO: 298) | CCTSICSLYQLENYCN |
| Insulin precursor (SEQ ID NO: 299) | EDLQVGQVELGGGPGAG |
| Insulin precursor (SEQ ID NO: 300) | FVNQHLCGSHLVEALYL |
| Insulin precursor (SEQ ID NO: 301) | GERGFFYTPKTRREAED |
| Insulin precursor (SEQ ID NO: 302) | GGGPGAGSLQPLALEGS |
| Insulin precursor (SEQ ID NO: 303) | GIVEQCCTSICSLYQ |
| Insulin precursor (SEQ ID NO: 304) | GQVELGGGPGAGSLQPL |
| Insulin precursor (SEQ ID NO: 305) | GSLQKRGIVEQCCTSIC |
| Insulin precursor (SEQ ID NO: 306) | PLALEGSLQKRGIVEQC |
| Insulin precursor (SEQ ID NO: 307) | TRREAEDLQVGQVELGG |
| Insulin precursor (SEQ ID NO: 308) | YLVCGERGFFYTPKT |
| Insulin precursor (SEQ ID NO: 309) | EAEDLQVGQVELGGGPGAGSLQPLALEGSLQ |
| Insulin precursor (SEQ ID NO: 310) | GSLQPLALEGSLQKRGIV |
| Insulin precursor (SEQ ID NO: 311) | PAAAFVNQHLCGSHLV |
| Insulin precursor (SEQ ID NO: 312) | EALYLVCGERG |
| Insulin precursor (SEQ ID NO: 313) | VCGERGFFYTPKTRREAEDLQVGQVELGGG |
| Insulin precursor (SEQ ID NO: 314) | FYTPKTRRE |
| Insulin precursor (SEQ ID NO: 315) | GERGFFYT |
| Insulin precursor (SEQ ID NO: 316) | ERGFFYTPK |
| Insulin precursor (SEQ ID NO: 317) | LVCGERGFFY |
| Insulin precursor (SEQ ID NO: 318) | LYLVCGERGF |
| Insulin precursor (SEQ ID NO: 319) | AEDLQVGQVE |
| Insulin precursor (SEQ ID NO: 320) | AGSLQPLAL |
| Insulin precursor (SEQ ID NO: 321) | AGSLQPLALE |
| Insulin precursor (SEQ ID NO: 322) | GAGSLQPLAL |
| Insulin precursor (SEQ ID NO: 323) | QPLALEGSL |
| Insulin precursor (SEQ ID NO: 324) | QPLALEGSLQ |
| Insulin precursor (SEQ ID NO: 325) | QVELGGGPG |
| Insulin precursor (SEQ ID NO: 326) | SLQPLALEGS |
| Insulin precursor (SEQ ID NO: 327) | VELGGGPGA |
| Insulin precursor (SEQ ID NO: 328) | GAGSLQPLALEGSLQKR |
| Insulin precursor (SEQ ID NO: 329) | FVNQHLCGSHLVEALY |
| Insulin precursor (SEQ ID NO: 330) | EAEDLQVGQVELGG |
| Insulin precursor (SEQ ID NO: 331) | LALEGSL |
| Insulin precursor (SEQ ID NO: 332) | PGAGSLQPLALE |
| Insulin precursor (SEQ ID NO: 333) | QVELGGGPGAG |
| Insulin precursor (SEQ ID NO: 334) | SLQPLALEGSL |
| Insulin precursor (SEQ ID NO: 335) | SLQPLALEGSLQ |
| Insulin precursor (SEQ ID NO: 336) | VELGGGPG |
| Insulin precursor (SEQ ID NO: 337) | PGAGSLQPLALEGSL |
| Insulin precursor (SEQ ID NO: 338) | GIVEQCCTSICSLYQL |
| Insulin precursor (SEQ ID NO: 339) | LLALWGPDPAAAFVNQ |
| Insulin precursor (SEQ ID NO: 340) | MRLLPLLALLALWGPD |
| Insulin precursor (SEQ ID NO: 341) | PLLALLALWGPDPAAA |
| Insulin precursor (SEQ ID NO: 342) | WGPDPAAAFVNQHLCG |
| Insulin precursor (SEQ ID NO: 343) | YLVCGERGFFYTPKTRR |
| Insulin precursor (SEQ ID NO: 344) | ALYLVCGERGFFYTPKT |
| Insulin precursor (SEQ ID NO: 345) | CGSHLVEALYLVCGERG |
| Insulin precursor (SEQ ID NO: 346) | ERGFFYTPKTRREAEDL |
| Insulin precursor (SEQ ID NO: 347) | LALEGSLQKRGIVEQCC |
| Insulin precursor (SEQ ID NO: 348) | PKTRREAEDLQVGQVEL |
| Insulin precursor (SEQ ID NO: 349) | QKRGIVEQCCTSICSLY |
| Insulin precursor (SEQ ID NO: 350) | VELGGGPGAGSLQPLAL |
| Insulin precursor (SEQ ID NO: 351) | GQVELGGGPGAGS |
| Islet amyloid polypeptide precursor | FLIVLSVAL |
| (SEQ ID NO: 352) | |
| Islet amyloid polypeptide precursor | KLQVFLIVL |
| (SEQ ID NO: 353) | |
| Islet amyloid polypeptide precursor | VGSNTYGKRNAVEVLKREPL + CITR(R9, R17) |
| (SEQ ID NO: 354) | |
| Islet amyloid polypeptide precursor | VALKLQVFL |
| (SEQ ID NO: 355) | |
| Islet cell autoantigen 1 (SEQ ID NO: 356) | AFIEFKADEKKEDE |
| Islet cell autoantigen 1 (SEQ ID NO: 357) | AFIKATGKKEDE |
| islet cell autoantigen 1 isoform g | QEPSQLISLEEENQR |
| (SEQ ID NO: 358) | |
| islet-specific glucose-6-phosphatase-related | FLWSVFMLI |
| protein (SEQ ID NO: 359) | |
| islet-specific glucose-6-phosphatase-related | FLFAVGFYL |
| protein isoform 1 (SEQ ID NO: 360) | |
| islet-specific glucose-6-phosphatase-related | RLLCALTSL |
| protein isoform 1 (SEQ ID NO: 361) | |
| islet-specific glucose-6-phosphatase-related | LNIDLLWSV |
| protein isoform 1 (SEQ ID NO: 362) | |
| islet-specific glucose-6-phosphatase-related | VLFGLGFAI |
| protein isoform 1 (SEQ ID NO: 363) | |
| islet-specific glucose-6-phosphatase-related | FLWSVFWLI |
| protein isoform 1 (SEQ ID NO: 364) | |
| islet-specific glucose-6-phosphatase-related | NLFLFLFAV |
| protein isoform 1 (SEQ ID NO: 365) | |
| islet-specific glucose-6-phosphatase-related | YLLLRVLNI |
| protein isoform 1 (SEQ ID NO: 366) | |
| islet-specific glucose-6-phosphatase-related | DWIHIDTTPFAGL |
| protein isoform 1 (SEQ ID NO: 367) | |
| islet-specific glucose-6-phosphatase-related | QHLQKDYRAYYTF |
| protein isoform 1 (SEQ ID NO: 368) | |
| islet-specific glucose-6-phosphatase-related | RVLNIDLLWSVPI |
| protein isoform 1 (SEQ ID NO: 369) | |
| islet-specific glucose-6-phosphatase-related | YTFLNFMSNVGDP |
| protein isoform 1 (SEQ ID NO: 370) | |
| islet-specific glucose-6-phosphatase-related | KDYRAYYTFLNFMSNVGDPR |
| protein isoform 1 (SEQ ID NO: 371) | |
| islet-specific glucose-6-phosphatase-related | KWCANPDWIHIDTTPFAGLV |
| protein isoform 1 (SEQ ID NO: 372) | |
| islet-specific glucose-6-phosphatase-related | GLVRNLGVL + CITR(R4) |
| protein isoform 1 (SEQ ID NO: 373) | |
| islet-specific glucose-6-phosphatase-related | HQVILGVIGGMLVAEAFEHT |
| protein isoform 1 (SEQ ID NO: 374) | |
| islet-specific glucose-6-phosphatase-related | QLYHFLQIPTHEEHLFYVLS |
| protein isoform 1 (SEQ ID NO: 375) | |
| MHC HLA-B7 heavy chain precursor | VMAPRTVLL |
| (SEQ ID NO: 376) | |
| myotonin-protein kinase isoform 3 | RLQQLVLDPGFLGLEPLLDL |
| (SEQ ID NO: 377) | |
| Phogrin (SEQ ID NO: 378) | LLLLLPPRV |
| phogrin (SEQ ID NO: 379) | GLSGLELDGMAELMA |
| proinsulin precursor (SEQ ID NO: 380) | HLCGSHLVEA |
| proinsulin precursor (SEQ ID NO: 381) | SHLVEALYLV |
| proinsulin precursor (SEQ ID NO: 382) | WMRLLPLLAL |
| proinsulin precursor (SEQ ID NO: 383) | LCGSHLVEAL |
| proinsulin precursor (SEQ ID NO: 384) | GGGPGAGSLQPLALEGSLQK |
| proinsulin precursor (SEQ ID NO: 385) | GAGSLQPLALEGSLQKRGIV |
| proinsulin precursor (SEQ ID NO: 386) | PLALEGSLQK |
| proinsulin precursor (SEQ ID NO: 387) | PLLALLALWG |
| proinsulin precursor (SEQ ID NO: 388) | TRREAEDLQVGQVELG |
| proinsulin precursor (SEQ ID NO: 389) | TRREAEDLQVGQVELG + DEAM(Q12) |
| proinsulin precursor (SEQ ID NO: 390) | TRREAEDLQVGQVELG + DEAM(Q9, Q12) |
| protein GNAS isoform GNASS | AMSNLVPPV |
| (SEQ ID NO: 391) | |
| protein tyrosine phosphatase, receptor type, N | ALTAVAEEV |
| precursor (SEQ ID NO: 392) | |
| protein tyrosine phosphatase, receptor type, N | SLYHVYEVNL |
| precursor (SEQ ID NO: 393) | |
| protein tyrosine phosphatase, receptor type, N | TIADFWQMV |
| precursor (SEQ ID NO: 394) | |
| protein tyrosine phosphatase, receptor type, N | VIVMLTPLV |
| precursor (SEQ ID NO: 395) | |
| protein tyrosine phosphatase, receptor type, N | CAYQAEPNTCATA |
| precursor (SEQ ID NO: 396) | |
| protein tyrosine phosphatase, receptor type, N | CTVIVMLTPLVED |
| precursor (SEQ ID NO: 397) | |
| protein tyrosine phosphatase, receptor type, N | DQFEFALTAVAEE |
| precursor (SEQ ID NO: 398) | |
| protein tyrosine phosphatase, receptor type, N | FYLKNVQTQETRTLTQFHF |
| precursor (SEQ ID NO: 399) | |
| protein tyrosine phosphatase, receptor type, N | GSFINISVVGPAL |
| precursor (SEQ ID NO: 400) | |
| protein tyrosine phosphatase, receptor type, N | IKLKVESSPSRSDYINASPI |
| precursor (SEQ ID NO: 401) | |
| protein tyrosine phosphatase, receptor type, N | LEILAEHVHMSSG |
| precursor (SEQ ID NO: 402) | |
| protein tyrosine phosphatase, receptor type, N | LYHVYEVNLVSEHIWCEDFL |
| precursor (SEQ ID NO: 403) | |
| protein tyrosine phosphatase, receptor type, N | MVWESGCTVIVMLTPLVEDGV |
| precursor (SEQ ID NO: 404) | |
| protein tyrosine phosphatase, receptor type, N | PAYIATQGPLSHT |
| precursor (SEQ ID NO: 405) | |
| protein tyrosine phosphatase, receptor type, N | PSLSYEPALLQPY |
| precursor (SEQ ID NO: 406) | |
| protein tyrosine phosphatase, receptor type, N | RSVLLTLVALAGV |
| precursor (SEQ ID NO: 407) | |
| protein tyrosine phosphatase, receptor type, N | SEHIWCEDFLVRSFYLKNVQ |
| precursor (SEQ ID NO: 408) | |
| protein tyrosine phosphatase, receptor type, N | SKDQFEFALTAVAEEVNAILK |
| precursor (SEQ ID NO: 409) | |
| protein tyrosine phosphatase, receptor type, N | SLYHVYEVNLVSE |
| precursor (SEQ ID NO: 410) | |
| protein tyrosine phosphatase, receptor type, N | TYILIDMVLNRMA |
| precursor (SEQ ID NO: 411) | |
| protein tyrosine phosphatase, receptor type, N | DRGEKPASPAVQPDAALQRLAAVL |
| precursor (SEQ ID NO: 412) | |
| protein tyrosine phosphatase, receptor type, N | LPGPSPAQLFQDSGLLYLAQE |
| precursor (SEQ ID NO: 413) | |
| protein tyrosine phosphatase, receptor type, N | SPLGQSQPTVAGQPSARPAAEEYGYIVTDQKP |
| precursor (SEQ ID NO: 414) | LSLAAGVK |
| protein tyrosine phosphatase, receptor type, N | LAKEWQALCAYQAEPNTCATAQGEG |
| precursor (SEQ ID NO: 415) | |
| protein tyrosine phosphatase, receptor type, N | VSSVSSQFSDAAQASPSSHSS |
| precursor (SEQ ID NO: 416) | |
| protein tyrosine phosphatase, receptor type, N | DQFEFALTAVAEEVNA |
| precursor (SEQ ID NO: 417) | |
| protein tyrosine phosphatase, receptor type, N | FQDSGLLYLAQELPA |
| precursor (SEQ ID NO: 418) | |
| protein tyrosine phosphatase, receptor type, N | GASSSLSPLQAELLP |
| precursor (SEQ ID NO: 419) | |
| protein tyrosine phosphatase, receptor type, N | RSDYINASPIIEHDPRM |
| precursor (SEQ ID NO: 420) | |
| Receptor-type tyrosine-protein phosphatase-like N | LLPPLLEHL |
| precursor (SEQ ID NO: 421) | |
| Receptor-type tyrosine-protein phosphatase-like N | SLAAGVKLL |
| precursor (SEQ ID NO: 422) | |
| Receptor-type tyrosine-protein phosphatase-like N | SLSPLQAEL |
| precursor (SEQ ID NO: 423) | |
| Receptor-type tyrosine-protein phosphatase-like N | LAKEWQALCAYQAEPNTCATAQG |
| precursor (SEQ ID NO: 424) | |
| Receptor-type tyrosine-protein phosphatase-like N | VSSQFSDAAQASPSSHSS |
| precursor (SEQ ID NO: 425) | |
| Receptor-type tyrosine-protein phosphatase-like N | KLKVESSPSRSDYINASPIIEHDP |
| precursor (SEQ ID NO: 426) | |
| Receptor-type tyrosine-protein phosphatase-like N | LAKEWQALCAYQAEPNTCATAQGEGNIK |
| precursor (SEQ ID NO: 427) | |
| Receptor-type tyrosine-protein phosphatase-like N | SFYLKNVQTQETRTLTQFHF |
| precursor (SEQ ID NO: 428) | |
| Receptor-type tyrosine-protein phosphatase-like N | SKDQFEFALTAVAEEVNAILKA |
| precursor (SEQ ID NO: 429) | |
| Receptor-type tyrosine-protein phosphatase-like N | SRVSSVSSQFSDAAQASPSSHSSTPSWCE |
| precursor (SEQ ID NO: 430) | |
| Receptor-type tyrosine-protein phosphatase-like N | MVWESGCTV |
| precursor (SEQ ID NO: 431) | |
| Receptor-type tyrosine-protein phosphatase-like N | DFWQMVWESGCTVIVMLTPLVEDGV |
| precursor (SEQ ID NO: 432) | |
| S100 calcium binding protein B | ECDFQEFMAFVAMVTTACHEFFEHE |
| (SEQ ID NO: 433) | |
| S100 calcium binding protein B | KAMVALIDVFHQYSGREGDK |
| (SEQ ID NO: 434) | |
| S100 calcium binding protein B | KHKLKKSELKELINNELSHFLE |
| (SEQ ID NO: 435) | |
| S100 calcium binding protein B | REGDKHKLKKSELKEL |
| (SEQ ID NO: 436) | |
| S100 calcium binding protein B | ALIDVFHQY |
| (SEQ ID NO: 437) | |
| S100 calcium binding protein B | GREGDKHKL |
| (SEQ ID NO: 438) | |
| Secretogranin V (7B2 protein) (SEQ ID NO: 439) | YLQGQRLDNV |
| similar to ribosomal protein L29 | AKSKNHTTHN |
| (SEQ ID NO: 440) | |
| solute carrier family 30 member 8 | ACERLLYPDYQIQATVMIIVSSCAVAA |
| (SEQ ID NO: 441) | |
| solute carrier family 30 member 8 | AKMHAFTLESVELQQKPVNKDQCPRER |
| (SEQ ID NO: 442) | |
| solute carrier family 30 member 8 | ANEYAYAKWKLCSASAICFIFMIAEVV |
| (SEQ ID NO: 443) | |
| solute carrier family 30 member 8 | ASRDSQVVRREIAKALSKSFTMHSLTIQMESP |
| (SEQ ID NO: 444) | VD |
| solute carrier family 30 member 8 | DGVLSVHSLHIWSLTMNQVILSAHVAT |
| (SEQ ID NO: 445) | |
| solute carrier family 30 member 8 | EELESGGMYHCHSGSKPTEKGANEYAY |
| (SEQ ID NO: 446) | |
| solute carrier family 30 member 8 | FGWHRAEILGALLSILCIWVVTGVLVYLACER |
| (SEQ ID NO: 447) | LLYPDYQIQ |
| solute carrier family 30 member 8 | FIFSILVLASTITILKDFSILLMEGVP |
| (SEQ ID NO: 448) | |
| solute carrier family 30 member 8 | GHIAGSLAVVTDAAHLLIDLTSFLLSL |
| (SEQ ID NO: 449) | |
| solute carrier family 30 member 8 | HLLIDLTSFLLSLFSLWLSSKPPSKRL |
| (SEQ ID NO: 450) | |
| solute carrier family 30 member 8 | HQRCLGHNHKEVQANASVRAAFVHALG |
| (SEQ ID NO: 451) | |
| solute carrier family 30 member 8 | LFQSISVLISALIIYFKPEYKIADPIC |
| (SEQ ID NO: 452) | |
| solute carrier family 30 member 8 | LKDFSILLMEGVPKSLNYSGVKELILA |
| (SEQ ID NO: 453) | |
| solute carrier family 30 member 8 | MEFLERTYLVNDKAAKMHAFTLESVEL |
| (SEQ ID NO: 454) | |
| solute carrier family 30 member 8 | MHSLTIQMESPVDQDPDCLFCEDPCD |
| (SEQ ID NO: 455) | |
| solute carrier family 30 member 8 | NASVRAAFVHALGDLFQSISVLISALI |
| (SEQ ID NO: 456) | |
| solute carrier family 30 member 8 | QKPVNKDQCPRERPEELESGGMYHCHS |
| (SEQ ID NO: 457) | |
| solute carrier family 30 member 8 | SAICFIFMIAEVVGGHIAGSLAVVTDA |
| (SEQ ID NO: 458) | |
| solute carrier family 30 member 8 | SCAVAANIVLTVVLHQRCLGHNHKEVQ |
| (SEQ ID NO: 459) | |
| solute carrier family 30 member 8 | SLNYSGVKELILAVDGVLSVHSLHIWS |
| (SEQ ID NO: 460) | |
| solute carrier family 30 member 8 | SLWLSSKPPSKRLTFGWHRAEILGALL |
| (SEQ ID NO: 461) | |
| solute carrier family 30 member 8 | TMNQVILSAHVATAASRDSQVVRREIA |
| (SEQ ID NO: 462) | |
| solute carrier family 30 member 8 | YFKPEYKIADPICTFIFSILVLASTIT |
| (SEQ ID NO: 463) | |
| solute carrier family 30 member 8 | ERTYLVNDKAAKMHA |
| (SEQ ID NO: 464) | |
| solute carrier family 30 member 8 | IFSILVLASTITILK |
| (SEQ ID NO: 465) | |
| solute carrier family 30 member 8 | YAYAKWKLCSASAI |
| (SEQ ID NO: 466) | |
| solute carrier family 30 member 8 | YKIADPICTFIFSIL |
| (SEQ ID NO: 467) | |
| tafazzin exon 7 deleted variant short form | PIILPLWHVGMND |
| (SEQ ID NO: 468) | |
| TAZ protein (SEQ ID NO: 469) | PIILPLWHVGEPG |
| tyrosine phosphatase (SEQ ID NO: 470) | VLNRMAKGV + CITR(R4) |
| tyrosine phosphatase (SEQ ID NO: 471) | GDRGEKPASPAVQPDA |
| tyrosine phosphatase (SEQ ID NO: 472) | VPRLPEQGSSSRAEDSPEG |
| tyrosine phosphatase (SEQ ID NO: 473) | TGLQILQTGVGQREEAAA + DEAM(Q4, Q7, |
| | Q12) |
| tyrosine phosphatase (SEQ ID NO: 474) | DKERLAALGPEGA |
| tyrosine phosphatase (SEQ ID NO: 475) | FYLKNVQTQETRT |
| tyrosine phosphatase (SEQ ID NO: 476) | MVWESGCTVIVML |
| tyrosine phosphatase (SEQ ID NO: 477) | TVIVMLTPLVEDG |
| tyrosine phosphatase (SEQ ID NO: 478) | VKEIDIAATLEHV |
| unknown protein eluted from human MHC allele | FLSGAVNRL |
| (SEQ ID NO: 479) | |
| unknown protein eluted from human MHC allele | VLSRNILLEL |
| (SEQ ID NO: 480) | |
| urocortin III (SEQ ID NO: 481) | MLMPVHFLL |
| Vitamin D-binding protein (SEQ ID NO: 482) | LLTTLSNRV |
| Vitamin D-binding protein (SEQ ID NO: 483) | NLIKLAQKV |
| zinc transporter 8 (SEQ ID NO: 484) | VMIIVSSLAV |
| zinc transporter 8 isoform a (SEQ ID NO: 485) | ALGDLFQSI |
| zinc transporter 8 isoform a (SEQ ID NO: 486) | AVAANIVLTV |
| zinc transporter 8 isoform a (SEQ ID NO: 487) | DLTSFLLSL |
| zinc transporter 8 isoform a (SEQ ID NO: 488) | EILGALLSI |
| zinc transporter 8 isoform a (SEQ ID NO: 489) | FLLSLFSLWL |
| zinc transporter 8 isoform a (SEQ ID NO: 490) | HIAGSLAVV |
| zinc transporter 8 isoform a (SEQ ID NO: 491) | ILAVDGVLSV |
| zinc transporter 8 isoform a (SEQ ID NO: 492) | ILGALLSIL |
| zinc transporter 8 isoform a (SEQ ID NO: 493) | ILKDFSILL |
| zinc transporter 8 isoform a (SEQ ID NO: 494) | ILSAHVATA |
| zinc transporter 8 isoform a (SEQ ID NO: 495) | ILVLASTITI |
| zinc transporter 8 isoform a (SEQ ID NO: 496) | LLIDLTSFL |
| zinc transporter 8 isoform a (SEQ ID NO: 497) | LLMEGVPKSL |
| zinc transporter 8 isoform a (SEQ ID NO: 498) | SISVLISAL |
| zinc transporter 8 isoform a (SEQ ID NO: 499) | SLNYSGVKEL |
| zinc transporter 8 isoform a (SEQ ID NO: 500) | SVHSLHIWSL |
| zinc transporter 8 isoform a (SEQ ID NO: 501) | VVTGVLVYL |
| zinc transporter 8 isoform a (SEQ ID NO: 502) | FIFSILVLA |
| zinc transporter 8 isoform a (SEQ ID NO: 503) | IQATVMIIV |
| zinc transporter 8 isoform a (SEQ ID NO: 504) | KMYAFTLES |
| zinc transporter 8 isoform a (SEQ ID NO: 505) | KSLNYSGVK |
| zinc transporter 8 isoform a (SEQ ID NO: 506) | LAVDGVLSV |
| zinc transporter 8 isoform a (SEQ ID NO: 507) | LLSLFSLWL |
| zinc transporter 8 isoform a (SEQ ID NO: 508) | RLLYPDYQI |
| zinc transporter 8 isoform a (SEQ ID NO: 509) | TMHSLTIQM |
| zinc transporter 8 isoform a (SEQ ID NO: 510) | VAANIVLTV |
| zinc transporter 8 isoform a (SEQ ID NO: 511) | FIFSILVLA + PHOS(54) |
| zinc transporter 8 isoform a (SEQ ID NO: 512) | CLGHNHKEV |
| zinc transporter 8 isoform a (SEQ ID NO: 513) | KIADPICTFI |
| zinc transporter 8 isoform a (SEQ ID NO: 514) | KMYAFTLESV |
| zinc transporter 8 isoform a (SEQ ID NO: 515) | LLIDLTSFLL |
| zinc transporter 8 isoform a (SEQ ID NO: 516) | ILKDFSILLMEGVPKSLNYS |
| zinc transporter 8 isoform a (SEQ ID NO: 517) | VRREIAKALSKSFTMHSLTI |
| zinc transporter 8 isoform a (SEQ ID NO: 518) | AKMYAFTLESVELQQ |
| Preproinsulin (SEQ ID NO: 519) | SHFSLKKGAAALGIGTDSVI |
| Preproinsulin (SEQ ID NO: 520) | AAALGIGTDSVILIKCDERG |
| Preproinsulin (SEQ ID NO: 521) | VSYQPLGDKVNFFRMVISNP |
| Preproinsulin (SEQ ID NO: 522) | MEFLERTYLVNDKAAKMYAF |
| Preproinsulin (SEQ ID NO: 523) | LVNDKAAKMYAFTLESVELQ |
| Preproinsulin (SEQ ID NO: 524) | MYAFTLESVELQQKPVNKDQ |
| Preproinsulin (SEQ ID NO: 525) | GHNHKEVQANASVRAAFVHA |
| Preproinsulin (SEQ ID NO: 526) | MALWMRLLPLLALLALWGPDPAAAFVNQHL |
| | CGSHLVEALYLVCGERGFFYTPKTRREAEDLQ |
| | VGQVELGGGPGAGSLQPLALEGSLQKRGIVEQ |
| | CCTSICSLYQLENYCN |
| Preproinsulin (SEQ ID NO: 527) | LVCGERGFF |
| Preproinsulin (SEQ ID NO: 528) | TPKTRREAEDL |
| Preproinsulin (SEQ ID NO: 529) | ALEGSLQKR |
| Preproinsulin (SEQ ID NO: 530) | IVEQCCTSI |
| Proinsulin (SEQ ID NO: 834) | GAGSLQPLALEGSLQKRGIVEQ |
| RASGRP2 (SEQ ID NO: 533) | AAAAARPAGGSARRWGRPGRCGLLAAGP |
| | KRVRSEPGGRLPERSLGPAHPAPAAMAGT |
| | LDLDKGCTVEELLRGCIEAFDDSGKVRDP |
| | QLVRMFLMMHPWYIPSSQLAAKLLHIYQ |
| | QSRKDNSNSLQVKTCHLVRYWISAFPAEF |
| | DLNPELAEQIKELKALLDQEGNRRHSSLID |
| | IDSVPTYKWKRQVTQRNPVGQKKRKMSL |
| | LFDHLEPMELAEHLTYLEYRSFCKILFQDY |
| | HSFVTHGCTVDNPVLERFISLFNSVSQWV |
| | QLMILSKPTAPQRALVITHFVHVAEKLLQL |
| | QNFNTLMAVVGGLSHSSISRLKETHSHVSP |
| | ETIKLWEGLTELVTATGNYGNYRRRLAAC |
| | VGFRFPILGVHLKDLVALQLALPD |
| | WLDPARTRLNGAKMKQLFSILEELAMVTS |
| | LRPPVQANPDLLSLLTVSLDQYQTEDELY |
| | QLSLQREPRSKSSPTSPTSCTPPPRPPVLEE |
| | WTSAAKPKLDQALWEHIEKMVESVFRNF |
| | DVDGDGHISQEEFQIIRGNFPYLSAFGDLD |
| | QNQDGCISREEMVSYFLRSSSVLGGRMGF |
| | VHNFQESNSLRPVACRHCKALILGIYKQG |
| | LKCRACGVNCHKQCKDRLSVECRRRAQS |
| | VSLEGSAPSPSPMHSHEIHRAFSFSLPRPGR |
| | RGSRPPEIREEEVQTVEDGVFDIHL |
| RASGRP2 (SEQ ID NO: 534) | MAGTLDLDKGCTVEELLRGCIEAFDDSGK |
| | VRDPQLVRMFLMMHPWYIPSSQLAAKLL |
| | HIYQQSRKDNSNSLQVKTCHLVRYWISAF |
| | PAEFDLNPELAEQIKELKALLDQEGNRRHS |
| | SLIDIDSVPTYKWKRQVTQRNPVGQKKRK |
| | MSLLFDHLEPMELAEHLTYLEYRSFCKILF |
| | QDYHSFVTHGCTVDNPVLERFISLFNSVSQ |
| | WVQLMILSKPTAPQRALVITHFVHVAEKL |
| | LQLQNFNTLMAVVGGLSHSSISRLKETHS |
| | HVSPETIKLWEGLTELVTATGNYGNYRRR |
| | LAACVGFRFPILGVHLKDLVALQLALPDW |
| | LDPARTRLNGAKMKQLFSILEELAMVTSL |
| | RPPVQANPDLLSLLTVSLDQYQTEDELYQ |
| | LSLQREPRSKSSPTSPTSCTPPPRPPVLEEW |
| | TSAAKPKLDQALVVEHIEKMVESVFRNFD |
| | VDGDGHISQEEFQIIRGNFPYLSAFGDLDQ |
| | NQDGCISREEMVSYFLRSSSVLGGRMGFV |
| | HNFQESNSLRPVACRHCKALILGIYKQGL |
| | KCRACGVNCHKQCKDRLSVECRRRAQSV |
| | SLEGSAPSPSPMHSHEIHRAFSFSLPRPGRR |
| | GSRPPEIREEEVQTVEDGVFDIHL |
| RASGRP2 (SEQ ID NO: 535) | MGTQRLCGRGTQGWPGSSEQHVQEATSS |
| | AGLHSGVDELGVRSEPGGRLPERSLGPAH |
| | PAPAAMAGTLDLDKGCTVEELLRGCIEAF |
| | DDSGKVRDPQLVRMFLMMHPWYIPSSQL |
| | AAKLLHIYQQSRKDNSNSLQVKTCHLVRY |
| | WISAFPAEFDLNPELAEQIKELKALLDQEG |
| | NRRHSSLIDIDSVPTYKWKRQVTQRNPVG |
| | QKKRKMSLLFDHLEPMELAEHLTYLEYRS |
| | FCKILFQDYHSFVTHGCTVDNPVLERFISL |
| | FNSVSQWVQLMILSKPTAPQRALVITHFV |
| | HVAEKLLQLQNFNTLMAVVGGLSHSSISR |
| | LKETHSHVSPETIKLWEGLTELVTATGNY |
| | GNYRRRLAACVGFRFPILGVHLKDLVALQ |
| | LALPDWLDPARTRLNGAKMKQLFSILEEL |
| | AMVTSLRPPVQANPDLLSLLTVSLDQYQT |
| | EDELYQLSLQREPRSKSSPTSPTSCTPPPRP |
| | PVLEEWTSAAKPKLDQALWEHIEKMVES |
| | VFRNFDVDGDGHISQEEFQIIRGNFPYLSA |
| | FGDLDQNQDGCISREEMVSYFLRSSSVLG |
| | GRMGFVHNFQESNSLRPVACRHCKALILG |
| | IYKQGLKCRACGVNCHKQCKDRLSVECR |
| | RRAQSVSLEGSAPSPSPMHSHEIHRAFSFSL |
| | PRPGRRGSRPPEIREEEVQTVEDGVFDIHL |
| RASGRP2 (SEQ ID NO: 536) | MAGTLDLDKGCTVEELLRGCIEAFDDSGK |
| | VRDPQLVRMFLMMHPWYIPSSQLAAKLL |
| | HIYQQSRKDNSNSLQVKTCHLVRYWISAF |
| | PAEFDLNPELAEQIKELKALLDQEGNRRHS |
| | SLIDIDSVPTYKWKRQVTQRNPVGQKKRK |
| | MSLLFDHLEPMELAEHLTYLEYRSFCKILF |
| | QDYHSFVTHGCTVDNPVLERFISLFNSVSQ |
| | WVQLMILSKPTAPQRALVITHFVHVAEKL |
| | LQLQNFNTLMAVVGGLSHSSISRLKETHS |
| | HVSPETIKLWEGLTELVTATGNYGNYRRR |
| | LAACVGFRFPILGVHLKDLVALQLALPDW |
| | LDPARTRLNGAKMKQLFSILEELAMVTSL |
| | RPPVQANPDLLSLLTVSLDQYQTEDE |
| | LYQLSLQREPRSKSSPTSPTSCTPPPRPPVL |
| | EEWTSAAKPKLDQALVVEHIEKMVESVFR |
| | NFDVDGDGHISQEEFQIIRGNFPYLSAFGD |
| | LDQNQDGCISREEMVSYFLRSSSVLGGRM |
| | GFVHNFQESNSLRPVACRHCKALILGIYKQ |
| | GLKCRACGVNCHKQCKDRLSVECRRRAQ |
| | SVSLEGSAPSPSPMHSHEIHRAFSFSL |
| RASGRP2 (SEQ ID NO: 537) | LVRYWISAFP |
| RASGRP2 (SEQ ID NO: 538) | LLFDHLEPMELAEHLTYLEYRSF |
| RASGRP2 (SEQ ID NO: 539) | NFNTLMAVVGGLSHSSISRLKETHSHVS |
| RASGRP2 (SEQ ID NO: 540) | PAAMAGTLDLDKGCT |
| RASGRP2 (SEQ ID NO: 541) | DKGCTVEELLRGCIE |
| RASGRP2 (SEQ ID NO: 542) | RGCIEAFDDSGKVRD |
| RASGRP2 (SEQ ID NO: 543) | GKVRDPQLVRMFLMM |
| RASGRP2 (SEQ ID NO: 544) | MFLMMHPWYIPSSQL |
| RASGRP2 (SEQ ID NO: 545) | PSSQLAAKLLHIYQQ |
| RASGRP2 (SEQ ID NO: 546) | HIYQQSRKDNSNSLQ |
| RASGRP2 (SEQ ID NO: 547) | SNSLQVKTCHLVRYW |
| RASGRP2 (SEQ ID NO: 548) | LVRYWISAFPAEFDL |
| RASGRP2 (SEQ ID NO: 549) | AEFDLNPELAEQIKE |
| RASGRP2 (SEQ ID NO: 550) | EQIKELKALLDQEGN |
| RASGRP2 (SEQ ID NO: 551) | DQEGNRRHSSLIDID |
| RASGRP2 (SEQ ID NO: 552) | LIDIDSVPTYKWKRQ |
| RASGRP2 (SEQ ID NO: 553) | KWKRQVTQRNPVGQK |
| RASGRP2 (SEQ ID NO: 554) | PVGQKKRKMSLLFDH |
| RASGRP2 (SEQ ID NO: 555) | LLFDHLEPMELAEHL |
| RASGRP2 (SEQ ID NO: 556) | LAEHLTYLEYRSFCK |
| RASGRP2 (SEQ ID NO: 557) | RSFCKILFQDYHSFV |
| RASGRP2 (SEQ ID NO: 558) | YHSFVTHGCTVDNPV |
| RASGRP2 (SEQ ID NO: 559) | VDNPVLERFISLFNS |
| RASGRP2 (SEQ ID NO: 560) | SLFNSVSQWVQLMIL |
| RASGRP2 (SEQ ID NO: 561) | QLMILSKPTAPQRAL |
| RASGRP2 (SEQ ID NO: 562) | PQRALVITHFVHVAE |
| RASGRP2 (SEQ ID NO: 563) | VHVAEKLLQLQNFNT |
| RASGRP2 (SEQ ID NO: 564) | QNFNTLMAVVGGLSH |
| RASGRP2 (SEQ ID NO: 565) | GGLSHSSISRLKETH |
| RASGRP2 (SEQ ID NO: 566) | LKETHSHVSPETIKL |
| RASGRP2 (SEQ ID NO: 567) | ETIKLWEGLTELVTA |
| RASGRP2 (SEQ ID NO: 568) | ELVTATGNYGNYRRR |
| RASGRP2 (SEQ ID NO: 569) | NYRRRLAACVGFRFP |
| RASGRP2 (SEQ ID NO: 570) | GFRFPILGVHLKDLV |
| RASGRP2 (SEQ ID NO: 571) | LKDLVALQLALPDWL |
| RASGRP2 (SEQ ID NO: 572) | LPDWLDPARTRLNGA |
| RASGRP2 (SEQ ID NO: 573) | RLNGAKMKQLFSILE |
| RASGRP2 (SEQ ID NO: 574) | FSILEELAMVTSLRP |
| RASGRP2 (SEQ ID NO: 575) | TSLRPPVQANPDLLS |
| RASGRP2 (SEQ ID NO: 576) | PDLLSLLTVSLDQYQ |
| RASGRP2 (SEQ ID NO: 577) | LDQYQTEDELYQLSL |
| RASGRP2 (SEQ ID NO: 578) | YQLSLQREPRSKSSP |
| RASGRP2 (SEQ ID NO: 579) | SKSSPTSPTSCTPPP |
| RASGRP2 (SEQ ID NO: 580) | CTPPPRPPVLEEWTS |
| RASGRP2 (SEQ ID NO: 581) | EEWTSAAKPKLDQAL |
| RASGRP2 (SEQ ID NO: 582) | LDQAVVEHIEKMVE |
| RASGRP2 (SEQ ID NO: 583) | EKMVESVFRNFDVDG |
| RASGRP2 (SEQ ID NO: 584) | FDVDGDGHISQEEFQ |
| RASGRP2 (SEQ ID NO: 585) | QEEFQIIRGNFPYLS |
| RASGRP2 (SEQ ID NO: 586) | FPYLSAFGDLDQNQD |
| RASGRP2 (SEQ ID NO: 587) | DQNQDGCISREEMVS |
| RASGRP2 (SEQ ID NO: 588) | EEMVSYFLRSSSVLG |
| RASGRP2 (SEQ ID NO: 589) | SSVLGGRMGFVHNFQ |
| RASGRP2 (SEQ ID NO: 590) | VHNFQESNSLRPVAC |
| RASGRP2 (SEQ ID NO: 591) | RPVACRHCKALILGI |
| RASGRP2 (SEQ ID NO: 592) | LILGIYKQGLKCRAC |
| RASGRP2 (SEQ ID NO: 593) | KCRACGVNCHKQCKD |
| RASGRP2 (SEQ ID NO: 594) | KQCKDRLSVECRRRA |
| RASGRP2 (SEQ ID NO: 595) | CRRRAQSVSLEGSAP |
| RASGRP2 (SEQ ID NO: 596) | EGSAPSPSPMHSHHH |
| RASGRP2 (SEQ ID NO: 597) | HSHEIHRAFSFSLPRP |
| RASGRP2 (SEQ ID NO: 598) | SLPRPGRRGSRPPEI |
| RASGRP2 (SEQ ID NO: 599) | RPPEIREEEVQTVED |
| RASGRP2 (SEQ ID NO: 600) | EEVQTVEDGVFDIHL |
| RASGRP2 (SEQ ID NO: 601) | AFSFSLPRPGR |
| RASGRP2 (SEQ ID NO: 602) | ALILGIYK |
| RASGRP2 (SEQ ID NO: 603) | ALLDQEGNRR |
| RASGRP2 (SEQ ID NO: 604) | ALVITHFVHVAEK |
| RASGRP2 (SEQ ID NO: 605) | DLVALQLALPDWLDPAR |
| RASGRP2 (SEQ ID NO: 606) | DNSNSLQVK |
| RASGRP2 (SEQ ID NO: 607) | HSSLIDIDSVPTYK |
| RASGRP2 (SEQ ID NO: 608) | KDNSNSLQVK |
| RASGRP2 (SEQ ID NO: 609) | LDQALVVEHIEK |
| RASGRP2 (SEQ ID NO: 610) | LLHIYQQSR |
| RASGRP2 (SEQ ID NO: 611) | LLQLQNFNTLMAVVGGLSHSSISR |
| RASGRP2 (SEQ ID NO: 612) | MFLMMHPWYIPSSQLAAK |
| RASGRP2 (SEQ ID NO: 613) | VRDPQLVR |
| RASGRP2 (SEQ ID NO: 614) | YWISAFPAEFDLNPELAEQIK |
| RASGRP2 (SEQ ID NO: 615) | PAAMAGTLDLDKGCT |
| RASGRP2 (SEQ ID NO: 616) | DKGCTVEELLRGCIE |
| RASGRP2 (SEQ ID NO: 617) | RGCIEAFDDSGKVRD |
| RASGRP2 (SEQ ID NO: 618) | GKVRDPQLVRMFLMM |
| RASGRP2 (SEQ ID NO: 619) | PSSQLAAKLLHIYQQ |
| RASGRP2 (SEQ ID NO: 620) | HIYQQSRKDNSNSLQ |
| RASGRP2 (SEQ ID NO: 621) | SNSLQVKTCHLVRYW |
| RASGRP2 (SEQ ID NO: 622) | LVRYWISAFPAEFDL |
| RASGRP2 (SEQ ID NO: 623) | AEFDLNPELAEQIKE |
| RASGRP2 (SEQ ID NO: 624) | EQIKELKALLDQEGN |
| RASGRP2 (SEQ ID NO: 625) | DQEGNRRHSSLIDID |
| RASGRP2 (SEQ ID NO: 626) | LIDIDSVPTYKWKRQ |
| RASGRP2 (SEQ ID NO: 627) | KWKRQVTQRNPVGQK |
| RASGRP2 (SEQ ID NO: 628) | PVGQKKRKMSLLFDH |
| RASGRP2 (SEQ ID NO: 629) | LLFDHLEPMELAEHL |
| RASGRP2 (SEQ ID NO: 630) | LAEHLTYLEYRSFCK |
| RASGRP2 (SEQ ID NO: 631) | RSFCKILFQDYHSFV |
| RASGRP2 (SEQ ID NO: 632) | YHSFVTHGCTVDNPV |
| RASGRP2 (SEQ ID NO: 633) | VDNPVLERFISLFNS |
| RASGRP2 (SEQ ID NO: 634) | SLFNSVSQWVQLMIL |
| RASGRP2 (SEQ ID NO: 635) | QLMILSKPTAPQRAL |
| RASGRP2 (SEQ ID NO: 636) | PQRALVITHFVHVAE |
| RASGRP2 (SEQ ID NO: 637) | VHVAEKLLQLQNFNT |
| RASGRP2 (SEQ ID NO: 638) | QNFNTLMAVVGGLSH |
| RASGRP2 (SEQ ID NO: 639) | GGLSHSSISRLKETH |
| RASGRP2 (SEQ ID NO: 640) | LKETHSHVSPETIKL |
| RASGRP2 (SEQ ID NO: 641) | ELVTATGNYGNYRRR |
| RASGRP2 (SEQ ID NO: 642) | NYRRRLAACVGFRFP |
| RASGRP2 (SEQ ID NO: 643) | GFRFPILGVHLKDLV |
| RASGRP2 (SEQ ID NO: 644) | LKDLVALQLALPDWL |
| RASGRP2 (SEQ ID NO: 645) | LPDWLDPARTRLNGA |
| RASGRP2 (SEQ ID NO: 646) | RLNGAKMKQLFSILE |
| RASGRP2 (SEQ ID NO: 647) | FSILEELAMVTSLRP |
| RASGRP2 (SEQ ID NO: 648) | TSLRPPVQANPDLLS |
| RASGRP2 (SEQ ID NO: 649) | PDLLSLLTVSLDQYQ |
| RASGRP2 (SEQ ID NO: 650) | LDQYQTEDELYQLSL |
| RASGRP2 (SEQ ID NO: 651) | YQLSLQREPRSKSSP |
| RASGRP2 (SEQ ID NO: 652) | SKSSPTSPTSCTPPP |
| RASGRP2 (SEQ ID NO: 653) | CTPPPRPPVLEEWTS |
| RASGRP2 (SEQ ID NO: 654) | EEWTSAAKPKLDQAL |
| RASGRP2 (SEQ ID NO: 655) | LDQALVVEHIEKMVE |
| RASGRP2 (SEQ ID NO: 656) | FDVDGDGHISQEEFQ |
| RASGRP2 (SEQ ID NO: 657) | QEEFQIIRGNFPYLS |
| RASGRP2 (SEQ ID NO: 658) | FPYLSAFGDLDQNQD |
| RASGRP2 (SEQ ID NO: 659) | DQNQDGCISREEMVS |
| RASGRP2 (SEQ ID NO: 660) | EEMVSYFLRSSSVLG |
| RASGRP2 (SEQ ID NO: 661) | SSVLGGRMGFVHNFQ |
| RASGRP2 (SEQ ID NO: 662) | VHNFQESNSLRPVAC |
| RASGRP2 (SEQ ID NO: 663) | RPVACRHCKALILGI |
| RASGRP2 (SEQ ID NO: 664) | LILGIYKQGLKCRAC |
| RASGRP2 (SEQ ID NO: 665) | KCRACGVNCHKQCKD |
| RASGRP2 (SEQ ID NO: 666) | KQCKDRLSVECRRRA |
| RASGRP2 (SEQ ID NO: 667) | CRRRAQSVSLEGSAP |
| RASGRP2 (SEQ ID NO: 668) | EGSAPSPSPMHSHHH |
| RASGRP2 (SEQ ID NO: 669) | HSHEIHRAFSFSLPRP |
| RASGRP2 (SEQ ID NO: 670) | SLPRPGRRGSRPPEI |
| RASGRP2 (SEQ ID NO: 671) | RPPEIREEEVQTVED |
| RASGRP2 (SEQ ID NO: 672) | EEVQTVEDGVFDIHL |
| GDP L-fucose synthse (SEQ ID NO: 673) | MGEPQGSMRILVTGG |
| GDP L-fucose synthse (SEQ ID NO: 674) | VVADGAGLPGEDWVF |
| GDP L-fucose synthse (SEQ ID NO: 675) | TAQTRALFEKVQPTH |
| GDP L-fucose synthse (SEQ ID NO: 676) | LFRNIKYNLDFWRKN |
| GDP L-fucose synthse (SEQ ID NO: 677) | VEIMNDNVLHSAFEVG |
| GDP L-fucose synthse (SEQ ID NO: 678) | DNVLHSAFEV |
| GDP L-fucose synthse (SEQ ID NO: 679) | NVLHSAFEVG |
| GDP L-fucose synthse (SEQ ID NO: 680) | NVLHSAFEVGARKVV |
| GDP L-fucose synthse (SEQ ID NO: 681) | VLHSAFEVGA |
| GDP L-fucose synthse (SEQ ID NO: 682) | KTTYPIDETMIHNGP |
| GDP L-fucose synthse (SEQ ID NO: 683) | IHNGPPHNSNFGYSY |
| GDP L-fucose synthse (SEQ ID NO: 684) | PHNSNFGYSYAKRMI |
| GDP L-fucose synthse (SEQ ID NO: 685) | AYFQQYGCTFTAVIP |
| GDP L-fucose synthse (SEQ ID NO: 686) | YGCTFTAVIPTNVFG |
| GDP L-fucose synthse (SEQ ID NO: 687) | LFIWVLREYNEVEPI |
| GDP L-fucose synthse (SEQ ID NO: 688) | LREYNEVEPIILSVG |
| GDP L-fucose synthse (SEQ ID NO: 689) | EVEPIILSVGEEDEV |
| GDP L-fucose synthse (SEQ ID NO: 690) | ILSVGEEDEVSIKEA |
| GDP L-fucose synthse (SEQ ID NO: 691) | EEDEVSIKEAAEAVV |
| GDP L-fucose synthse (SEQ ID NO: 692) | SIKEAAEAVVEAMDF |
| GDP L-fucose synthse (SEQ ID NO: 693) | AEAVVEAMDFHGEVT |
| GDP L-fucose synthse (SEQ ID NO: 694) | FDTTKSDGQFKKTAS |
| GDP L-fucose synthse (SEQ ID NO: 695) | FRFTPFKQAVKETCA |
| GDP L-fucose synthse (SEQ ID NO: 696) | KLLLHSGVEN |
| GDP L-fucose synthse (SEQ ID NO: 697) | GSMRILVTGGSGLVG |
| GDP L-fucose synthse (SEQ ID NO: 698) | LVTGGSGLVGKAIQK |
| GDP L-fucose synthse (SEQ ID NO: 699) | SGLVGKAIQKVVADG |
| GDP L-fucose synthse (SEQ ID NO: 700) | KAIQKVVADGAGLPG |
| GDP L-fucose synthse (SEQ ID NO: 701) | VVADGAGLPGEDWVF |
| GDP L-fucose synthse (SEQ ID NO: 702) | AGLPGEDWVFVSSKD |
| GDP L-fucose synthse (SEQ ID NO: 703) | EDWVFVSSKDADLTD |
| GDP L-fucose synthse (SEQ ID NO: 704) | VSSKDADLTDTAQTR |
| GDP L-fucose synthse (SEQ ID NO: 705) | ADLTDTAQTRALFEK |
| GDP L-fucose synthse (SEQ ID NO: 706) | ALFEKVQPTHVIHLA |
| GDP L-fucose synthse (SEQ ID NO: 707) | VQPTHVIHLAAMVGG |
| GDP L-fucose synthse (SEQ ID NO: 708) | VIHLAAMVGGLFRNI |
| GDP L-fucose synthse (SEQ ID NO: 709) | AMVGGLFRNIKYNLD |
| GDP L-fucose synthse (SEQ ID NO: 710) | KYNLDFWRKNVEIMND |
| GDP L-fucose synthse (SEQ ID NO: 711) | FWRKNVHMNDNVLHS |
| GDP L-fucose synthse (SEQ ID NO: 712) | NVLHSAFEVGARKVV |
| GDP L-fucose synthse (SEQ ID NO: 713) | AFEVGARKVVSCLST |
| GDP L-fucose synthse (SEQ ID NO: 714) | ARKVVSCLSTCIFPD |
| GDP L-fucose synthse (SEQ ID NO: 715) | SCLSTCIFPDKTTYP |
| GDP L-fucose synthse (SEQ ID NO: 716) | CIFPDKTTYPIDETM |
| GDP L-fucose synthse (SEQ ID NO: 717) | IDETMIHNGPPHNSN |
| GDP L-fucose synthse (SEQ ID NO: 718) | FGYSYAKRMIDVQNR |
| GDP L-fucose synthse (SEQ ID NO: 719) | AKRMIDVQNRAYFQQ |
| GDP L-fucose synthse (SEQ ID NO: 720) | DVQNRAYFQQYGCTF |
| GDP L-fucose synthse (SEQ ID NO: 721) | TAVIPTNVEGPHDNE |
| GDP L-fucose synthse (SEQ ID NO: 722) | TNVFGPHDNFNIEDG |
| GDP L-fucose synthse (SEQ ID NO: 723) | PHDNFNIEDGHVLPG |
| GDP L-fucose synthse (SEQ ID NO: 724) | NIEDGHVLPGLIHKV |
| GDP L-fucose synthse (SEQ ID NO: 725) | HVLPGLIHKVHLAKS |
| GDP L-fucose synthse (SEQ ID NO: 726) | LIHKVHLAKSSGSAL |
| GDP L-fucose synthse (SEQ ID NO: 727) | HLAKSSGSALTVWGT |
| GDP L-fucose synthse (SEQ ID NO: 728) | SGSALTVWGTGNPRR |
| GDP L-fucose synthse (SEQ ID NO: 729) | TVWGTGNPRRQFIYS |
| GDP L-fucose synthse (SEQ ID NO: 730) | GNPRRQFIYSLDLAQ |
| GDP L-fucose synthse (SEQ ID NO: 731) | QFIYSLDLAQLFIWV |
| GDP L-fucose synthse (SEQ ID NO: 732) | LDLAQLFIWVLREYN |
| GDP L-fucose synthse (SEQ ID NO: 733) | EEDEVSIKEAAEAVV |
| GDP L-fucose synthse (SEQ ID NO: 734) | EAMDFHGEVTFDTTK |
| GDP L-fucose synthse (SEQ ID NO: 735) | HGEVTFDTTKSDGQF |
| GDP L-fucose synthse (SEQ ID NO: 736) | SDGQFKKTASNSKLR |
| GDP L-fucose synthse (SEQ ID NO: 737) | KKTASNSKLRTYLPD |
| GDP L-fucose synthse (SEQ ID NO: 738) | NSKLRTYLPDFRFTP |
| GDP L-fucose synthse (SEQ ID NO: 739) | TYLPDFRFTPFKQAV |
| GDP L-fucose synthse (SEQ ID NO: 740) | FKQAVKETCAWFTDN |
| GDP L-fucose synthse (SEQ ID NO: 741) | KETCAWFTDNYEQARK |
| GDP L-fucose synthse (SEQ ID NO: 742) | LWEGLTELVTATGNYGNYR |
| GDP L-fucose synthse (SEQ ID NO: 743) | ILVTGGSGLVGK |
| GDP L-fucose synthse (SEQ ID NO: 744) | VVADGAGLPGEDWVFVSSK |
| GDP L-fucose synthse (SEQ ID NO: 745) | DADLTDTAQTR |
| GDP L-fucose synthse (SEQ ID NO: 746) | VQPTHVIHLAAMVGGLFR |
| GDP L-fucose synthse (SEQ ID NO: 747) | YNLDFWR |
| GDP L-fucose synthse (SEQ ID NO: 748) | YNLDFWRK |
| GDP L-fucose synthse (SEQ ID NO: 749) | NVHMNDNVLHSAFEVGAR |
| GDP L-fucose synthse (SEQ ID NO: 750) | NVHMNDNVLHSAFEVGARK |
| GDP L-fucose synthse (SEQ ID NO: 751) | VVSCLSTCIFPDK |
| GDP L-fucose synthse (SEQ ID NO: 752) | MIDVQNR |
| GDP L-fucose synthse (SEQ ID NO: 753) | RMIDVQNR |
| GDP L-fucose synthse (SEQ ID NO: 754) | SSGSALTVWGTGNPR |
| GDP L-fucose synthse (SEQ ID NO: 755) | SSGSALTVWGTGNPRR |
| GDP L-fucose synthse (SEQ ID NO: 756) | TTYPIDETMIHNGPPHNSNFGYSYAK |
| GDP L-fucose synthse (SEQ ID NO: 757) | EYNEVEPIILSVGEEDEVSIK |
| GDP L-fucose synthse (SEQ ID NO: 758) | TYLPDFR |
| GDP L-fucose synthse (SEQ ID NO: 759) | LRTYLPDFR |

In some embodiments, the at least one exogenous autoantigenic polypeptide does not include a HLA-G protein sequence or a functional fragment thereof, or an MHC protein sequence or a functional fragment thereof.

In some embodiments, one or more of the at least one exogenous autoantigenic polypeptides may include at least one Ii key peptide (e.g., positioned between a membrane anchor and an autoantigen). For example, in some embodiments, the Ii key peptide comprises or consists of the amino acid sequence LRMKLPKPPKPVSKMR (SEQ ID NO: 765), YRMKLPKPPKPVSKMR (SEQ ID NO: 766), LRMK (SEQ ID NO: 767), YRMK (SEQ ID NO: 768), LRMKLPK (SEQ ID NO: 769), YRMKLPK (SEQ ID NO: 770), YRMKLPKP (SEQ ID NO; 771), LRMKLPKP (SEQ ID NO; 772), LRMKLPKS (SEQ ID NO; 773), YRMKLPKS (SEQ ID NO; 774), LRMKLPKSAKP (SEQ ID NO: 775), or LRMKLPKSAKPVSK (SEQ ID NO: 776). In some embodiments, the at least one Ii key peptide comprises an amino acid sequence that is at least 90%, at least 92%, at least 94%, at least 95%, at least 96%, at least 97%, at least 98%, at least 99% identical, or 100% identical to any one of SEQ ID NOs. 765-776.

In some embodiments, one or more of the at least one exogenous autoantigenic polypeptide is on the cell surface. In some embodiments, one or more of the at least one exogenous autoantigenic polypeptide further comprises a membrane anchor or is tethered to the plasma membrane of the cell via attachment to a lipid moiety.

In some embodiments, the exogenous autoantigenic polypeptide comprises Formula I in an N-terminal to a C-terminal direction: X₁-X₂-X₃ (Formula I), where: X₁ comprises a type II membrane protein or a transmembrane domain thereof (e.g., any of the exemplary type II membrane proteins described herein or transmembrane domains thereof, e.g., a SMIM1 transmembrane domain or a transferrin receptor ((TfR1), also known as CD71) transmembrane domain); X₂ comprises a Ii key peptide (e.g., any of the exemplary Ii key peptides described herein or known in the art); and X₃ comprises an autoantigen (e.g., any of the exemplary autoantigens described herein or known in the art). In some embodiments, the exogenous autoantigenic polypeptide comprises Formula II in an N-terminal to C-terminal direction: X₁-X₂-X₃-X₄ (Formula II), where: X₁ comprises a type II membrane protein or a transmembrane domain thereof (e.g., any of the exemplary type II receptor transmembrane domains described herein or known in the art, e.g., a SMIM1 transmembrane domain or a TfR1 transmembrane domain); X₂ comprises a linker (e.g., any of the exemplary linkers described herein or known in the art); X₃ comprises a Ii key peptide (e.g., any of the exemplary Ii key peptides described herein or known in the art); and X₄ comprises an autoantigen (e.g., any of the exemplary autoantigens described herein or known in the art). In some embodiments, the linker is a polyGS linker. In some embodiments, the polyGS linker comprises (GS)ₙ, where n is 1, 2, 3, 4, 5, 6, 7, 8, 9, or 10. In some embodiments, the linker comprises or consists of GSGSGSGSGSGSGSGSGS (SEQ ID NO: 840) or GPGPG (SEQ ID NO: 841). In some embodiments, the Ii key peptide comprises a sequence selected from the group of: LRMKLPKPPKPVSKMR (SEQ ID NO: 765); YRMKLPKPPKPVSKMR (SEQ ID NO: 766); LRMK (SEQ ID NO: 767); YRMK (SEQ ID NO: 768); LRMKLPK (SEQ ID NO: 769); YRMKLPK (SEQ ID NO: 770); YRMKLPKP (SEQ ID NO: 771); LRMKLPKP (SEQ ID NO: 772); LRMKLPKS (SEQ ID NO: 773); YRMKLPKS (SEQ ID NO: 774); LRMKLPKSAKP (SEQ ID NO: 775); and LRMKLPKSAKPVSK (SEQ ID NO: 776). In some embodiments, the exogenous autoantigenic polypeptide further comprises, at its C-terminus, one or more (e.g., two, three, four, five, six, seven, eight, nine, or ten) additional autoantigens (e.g., the same or different autoantigens). In some embodiments, any two autoantigens are separated by a linker. In some embodiments, the linker is a polyGS linker. In some embodiments, the linker comprises or consists of GSGSGSGSGSGSGSGSGS (SEQ ID NO: 840) or GPGPG (SEQ ID NO: 841).

In some embodiments, the exogenous autoantigenic polypeptide is within the cell. In some embodiments, the exogenous autoantigenic polypeptide is on the intracellular side of the plasma membrane. In some embodiments, the exogenous autoantigenic polypeptide further comprises a membrane anchor or is tethered to the plasma membrane of the cell via attachment to a lipid moiety. In some embodiments, the exogenous autoantigenic polypeptide comprises Formula III in an N-terminal to a C-terminal direction: X₁-X₂-X₃ (Formula III), where: X₁ comprises a type I membrane protein transmembrane domain (e.g., any of the exemplary type I membrane proteins or transmembrane domains thereof described herein or known in the art, e.g., a GPA transmembrane domain); X₂ comprises a Ii key peptide (e.g., any of the Ii key peptides described herein or known in the art); and X₃ comprises an autoantigen (e.g., any of the autoantigens described herein or known in the art). In some embodiments, the exogenous autoantigenic polypeptide comprises Formula IV in an N-terminal to C-terminal direction: X₁-X₂-X₃-X₄ (Formula IV), where: X₁ comprises a type I membrane protein or a transmembrane domain thereof (e.g., any of the type I membrane proteins or transmembrane domains thereof described herein or known in the art, e.g., a GPA transmembrane domain); X₂ comprises a linker (e.g., any of the exemplary linkers described herein or known in the art); X₃ comprises a Ii key peptide (e.g., any of the exemplary linkers described herein or known in the art); and X₄ comprises an autoantigen (e.g., any of the autoantigens described herein or known in the art). In some embodiments, the linker is a polyGS linker (e.g., any of the exemplary polyGS linkers described herein. In some embodiments, the linker comprises or consists of GSGSGSGSGSGSGSGSGS (SEQ ID NO: 840) or GPGPG (SEQ ID NO: 841). In some embodiments, the exogenous autoantigenic polypeptide further comprises, at its N-terminus, a signal peptide. In some embodiments, the signal peptide is a GPA signal peptide. In some embodiments, the Ii key peptide is selected from the group of: LRMKLPKPPKPVSKMR (SEQ ID NO: 765); YRMKLPKPPKPVSKMR (SEQ ID NO: 766); LRMK (SEQ ID NO: 767); YRMK (SEQ ID NO: 768); LRMKLPK (SEQ ID NO: 769); YRMKLPK (SEQ ID NO: 770); YRMKLPKP (SEQ ID NO: 771); LRMKLPKP (SEQ ID NO: 772); LRMKLPKS (SEQ ID NO: 773); YRMKLPKS (SEQ ID NO: 774); LRMKLPKSAKP (SEQ ID NO: 775); and RMKLPKSAKPVSK (SEQ ID NO: 776). In some embodiments, the exogenous autoantigenic polypeptide further comprises, at its C-terminus, one or more (e.g., two, three, four, five, six, seven, eight, nine, or ten) additional autoantigens (e.g., the same or different autoantigens). In some embodiments, any two autoantigens are separated by a linker (e.g., any of the exemplary linkers described herein). In some embodiments, the linker is a polyGS linker. In some embodiments, the linker comprises GSGSGSGSGSGSGSGSGS (SEQ ID NO: 840) or GPGPG (SEQ ID NO: 841).

In some embodiments, the exogenous autoantigenic polypeptide comprises Formula VII in an N-terminal to C-terminal direction: X₁-X₂-X₃-X₄ (Formula VII), where: X₁ comprises a type I membrane protein or a transmembrane domain thereof; X₂ comprises a linker; X₃ comprises a cytoplasmic portion of CD74 or a fragment thereof; and X₄ comprises an autoantigen. In some embodiments, the linker comprises GSGSGSGSGSGSGSGSGS (SEQ ID NO: 840). In some embodiments, the cytoplasmic portion of CD74 comprises

(SEQ ID NO: 845)
QQQGRLDKLTVTSQNLQLENLRMKLPKPPKPVSKMRMATPLLMQALPMGA
LPQGPMQNATKYGNMTEDHVMHLLQNADPLKVYPPLKGSFPENLRHLKNT
METIDWKVFESWMHHWLLFEMSRHSLEQKPTDAPPKESLELEDPSSGLGV
TKQDLGPVPM.

In some embodiments, the N-terminus of the exogenous autoantigenic polypeptide further comprises a signal peptide.

In some embodiments, the exogenous autoantigenic polypeptide comprises Formula VIII in an N-terminal to C-terminal direction: X₁-X₂-X₃-X₄-X₅ (Formula VIII), where: X₁ comprises a type I membrane protein or a transmembrane domain thereof; X₂ comprises a linker; X₃ comprises a N-terminal cytoplasmic portion of CD74 or a fragment thereof; X₄ comprises an autoantigen; and X₅ comprises a C-terminal cytoplasmic portion of CD74. In some embodiments, the linker comprises GSGSGSGSGSGSGSGSGS (SEQ ID NO: 840). In some embodiments, the N-terminal cytoplasmic portion of CD74 comprises QQQGRLDKLTVTSQNLQLENLRMK (SEQ ID NO: 847). In some embodiments, the C-terminal cytoplasmic portion of CD74 comprises GALPQGPMQNATKYGNMTEDHVMHLLQNADPLKVYPPLKGSFPENLRHLKNTMETID WKVFESWMHHWLLFEMSRHSLEQKPTDAPPKESLELEDPSSGLGVTKQDLGPVPM (SEQ ID NO: 849). In some embodiments, the N-terminus of the exogenous autoantigenic polypeptide further comprises a signal peptide.

In some embodiments, the exogenous autoantigenic polypeptide comprises Formula XI in an N-terminal to C-terminal direction: X₁-X₂-X₃-X₄ (Formula XI), where: X₁ comprises a cytosolic protein or a fragment thereof; X₂ comprises a linker; X₃ comprises a cytoplasmic portion of CD74 or a fragment thereof; and X₄ comprises an autoantigen. In some embodiments, the cytosolic protein comprises MAGWNAYIDNLMADGTCQDAAIVGYKDSPSVWAAVPGKTFVNITPAEVGVLVGKDRS SFYVNGLTLGGQKCSVIRDSLLQDGEFSMDLRTKSTGGAPTFNVTVTKTDKTLVLLMG KEGVHGGLINKKCYEMASHLRRSQY (SEQ ID NO: 846). In some embodiments, the linker comprises GSGSGSGSGSGSGSGSGS (SEQ ID NO: 840). In some embodiments, the cytoplasmic portion of CD74 comprises

(SEQ ID NO: 845)
QQQGRLDKLTVTSQNLQLENLRMKLPKPPKPVSKMRMATPLLMQALPMGA
LPQGPMQNATKYGNMTEDHVMHLLQNADPLKVYPPLKGSFPENLRHLKNT
METIDWKVFESWMEIHWLLFEMSRHSLEQKPTDAPPKESLELEDPSSGLG
VTKQDLGPVPM.

In some embodiments, the N-terminus of the exogenous autoantigenic polypeptide further comprises a signal peptide.

In some embodiments, the exogenous autoantigenic polypeptide comprises Formula XII in an N-terminal to C-terminal direction: X₁-X₂-X₃-X₄-X₅ (Formula XII), where: X₁ comprises a cytoplasmice protein or a fragment thereof; X₂ comprises a linker; X₃ comprises a N-terminal cytoplasmic portion of CD74 or a fragment thereof; X₄ comprises an autoantigen; and X₅ comprises a C-terminal cytoplasmic portion of CD74. In some embodiments, the cytoplasmic protein comprises MAGWNAYIDNLMADGTCQDAAIVGYKDSPSVWAAVPGKTFVNITPAEVGVLVGKDRS SFYVNGLTLGGQKCSVIRDSLLQDGEFSMDLRTKSTGGAPTFNVTVTKTDKTLVLLMG KEGVHGGLINKKCYEMASHLRRSQY (SEQ ID NO: 846). In some embodiments, the linker comprises GSGSGSGSGSGSGSGSGS (SEQ ID NO: 840). In some embodiments, the N-terminal cytoplasmic portion of CD74 comprises: QQQGRLDKLTVTSQNLQLENLRMK (SEQ ID NO: 847). In some embodiments, the C-terminal cytoplasmic portion of CD74 comprises: GALPQGPMQNATKYGNMTEDHVMHLLQNADPLKVYPPLKGSFPENLRHLKNTMETID WKVFESWMHHWLLFEMSRHSLEQKPTDAPPKESLELEDPSSGLGVTKQDLGPVPM (SEQ ID NO: 849). In some embodiments, the N-terminus of the exogenous autoantigenic polypeptide further comprises a signal peptide.

In some embodiments, the exogenous autoantigenic polypeptide is present on the cell surface. In some embodiments, the exogenous autoantigenic polypeptide comprises Formula IX in an N-terminal to C-terminal direction: X₁-X₂-X₃ (Formula IX), where: X₁ comprises a type II membrane protein or a transmembrane domain thereof; X₂ comprises a cytoplasmic portion of CD74 or a fragment thereof; and X₃ comprises an autoantigen. In some embodiments, the cytoplasmic portion of CD74 comprises

(SEQ ID NO: 845)
QQQGRLDKLTVTSQNLQLENLRMKLPKPPKPVSKMRMATPLLMQALPMGA
LPQGPMQNATKYGNMTEDHVMHLLQNADPLKVYPPLKGSFPENLRHLKNT
METIDWKVFESWMEIHWLLFEMSRHSLEQKPTDAPPKESLELEDPSSGLG
VTKQDLGPVPM.

In some embodiments, the N-terminus of the exogenous autoantigenic polypeptide further comprises a signal peptide.

In some embodiments, the exogenous autoantigenic polypeptide comprises Formula X in an N-terminal to C-terminal direction: X₁-X₂-X₃-X₄-X₅ (Formula X), where: X₁ comprises a type II membrane protein or a transmembrane domain thereof; X₂ comprises a linker; X₃ comprises a N-terminal cytoplasmic portion of CD74 or a fragment thereof; X₄ comprises an autoantigen; and X₅ comprises a C-terminal cytoplasmic portion of CD74. In some embodiments, the linker comprises GGGGSGGGGSGGGGSGGGGS (SEQ ID NO: 850). In some embodiments, the N-terminal cytoplasmic portion of CD74 comprises QQQGRLDKLTVTSQNLQLENLRMK (SEQ ID NO: 847). In some embodiments, the C-terminal cytoplasmic portion of CD74 comprises ALPQGPMQNATKYGNMTEDHVMHLLQNADPLKVYPPLKGSFPENLRHLKNTMETIDW KVFESWMHHWLLFEMSRHSLEQKPTDAPPKESLELEDPSSGLGVTKQDLGPVPM (SEQ ID NO: 848). In some embodiments, the N-terminus of the exogenous autoantigenic polypeptide further comprises a signal peptide.

In some embodiments, the exogenous autoantigenic polypeptide comprises Formula XIII in an N-terminal to C-terminal direction: X₁-X₂-X₃-X₄ (Formula XIII), where: X₁ comprises an Ii key peptide; X₂ comprises an autoantigen; X₃ comprises a linker; and X₄ comprises a Type I membrane protein or a transmembrane domain thereof. In some embodiments, the linker comprises GPGPG (SEQ ID NO: 841). In some embodiments, X₁ comprises two or more (e.g., three, four, five, or six) Ii key peptides. In some embodiments, the N-terminus of the exogenous autoantigenic polypeptide further comprises a signal peptide.

In some embodiments, the exogenous antigenic polypeptide is in the cytosol of the cell. In some embodiments, the exogenous antigenic polypeptide comprises Formula V in an N-terminal to a C-terminal direction: X₁-X₂-X₃ (Formula V), where: X₁ comprises a cytosolic polypeptide (e.g., any of the exemplary cytosolic polypeptides described herein, e.g., profiling (SEQ ID NO: 833) or a fragment thereof); X₂ comprises a Ii key peptide (e.g., any of the exemplary Ii key peptides described herein or known in the art); and X₃ comprises the exogenous antigenic polypeptide (e.g., any of the exemplary antigenic polypeptides described herein or known in the art).

In some embodiments, the exogenous autoantigenic polypeptide comprises Formula VI in an N-terminal to C-terminal direction: X₁-X₂-X₃-X₄ (Formula VI), where: X₁ comprises a cytosolic polypeptide or a fragment thereof (e.g., any of the exemplary cytosolic polypeptides described herein, e.g., profilin, ferritin, or a fragment thereof); X₂ comprises a linker (e.g., any of the exemplary linkers described herein or known in the art); X₃ comprises a Ii key peptide (e.g., any of the exemplary Ii key peptides described herein or known in the art); and X₄ comprises an autoantigen (e.g., any of the exemplary autoantigens described herein or known in the art). In some embodiments, the linker is a polyGS linker. In some embodiments, the linker comprises GSGSGSGSGSGSGSGSGS (SEQ ID NO: 840) or GPGPG (SEQ ID NO: 841). In some embodiments, the Ii key peptide is selected from the group of: LRMKLPKPPKPVSKMR (SEQ ID NO: 765); YRMKLPKPPKPVSKMR (SEQ ID NO: 766); LRMK (SEQ ID NO: 767); YRMK (SEQ ID NO: 768); LRMKLPK (SEQ ID NO: 769); YRMKLPK (SEQ ID NO: 770); YRMKLPKP (SEQ ID NO: 771); LRMKLPKP (SEQ ID NO: 772); LRMKLPKS (SEQ ID NO: 773); YRMKLPKS (SEQ ID NO: 774); LRMKLPKSAKP (SEQ ID NO: 775); and LRMKLPKSAKPVSK (SEQ ID NO: 776). In some embodiments, the exogenous autoantigenic polypeptide further comprises, at its C-terminus, one or more (e.g., two, three, four, five, six, seven, eight, nine, or ten) additional autoantigens (e.g., the same or different exogenous antigenic polypeptides). In some embodiments, any two autoantigens are separated by a linker (e.g., a linker comprising GSGSGSGSGSGSGSGSGS (SEQ ID NO: 840) or GPGPG (SEQ ID NO: 841)).

In some embodiments, an exogenous autoantigenic polypeptide can include CD74 or a portion thereof (e.g., SEQ ID NO: 835 or 836).

Non-limiting examples of linkers that can be used in any of the exogenous autoantigenic polypeptides described herein include SEQ ID NOs: 532, 812, or 815. A non-limiting example of a signal peptide that can be used in any of the exogenous autoantigenic polypeptides described herein is a GPA signal peptide (e.g., SEQ ID NO: 811). Non-limiting examples of transmembrane domains that can be included in any of the exogenous autoantigenic polypeptides described herein are SEQ ID NO: 813 and 814.

In some embodiments, one of the at least one exogenous autoantigenic polypeptides comprises a sequence that is at least 80% identical (e.g., at least 85% identical, at least 90% identical, at least 92% identical, at least 94% identical, at least 96% identical, at least 98% identical, at least 99% identical, or 100% identical) to any one of SEQ ID NOs: 777-810 and 824-832.

### Exogenous Coinhibitory Polypeptides

In certain embodiments, the engineered erythroid cells (e.g., engineered enucleated erythroid cells) or enucleated cells (e.g., modified enucleated cells) described herein further include at least one (e.g., one, two, three, or more) exogenous immunogenic polypeptide, at least one (e.g., one, two, three, or more) exogenous HLA-G polypeptide, and at least one (e.g., one, two, three, or more) exogenous coinhibitory polypeptide, and optionally, at least one (e.g., one, two, three, or more) exogenous antigenic polypeptide.

In other aspects, the engineered erythroid cells (e.g., engineered enucleated erythroid cells) or enucleated cells (e.g., modified enucleated cells) described herein include at least one exogenous autoantigenic polypeptide (e.g., one or more of any of the exemplary autoantigenic polypeptides described herein) and at least one coinhibitory polypeptide (e.g., one or more of any of the exemplary coinhibitory polypeptides described herein or known in the art).

In some embodiments, one or more of the at least one exogenous coinhibitory polypeptides is present on the cell surface of the engineered erythroid cell or enucleated cell. In some embodiments, one or more of the at least one exogenous coinhibitory polypeptide further comprises a transmembrane domain (e.g., a glycophorin A (GPA) transmembrane domain, a small integral membrane protein 1 (SMIM1) transmembrane domain, or a transferrin receptor (TfR1) transmembrane domain, or any of the other exemplary transmembrane domains described herein or known in the art).

In some embodiments, one or more of the at least one exogenous coinhibitory polypeptide is present within the cell. In some embodiments, one or more of the at least one exogenous coinhibitory polypeptide is present in the cytosol of the cell. In some embodiments, one or more of the at least one exogenous coinhibitory polypeptide is attached to the intracellular side of the plasma membrane. In some embodiments, one or more of the at least one exogenous coinhibitory polypeptide can be secreted or released by the cell. In some embodiments, one or more of the at least one exogenous coinhibitory polypeptide can be tethered to the plasma membrane via attachment to a lipid moiety (e.g., N-myristoylation, S-palmitoylation, farnesylation, geranylgeranylation, and glycosylphosphatidyl inositol (GPI) anchor).

In some embodiments, the at least one exogenous coinhibitory polypeptide is IL-10, IL-27, IL-37, TGFβ, CD39, CD73, arginase 1 (ARG1), Annexin 1, fibrinogen-like protein 2 (FGL2), or PD-L1. For example, in some embodiments, the exogenous coinhibitory polypeptide is IL-10. In some embodiments, the exogenous coinhibitory polypeptide is a mutant IL-10, e.g., IL-10 protein comprising an amino acid substitution, whereby isoleucine at position 87 is replaced with an amino acid other than leucine (e.g., alanine or glycine; see e.g., Ding et al. (2000) J. Exp. Med. 191(2): 213-223). In some embodiments, the exogenous coinhibitory polypeptide comprises a monomeric form of human IL-10 (see, e.g., Josephson et al., (2000) J. Biol. Chem. 275:13552-13557). In some embodiments, the monomeric human IL-10 comprises an amino acid substitution whereby isoleucine at position 87 is replaced with an amino acid other than leucine (e.g., alanine or glycine).

In some embodiments, the exogenous coinhibitory polypeptide comprises or consists of the amino acid sequence of any one of SEQ ID NOs. 760-764. In some embodiments, the exogenous coinhibitory polypeptide comprises an amino acid sequence that is least 90%, at least 92%, at least 94%, at least 95%, at least 96%, at least 97%, at least 98%, at least 99%, or 100% identical to SEQ ID NO: 760, 761, 762, 763, or 764. In some embodiments, the exogemous coinhibitory polypeptide includes a signal peptide. In other embodiments, the exogenous coinhibitory polypeptide does not include a signal peptide. In some embodiments, the exogenonus coinhibitory polypeptide is fused to a membrane anchor (e.g., a transmembrane protein or a transmembrane fragment thereof). In some embodiments, the exogenous coinhibitory polypeptide is fused to a human glycophorin A (GPA) protein or fragment thereof (e.g., a fragment including the GPA transmembrane domain, e.g., SEQ ID NO: 813). In some embodiments, the exogenous coinhibitory polypeptide is cleavable. In some embodiments, the exogenous coinhibitory polypeptide is fused to a small integral membrane protein 1 (SMIM1) or a fragment thereof (e.g., a fragment including the SMIM1 transmembrane domain, e.g., SEQ ID NO: 814). In some embodiments, the exogenous coinhibitory polypeptide is fused to transferrin receptor or a fragment thereof (e.g., a fragment including the transferrin receptor transmembrane domain). In some embodiments, the exogenous coinhibitory polypeptide comprises IL-10 (e.g., a sequence at least 90%, at least 92%, at least 94%, at least 96%, at least 98%, at least 99%, or 100% identical to any one of SEQ ID NOs. 760-763). In some embodiments, the exogenous coinhibitory polypeptide can further include a signal peptide (e.g., a GPA signal peptide (e.g., SEQ ID NO: 811)) and/or a transmembrane domain (e.g., a GPA transmembrane domain (SEQ ID NO: 813)).

In some embodiments, the exogenous coinhibitory polypeptide comprises PD-L1. In some embodiments, the exogenous coinhibitory polypeptide comprises an amino acid sequence that is at least 90%, at least 92%, at least 94%, at least 96%, at least 98%, at least 99%, or 100% identical to SEQ ID NO: 764. In some embodiments, the exogenous coinhibitory polypeptide further comprises a signal peptide (e.g., a GPA signal peptide (e.g., SEQ ID NO: 811)) and/or a transmembrane domain (e.g., a GPA transmembrane domain (SEQ ID NO: 813) or a SMIM1 transmembrane domain (SEQ ID NO: 814) or a transferrin receptor transmembrane domain).

In some embodiments, one of the at least one exogenous inhibitory polypeptides comprises a sequence that is at least 80%, at least 85%, at least 90%, at least 92%, at least 94%, at least 96%, at least 98%, at least 99%, or 100% identical to any of SEQ ID NOs: 816-823.

In some embodiments, the exogenous coinhibitory polypeptide comprises or consists of a soluble cytokine (e.g., IL-10, IL-27, IL-37, and TGFβ). In some embodiments, the exogenous coinhibitory polypeptide comprises or consists of an enzyme (e.g., CD39, CD73, and ARG1). In some embodiments, the exogenous coinhibitory polypeptide comprises a cellular receptor (e.g., PD-L1).

In some embodiments, the exogenous coinhibitory polypeptide comprises or consists of a polypeptide listed in Table 4. In some embodiments, the exogenous coinhibitory polypeptide comprises or consists of B7-1, B7-2, B7DC, B7H1, HVEM, collagen, galectin-9, CD48, TIM4, CD155, CD112, CD113, PDL1, IL-35, IL-10, IL-27, VSIG-3, IL-1Ra, IL-4, IL-11, IL-13, TGFβ, IL-33, IL-37, CD39, CD73, ARG1, Annexin 1, FGL2, or a functional fragment of any of the foregoing.

In some embodiments, the exogenous coinhibitory polypeptide comprises an agonist polypeptide (e.g., an antibody or a functional fragment thereof) that specifically binds to a coinhibitory receptor on an immune cell (e.g., a T cell, a B cell, a macrophage, DC, or an NK cell). For example, in some embodiments, the exogenous coinhibitory polypeptide comprises an antibody that binds to a receptor selected from the group consisting of: PD1, CTLA4, TIM3, TGFβ, a CEACAM (e.g., CEACAM-1, CEACAM-3 and/or CEACAM-5), LAG3, VISTA, BTLA, TIGIT, LAIR1, CD160, and 2B4. In some embodiments, the exogenous coinhibitory polypeptide comprises or consists of an antibody that binds to a target receptor (e.g., as listed in Table 4) on an immune cell (e.g., a T cell, a B cell, a macrophage, DC, or an NK cell).

In some embodiments, the exogenous coinhibitory polypeptide comprises or consists of a checkpoint molecule (e.g., PD-L1, PD-L2, and OX40L). In some embodiments, the exogenous coinhibitory polypeptide comprises or consists of an agonist (e.g., an agonist antibody or a functional fragment thereof) of PD-1, CTLA4, TIM3, or LAG3.

TABLE 4
Coinhibitory Polypeptides

| Inhibitory Polypeptide | Target Receptor |
|---|---|
| B7-1 | CTLA4, B7H1 |
| B7-2 | CTLA4 |
| B7DC | PD1 |
| B7H1 | PD1, B7-1 |
| HVEM | CD160, BTLA |
| COLLAGEN | LAIR1 |
| GALECTIN-9 | TIM3 |
| CD48, TIM4 | TIM4R |
| CD48 | 2B4 |
| CD155, CD112, CD113 | TIGIT |
| PDL1 | PD1 |
| | LAG3 |

In some embodiments, the exogenous coinhibitory polypeptide comprises an antibody that blocks binding of a costimulatory polypeptide to its cognate costimulatory receptor. In some embodiments, the exogenous coinhibitory polypeptide comprises an antibody (or a functional fragment thereof) that blocks binding of 4-1BBL, LIGHT, CD80, CD86, CD70, OX40L, GITRL, TIM4, SLAM, CD48, CD58, CD83, CD155, CD112, IL-15Ra fused to IL-15, IL-2, IL-21, ICAM, a ligand for LFA-1, an anti-CD3 antibody, or an anti-CD28 antibody, to its receptor. In some embodiments, the exogenous coinhibitory polypeptide comprises or consists of an anti-ICOSL antibody (e.g., an anti-ICOSL antibody capable of blocking the binding of ICOSL to ICOS).

In some embodiments of any of the exogenous coinhibitory polypeptides described herein, the exogenous coinhibitory polypeptide comprises a sequence that is at least 80% identical, at least 85% identical, at least 90% identical, at least 92% identical, at least 94% identical, at least 96% identical, at least 98% identical, at least 99% identical, or 100% identical to any one of SEQ ID NOs: 760-764.

In some embodiments, the exogenous coinhibitory polypeptide comprises a membrane anchor (e.g., a transmembrane domain, e.g., the transmembrane domain, such as a Type I membrane protein transmembrane domain (e.g., a GPA transmembrane domain), or a Type II membrane protein transmembrane domain (e.g., a Kell transmembrane domain or a SMIM1 transmembrane domain)), as either an N-terminal or C-terminal fusion). In some embodiments, the exogenous coinhibitory polypeptide comprises a transferrin receptor transmembrane domain.

In some embodiments, the exogenous co-inhibitory polypeptide comprises a linker. The exogenous coinhibitory polypeptide may comprise any of the linkers provided herein. The linker may be greater than 20 amino acids long. In some embodiments, the linker peptide sequence is generally from about 3 to about 30 amino acids long, for example about 5 to about 20 amino acids long, about 5 to about 15 amino acids long, about a to about 10 amino acids long. However, longer or shorter linker may be used or the linker may be dispensed with entirely. In some embodiments, the exogenous co-inhibitory polypeptide comprises a flexible linker (e.g. (Gly₄Ser)₃) (SEQ ID NO: 29). Additional linkers which are known in the art may be used (see, e.g., Huston et al. (1988) Proc. Nat. Acad. Sci. USA 85: 5879-83; U.S. Pat. Nos. 5,091,513, 5,132,405, 4,956,778, 5,258,498, and 5,482,858.

Any of the exogenous polypeptides described herein (e.g., an exogenous immunogenic polypeptide, an exogenous HLA-G polypeptide, an exogenous antigenic polypeptide, and an exogenous coinhibitory polypeptide) can include one or more (e.g., two, three, four, or more) epitope tags at the N-terminal, C-terminal, or disposed within the exogenous polypeptide. The epitope tag(s) may be used for the detection, quantification, and/or isolation of the exogenous polypeptide (e.g., using flow cytometry, Western blot, or immunoprecipitation). Exemplary epitope tags include HA-tag (e.g., YPYDVPDYA (SEQ ID NO:26)), green fluorescent protein (GFP), myc-tag (e.g., EQKLISEEDL (SEQ ID NO:27)), chitin binding protein, maltose binding protein, glutathione-S-transferase, poly(His)tag, thioredoxin, poly(NANP), FLAG-tag (e.g., DYKDDDDK (SEQ ID NO:28)), V5-tag, AviTag™, calmodulin-tag, polyglutamate-tag, E-tag, S-tag, SBP-tag, Softag-1, Softag-3, Strep-tag®, TC-tag, VSV-tag, Xpress-tag, Isopeptag, SpyTag, biotin carboxyl carrier protein, Nus-tag, Fc-tag, or Ty-tag.

### Circulation Time

In some embodiments, an engineered erythroid cell or enucleated cell (or a population of the cells) of the present disclosure resides in circulation after administration to a subject for at least about 1 day to about 240 days (e.g., for at least about 1, 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, or 240 days).

In some embodiments, the engineered enucleated erythroid cell or enucleated cell comprising at least one exogenous immunogenic polypeptide and at least one exogenous HLA-G polypeptide (and optionally, comprising at least one exogenous antigenic polypeptide and/or at least one exogenous coinhibitory polypeptide), exhibits increased circulation time (e.g., by at least 10%, 25%, 50%, 75%, 100%, 150%, 200%, 250%, 300%, or more) in a subject following administration as compared to the circulation time of an engineered enucleated erythroid cell or enucleated cell comprising the same exogenous immunogenic polypeptide (and optionally, the same exogenous antigenic polypeptide(s) and/or the same exogenous coinhibitory polypeptide(s)), but lacking the exogenous HLA-G polypeptide.

### Modifications

One or more of the exogenous polypeptides present in the engineered enucleated erythroid cells or enucleated cells described herein may include a post-translational modification characteristic of eukaryotic cells, e.g., mammalian cells, e.g., human cells. In some embodiments, one or more (e.g., 1, 2, 3, 4, 5, 6, 7, 8, 9, 10, or more) of the exogenous polypeptides are glycosylated (e.g., O-linked glycosylation or N-linked glycosylation), phosphorylated, or both. In some embodiments, one or more of the exogenous polypeptides comprise one or more post-translation modifications selected from conjugation to a hydrophobic group (e.g., myristoylation, palmitoylation, isoprenylation, prenylation, or glypiation), conjugation to a cofactor (e.g., lipoylation, flavin moiety (e.g., FMN or FAD), heme C attachment, phosphopantetheinylation, or retinylidene Schiff base formation), diphthamide formation, ethanolamine phosphoglycerol attachment, hypusine formation, acylation (e.g. O-acylation, N-acylation, or S-acylation), formylation, acetylation, alkylation (e.g., methylation or ethylation), amidation, butyrylation, gamma-carboxylation, malonylation, hydroxylation, iodination, nucleotide addition (e.g., ADP-ribosylation), oxidation, phosphate ester (O-linked) or phosphoramidate (N-linked) formation, (e.g., phosphorylation or adenylylation), propionylation, pyroglutamate formation, S-glutathionylation, S-nitrosylation, succinylation, sulfation, ISGylation, SUMOylation, ubiquitination, Neddylation, or a chemical modification of an amino acid (e.g., citrullination, deamidation, eliminylation, or carbamylation), formation of a disulfide bridge, racemization (e.g., of proline, serine, alanine, or methionine).

### Copy Number

In some embodiments, the engineered erythroid cell (e.g., engineered enucleated erythroid cell) or enucleated cell (e.g., modified enucleated cell) comprises at least about 10, 100, 1,000, 5,000, 10,000, 25,000, 50,000, 60,000, 70,000, 80,000, 90,000, 100,000, 150,000, 200,000, 250,000, 300,000, 350,000, 400,000, 450,000, 500,000, 550,000, 600,000, or more copies of one or more of the exogenous polypeptides described herein.

### Physical Characteristics of Engineered Erythroid Cells

In some embodiments, the engineered erythroid cells or enucleated cells described herein have one or more (e.g., 2, 3, 4, or more) physical characteristics described herein, e.g., osmotic fragility, cell size, hemoglobin concentration, or phosphatidylserine content. In some embodiments, an engineered erythroid cell or an enucleated cell that includes one or more of the exogenous polypeptide described herein has physical characteristics of a wild-type, untreated erythroid cell or enucleated cell.

In some embodiments, the engineered erythroid cell or enucleated cell exhibits substantially the same osmotic membrane fragility as an isolated, uncultured erythroid cell that does not comprise an exogenous polypeptide described herein. In some embodiments, the engineered erythroid cell or enucleated cell has an osmotic fragility of less than 50% cell lysis at 0.3%, 0.35%, 0.4%, 0.45%, or 0.5% NaCl. Osmotic fragility can be assayed using the method of Example 59 of International Application Publication No WO 2015/073587, which is herein incorporated by reference in its entirety.

In some embodiments, the engineered erythroid cell or enucleated cell has approximately the diameter or volume as a wild-type, untreated enucleated erythroid cell.

In some embodiments, a population of engineered erythroid cells or enucleated cells described herein has an average diameter of about 4, 5, 6, 7, or 8 microns, and optionally the standard deviation of the population is less than 1, 2, or 3 microns. In some embodiments, one or more engineered erythroid cells or enucleated cells in the population has a diameter of about 4-8, 5-7, or about 6 microns. In some embodiments, the diameter of the engineered erythroid cells or enucleated cells in the population is less than about 1 micron, larger than about 20 microns, between about 1 micron and about 20 microns, between about 2 microns and about 20 microns, between about 3 microns and about 20 microns, between about 4 microns and about 20 microns, between about 5 microns and about 20 microns, between about 6 microns and about 20 microns, between about 5 microns and about 15 microns or between about 10 microns and about 30 microns. Cell diameter is measured, in some embodiments, using an Advia 120 hematology system, a Vi-cell™ Cell Viability Analyzer (Beckman Coulter), or a Moxi Z cell counter (Orflo). In some embodiment the volume of the mean corpuscular volume of the engineered erythroid cells or enucleated cells is greater than 10 fL, 20 fL, 30 fL, 40 fL, 50 fL, 60 fL, 70 fL, 80 fL, 90 fL, 100 fL, 110 fL, 120 fL, 130 fL, 140 fL, 150 fL, or greater than 150 fL. In some embodiments, the mean corpuscular volume of the engineered erythroid cells or enucleated cells is less than 30 fL, 40 fL, 50 fL, 60 fL, 70 fL, 80 fL, 90 fL, 100 fL, 110 fL, 120 fL, 130 fL, 140 fL, 150 fL, 160 fL, 170 fL, 180 fL, 190 fL, 200 fL, or less than 200 fL. In some embodiments, the mean corpuscular volume of the engineered erythroid cells or enucleated cells is between 80-100, 100-200, 200-300, 300-400, or 400-500 femtoliters (fL). In some embodiments, a population of engineered erythroid cells (e.g., engineered enucleated erythroid cells) or enucleated cells (e.g., modified enucleated cells) has a mean corpuscular volume set out in this paragraph and the standard deviation of the population is less than 50, 40, 30, 20, 10, 5, or 2 fL. The mean corpuscular volume is measured, in some embodiments, using a hematological analysis instrument, e.g., a Coulter counter, a MoxiZ cell counter (Orflo), or a Sysmex hematology analyzer.

In some embodiments, the engineered erythroid cell (e.g., engineered enucleated erythroid cell) or enucleated cell (e.g., modified enucleated cell) described herein has a hemoglobin content similar to a wild-type, untreated enucleated erythroid cell or enucleated cell. In some embodiments, the engineered erythroid cells or enucleated cells comprise at least about 20, 22, 24, 26, 28, or 30 pg, and optionally up to about 30 pg, of total hemoglobin. Hemoglobin levels are determined, in some embodiments, using the Drabkin's reagent method of Example 33 of International Application Publication No. WO2015/073587, which is herein incorporated by reference in its entirety.

In some embodiments, the engineered erythroid cells (e.g., engineered enucleated erythroid cells) or enucleated cells (e.g., modified enucleated cells) described herein has approximately the same phosphatidylserine content on the outer leaflet of its cell membrane as a wild-type, untreated erythroid cell or enucleated cell. In some embodiments, a population of engineered erythroid cells (e.g., engineered enucleated erythroid cells) or enucleated cells (e.g., modified enucleated cells) described herein comprises less than about 30, 25, 20, 15, 10, 9, 8, 6, 5, 4, 3, 2, or 1% of cells that are positive for annexin V staining. Phosphatidylserine exposure is assessed, in some embodiments, by staining for annexin-V-FITC, which binds preferentially to PS, and measuring FITC fluorescence by flow cytometry, e.g., using the method of Example 54 of International Application Publication No. WO2015/073587, which is herein incorporated by reference in its entirety.

In some embodiments, an engineered erythroid cell or enucleated cell described herein, or a population of engineered erythroid cells or enucleated cells described herein, comprises one or more of (e.g., all of) endogenous GPA (C235a), transferrin receptor (CD71), Band 3 (CD233), or integrin alpha4 (C49d). These proteins can be measured, e.g., as described in Example 10 of International Application Publication No. WO2018/009838, which is herein incorporated by reference in its entirety. The percentage of GPA-positive cells and Band 3-positive cells typically increases during maturation of an erythroid cell, and the percentage of integrin alpha4-positive typically remains high throughout maturation.

In some embodiments, the population of engineered enucleated erythroid cells or enucleated cells comprises at least about 50%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% GPA⁺ (i.e., CD235a⁺) cells. In some embodiments, the population of engineered enucleated erythroid cells or enucleated cells comprises between about 50% and about 100% (e.g., from about 60% and about 100%, from about 65% and about 100%, from about 70% and about 100%, from about 75% to about 100%, from about 80% to about 100%, from about 85% to about 100%, from about 90% to about 100%, from about 95% to about 100%, from about 75% to about 99%, from about 80% to about 99%, from about 85% to about 99%, from about 90% to about 99%, from about 95% to about 99%, from about 75% to about 95%, from about 80% to about 95%, from about 85% to about 95%, from about 90% to about 95%, from about 95% to about 98%) GPA⁺ cells. The presence of GPA is detected, in some embodiments, using FACS.

In some embodiments, the population of engineered enucleated erythroid cells or enucleated cells comprises at least about 50%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% CD71⁺ cells. In some embodiments, the population of engineered enucleated erythroid cells or enucleated cells comprises between about 70% and about 100% (e.g., from about 75% to about 100%, from about 80% to about 100%, from about 85% to about 100%, from about 90% to about 100%, from about 95% to about 100%, from about 75% to about 99%, from about 80% to about 99%, from about 85% to about 99%, from about 90% to about 99%, from about 95% to about 99%, from about 75% to about 95%, from about 80% to about 95%, from about 85% to about 95%, from about 90% to about 95%, from about 95% to about 98%) CD71⁺ cells. The presence of CD71 (transferrin receptor) is detected, in some embodiments, using FACS.

In some embodiments, the population of engineered enucleated erythroid cells or enucleated cells comprises at least about 50%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% CD233⁺ cells. In some embodiments, the population of engineered enucleated erythroid cells or enucleated cells comprises between about 70% and about 100% (e.g., from about 75% to about 100%, from about 80% to about 100%, from about 85% to about 100%, from about 90% to about 100%, from about 95% to about 100%, from about 75% to about 99%, from about 80% to about 99%, from about 85% to about 99%, from about 90% to about 99%, from about 95% to about 99%, from about 75% to about 95%, from about 80% to about 95%, from about 85% to about 95%, from about 90% to about 95%, from about 95% to about 98%) CD233⁺ cells. The presence of CD233 (Band 3) is detected, in some embodiments, using FACS.

In some embodiments, the population of engineered enucleated erythroid cells or enucleated cells comprises at least about 50%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% CD47⁺ cells. In some embodiments, the population of engineered enucleated erythroid cells or enucleated cells comprises between about 70% and about 100% (e.g., from about 75% to about 100%, from about 80% to about 100%, from about 85% to about 100%, from about 90% to about 100%, from about 95% to about 100%, from about 75% to about 99%, from about 80% to about 99%, from about 85% to about 99%, from about 90% to about 99%, from about 95% to about 99%, from about 75% to about 95%, from about 80% to about 95%, from about 85% to about 95%, from about 90% to about 95%, from about 95% to about 98%) CD47⁺ cells. The presence of CD47 (integrin associate protein) is detected, in some embodiments, using FACS.

In some embodiments, the population of engineered enucleated erythroid cells or enucleated cells comprises at least about 50%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% CD36⁻ (CD36-negative) cells. In some embodiments, the population of engineered enucleated erythroid cells or enucleated cells comprises between about 70% and about 100% (e.g., from about 75% to about 100%, from about 80% to about 100%, from about 85% to about 100%, from about 90% to about 100%, from about 95% to about 100%, from about 75% to about 99%, from about 80% to about 99%, from about 85% to about 99%, from about 90% to about 99%, from about 95% to about 99%, from about 75% to about 95%, from about 80% to about 95%, from about 85% to about 95%, from about 90% to about 95%, from about 95% to about 98%) CD36″ (CD36-negative) cells. The presence of CD36 is detected, in some embodiments, using FACS.

In some embodiments, the population of engineered enucleated erythroid cells or enucleated cells comprises at least about 50%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% CD34″ (CD34-negative) cells. In some embodiments, the population of engineered enucleated erythroid cells or enucleated cells comprises between about 70% and about 100% (e.g., from about 75% to about 100%, from about 80% to about 100%, from about 85% to about 100%, from about 90% to about 100%, from about 95% to about 100%, from about 75% to about 99%, from about 80% to about 99%, from about 85% to about 99%, from about 90% to about 99%, from about 95% to about 99%, from about 75% to about 95%, from about 80% to about 95%, from about 85% to about 95%, from about 90% to about 95%, from about 95% to about 98%) CD34⁻ (CD34-negative) cells. The presence of CD34 is detected, in some embodiments, using FACS.

In some embodiments, the population of engineered enucleated erythroid cells or enucleated cells comprises at least about 50%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% CD235a⁺/CD47⁺/CD233⁺ cells. In some embodiments, the population of engineered enucleated erythroid cells or enucleated cells comprises between about 70% and about 100% (e.g., from about 75% to about 100%, from about 80% to about 100%, from about 85% to about 100%, from about 90% to about 100%, from about 95% to about 100%, from about 75% to about 99%, from about 80% to about 99%, from about 85% to about 99%, from about 90% to about 99%, from about 95% to about 99%, from about 75% to about 95%, from about 80% to about 95%, from about 85% to about 95%, from about 90% to about 95%, from about 95% to about 98%) CD235a⁺/CD47⁺/CD233⁺ cells.

In some embodiments, the population of engineered enucleated erythroid cells or enucleated cells comprises at least about 50%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% CD235a⁺/CD47⁺/CD233⁺/CD34⁻/CD36⁻ cells. In some embodiments, the population of engineered enucleated erythroid cells or enucleated cells comprises between about 70% and about 100% (e.g., from about 75% to about 100%, from about 80% to about 100%, from about 85% to about 100%, from about 90% to about 100%, from about 95% to about 100%, from about 75% to about 99%, from about 80% to about 99%, from about 85% to about 99%, from about 90% to about 99%, from about 95% to about 99%, from about 75% to about 95%, from about 80% to about 95%, from about 85% to about 95%, from about 90% to about 95%, from about 95% to about 98%) CD235a⁺/CD47⁺/CD233⁺/CD34⁻/CD36⁻ cells.

In some embodiments, a population of engineered enucleated erythroid cells or enucleated cells comprising erythroid cells comprises less than about 10%, 9%, 8%, 7%, 6%, 5%, 4%, 3%, 2%, or 1% echinocytes. In some embodiments, a population of engineered enucleated erythroid cells or enucleated cells comprising comprises less than about 10%, 9%, 8%, 7%, 6%, 5%, 4%, 3%, 2%, or 1% pyrenocytes.

### Populations of Engineered Erythroid Cells

In one aspect, the disclosure features populations of the engineered erythroid cells or enucleated cells described herein, e.g., a plurality or population of the engineered enucleated erythroid cells. The terms “plurality” and “population” are used interchangeably herein. In some embodiments, a population of engineered erythroid cells or enucleated cells may comprise predominantly enucleated cells (e.g., greater than 70%), predominantly nucleated cells (e.g., greater than 70%), or any mixture of enucleated and nucleated cells. In some embodiments, a population of engineered erythroid cells or enucleated cells may comprise reticulocytes, erythrocytes, or a mixture of reticulocytes and erythrocytes. In some embodiments, a population of engineered erythroid cells or enucleated cells may predominantly comprise reticulocytes. In some embodiments, a population of engineered erythroid cells or enucleated cells may predominantly comprise erythrocytes (e.g., immature or mature erythrocytes).

In some embodiments, a population of engineered erythroid cells consists essentially of enucleated cells. In some embodiments, a population of engineered erythroid cells comprises predominantly or substantially enucleated cells. For example, in some embodiments, a population of engineered erythroid cells comprises at least about 70% or more enucleated cells. In some embodiments, the population provided herein comprises at least about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99, or about 100% enucleated cells. In some embodiments, the population provided herein comprises greater than about 70% enucleated cells. In some embodiments, the population of engineered erythroid cells comprises greater than about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% enucleated cells. In some embodiments, the population of engineered erythroid cells comprises between about 80% and about 100% enucleated cells, for example between about 80% and about 95%, about 80% and about 90%, about 80% and about 85%, about 85% and about 100%, about 85% and about 95%, about 85% and about 90%, about 90% and about 100%, about 90% and about 95%, or about 95% and about 100% of enucleated cells.

In some embodiments, the population of engineered erythroid cells comprises less than about 30% nucleated cells. For example, in embodiments, the population of engineered erythroid cells comprises less than about 1%, about 2%, about 3%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or less than about 30% nucleated cells. In some embodiments, the population of engineered erythroid cells comprises less than about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, or about 19%, about 21%, about 22%, about 23%, about 24%, about 25%, about 26%, about 27%, about 28%, about 29%, or about 30% nucleated cells. In some embodiments, the population of engineered erythroid cells comprises between 0% and 30% nucleated cells. In some embodiments, the populations of engineered erythroid cells comprise between about 0% and 20% nucleated cells, for example between about 0% and 19%, between about 0% and 15%, between about 0% and 10%, between about 0% and 5%, between about 0% and 4%, between about 0% and 3%, between about 0% and 2% nucleated cells, or between about 5% and 20%, between about 10% and 20%, or between about 15% and 20% nucleated cells.

In some embodiments, the disclosure features a population of the engineered erythroid cells as described herein, wherein the population of engineered erythroid cells comprises less than 30% nucleated cells and at least 70% enucleated cells, or comprises less than 20% nucleated cells and at least 80% enucleated cells, or comprises less than 15% nucleated cells and at least 85% nucleated cells, or comprises less than 10% nucleated cells and at least 90% enucleated cells, or comprises less than 5% nucleated cells and at least 95% enucleated cells. In some embodiments, the disclosure features populations of the engineered erythroid cells as described herein, wherein the population of engineered erythroid cells comprises about 0% nucleated cells and about 100% enucleated cells, about 1% nucleated cells and about 99% enucleated cells, about 2% nucleated cells and about 98% enucleated cells, about 3% nucleated cells and about 97% enucleated cells, about 4% nucleated cells and about 96% enucleated cells, about 5% nucleated cells and about 95% enucleated cells, about 6% nucleated cells and about 94% enucleated cells, about 7% nucleated cells and about 93% enucleated cells, about 8% nucleated cells and about 92% enucleated cells, about 9% nucleated cells and about 91% enucleated cells, about 10% nucleated cells and about 90% enucleated cells, about 11% nucleated cells and about 89% enucleated cells, about 12% nucleated cells and about 88% enucleated cells, about 13% nucleated cells and about 87% enucleated cells, about 14% nucleated cells and about 86% enucleated cells, about 85% nucleated cells and about 85% enucleated cells, about 16% nucleated cells and about 84% enucleated cells, about 17% nucleated cells and about 83% enucleated cells, about 18% nucleated cells and about 82% enucleated cells, about 19% nucleated cells and about 81% enucleated cells, or about 20% nucleated cells and about 80% enucleated cells.

In other embodiments, the engineered erythroid cell population comprises predominantly or substantially nucleated cells. In some embodiments, the engineered erythroid cell population consists essentially of nucleated cells. In various embodiments, the nucleated cells in the engineered erythroid cell population are erythroid precursor cells. In some embodiments, the erythroid precursor cells are selected from the group consisting of pluripotent hematopoietic stem cells (HSCs), multipotent myeloid progenitor cells, CFU-S cells, BFU-E cells, CFU-E cells, pronormoblasts, basophilic normoblasts, polychromatophilic normoblasts and orthochromatophilic normoblasts.

In certain embodiments, the population of engineered erythroid cells comprises at least about 10%, at least about 20%, at least about 30%, at least about 40%, at least about 50%, at least about 60%, at least about 70%, at least about 75%, at least about 80%, at least about 85%, at least about 90%, at least about 95%, at least about 98%, at least about 99% or 100% nucleated cells.

In some embodiments, a population of erythroid cells or enucleated cells comprises about 1×10⁹-2×10⁹, 2×10⁹-5×10⁹, 5×10⁹-1×10¹⁰, 1×10¹⁰-2×10¹⁰, 2×10¹⁰-5×10¹⁰, 5×10¹⁰-1×10¹¹, 1×10¹¹-2×10¹¹, 2×10¹¹-5×10¹¹, 5×10¹¹-1×10¹², 1×10¹²-2×10¹², 2×10¹²-5×10¹², or 5×10¹²-1×10¹³ cells.

It will be understood that during the preparation of the engineered erythroid cells or enucleated cells of the as described herein, some fraction of cells may not include an exogenous polypeptide (e.g., due to lack of expression or transduction or conjugation with an exogenous nucleic acid). Accordingly, in some embodiments, a population of engineered erythroid cells or enucleated cells provided herein comprises a mixture of engineered erythroid cells and unmodified erythroid cells, or a mixture of modified enucleated cells and unmodified enucleate cells, i.e., some fraction of cells in the population will not include (e.g., express) an exogenous polypeptide. For example, a population of engineered erythroid cells or enucleated cells can comprise, in various embodiments, at least about 10%, 20%, 30%, 40%, 50%, 60%, 70%, 80%, 90%, 91%, 92%, 93%, 94%, 95%, 96%, 97%, 98% or 99% erythroid cells or enucleated cells that include an exogenous polypeptide, wherein the remaining erythroid cells or enucleated cells in the population are do not include an exogenous polypeptide. In some embodiments, a single unit dose of engineered erythroid cells (e.g., engineered enucleated erythroid cells) or enucleated cells comprises at least about 10%, 20%, 30%, 40%, 50%, 60%, 65%, 70%, 75%, 80%, 85%, 90%, 91%, 92%, 93%, 94%, 95%, 96%, 97%, 98% or 99% erythroid cells or enucleated cells including an exogenous polypeptide, wherein the remaining erythroid cells or enucleated cells in the dose do not include an exogenous polypeptide.

In some embodiments, the engineered erythroid cells or enucleated cells described herein are autologous and/or allogeneic to the subject to which the cells will be administered. In some embodiments, the engineered erythroid cells or enucleated cells described herein do not include one or more blood group antigens, e.g., Le(a-b-) (for Lewis antigen system), Fy(a-b-) (for Duffy system), Jk(a-b-) (for Kidd system), M-N- (for MNS system), K-k- (for Kell system), Lu(a-b-) (for Lutheran system), and H-antigen negative (Bombay phenotype), or any combination thereof. In some embodiments, the engineered erythroid cells or enucleated cells are also Type O and/or Rh−. Minor blood groups are described, e.g., in Agarwal et al. (2013) Blood Res. 48(1): 51-4, and Mitra et al. (2014) Indian J Anaesth. 58(5): 524-8, each of which is incorporated herein by reference in its entirety.

### II. Methods of Making Engineered Erythroid Cells

In some aspects, the present disclosure provides a method of making an engineered erythroid cell (e.g., engineered enucleated erythroid cell) or enucleated cell (e.g., modified enucleated cell) comprising at least one exogenous immunogenic polypeptide and at least one exogenous HLA-G polypeptide (and optionally, at least one exogenous antigenic polypeptide and/or at least one exogenous coinhibitory polypeptide). In other aspects, the present disclosure provides a method of making an engineered erythroid cell (e.g., engineered enucleated erythroid cell) or enucleated cell (e.g., modified enucleated cell) comprising at least one exogenous autoantigenic polypeptide and at least one exogenous coinhibitory polypeptide.

Methods of manufacturing engineered erythroid cells and enucleated cells comprising an exogenous polypeptide are described, e.g., in International Application Publication Nos. WO 2015/073587 and WO 2015/153102, each of which is incorporated by reference in its entirety.

In some aspects, the description provides a method of producing the engineered erythroid cell (e.g., engineered enucleated erythroid cell) or enucleated cell (e.g., modified enucleated cell) comprising at least one exogenous immunogenic polypeptide and at least one exogenous HLA-G polypeptide, the method comprising introducing an exogenous nucleic acid encoding the exogenous immunogenic polypeptide into a nucleated erythroid precursor cell; introducing an exogenous nucleic acid encoding the exogenous HLA-G polypeptide into the nucleated erythroid precursor cell; and culturing the nucleated erythroid precursor cell under conditions suitable for enucleation and for production of both the exogenous immunogenic polypeptide and the exogenous HLA-G polypeptide, thereby making the engineered erythroid cell or enucleated cell. In some embodiments, the method further comprises introducing an exogenous nucleic acid encoding an exogenous antigenic polypeptide and/or an exogenous coinhibitory polypeptide into the nucleated erythroid precursor cell. In some embodiments, one or more of the exogenous immunogenic polypeptide, the exogenous HLA-G polypeptide, the exogenous antigenic polypeptide, and the exogenous coinhibitory polypeptide are encoded by the same exogenous nucleic acid. In some embodiments, one or more of the exogenous immunogenic polypeptide, the exogenous HLA-G polypeptide, the exogenous antigenic polypeptide, and the exogenous coinhibitory polypeptide are encoded by different exogenous nucleic acids.

In some embodiments, the description provides a method of producing the engineered erythroid cell (e.g., engineered enucleated erythroid cell) or enucleated cell (e.g., modified enucleated cell) comprising at least one exogenous immunogenic polypeptide and at least one exogenous HLA-G polypeptide, the method comprising introducing an exogenous nucleic acid encoding an exogenous immunogenic polypeptide into a nucleated erythroid precursor cell; introducing an exogenous nucleic acid encoding an exogenous HLA-G polypeptide into the nucleated erythroid precursor cell; culturing the nucleated erythroid precursor cell under conditions suitable for enucleation and for production of both the exogenous immunogenic polypeptide and the exogenous HLA-G polypeptide, thereby making an engineered enucleated erythroid cell; and contacting the engineered enucleated erythroid cell or enucleated cell with at least one exogenous antigenic polypeptide, wherein the at least one exogenous antigenic polypeptide binds to the exogenous HLA-G polypeptide on the surface of the engineered enucleated erythroid cell or enucleated cell. In some embodiments, the method further comprises introducing an exogenous nucleic acid encoding an exogenous antigenic polypeptide and/or an exogenous coinhibitory polypeptide into the nucleated erythroid precursor cell. In some embodiments, one or more of the exogenous immunogenic polypeptide, the exogenous HLA-G polypeptide, the exogenous antigenic polypeptide, and the exogenous coinhibitory polypeptide are encoded by the same exogenous nucleic acid. In some embodiments, one or more of the exogenous immunogenic polypeptide, the exogenous HLA-G polypeptide, the exogenous antigenic polypeptide, and the exogenous coinhibitory polypeptide are encoded by different exogenous nucleic acids.

In some aspects, the description provides a method of producing the engineered erythroid cell (e.g., engineered enucleated erythroid cell) or enucleated cell (e.g., modified enucleated cell) comprising at least one exogenous autoantigenic polypeptide and at least one exogenous coinhibitory polypeptide, the method comprising introducing an exogenous nucleic acid encoding the exogenous autoantigenic polypeptide into a nucleated erythroid precursor cell; introducing an exogenous nucleic acid encoding the exogenous coinhibitory polypeptide into the nucleated erythroid precursor cell; and culturing the nucleated erythroid precursor cell under conditions suitable for enucleation and for production of the at least one exogenous autoantigenic polypeptide and the at least one exogenous coinhibitory polypeptide, thereby making the engineered erythroid cell or enucleated cell.

In some embodiments, the erythroid precursor cells are immortalized, e.g., comprise a human papilloma virus (HPV; e.g., HPV type 16) E6 and/or E7 gene. In some embodiments, the immortalized erythroid precursor cell is a BEL-A cell line cell (see Trakarnasanga et al. (2017) Nat. Commun. 8: 14750). Additional immortalized erythroid precursor cells are described in U.S. Pat. Nos. 9,951,350, and 8,975,072.

In some embodiments, erythroid precursor cells, e.g., CD34⁺ hematopoietic progenitor cells (e.g., human (e.g., adult human) or mouse cells), are contacted with an exogenous nucleic acid or exogenous nucleic acids encoding one or more exogenous polypeptide(s) described herein, and the cells are allowed to expand and differentiate in culture. Thus, also provided herein are engineered erythroid precursor cells comprising an exogenous nucleic acid and/or an exogenous polypeptide described herein. In some embodiments, the cells (e.g., erythroid precursor cells and erythroid cells) are expanded at least 1,000-, 2,000-, 5,000-, 10,000-, 20,000-, 50,000-, or 100,000-fold or more (and optionally, up to 100,000-, 200,000-, or 500,000-fold). The number of cells is measured, in some embodiments, using an automated cell counter.

The modified erythroid precursor cells provided herein can be differentiated in vitro into engineered enucleated erythroid cells (e.g., reticulocytes or erythrocytes) using methods known in the art (see, e.g., Giarratana et al. (2011) Blood 118: 5071-9, Huang et al. (2014), Kurita et al., PLOS One 2013, 8:e59890, and International Application Publication No. WO 2014/183071). For example, erythroid cells can be cultured from erythroid precursor cells, including CD34⁺ hematopoietic progenitor cells (Giarratana et al. (2011)), induced pluripotent stem cells (Kurita et al. (2013) PLOS One 8:e59890), and embryonic stem cells (Hirose et al. (2013) Stem Cell Reports 1:499-508).

Cocktails of growth and differentiation factors that are suitable to expand and differentiate progenitor cells into erythroid cells or platelets are known in the art. Suitable expansion and differentiation factors include, but are not limited to, stem cell factor (SCF), an interleukin (IL) such as IL-1, IL-2, IL-3, IL-4, IL-5, IL-6, IL-7, IL-8, IL-9, IL-11, IL-12, CSF, G-CSF, thrombopoietin (TPO), granulocyte-macrophage colony-stimulating factor (GM-CSF), erythropoietin (EPO), Flt3, Flt2, PIXY 321, and leukemia inhibitory factor (LIF).

Erythroid cells can be cultured using a multi-step culture process. For example, in some embodiments, erythroid precursor cells (e.g., CD34⁺ HSCs) may be subjected to a three-step culture process, as outlined below.

The first step may include contacting the cells in culture with SCF at 1-1000 ng/mL, EPO at 1-100 U/mL, and IL-3 at 0.1-100 ng/mL. Optionally, the first step includes contacting the cells in culture with a ligand that binds and activates a nuclear hormone receptor (e.g., the glucocorticoid receptor, the estrogen receptor, the progesterone receptor, the androgen receptor, or the pregnane x receptor). Ligands for these receptors include a corticosteroid (e.g., dexamethasone or hydrocortisone (e.g., each at 10 nM-100 μM)), an estrogen (e.g., beta-estradiol at 10 nM-100 μM); a progestogen (e.g., progesterone, hydroxyprogesterone, 5a-dihydroprogesterone, or 11-deoxycorticosterone (e.g., each at 10 nM-100 μM)), or a synthetic progestin (e.g., chlormadinone acetate at 10 nM-100 μM); an androgen (e.g., testosterone, dihydrotestosterone, or androstenedione (e.g., each at 10 nM-100 or a pregnane x receptor ligand (e.g., rifampicin, hyperforin, hypericin (e.g., each at 10 nM-100 or a vitamin E-like molecule (e.g., tocopherol at 10 nM-100). The first step may also optionally comprise contacting the cells in culture with an insulin-like molecule, such as, e.g., insulin at 1-50 μg/mL, insulin-like growth factor 1 (IGF-1) at 1-50 μg/mL, insulin-like growth factor 2 (IGF-2) at 1-50 μg/mL, or mechano-growth factor at 1-50 μg/mL. The first step may optionally include contacting the cells in culture with transferrin (e.g., holotransferrin, apotransferrin, or a combination thereof, e.g., at 0.1 mg/mL-5 mg/mL). The first step may optionally include contacting the cells in culture with one or more interleukins or growth factors (e.g., IL-1, IL-2, IL-4, IL-5, IL-6, IL-7, IL-8, IL-9, IL-11, IL-12, granulocyte colony-stimulating factor (G-CSF), macrophage colony-stimulating factor (M-CSF), GM-CSF, TPO, fibroblast growth factor (FGF), platelet-derived growth factor (PDGF), transforming growth factor beta (TGF-B), tumor necrosis factor alpha (TNF-α), megakaryocyte growth and development factor (MGDF), leukemia inhibitory factor (LIF), and Flt3 ligand. Each interleukin or growth factor may be supplied at a concentration of 0.1-100 ng/mL. The first step may also optionally include contacting the cells in culture with serum proteins or non-protein molecules (e.g., fetal bovine serum (FBS) (1-20%), human plasma (1-20%), plasmanate (1-20%), human serum (1-20%), albumin (0.1-100 mg/mL), or heparin (0.1-10 U/mL)).

The second step may include contacting the cells in culture with SCF at 1-1000 ng/mL, and EPO at 1-100 U/mL. The second step may also optionally include contacting the cells in culture with an insulin-like molecule (e.g., insulin, IGF-1, IGF-2, or mechano-growth factor (e.g., each at 1-50 μg/mL)). The second step may further optionally include contacting the cells in culture with transferrin (e.g., holotransferrin, apotransferrin, or a combination thereof, e.g., at 0.1 mg/mL-5 mg/mL). The second may also optionally include contacting the cells in culture with serum proteins or non-protein molecules (e.g., FBS (1-20%), human plasma (1-20%), plasmanate (1-20%), human serum (1-20%), albumin (0.1-100 mg/mL), or heparin (0.1-10 U/mL)).

The third step may include contacting the cells in culture with EPO at 1-100 U/mL. The third step may optionally include contacting the cells in culture with SCF at 1-1000 ng/mL. The third step may further optionally include contacting the cells in culture with an insulin-like molecule (e.g., insulin, IGF-1, IGF-2, or mechano-growth factor (e.g., each at 1-50 μg/mL). The third step may also optionally include contacting the cells in culture with transferrin (e.g., holotransferrin, apotransferrin, or a combination thereof, e.g., at 0.1 mg/mL-5 mg/mL). The third step may also optionally include contacting the cells in culture with serum proteins or non-protein molecules (e.g., FBS (1-20%), human plasma (1-20%), plasmanate (1-20%), human serum (1-20%), albumin (0.1-100 mg/mL), or heparin (0.1-10 U/mL)).

The culture process may optionally include contacting the cells by a method known in the art with a molecule (e.g., DNA, RNA, mRNA, siRNA, microRNA, lncRNA, shRNA, hormone, or small molecule) that activates or knocks down one or more genes (e.g., genes encoding a transcription factor, a growth factor, or a growth factor receptor (e.g., GATA1, GATA2, cMyc, hTERT, p53, EPO, SCF, insulin, EPO-R, SCF-R, transferrin-R, insulin-R).

In some embodiments, the modified erythroid precursor cells or modified erythroid cells are expanded at least 100, 1000, 2000, 5000, 10,000, 20,000, 50,000, or 100,000 fold (and optionally up to 100,000, 200,000, or 500,000 fold). Number of cells is measured, in some embodiments, using an automated cell counter.

In some embodiments, it may be desirable during culturing to only partially differentiate the modified erythroid precursor cells, e.g., modified HSCs, in vitro, allowing further differentiation, e.g., differentiation into reticulocytes or erythrocytes, to occur after administration of the cells to a subject in vivo (See, e.g., Neildez-Nguyen et al. (2002) Nat. Biotech. 20: 467-72). Thus, in some embodiments, in vitro differentiation and/or maturation of the cells described herein may be arrested at any stage desired. In some embodiments, the modified erythroid precursor cells or modified erythroid cells are partially differentiated to any stage prior to, but not including enucleation, and thus remain nucleated cells, e.g., erythroid cells. In some embodiments, the resulting cells are nucleated and erythroid lineage restricted. In some embodiments, the resulting cells are selected from multipotent myeloid progenitor cells, CFU-S cells, BFU-E cells, CFU-E cells, pronormoblasts (proerythroblast), basophilic normoblasts, polychromatophilic normoblasts and orthochromatophilic normoblasts.

In some embodiments, the modified erythroid precursor cells or modified erythroid cells are differentiated in vitro through the stage of enucleation where they become reticulocytes. In such embodiments, the reticulocytes can be administered to a subject (e.g., in a pharmaceutical composition) and allowed to finally mature to become erythrocytes in vivo after administration to the subject. In some embodiments, the modified erythroid precursor cells or modified erythroid cells are differentiated in vitro until becoming erythrocytes.

In some embodiments, modified erythroid precursor cells, e.g., HSCs, may be expanded and differentiated in vitro to become hematopoietic cells of different lineage, e.g., platelets. In some embodiments, an enucleated cell provided herein is a platelet. Methods for culturing and differentiating hematopoietic cells of various lineages are known in the art. For example, methods of generating platelets in vitro are known in the art (see, e.g., Wang and Zheng (2016) Springerplus 5(1): 787, and U.S. Pat. No. 9,574,178). Methods of producing platelets including an exogenous polypeptide are described, e.g., in International Patent Application Publication Nos. WO 2015/073587 and WO 2015/153102, each of which is incorporated by reference in its entirety.

In some embodiments, engineered platelets are generated from hematopoietic progenitor cells, such as CD34⁺ HSCs, induced pluripotent stem cells or embryonic stem cells. In some embodiments, platelets are generated by contacting the hematopoietic progenitor cells with defined factors in a multi-step culture process. In some embodiments, the multi-step culture process includes: culturing a population of hematopoietic progenitor cells under conditions suitable to produce a population of megakaryocyte progenitor cells, and culturing the population of megakaryocyte progenitor cells under conditions suitable to produce platelets. Cocktails of growth and differentiation factors that are suitable to expand and differentiate hematopoietic progenitor cells and produce platelets are known in the art. Suitable expansion and differentiation factors include, but are not limited to, SCF, Flt-3/Flk-2 ligand (FL), TPO, IL-11, IL-3, IL-6, and IL-9. In some embodiments, platelets may be produced by seeding CD34⁺ HSCs in a serum-free medium at 2-4×10⁴ cells/mL, and refreshing the medium on culture day 4 by adding an equal volume of media. On culture day 6, cells are counted and analyzed: 1.5×10⁵ cells are washed and placed in 1 mL of the same medium supplemented with a cytokine cocktail comprising TPO (30 ng/mL), SCF (1 ng/mL), IL-6 (7.5 ng/mL), and IL-9 (13.5 ng/mL) to induce megakaryocyte differentiation. At culture day 10, from about one quarter to about half of the suspension culture is replaced with fresh media. The cells are cultured in a humidified atmosphere (10% CO₂) at 39° C. for the first 6 culture days, and at 37° C. for the last 8 culture days. Viable nucleated cells are counted with a hemocytometer following trypan blue staining. The differentiation state of platelets in culture can be assessed by flow cytometry or quantitative PCR as described in Examples 44 and 45 of in International Patent Application Publication No. WO2015/073587, incorporated herein by reference.

In some embodiments, the engineered erythroid cells described herein can be generated by introducing an exogenous nucleic acid encoding an exogenous polypeptide of the disclosure (e.g., an exogenous immunogenic polypeptide, an exogenous antigenic polypeptide, an exogenous HLA-G polypeptide, and/or an exogenous coinhibitory polypeptide) into a suitable isolated cell, e.g., a nucleated erythroid cell, an erythroid precursor cell, or a nucleated platelet precursor cell. In some embodiments, the exogenous nucleic acid is a DNA or an RNA (e.g., an mRNA). Exemplary methods for introducing a nucleic acid into a cell include, but are not limited to, liposome-mediated transfer, transformation, gene guns, transfection, and transduction (e.g., performed using viral vectors including adenovirus vectors, adeno-associated viral vectors, lentiviral vectors, herpes viral vectors, and retroviral based vectors). Additional exemplary methods for introducing nucleic acids into cells include the use of, e.g., naked DNA, CaPO₄ precipitation, DEAE dextran, electroporation, protoplast fusion, lipofection, and cell microinjection.

In some embodiments, an erythroid cell or a progenitor cell can be tranfected with mRNA encoding an exogenous polypeptide described herein to generate an engineered erythroid cells or enucleated cells. mRNA can be derived from in vitro transcription of a cDNA plasmid construct containing a sequence encoding one or more exogenous polypeptide(s). For example, the cDNA sequence encoding an exogenous polypeptide may be inserted into a cloning vector containing a promoter sequence compatible with specific RNA polymerases. For example, the cloning vector ZAP Express® pBK-CMV (Stratagene, La Jolla, Calif., USA) contains T3 and T7 promoter sequences compatible with the T3 and T7 RNA polymerase, respectively. For in vitro transcription of sense mRNA, the plasmid is linearized at a restriction site downstream of the stop codon(s) corresponding to the end of the sequence encoding the exogenous polypeptide. The mRNA is transcribed from the linear DNA template using a commercially available kit such as, for example, the RNAMaxx® High Yield Transcription Kit (Stratagene, La Jolla, Calif., USA). In some instances, it may be desirable to generate 5′-m7GpppG-capped mRNA. As such, transcription of a linearized cDNA template may be carried out using, for example, the mMES SAGE mMACHINE High Yield Capped RNA Transcription Kit from Ambion (Austin, Tex., USA). Transcription may be carried out in a reaction volume of 20-100 μL at 37° C. for 30 min. to 4 hours. The transcribed mRNA is purified from the reaction mix by a brief treatment with DNase I to eliminate the linearized DNA template followed by precipitation in 70% ethanol in the presence of lithium chloride, sodium acetate, or ammonium acetate. The integrity of the transcribed mRNA may be assessed using electrophoresis with an agarose-formaldehyde gel or commercially available Novex pre-cast TBE gels (Novex, Invitrogen, Carlsbad, Calif., USA).

Messenger RNA encoding the exogenous polypeptides may be introduced into erythroid cells or erythroid precursor cells (e.g., CD34⁺ HSCs) using a variety of approaches including, for example, lipofection and electroporation (van Tandeloo et al. (2001) Blood 98:49-56). For lipofection, for example, 5 μg of in vitro transcribed mRNA in Opti-MEM (Invitrogen, Carlsbad, Calif., USA) is incubated for 5-15 min. at a 1:4 ratio with the cationic lipid DMRIE-C (Invitrogen). Alternatively, a variety of other cationic lipids or cationic polymers may be used to transfect cells with mRNA including, for example, DOTAP, various forms of polyethylenimine, and polyL-lysine (Sigma-Aldrich, Saint Louis, Mo., USA), and Superfect (Qiagen, Inc., Valencia, Calif., USA; see, e.g., Bettinger et al., Nucleic Acids Res. 29:3882-3891 (2001)). The resulting mRNA/lipid complexes are incubated with cells (1-2×10⁶ cells/mL) for 2 hours at 37° C., washed and returned to culture. For electroporation, for example, about 5 to 20×10⁶ cells in 500 μl of Opti-MEM (Invitrogen, Carlsbad, Calif., USA) are mixed with about 20 μg of in vitro transcribed mRNA and electroporated in a 0.4-cm cuvette using, for example, an Easyject Plus device (EquiBio, Kent, United Kingdom). In some instances, it may be necessary to test various voltages, capacitances and electroporation volumes to determine the useful conditions for transfection of a particular mRNA into a cell.

Alternatively, mRNA may be transfected into an erythroid precursor cells (e.g., a CD34⁺ cell) or erythroid cell using a peptide-mediated RNA delivery strategy (see, e.g., Bettinger et al., (2001) Nucleic Acids Res. 29: 3882-91). For example, the cationic lipid polyethylenimine (PEI) 2 kDA (Sigma-Aldrich, Saint Louis, Mo., USA) may be combined with the melittin peptide (Alta Biosciences, Birmingham, UK) to increase the efficiency of mRNA transfection, particularly in post-mitotic primary cells. The mellitin peptide may be conjugated to the PEI using a disulfide cross-linker such as, for example, the hetero-bifunctional cross-linker succinimidyl 3-(2-pyridyldithio) propionate. In vitro transcribed mRNA is preincubated for 5 to 15 minutes with the mellitin-PEI to form an RNA/peptide/lipid complex. This complex is then added to cells in serum-free culture medium for 2 to 4 hours at 37° C. in a 5% CO₂ humidified environment, then removed, and the transfected cells further cultured.

In some embodiments, the engineered erythroid cells or enucleated cells are generated by introducing a nucleic acid (e.g., any of the exemplary nucleic acids described herein) encoding one or more exogenous polypeptide(s) into a nucleated precursor cell (e.g., a nucleated erythroid precursor cell). In some embodiments the exogenous polypeptide is encoded by a DNA, which is introduced into a nucleated precursor cell. In some embodiments, the exogenous polypeptide is encoded by an RNA, which is introduced into a nucleated precursor cell (e.g., a nucleated erythroid precursor cell or a nucleated platelet precursor cell), or a platelet.

Nucleic acids encoding one or more exogenous polypeptide(s) can be introduced into erythroid precursor cells and platelet precursor cells prior to terminal differentiation enucleated erythroid cells and platelets, respectively, using a variety of techniques, including, e.g., transient or stable transfections and gene therapy approaches (e.g., using nucleases (e.g., CRISPR/Cas systems)).

Viral gene transfer can be used to transfect the cells with a nucleic acid encoding one or more exogenous polypeptide(s) provided herein. A number of viruses can be used as gene transfer vehicles including, e.g., Moloney murine leukemia virus (MMLV), adenovirus, adeno-associated virus (AAV), herpes simplex virus (HSV), lentiviruses (e.g., human immunodeficiency virus 1 (HIV 1)), and spumaviruses (e.g., foamy viruses, see, e.g., Osten et al. (2007) HEP 178: 177-202).

A nucleic acid encoding one or more exogenous polypeptide(s) can be transfected into erythroid precursor cells and platelet precursor cells. A suitable vector is the Moloney murine leukemia virus (MMLV) vector (see, e.g., Malik et al. (1998) Blood 91:2664-71). For example, a DNA construct containing cDNA encoding an exogenous polypeptide can be incorporated into the MMLV vector backbone using standard molecular biology techniques. The construct is transfected into a packaging cell line (e.g., PA317 cells), and viral particles obtained from the culture supernatant are used to transfect producer cells (e.g., PG13 cells). The PG13 viral supernatant (or viral particles purified therefrom) is incubated with an erythroid precursor cell or a platelet precursor cell. Exogenous polypeptide expression can be monitored using fluorescence-activated cell sorting (FACS) analysis, e.g., with a fluorescently-labeled antibody directed against the exogenous polypeptide.

Nonviral vectors can be used to introduce exogenous nucleic acids encoding one or more exogenous polypeptide(s) into erythroid precursor cells or platelet precursor cells. A number of delivery methods can be used to introduce nonviral vectors into erythroid precursor cells or platelet precursor cells including chemical and physical methods. For example, a nonviral vector (e.g., plasmid DNA) encoding one or more exogenous polypeptide(s) can be introduced into erythroid precursor cells or platelet precursor cells using synthetic macromolecules, such as cationic lipids and polymers (see, e.g., Papapetrou et al. (2005) Gene Therapy 12: S118-30). Alternatively, commercially available liposome transfection reagents can be used. Optionally, a cationic polymer, e.g., PEI can be used to efficiently transfect erythroid precursor cells or platelet precursor cells (e.g., hematopoietic and umbilical cord blood-derived CD34⁺ cells; see, e.g., Shin et al. (2005) Biochim. Biophys. Acta 1725: 377-84). Other methods that can be used to introduce a plasmid vector encoding one or more exogenous polypeptide(s) include particle-mediated transfection, a gene gun, biolistics, or particle bombardment technology (see, e.g., Papapetrou et al. (2005)), and electroporation (e.g., nucleofection). Optionally, erythroid precursor cells and platelet precursor cells can be non-virally transfected with a conventional expression vector that is unable to self-replicate in mammalian cells unless it is integrated into the host genome. Alternatively, erythroid precursor cells and platelet precursor cells can be transfected with an episomal vector that may persist in the host cell nucleus as autonomously replicating genetic units without integration into the host cell's chromosomes (see, e.g., Papapetrou et al. (2005)). Mammalian artificial chromosomes may also be used for nonviral introduction of exogenous nucleic acids (Vanderbyl et al. (2005) Exp. Hematol. 33: 1470-6). Exogenous nucleic acids encoding one or more exogenous polypeptide(s) can be assembled into a nonviral vector using standard molecular biology methods, e.g., restriction digestion, overlap-extension PCR, and Gibson assembly.

In some embodiments, the exogenous nucleic acid encoding an exogenous polypeptide described herein is operatively linked to a constitutive promoter. In some embodiments, the exogenous nucleic acid is operatively linked to an inducible or repressible promoter.

The erythroid cells and enucleated cells described herein can be produced by chemically or enzymatically conjugating an exogenous polypeptide described herein onto the cells (e.g., onto a native protein present on or in the cell). In addition, the erythroid cells and enucleated cells described herein can also be produced by chemically or enzymatically conjugating an exogenous polypeptide onto a different exogenous polypeptide present on or in the cell). In some embodiments, the erythroid cells and enucleated cells described herein are produced using click chemistry to click-conjugate one or more exogenous polypeptides described herein to the cell (e.g., to the cell surface), or by click-conjugating one exogenous polypeptide present on or in the cell to another exogenous polypeptide (e.g., by click-conjugating an exogenous HLA-G polypeptide to an exogenous antigenic polypeptide). Multiple (e.g., two, three, four, or more) exogenous polypeptides can be conjugated to the cells using click chemistry. Methods of using click chemistry to conjugate exogenous polypeptides are known in the art (see, e.g., U.S. Patent Publication No. 2018/0344770, the entire contents of which are incorporated herein by reference). For example, the erythroid cells or enucleated cells described herein can be made by: a) coupling a first click chemistry handle to an erythroid cell, and b) contacting the cell with an exogenous polypeptide coupled to a second click chemistry handle, e.g., under conditions suitable for the first click chemistry handle to react with the second click chemistry handle. Alternatively, the erythroid cells or enucleated cells described herein can be made by: a) coupling a first click chemistry handle to a first exogenous polypeptide (e.g., an exogenous HLA-G polypeptide) on or in the erythroid cells or enucleated cells, and b) contacting the cell with a second exogenous polypeptide (e.g., an exogenous antigenic polypeptide) coupled to a second click chemistry handle, e.g., under conditions suitable for the first coupling reagent to react with the second click chemistry handle. Any click chemistry handle known in the art and can be used to click-conjugate an exogenous polypeptide to a cell or to click conjugate one exogenous polypeptide on a cell provided herein to another exogenous polypeptide. Exemplary click chemistry handles include azides coupling reagents including 3-azidopropionic acid sulfo-NHS ester, azidoacetic acid NHS ester, azido-PEG-NHS ester, azidopropylamine, azido-PEG-amine, azido-PEG-maleimide, bis-sulfone-PEG-azide, or a derivative thereof. In some embodiments, the azide coupling reagent comprises an azidoalkyl moiety, azidoaryl moiety, or an azidoheteroaryl moiety. Additional click chemistry handles are described in McKay and Finn (2014) Chem. Biol. 21(9): 1075-101, and Lahann, J. (ed.) Click Chemistry for Biotechnology and Materials Science, John Wiley & Sons, West Sussex, 2009, each of which is incorporated herein by reference in its entirety.

The erythroid cells and enucleated cells described herein can also be produced by conjugating one or more exogenous polypeptides described herein to the cells or by conjugating one exogenous polypeptide present on or in the cells to another exogenous polypeptide (e.g., conjugating an exogenous HLA-G polypeptide to an exogenous antigenic polypeptide) using a coupling compound containing an electrophilic group (e.g., a mixed anhydride) that will react with a nucleophile on the cell or on an exogenous polypeptide present on the cell, to form an interbonded relationship. Representative electrophilic groups include αβ unsaturated carbonyls, alkyl halides, and thiols such as substituted maleimides. The coupling compound can be attached to an exogenous polypeptide via one or more of the functional groups in the polypeptide (e.g., an amino, carboxyl, or tryosine group). Exogenous polypeptide for conjugation can be prepared using carboxyl groups on coupling agents to form mixed anhydrides which react with the exogenous polypeptide in the presence of an activator (e.g., isobutylchloroformate, 5,5′-(dithiobis(2-nitrobenzoic acid) (DTNB), p-chloromercuribenzoate (CMB), and m-maleimidobenzoic acid (MBA)).

Exogenous polypeptides can also be conjugated to an erythroid cell or enucleated cell described herein, or to another exogenous polypeptide on the cells, using a bridging reagent. Functional groups (e.g., carboxyl groups) on an exogenous polypeptide can be activated using carbodiimides or other known activators. Bridging reagents (e.g., amino groups) can be reacted with the activated functional group(s) to form reactive derivatives. Coupling agent having a second reactive group that can react with appropriate nucleophilic group on the erythroid cell or enucleated cell can be used to form a bridge. Such reactive groups include alkylating agents such as iodoacetic acid, αβ unsaturated carbonyl compounds (e.g., acrylic acid), thiol reagents (e.g., mercurial and substituted maleimides).

Alternatively, exogenous polypeptides can be attached to an erythroid cell or enucleated cell described herein, or to another exogenous polypeptide on the cells, without using a bridging reagent. Functional groups on an exogenous polypeptide (e.g., an exogenous antigenic polypeptide) can be activated to react directly with nucleophiles on erythroid cells or enucleated cells described herein, or on other exogenous polypeptides, using an activator (e.g., Woodward's Reagent K) to form enol ester derivatives on exogenous polypeptides which can subsequently react with nucleophilic groups on the cells or other exogenous polypeptides.

Exogenous polypeptides can also be conjugated to an erythroid cell or enucleated cell described herein, or to another exogenous polypeptide on the cells, using enzyme-mediated conjugation. For example, an exogenous polypeptide can be conjugated to a cell (e.g., to a protein present on the membrane of the cell) using a sortase. Methods of conjugating an exogenous polypeptide to a cell using a sortase are described, e.g., in U.S. Pat. Nos. 10,260,038 and 10,471,099, both of which are incorporated by reference.

The engineered erythroid cells and enucleated cells provided herein can include an exogenous HLA-G polypeptide enzymatically or chemically conjugated to one or more exogenous antigenic polypeptides using methods described herein or otherwise known in the art. Chemical conjugation can be performed by creating a covalent bond between the exogenous HLA-G polypeptide and one or more exogenous antigenic polypeptides, e.g., using a method described above. Exogenous antigenic polypeptide(s) can also be conjugated to exogenous HLA-G polypeptide(s) by any chemical and enzymatic means, including but not limited to, chemical conjugation using bifunctional cross-linking agents (e.g., a NHS ester-maleimide heterobifunctional crosslinker) and click chemistry, and enzymatic conjugation using a transpeptidase, an isopeptidase, a transglutaminase (see, e.g., Steffen et al. (2017) J. Biol. Chem. 292(38): 15622-35), a sortase (e.g., a sortase A or a sortase B), or a butelase (e.g., butelase 1).

Optionally, an exogenous HLA-G polypeptide of an engineered erythroid cell or enucleated cell provided herein can be conjugated to one or more exogenous antigenic polypeptides through a biotin-streptavidin bridge. For example, a biotinylated exogenous antigenic polypeptide can be linked to a non-specifically biotinylated surface of the exogenous HLA-G polypeptide through a streptavidin bridge. Biotin conjugation can be performed by any known chemical means (see, e.g., Hirsch et al. (2004) Methods Mol. Biol. 295: 135-54). The exogenous HLA-G polypeptide can be biotinylated using an amine reactive biotinylation reagent, e.g., EZ-Link Sulfo-NHS—SS-Biotin (sulfosuccinimidyl 2-(biotinamido)-ethyl-1,3-dithiopropionate; Pierce-Thermo Scientific, Rockford, Ill., USA; see, e.g., Jaiswal et al., (2003) Nature Biotech. 21: 47-51).

Exogenous antigenic polypeptides may also be conjugated to an exogenous HLA-G polypeptide of an engineered erythroid cell or enucleated cell provided herein using a sortase (e.g., a sortase A). For example, a first exogenous polypeptide (e.g., an exogenous HLA-G polypeptide on the cells or an exogenous antigenic polypeptide(s)) comprises or is engineered to include either an acceptor sequence (e.g., LPXTG (SEQ ID NO: 32) or LPXTA (SEQ ID NO: 33)), and the second exogenous polypeptide (e.g., an exogenous HLA-G polypeptide on the cells or an exogenous antigenic polypeptide(s)) comprises or is engineered to include an N-terminal donor sequence (e.g., G, GG, GGG, A, AA, and AAA). When contacted with a suitable sortase (e.g., a Streptococcus aureus sortase A or a S. pyogenes sortase A) a transpeptidation reaction occurs such that both exogenous polypeptides are conjugated (see, e.g., Swee et al. (2013) Proc. Nat'l. Acad. Sci. USA 110(4): 1428-33, incorporated herein by reference). In some embodiments, the N-terminus of the exogenous HLA-G polypeptide comprises an N-terminal donor sequence G, GG, GGG, A, AA, or AAA. In some embodiments, N-terminal donor sequence (e.g., GG, GGG) of the exogenous HLA-G polypeptide is conjugated to an exogenous antigenic polypeptide containing the acceptor sequence LPXTG (SEQ ID NO: 32) or LPXTA (SEQ ID NO: 33), via a sortase-mediated reaction (e.g., a sortase A-mediated reaction). Additional acceptor sequences and donor sequences that can be used for sortase-mediated conjugation reactions and methods of utilizing sortagging are described in Antos et al. (2016) Curr Opin Struct Biol. 38: 111-8, the contents of which are hereby incorporated herein by reference.

Exogenous antigenic polypeptide(s) can be conjugated to an exogenous HLA-G polypeptide on an engineered erythroid cell or enucleated cell using a butelase 1, e.g., Clitoria ternatea butelase 1 (UniProtKB Accession No. A0A060D9Z7). For example, a first exogenous polypeptide (e.g., the exogenous HLA-G polypeptide on the cells or the exogenous antigenic polypeptide(s)) comprises or is engineered to include a C-terminal butelase-1 tripeptide recognition sequence Asx-His-Val (wherein Asx is Asp or Asn). The second exogenous polypeptide (e.g., the exogenous HLA-G polypeptide on the cells or the exogenous antigenic polypeptide(s)) is engineered to include an N-terminal X₁X₂, wherein X₁ is any amino acid and X₂ is I, L, V, or C. When contacted with butelase 1, both exogenous polypeptides are conjugated by the enzyme (see, e.g., Nguyen et al. (2016) Nature Protocols 11: 1977-88).

Alternatively, exogenous polypeptides can be conjugated onto the erythroid cells and enucleated cells described herein, and exogenous polypeptides can be conjugated to one another, using a catalytic bond-forming polypeptide (e.g., a SpyTag/SpyCatcher system). For example, the erythroid cells or enucleated cells provided herein can be engineered to include an exogenous polypeptide comprising either a SpyTag or a SpyCatcher polypeptide (e.g., on the extracellular portion of the exogenous polypeptide). Alternatively, an exogenous polypeptide described herein (e.g., an exogenous HLA-G polypeptide or an exogenous antigenic polypeptide can be engineered to include either a SpyTag or SpyCatcher polypeptide). For example, in some embodiments, an exogenous HLA-G polypeptide comprises an N-terminal SpyCatcher polypeptide and an exogenous antigenic polypeptide comprises a SpyTag polypeptide. Upon contacting of the SpyTag and SpyCatcher polypeptides, a covalent bond can be formed (see, e.g., Zakeri et al. (2012) Proc. Nat'l. Acad. Sci. U.S.A. 109: E690-7.

Exogenous polypeptides can be conjugated onto the erythroid cells and enucleated cells described herein, and exogenous polypeptides can be conjugated to one another, using combination methods (e.g., an enzymatic combination and click chemistry). For example, a sortase-mediated conjugation can be used to attach a click-chemistry handles (e.g., an azide or an alkyne) onto a cell or an exogenous polypeptide. Subsequently, click chemistry (e.g., a cyclo-addition reaction) can be used to conjugate an additional exogenous polypeptide onto the cell, or onto an exogenous polypeptide (e.g., an exogenous antigenic polypeptide to an exogenous HLA-G polypeptide; see, e.g., Neves et al. (2013) Bioconjugate Chemistry 24(6): 934-41.

In some embodiments, the erythroid cells and enucleated cells provided herein are produced using methods that does not include one or more of sortase-mediated conjunction, hypotonic loading, a hypotonic dialysis step, and/or controlled cell deformation.

The erythroid cells and enucleated cells provided herein can be isolated using methods known in the art, such as but not limited to, centrifugation (e.g., density-gradient centrifugation), fluorescence-activated cell sorting (FACS), and magnetic-activated cell sorting (MACS). The isolated erythroid cells and enucleated cells can be formulated (e.g., by mixing an isolated population of engineered erythroid cells or enucleated cells with one of more pharmaceutically acceptable carriers (e.g., phosphate buffered saline).

While in many embodiments herein, one or more (e.g., two or more) exogenous polypeptides described herein are situated on or in an erythroid cell (e.g., engineered enucleated erythroid cell) or enucleated cell (e.g., modified enucleated cell), any exogenous polypeptide(s) described herein can also be situated on or in another vehicle. The vehicle can comprise, e.g., a cell, a corpuscle, a nanoparticle, a micelle, a liposome, or an exosome. For instance, in some aspects, the present disclosure provides a vehicle (e.g., a cella corpuscle, a nanoparticle, a micelle, a liposome, or an exosome) including, e.g., on its surface, one or more (e.g., one, two, three, four, five, or more) exogenous polypeptides described herein.

In some embodiments, the engineered erythroid cells (e.g. engineered enucleated erythroid cells), the enucleated cells (e.g., modified enucleated cells), or other vehicles described herein, can be encapsulated in a membrane, e.g., semi-permeable membrane. In some embodiments, the membrane comprises a polysaccharide, e.g., an anionic polysaccharide alginate. In some embodiments, the semipermeable membrane does not allow cells to pass through, but allows passage of small molecules or macromolecules, e.g., metabolites, proteins, or DNA. Multiple suitable membranes are known in the art and can be used for these purposes (see, e.g., Lienert et al. (2014) Nat. Rev. Mol. Cell Biol. 15: 95-107, incorporated herein by reference in its entirety.

### III. Methods of Using Engineered Erythroid Cells

The engineered erythroid cells (e.g., engineered enucleated erythroid cells) or enucleated cells (e.g., modified enucleated cells) including an HLA-G polypeptide and an exogenous immunogenic polypeptide (e.g., on the cell surface, within the cell (e.g., in the cytoplasm or on the intracellular side of the plasma membrane), or secreted or released by the cell) described herein can be used in a variety of therapeutic methods. The engineered erythroid cells (e.g., engineered enucleated erythroid cells) or enucleated cells (e.g., modified enucleated cells) including at least one exogenous autoantigenic polypeptide and at least one exogenous coinhibitory polypeptide also can be used in a variety of therapeutic methods. In particular, these cells can be used to treat a variety of diseases and disorders where it is desirable to provide immune tolerance to or a reduced immune response against an exogenous immunogenic polypeptide and/or an exogenous autoantigenic polypeptide.

In some embodiments, the disclosure features methods of treating a disease in a subject in need thereof by administering to the subject a plurality of any of the engineered erythroid cells or enucleated cells provided herein, or a pharmaceutical composition comprising the cells, thereby treating the subject.

In some embodiments, the engineered erythroid cells or enucleated cells include at least one (e.g., one, two, three, or more) exogenous immunogenic polypeptide, at least one (e.g., one, two, three, or more) exogenous HLA-G polypeptide, and optionally: at least one (e.g., one, two, three, or more) exogenous coinhibitory polypeptide (e.g., present on the cell surface, in the cytoplasm, on the intracellular side of the plasma membrane, or secreted or released by the cell) and/or at least one (e.g., one, two, three, or more) exogenous autoantigenic polypeptide (e.g., present on the cell surface, in the cytoplasm, on the intracellular side of the plasma membrane, or secreted or released by the cell).

In some embodiments, the disease is a disease modulated by the exogenous immunogenic polypeptide, e.g., a cancer, a homocysteine-related disease, a uric acid-related disease, hyperoxaluria, e.g., primary hyperoxaluria, or phenylketonuria (PKU).

In some embodiments, an immune response in the subject to the exogenous immunogenic polypeptide included on the engineered erythroid cells or enucleated cells is reduced, as compared to either (a) an immune response in the subject to the exogenous immunogenic polypeptide when the exogenous immunogenic polypeptide is administered to the subject alone, or (b) an immune response in the subject to the exogenous immunogenic polypeptide when the exogenous immunogenic polypeptide is administered to the subject when present on the surface of a comparable engineered erythroid cell or enucleated cell lacking the exogenous HLA-G polypeptide.

In some embodiments, an immune response in the subject to the exogenous autoantigenic polypeptide included on the engineered erythroid cells or enucleated cells is reduced, as compared to either (a) an immune response in the subject to the exogenous autoantigenic polypeptide when the exogenous antigenic polypeptide is administered to the subject alone, or (b) an immune response in the subject to the exogenous autoantigenic polypeptide when the exogenous autoantigenic polypeptide is administered to the subject when present on the surface of a comparable engineered erythroid cell or enucleated cell lacking an exogenous coinhibitory polypeptide.

In some embodiments, the disclosure provides methods of reducing an immune response in a subject to an exogenous immunogenic polypeptide, the method comprising administering to the subject a plurality of the engineered erythroid cells (e.g., engineered enucleated erythroid cells) or enucleated cells (e.g., modified enucleated cells) described herein, or a pharmaceutical composition comprising the cells described herein, wherein the immune response to the exogenous immunogenic polypeptide is reduced by at least 5%, 10%, 15%, 20%, 25%, 30%, 35%, 40%, 45%, 50%, 55%, 60%, 65%, 70%, 75%, 80%, 85%, 90% or more, in the subject, as compared to either (a) an immune response in the subject to the exogenous immunogenic polypeptide when the exogenous immunogenic polypeptide is administered to the subject alone, or (b) an immune response in the subject to the exogenous immunogenic polypeptide when the exogenous immunogenic polypeptide is administered to the subject when present on the surface of a comparable engineered erythroid cell or enucleated cell lacking the exogenous HLA-G polypeptide.

In some embodiments, the disclosure provides methods of reducing an immune response in a subject to an exogenous autoantigenic polypeptide, the method comprising administering to the subject a plurality of the engineered erythroid cells (e.g., engineered enucleated erythroid cells) or enucleated cells (e.g., modified enucleated cells) described herein, or a pharmaceutical composition comprising the cells described herein, wherein the immune response to the exogenous autoantigenic polypeptide is reduced by at least 5%, 10%, 15%, 20%, 25%, 30%, 35%, 40%, 45%, 50%, 55%, 60%, 65%, 70%, 75%, 80%, 85%, 90% or more, in the subject, as compared to either (a) an immune response in the subject to the exogenous autoantigenic polypeptide when the exogenous autoantigenic polypeptide is administered to the subject alone, or (b) an immune response in the subject to the exogenous autoantigenic polypeptide when the exogenous autoantigenic polypeptide is administered to the subject when present on the surface of a comparable engineered erythroid cell or enucleated cell lacking an exogenous coinhibitory polypeptide.

In some embodiments, the disclosure provides a method of inducing immune tolerance in a subject, e.g., long-term immune tolerance or short-term immune tolerance, to an exogenous immunogenic polypeptide, the method comprising administering to the subject a plurality of the engineered erythroid cells (e.g., engineered enucleated erythroid cells) or enucleated cells (e.g., modified enucleated cells) described herein, or a pharmaceutical composition comprising the cells. In some embodiments, the disclosure provides a method of inducing immune tolerance in a subject, e.g., long-term immune tolerance or short-term immune tolerance, to an exogenous immunogenic polypeptide, the method comprising contacting immune cells of the subject with an engineered erythroid cell (e.g., engineered enucleated erythroid cell) or enucleated cell (e.g., modified enucleated cell) as described herein. In some embodiments, the engineered erythroid cells or enucleated cells include at least one (e.g., one, two, three, or more) exogenous immunogenic polypeptide, at least one (e.g., one, two, three, or more) exogenous HLA-G polypeptide, and optionally: at least one (e.g., one, two, three, or more) exogenous coinhibitory polypeptide and/or at least one (e.g., one, two, three, or more) exogenous antigenic polypeptide. In some embodiments, the exogenous HLA-G polypeptide is bound to an exogenous antigenic polypeptide. In some embodiments, the contacting is performed in vitro, ex vivo, or in vivo. In some embodiments, the contacting is performed in vitro. In some embodiments, the contacting is performed ex vivo. In some embodiments, the contacting is performed in vivo.

In some embodiments, the disclosure provides a method of inducing immune tolerance in a subject, e.g., long-term immune tolerance or short-term immune tolerance, to an exogenous autoantigenic polypeptide, the method comprising contacting immune cells of the subject with an engineered erythroid cell (e.g., engineered enucleated erythroid cell) or enucleated cell (e.g., modified enucleated cell) as described herein.

In some embodiments, the immune tolerance induced by the methods provided herein is short-term immune tolerance. In some embodiments, the short-term immune tolerance comprises inhibiting the activation, differentiation, and/or proliferation of an immune cell that is contacted by the engineered erythroid cell or enucleated cell provided herein, wherein the immune cell is selected from the group consisting of a T cell, a NK cells, or a B cell. In some embodiments, the short-term immune tolerance comprises inhibiting the cytotoxicity of a T cell or a NK cell that is contacted by the engineered erythroid cell or enucleated cell provided herein. In some embodiments, the short-term immune tolerance comprises inhibiting antibody secretion by a B cell that is contacted by the engineered erythroid cell or enucleated cell provided herein.

In some embodiments, the immune tolerance induced by the methods provided herein is long-term immune tolerance. In some embodiments, the long-term immune tolerance comprises inhibiting the maturation of a dendritic cell (DC) that is contacted by the engineered enucleated erythroid cell. In some embodiments, the long-term immune tolerance comprises inducing anergy of a dendritic cell (DC) that is contacted by the engineered enucleated erythroid cell. In some embodiments, the long-term immune tolerance comprises inducing the differentiation of CD4⁺ T cell that is contacted by the engineered enucleated erythroid cell into a regulatory T cell (Treg); or inducing the differentiation of CD8⁺ T cell that is contacted by the engineered enucleated erythroid cell into a regulatory T cell (Treg).

In another aspect, the disclosure provides a method of treating a subject in need of a reduced immune response, the method comprising contacting immune cells of the subject with an engineered erythroid cell (e.g., engineered enucleated erythroid cell) or enucleated cell (e.g., modified enucleated cell) described herein, thereby treating the subject in need of the reduced immune response. In some embodiments, the subject in need of a reduced immune response is a subject suffering from a disease modulated by an exogenous immunogenic polypeptide on the surface of the engineered erythroid cell or enuclated cell (e.g., a cancer, a homocysteine-related disease, a uric acid-related disease, hyperoxaluria, e.g., primary hyperoxaluria, or phenylketonuria (PKU)).

Methods of administering engineered erythroid cells and enucleated cells comprising an exogenous agent polypeptides are described, e.g., in International Patent Publication Nos. WO 2015/073587 and WO 2015/153102, each of which is incorporated by reference in its entirety. The engineered erythroid cells and enucleated cells, or pharmaceutical compositions including the cells, can be administered to a subject using any convenient manner, including injection, ingestion, transfusion, implantation, or transplantation. For example, the engineered erythroid cells and enucleated cells, or pharmaceutical compositions including the cells can be administered to a subject subcutaneously, intradermally, intramuscularly, by intravenous (i.v.) injection, intraperitoneally, or by injection directly into a tumor or lymph node. In some embodiments, the engineered erythroid cells or enucleated cells are administered directly into the circulation (e.g., intravenously) or the spleen of a subject.

In another aspect, the disclosure features a method of treating a subject in need of a reduced immune response, the method comprising a) determining an HLA status of the subject, b) selecting an engineered erythroid cell (e.g., engineered enucleated erythroid cell) or enucleated cell (e.g., modified enucleated cell) that is immunologically compatible with the subject, wherein the cell is an engineered erythroid cell or enucleated cell includes an exogenous HLA-G polypeptide and an exogenous immunogenic polypeptide, and optionally an exogenous coinhibitory polypeptide and/or an exogenous antigenic polypeptide, and c) administering the engineered erythroid cell or enucleated cell to the subject, thereby treating the subject in need of the reduced immune response.

In some embodiments, a dose of the engineered erythroid cells or the enucleated cells provided herein comprises about 1×10⁹-2×10⁹, 2×10⁹-5×10⁹, 5×10⁹-1×10¹⁰, 1×10¹⁰-2×10¹⁰, 2×10¹⁰-5×10¹⁰, 5×10¹⁰-1×10¹¹, 1×10¹¹-2×10¹¹, 2×10¹¹-5×10¹¹, 5×10¹¹-1×10¹², 1×10¹²-2×10¹², 2×10¹²-5×10¹², or 5×10¹²-1×10¹³ cells.

In some aspects, the disclosure provides a method of treating a disease in a subject in need thereof, the method comprising administering to a subject in need thereof an engineered erythroid cell or enucleated cell described herein, or a pharmaceutical composition comprising a population of the engineered erythroid cells or enucleated cells. In some embodiments, the disease is a cancer, a homocysteine-related disease, a uric acid-related disease, hyperoxaluria, e.g., primary hyperoxaluria, or phenylketonuria (PKU).

In some aspects, the disclosure provides use of an engineered erythroid cell or enucleated cell described herein, or a pharmaceutical compositions comprising the cells, for treating a disease provided herein, e.g., a cancer, a homocysteine-related disease, a uric acid-related disease, hyperoxaluria, e.g., primary hyperoxaluria, or phenylketonuria (PKU).

In some embodiments the plurality of any of the engineered enucleated erythroid cells described herein or any of the pharmaceutical compositions described herein can be contacted with a phagocytosis-inducing agent or an agent that increases the presence of phosphatidylserine on the outer leaflet of the plasma membrane (e.g., a calcium ionophore, e.g., ionomycin, A23187, and bissulfosuccinimidyl suberate (BS3)). See, e.g., WO 2015/153102A1.

In other aspects, the disclosure provides use of an engineered erythroid cell or enucleated cell described herein for manufacture of a medicament for treating a disease described herein, e.g., a cancer, a homocysteine-related disease, a uric acid-related disease, hyperoxaluria, e.g., primary hyperoxaluria, or phenylketonuria (PKU).

### Cancer

In some aspects, the present disclosure provides a method of treating a cancer in a subject in need thereof, the method comprising administering to the subject an engineered erythroid cell or enucleated cell, a population of the cells, or a pharmaceutical composition comprising the population, wherein the engineered erythroid cell or enucleated cell include at least one exogenous immunogenic polypeptide, at least one exogenous HLA-G polypeptide, and optionally: at least one exogenous coinhibitory polypeptide and/or at least one exogenous antigenic polypeptide. In some embodiments, the exogenous immunogenic polypeptide comprises an amino acid-degrading polypeptide (e.g., asparaginase or glutaminase).

In some aspects, the present disclosure provides a method of treating a cancer in a subject in need thereof, the method comprising administering to the subject an engineered erythroid cell or enucleated cell, a population of the cells, or a pharmaceutical composition comprising the population, wherein the engineered erythroid cell or enucleated cell include at least one exogenous autoantigenic polypeptide and at least one exogenous coinhibitory polypeptide.

In some embodiments, the cancer is chosen from acute lymphoblastic leukemia (ALL), an acute myeloid leukemia (AML), an anal cancer, a bile duct cancer, a bladder cancer, a bone cancer, a bowel cancer, a brain tumor, a breast cancer, a carcinoid, a cervical cancer, a choriocarcinoma, a chronic lymphocytic leukemia (CLL), a chronic myeloid leukemia (CIVIL), a colon cancer, a colorectal cancer, an endometrial cancer, an eye cancer, a gallbladder cancer, a gastric cancer, a gestational trophoblastic tumor (GTT), a hairy cell leukemia, a head and neck cancer, a Hodgkin lymphoma, a kidney cancer, a laryngeal cancer, a liver cancer, a lung cancer, a lymphoma, a melanoma, a skin cancer, a mesothelioma, a mouth or oropharyngeal cancer, a myeloma, a nasal or sinus cancer, a nasopharyngeal cancer, a non-Hodgkin lymphoma (NHL), an esophageal cancer, an ovarian cancer, a pancreatic cancer, a penile cancer, a prostate cancer, a rectal cancer, a salivary gland cancer, a non-melanoma skin cancer, a soft tissue sarcoma, a stomach cancer, a testicular cancer, a thyroid cancer, a uterine cancer, a vaginal cancer, and a vulvar cancer.

In some embodiments, cancer cells of the subject are auxotrophic, e.g., at least a sub-population of cancer cells in the subject are auxotrophic. In some embodiments, one or more cancer cells in the subject have impaired synthesis of an amino acid, e.g., asparagine and/or glutamine. In some embodiments, the cancer has a mutation in an amino acid synthesis gene, e.g., wherein the mutation reduces or eliminates activity of the gene product. In some embodiments, the amino acid synthesis gene encodes a protein that contributes to biosynthesis of the amino acid, e.g., catalyzes formation of the amino acid from a precursor molecule.

In some embodiments, the engineered erythroid cell or enucleated cell includes an exogenous immunogenic polypeptide comprising an asparaginase polypeptide, as well as an exogenous polypeptide comprising an anti-CD33 targeting moiety (e.g., an anti-CD33 antibody or a specific binding partner for CD33, e.g., a CD33-binding fragment or a CD33 ligand, e.g., a naturally-occurring CD33 ligand). These cells and pharmaceutical compositions including the cells can be used for the treatment of cancer (e.g., leukemia, e.g., ALL or CLL).

In some embodiments, the engineered erythroid cell or enucleated cell provided herein is administered together with a second therapy. The second therapy may comprise, e.g., chemotherapy, radiation therapy, surgery, or an antibody therapy.

Efficacy can be assayed, for example, by contacting engineered erythroid cells or enucleated cells described herein with cancer cells (e.g., one or more of MV4-11, MOLM-13, THP1, HL60, B16-F10, RPMI 8226) in vitro, and assaying one or more of the following: number of cancer cells, division rate of cancer cells, and replication of cancer cell DNA (e.g., after incubation, e.g., for 68 or 87 hours). Anti-cancer efficacy can also be assayed using animal models known in the art, e.g., for AML, a disseminated MV4-11 AML mouse model can be used.

### Homocysteine-Related Diseases

In some aspects, the present disclosure provides a method of treating a homocysteine-related disease (e.g., homocystinuria) in a subject in need thereof, the method comprising administering to the subject an engineered erythroid cell or enucleated cell, a population of the cells, or a pharmaceutical composition comprising the population, wherein the engineered erythroid cell or enucleated cell include at least one exogenous immunogenic polypeptide, at least one exogenous HLA-G polypeptide, and optionally: at least one exogenous coinhibitory polypeptide and/or at least one exogenous antigenic polypeptide. In some embodiments, the exogenous immunogenic polypeptide comprises an homocysteine-reducing polypeptide or a homocysteine degrading polypeptide. In some embodiments, the exogenous immunogenic polypeptide comprises a homocysteine-reducing polypeptide selected from a methionine adenosyltransferase, an alanine transaminase, an L-alanine-L-anticapsin ligase, an L-cysteine desulfidase, a methylenetetrahydrofolate reductase, a 5-methyltetrahydrofolate-homocysteine methyltransferase reductase, and a methylmalonic aciduria or a homocystinuria, cblD type, or a variant thereof. In some embodiments, the exogenous immunogenic polypeptide comprises a homocysteine-degrading polypeptide selected from a CBS, a methionine gamma-lyase, a sulfide:quinone reductase, a methionine synthase, a 5-methyltetrahydropteroyltriglutamate-homocysteine S-methyltransferase, an adenosylhomocysteinase, a cystathionine gamma-lyase, a methionine gamma-lyase, an L-amino-acid oxidase, a thetin-homocysteine S-methyltransferase, a betaine-homocysteine S-methyltransferase, a homocysteine S-methyltransferase, a 5-methyltetrahydropteroyltriglutamate-homocysteine S-methyltransferase, a selenocysteine Se-methyltransferase, a cystathionine gamma-synthase, an O-acetylhomoserine aminocarboxypropyltransferase, an asparagine-oxo-acid transaminase, a glutamine-phenylpyruvate transaminase, a 3-mercaptopyruvate sulfurtransferase, a homocysteine desulfhydrase, a cystathionine beta-lyase, an amino-acid racemase, a methionine-tRNA ligase, a glutamate-cysteine ligase, an N-(5-amino-5-carboxypentanoyl)-L-cysteinyl-D-valine synthase, an L-isoleucine 4-hydroxylase, an L-lysine N6-monooxygenase (NADPH), a methionine decarboxylase, 2,2-dialkylglycine decarboxylase (pyruvate), and a CysO, or a variant thereof.

In some aspects, the present disclosure provides a method of treating a homocysteine-related disease (e.g., homocystinuria) in a subject in need thereof, the method comprising administering to the subject an engineered erythroid cell or enucleated cell, a population of the cells, or a pharmaceutical composition comprising the population, wherein the engineered erythroid cell or enucleated cell include at least one exogenous autoantigenic polypeptide and at least one exogenous coinhibitory polypeptide.

In some embodiments, the homocysteine-related disease is homocystinuria (e.g., CBS-deficient homocystinuria, symptomatic homocystinuria, or asymptomatic homocystinuria). In some embodiments, administration of the engineered erythroid cells, enucleated cells, or pharmaceutical compositions comprising the cells, to a subject reduces the level of plasma total homocysteine (tHcy) in the subject to a normal level (e.g., between about 5 to about 15 μM, from about 5 to about 50 μM, from about 10 to about 50 μM, or from about 15 to about 50 μM).

In some embodiments, administration of the engineered erythroid cells, enucleated cells or pharmaceutical compositions comprising the cells, to a subject decreases total plasma homocysteine levels by about 5%, 10%, 15%, 20%, 25%, 30%, 35%, 40%, 45%, 50%, 55%, 60%, 70%, 80%, 90%, 100%, 150%, 200%, or more, as compared to the level of total plasma homocysteine in the subject prior to administering the cells or the pharmaceutical composition.

### Phenylketonuria

In some aspects, the present disclosure provides a method of treating phenylketonuria (PKU) in a subject in need thereof, the method comprising administering to the subject an engineered erythroid cell or enucleated cell, a population of the cells, or a pharmaceutical composition comprising the population, wherein the engineered erythroid cell or enucleated cell include at least one exogenous immunogenic polypeptide, at least one exogenous HLA-G polypeptide, and optionally: at least one exogenous coinhibitory polypeptide and/or at least one exogenous antigenic polypeptide. In some embodiments, the exogenous immunogenic polypeptide comprises a PAL or a PAH.

In some aspects, the present disclosure provides a method of treating phenylketonuria (PKU) in a subject in need thereof, the method comprising administering to the subject an engineered erythroid cell or enucleated cell, a population of the cells, or a pharmaceutical composition comprising the population, wherein the engineered erythroid cell or enucleated cell include at least one autoantigenic immunogenic polypeptide and at least one exogenous coinhibitory polypeptide and/or at least one exogenous antigenic polypeptide.

### Uric Acid-Related Diseases

In some aspects, the present disclosure provides a method of treating a uric acid-related disease (e.g., gout) in a subject in need thereof, the method comprising administering to the subject an engineered erythroid cell or enucleated cell, a population of the cells, or a pharmaceutical composition comprising the population, wherein the engineered erythroid cell or enucleated cell include at least one exogenous immunogenic polypeptide, at least one exogenous HLA-G polypeptide, and optionally: at least one exogenous coinhibitory polypeptide and/or at least one exogenous antigenic polypeptide. In some embodiments, the exogenous immunogenic polypeptide comprises a uric acid-degrading polypeptide (e.g., urate oxidase, allantoinase or allantoicase).

In some aspects, the present disclosure provides a method of treating a uric acid-related disease (e.g., gout) in a subject in need thereof, the method comprising administering to the subject an engineered erythroid cell or enucleated cell, a population of the cells, or a pharmaceutical composition comprising the population, wherein the engineered erythroid cell or enucleated cell include at least one exogenous autoantigenic polypeptide and at least one exogenous coinhibitory polypeptide.

In some embodiments, the uric acid-related disease is selected from hyperuricemia, asymptomatic hyperuricemia, hyperuricosuria, gout (e.g., chronic refractory gout), lesch-nyhan syndrome, uric acid nephrolothiasis, vascular conditions, diabetes, metabolic syndrome, inflammatory responses, cognitive impairment, rheumatoid arthritis, osteoarthritis, cerebral stroke, ischemic heart disease, arrhythmia, and chronic renal disease.

In some embodiments, administration of the engineered erythroid cells, enucleated cells or pharmaceutical compositions comprising the cells, to a subject decreases uric acid levels in the blood of a subject by about 5%, 10%, 15%, 20%, 25%, 30%, 35%, 40%, 45%, 50%, 55%, 60%, 70%, 80%, 90%, 100%, 150%, 200%, or more, as compared to the uric acid levels in the blood of the subject prior to administering the cells or the pharmaceutical composition.

### Hyperoxaluria

In some aspects, the present disclosure provides a method of treating a hyperoxaluria (e.g., primary hyperoxaluria) in a subject in need thereof, the method comprising administering to the subject an engineered erythroid cell or enucleated cell, a population of the cells, or a pharmaceutical composition comprising the population, wherein the engineered erythroid cell or enucleated cell include at least one exogenous immunogenic polypeptide, at least one exogenous HLA-G polypeptide, and optionally: at least one exogenous coinhibitory polypeptide and/or at least one exogenous antigenic polypeptide. In some embodiments, the exogenous immunogenic polypeptide comprises an oxolate oxidase.

In some aspects, the present disclosure provides a method of treating a hyperoxaluria (e.g., primary hyperoxaluria) in a subject in need thereof, the method comprising administering to the subject an engineered erythroid cell or enucleated cell, a population of the cells, or a pharmaceutical composition comprising the population, wherein the engineered erythroid cell or enucleated cell include at least one exogenous autoantigenic polypeptide and at least one exogenous coinhibitory polypeptide.

In some embodiments, administration of the engineered erythroid cells, enucleated cells or pharmaceutical compositions comprising the cells, to a subject decreases oxolate levels in the blood of a subject by about 5%, 10%, 15%, 20%, 25%, 30%, 35%, 40%, 45%, 50%, 55%, 60%, 70%, 80%, 90%, 100%, 150%, 200%, or more, as compared to the oxolate levels in the blood of the subject prior to administering the cells or the pharmaceutical composition. In some embodiments, administration of the engineered erythroid cells, enucleated cells or pharmaceutical compositions comprising the cells, to a subject decreases oxolate levels in the urine of a subject by about 5%, 10%, 15%, 20%, 25%, 30%, 35%, 40%, 45%, 50%, 55%, 60%, 70%, 80%, 90%, 100%, 150%, 200%, or more, as compared to the oxolate levels in the uine of the subject prior to administering the cells or the pharmaceutical composition.

### Autoimmune Diseases

In some aspects, the present disclosure provides a method of treating an autoimmune disease (e.g., cellular immunity-driven diseases, humoral immunity-driven diseases, other autoimmune diseases) in a subject in need thereof, the method comprising administering to the subject an engineered erythroid cell or enucleated cell (e.g., any of the exemplary cells described herein), a population of the cells (e.g., any of the exemplary populations of cells described herein), or a pharmaceutical composition comprising the population (e.g., any of the exemplary pharmaceutical compositions described herein), wherein the engineered erythroid cell or enucleated cell includes at least one exogenous immunogenic polypeptide (e.g., any of the exemplary exogenous immunogenic polypeptides described herein or known in the art), at least one exogenous HLA-G polypeptide (e.g., any of the exemplary exogenous HLA-G polypeptides described herein or known in the art), and optionally, at least one exogenous coinhibitory polypeptide (e.g., any of the exemplary exogenous HLA-G polypeptides described herein or known in the art) and/or at least one exogenous antigenic polypeptide (e.g., any of the exogenous antigenic polypeptides described herein or known in the art).

In some aspects, the present disclosure provides a method of treating an autoimmune disease (e.g., cellular immunity-driven diseases, humoral immunity-driven diseases, other autoimmune diseases) in a subject in need thereof, the method comprising administering to the subject an engineered erythroid cell or enucleated cell (e.g., any of the exemplary cells described herein), a population of the cells (e.g., any of the exemplary populations of cells described herein), or a pharmaceutical composition comprising the population (e.g., any of the exemplary pharmaceutical compositions described herein), wherein the engineered erythroid cell or enucleated cell includes at least one autoantigenic polypeptide (e.g., any of the exemplary exogenous autoantigenic polypeptides described herein or known in the art) and at least one exogenous coinhibitory polypeptide (e.g., any of the exemplary exogenous coinhibitory polypeptides described herein or known in the art).

Non-limiting example of autoimmune diseases include achalasia, Addison's disease, adult Still's disease, agammaglobulinemia, alopecia areata, amyloidosis, ankylosing spondylitis, anti-GBM/anti-TBM nephritis, antiphospholipid syndrome, autoimmune angioedema, autoimmune dysautonomia, autoimmune encephalomyelitis, autoimmune hepatitis, autoimmune inner ear disease (AIED), autoimmune myocarditis, autoimmune oophoritis, autoimmune orchitis, autoimmune pancreatitis, autoimmune retinopathy, autoimmune urticarial, axonal & neuronal neuropathy (AMAN), Baló disease, Behcet's disease, benign mucosal pemphigoid, bullous pemphigoid, Castleman disease (CD), Celiac disease, Chagas disease, chronic inflammatory demyelinating polyneuropathy (CIDP), chronic recurrent multifocal osteomyelitis (CRMO), Churg-Strauss syndrome (CSS) or eosinophilic granulomatosis (EGPA), cicatricial pemphigoid, Cogan's syndrome, cold agglutinin disease, congenital heart block, Coxsackie myocarditis, CREST syndrome, Crohn's disease, dermatitis herpetiformis, dermatomyositis, Devic's disease (neuromyelitis optica), discoid lupus, Dressler's syndrome, endometriosis, eosinophilic esophagitis (EoE), eosinophilic fasciitis, erythema nodosum, essential mixed cryoglobulinemia, Evans syndrome, fibromyalgia, fibrosing alveolitis, giant cell arteritis (temporal arteritis), giant cell myocarditis, glomerulonephritis, Goodpasture's syndrome, granulomatosis with Polyangiitis, Graves' disease, Guillain-Barre syndrome, Hashimoto's thyroiditis, hemolytic anemia, Henoch-Schonlein purpura (HSP), herpes gestationis or pemphigoid gestationis (PG), hidradenitis suppurativa (HS) (acne inversa), hypogammalglobulinemia, IgA nephropathy, IgG4-related sclerosing disease, immune thrombocytopenic purpura (ITP), inclusion body myositis (IBM), interstitial cystitis (IC), juvenile arthritis, juvenile diabetes (Type 1 diabetes), juvenile myositis (JM), Kawasaki disease, Lambert-Eaton syndrome, leukocytoclastic vasculitis, lichen planus, lichen sclerosus, ligneous conjunctivitis, linear IgA disease (LAD), lupus, Lyme disease chronic, Meniere's disease, microscopic polyangiitis (MPA), mixed connective tissue disease (MCTD), Mooren's ulcer, Mucha-Habermann disease, Multifocal Motor Neuropathy (MMN) or MMNCB, multiple sclerosis, myasthenia gravis, myositis, narcolepsy, neonatal lupus, neuromyelitis optica, neutropenia, ocular cicatricial pemphigoid, optic neuritis, palindromic rheumatism (PR), PANDAS, paraneoplastic cerebellar degeneration (PCD), paroxysmal nocturnal hemoglobinuria (PNH), Parry Romberg syndrome, pars planitis (peripheral uveitis), Parsonage-Turner syndrome, pemphigus, peripheral neuropathy, perivenous encephalomyelitis, pernicious anemia (PA), POEMS syndrome, polyarteritis nodosa, polyglandular syndromes type I, II, III, polymyalgia rheumatic, polymyositis, postmyocardial infarction syndrome, postpericardiotomy syndrome, primary biliary cirrhosis, primary sclerosing cholangitis, progesterone dermatitis, psoriasis, psoriatic arthritis, pure red cell aplasia (PRCA), pyoderma gangrenosum, Raynaud's phenomenon, reactive arthritis, reflex sympathetic dystrophy, relapsing polychondritis, restless legs syndrome (RLS), retroperitoneal fibrosis, rheumatic fever, rheumatoid arthritis, sarcoidosis, Schmidt syndrome, scleritis, scleroderma, Sjögren's syndrome, sperm & testicular autoimmunity, stiff person syndrome (SPS), subacute bacterial endocarditis (SBE), Susac's syndrome, sympathetic ophthalmia (SO), Takayasu's arteritis, temporal arteritis/giant cell arteritis, thrombocytopenic purpura (TTP), Tolosa-Hunt syndrome (THS), transverse myelitis, type 1 diabetes, ulcerative colitis (UC), undifferentiated connective tissue disease (UCTD), uveitis, vasculitis, vitiligo, and Vogt-Koyanagi-Harada disease.

Non-limiting examples of cellular immunity-driven diseases include: type 1 diabetes, multiple sclerosis, connective tissue disorder, and Celiac disease.

In some embodiments, where the autoimmune disease is type 1 diabetes, the exogenous immunogenic polypeptide(s) and/or exogenous autoantigenic polypeptides and/or autoantigen(s) is/are selected from one or more of: insulin, proinsulin, preproinsulin, islet antigen 2 (IA-2), glutamic acid decarboxylase (e.g., GAD1, GAD2, GAD65, or GAD67), Zinc transporter 8 (ZnT8), islet-specific glucose-6-phosphatase catalytic subunit-related protein (IGRP), peripherin, aGlia, alpha/beta-gliadin, PDC-E2, dihydrolipoamide S-acetyltransferase, DG1 EC2, desmosomal glycoprotein 1, DG3 (desmoglein 3), AQP4 (aquaporin 4), and chromogranin A.

In some embodiments, where the autoimmune disease is multiple sclerosis, the exogenous immunogenic polypeptide(s) and/or exogenous autoantigenic polypeptides and/or autoantigen(s) is/are selected from one or more of: myelin oligodendrocyte glycoprotein (MOG), myelin basic protein (MBP), proteolipid protein (PLP), myelin associated glycoprotein (MAG), myelin associated oligodendrocyte basic protein (MOBP), 2′,3′-cyclic nucleotide 3′-phosphodiesterase (CNPase), 5100 calcium binding protein B (S100beta), and transaldolase.

In some embodiments, where the autoimmune disease is mixed connective tissue disorder, the exogenous immunogenic polypeptide(s) and/or exogenous autoantigenic polypeptide(s) and/or autoantigen(s) is/are selected from one or more of: U1 small nuclear ribonucleoprotein (U1snRNP), 73 kDa heat shock protein, and casein kinase.

In some embodiments, where the autoimmune disease is Celiac disease, the exogenous immunogenic polypeptide and/or exogenous autoantigenic polypeptide comprises gluten.

Non-limiting examples of humoral immunity-driven diseases include: bullous pemphigoid, membranous glomerulonephritis, neuromyelitis optica, and pemphigus vulgaris.

In some embodiments, where the autoimmune disease is bullous pemphigoid, the exogenous immunogenic polypeptide(s) and/or exogenous autoantigenic polypeptides and/or autoantigen(s) is/are selected from one or more of: collagen type XVII alpha 1 (BP180), bullous pemphigoid antigen 230 (BP230), laminin 332, α₆-β₄ integrin, and type VII collagen.

In some embodiments, where the autoimmune disease is membranous glomerulonephritis, the exogenous immunogenic polypeptide(s) and/or exogenous autoantigenic polypeptides and/or autoantigen(s) is/are selected from one or more of: phospholipase A2 receptor (PLA2R), neutral endopeptidase (NEP), and thrombospondin type 1 domain containing 7A (THSD7A).

In some embodiments, where the autoimmune disease is neuromyelitis optica the exogenous immunogenic polypeptide(s) and/or exogenous autoantigenic polypeptides and/or autoantigen(s) is/are selected from one or both of aquaporin 4 (AQP-4) and MOG.

In some embodiments, where the autoimmune disease is pemphigus vulgaris, the exogenous immunogenic polypeptide(s) and/or exogenous autoantigenic polypeptide(s) and/or autoantigen(s) is/are selected from one or both of desmoglein 1 (DSG1) and desmoglein 3 (DSG3).

Additional non-limiting examples of autoimmune diseases include: autoimmune encephalitis, autoimmune hepatitis, chronic inflammatory demyelinating polyneuropathy (CIPD), polymyositis and dermatomyositis (PM/DM), mixed connective tissue disease (MCTD), myasthenia gravis, rheumatoid arthritis, autoimmune liver disease, uveitis, autoimmune myocarditis, vitiligo, alopecis areata, and scleroderma.

In some embodiments, where the autoimmune disease is autoimmune encephalitis, the exogenous immunogenic polypeptide(s) and/or exogenous autoantigenic polypeptides is/are selected from one or more of: N-methyl-D-aspartate receptor (NMDAR), histone-like DNA binding protein (Hu), Ma/Ta, CV2, glutamic acid decarboxylase (GAD), voltage-gated potassium channel-complex (VGKC), voltage-gated calcium channel (VGCC), leucine-rich, glioma inactivated 1 (LGI1), α-amino-3-hydroxy-5-methyl-4-isoxazolepropionic acid receptor (AMPAR), gamma-aminobutyric acid A (GABA-A) receptor, GABA-B receptor, contactin associated protein 2 (Caspr2), IgLON5, dipeptidyl-peptidase-like protein 6 (DPPX), glycine receptor (GlyR), metabotropic glutamate receptor 5 (mGluR5), glutamate metabotropic receptor 1 (mGluR1), neurexin 3-alpha, or dopamine-2 receptor.

In some embodiments, where the autoimmune disease is autoimmune hepatitis, the exogenous immunogenic polypeptide(s) and/or exogenous autoantigenic polypeptides and/or autoantigen(s) is/are selected from one or more of: liver kidney microsomal type 1 (LKM1), (SMA), (ANA), liver kidney microsomal type 2 (LKM2), Src-like-adapter (SLA), dynein light chain 1 (LC1), asialoglycoprotein receptor 1 (ASGPR), and perinuclear anti-neutrophil cytoplasmic antibody (pANCA).

In some embodiments, where the autoimmune disease is chronic inflammatory demyelinating polyneuropathy (CIPD), the exogenous immunogenic polypeptide(s) and/or exogenous autoantigenic polypeptides and/or autoantigen(s) is/are selected from one or more of: contactin 1 (CNTN), neurofascin-155 (NF155), or intravenous immunoglobulins (IVIG). In some embodiments, where the autoimmune disease is polymyositis and dermatomyositis (PM/DM), the exogenous immunogenic polypeptide(s) and/or exogenous autoantigenic polypeptide(s) and/or autoantigen(s) is/are selected from one or more of comprises histidyl tRNA synthetase (Jo-1), melanoma differentiation-associated gene 5 (MDA5/CADM140), or TF181alpha.

In some embodiments, where the autoimmune disease is mixed connective tissue disease (MCTD), the exogenous immunogenic polypeptide and/or exogenous autoantigenic polypeptide and/or autoantigen(s) comprises U1 small nuclear 1 (U1-RNA).

In some embodiments, where the autoimmune disease is myasthenia gravis, the exogenous immunogenic polypeptide is nicotine acetylcholine receptor (nAchR).

In some embodiments, where the autoimmune disease is rheumatoid arthritis, the exogenous immunogenic polypeptide(s) and/or exogenous autoantigenic polypeptide(s) and/or autoantigen(s) is/are selected from one or more of: collagen, heat shock proteins, and human T cell antigen gp39.

In some embodiments, where the autoimmune disease is autoimmune liver disease, the exogenous immunogenic polypeptide or exogenous autoantigenic polypeptide or autoantigen is pyruvate dehydrogenase complex-E2 (PDC-E2).

In some embodiments, where the autoimmune disease is uveitis, the exogenous immunogenic polypeptide(s) and/or exogenous autoantigenic polypeptide(s) and/or autoantigen(s) is/are one or both of retinol binding protein 3 (IRBP) and S-arrestin. In some embodiments, where the autoimmune disease is autoimmune myocarditis, the exogenous immunogenic polypeptide(s) and/or exogenous autoantigenic polypeptide(s) and/or autoantigen(s) is/are selected from one or more of: cardiac myosin (e.g., αMyHC), myosin-binding protein-C (MYBC), fast-type RNA-binding protein 20 (RBM20), and dystrophin.

In some embodiments, where the autoimmune disease is vitiligo, the exogenous immunogenic polypeptide(s) and/or exogenous autoantigenic polypeptide(s) and/or autoantigen(s) is/are selected from one or more of: melan-A (MART1), gp100, tyrosinase, or tyrosinase-related protein 1 (TRP-1), and tyrosinase-related protein 2 (TRP-2).

In some embodiments, where the autoimmune disease is alopecis areata, the exogenous immunogenic polypeptide(s) and/or exogenous autoantigenic polypeptide(s) and/or autoantigen(s) is/are selected from one or more of: trichohyalin, TRP-2, gp100, or MART1.

In some embodiments, where the autoimmune disease is scleroderma, the exogenous immunogenic polypeptide(s) and/or exogenous autoantigenic polypeptide(s) and/or autoantigen(s) is/are selected from one or more of: topoisomerase, RNA binding region containing 3 (RNPC3), and RNA polymerase III (POLR3).

In some embodiments, the engineered erythroid cell or enucleated cell includes at least one exogenous immunogenic polypeptide (e.g., any of the exemplary exogenous immunogenic polypeptides described herein or known in the art), at least one exogenous HLA-G polypeptide (e.g., any of the exemplary exogenous HLA-G polypeptides described herein or known in the art), and optionally, (i) at least one exogenous coinhibitory polypeptide (e.g., any of the exemplary exogenous coinhibitory polypeptides described herein or known in the art) (e.g., one or more of IL-10, IL-27, IL-37, CD39, CD73, arginase 1 (ARG1), Annexin 1, fibrinogen-like protein 2 (FGL2), PD-L1, and TGFβ), and/or (ii) at least one exogenous antigenic polypeptide (e.g., any of the exemplary exogenous antigenic polypeptides described herein or known in the art).

In some embodiments, the engineered erythroid cell or enucleated cell provided herein (e.g., any of the exemplary engineered erythroid cells or enucleated cells described herein) is administered together with a second therapy or therapeutic agent. The second therapy may comprise, e.g., surgery, a biologic (e.g., a recombinant antibody), a cell-based therapy (e.g., CAR-T cell or CAR NK cell), and an immunosuppressant drug or agent. Non-limiting examples of immunosuppressant drugs and agents include: a corticosteroid (e.g., prednisone, budesonide, and prednisolone), a Janus kinase inhibitor (e.g., tofacitinib), a calcineurin inhibitor (e.g., cyclosporin or tacrolimus), an mTOR inhibitor (e.g., sirolimus and everolimus), an IMDH inhibitor (e.g., azathioprine, leflunomide, and mycophenolate), and a biologic (e.g., abatacept, adalimumab, anakinra, certolizumab, etanercept, golimumab, infliximab, ixekizumab, natalizumab, rituximab, secukinumab, tocilizumab, ustekinumab, vedolizumab, basilixumab, and daclizumab).

All publications and patent applications cited in this specification are herein incorporated by reference in their entirety for all purposes as if each individual publication or patent application were specifically and individually indicated to be incorporated by reference for all purposes. The publications discussed herein are provided solely for their disclosure prior to the filing date of the present application. Nothing herein is to be construed as an admission that the inventors described herein are not entitled to antedate such disclosure by virtue of prior disclosure or for any other reason.

### EXAMPLES

### Example 1. Generation of Engineered Enucleated Erythroid Cells Including Peptide-HLA-G-GPA and a Non-Human Immunogenic Polypeptide

Erythroid cells are transduced to include an exogenous HLA-G polypeptide that is a single chain fusion protein comprising an exogenous antigenic polypeptide HLA-G polypeptide, and the glycophorin A (GPA) transmembrane domain (peptide-HLA-G-GPA fusion protein). Optionally, the peptide-HLA-G-GPA fusion protein or the non-human immunogenic polypeptide comprises a detectable tag (e.g., a FLAG-tag or a myc-tag) that can be used to detect the protein(s). The erythroid cells are co-transduced to additionally include a non-human immunogenic polypeptide (e.g., a non-human amino-acid degrading polypeptide such as asparaginase). Cell culture and transduction is performed as described in the “Methods” section below to yield engineered enucleated erythroid cells including both the peptide-HLA-G-GPA fusion protein and the non-human immunogenic polypeptide on the cell surface.

The presence of peptide-HLA-G-GPA fusion protein and non-human immunogenic polypeptide on the surface of the engineered enucleated erythroid cells is determined by binding and detecting allophycocyanin (APC)-labelled or phycoerythrin (PE)-labelled anti-HLA-G and anti-immunogenic polypeptide antibodies.

### Methods

### Production of Lentiviral Vector

The nucleic acid encoding the peptide-HLA-G-GPA fusion protein and a non-human immunogenic polypeptide are generated and cloned into the multiple cloning site of the lentivirus vector pCDH (each under the control of the MSCV promoter (SYSTEM BIOSCIENCES), such that one lentivirus vector comprises genes encoding both proteins. Lentivirus is produced in 293T cells by transfecting the cells with pPACKH1 (SYSTEM BIOSCIENCES) and pCDH lentivirus vector containing the genes encoding peptide-HLA-G-GPA and the non-human immunogenic polypeptide using TransIT-LTI transfection reagent (MIRUS). After 12-14 hour incubation, cells are placed in fresh culturing medium. The supernatant comprising virus particles is collected 48 hours post-medium change by centrifugation at about 600×g for 5 minutes. The virus particles are concentrated by ultracentrifugation or tangential flow filtration (TFF) accompanied by ultracentrifugation. The supernatant is collected, filtered through a 0.45 μm filter, and frozen in aliquots at −80° C.

### Expansion and Differentiation of Erythroid Cells

Human CD34+ cells derived from mobilized peripheral blood cells from normal human donors are purchased frozen from AllCells Inc. The expansion/differentiation procedure comprises 3 stages. In the first stage, thawed CD34+ erythroid precursor cells are cultured in Iscove's MDM medium comprising recombinant human insulin, human transferrin, recombinant human recombinant human SCF, and recombinant human IL-3. In the second stage, erythroid cells are cultured in Iscove's MDM medium supplemented with human serum albumin, recombinant human insulin, human transferrin, human recombinant SCF, human recombinant EPO, and L-glutamine. In the third stage, erythroid cells are cultured in Iscove's MDM medium supplemented with human transferrin, recombinant human insulin, human recombinant EPO, and heparin. The cultures are maintained at 37° C. in 5% CO2 incubator.

### Transduction of Erythroid Precursor Cells

Erythroid precursor cells are transduced during step 1 of the culture process described above. Erythroid cells in culturing medium are combined with lentiviral supernatant and polaxamer 338. Infection is achieved by spinoculation, spinning the plate at 2000 rpm for 90 minutes at room temperature. After spinoculation, the cells are incubated at 37° C. overnight.

### Detection of Peptide-HLA-G-GPA and a Non-Human Immunogenic Polypeptide

The presence of the peptide-HLA-G-GPA fusion protein and of the non-human immunogenic polypeptide on the engineered enucleated erythroid cells is detected using allophycocyanin (APC)-labelled or phycoerythrin (PE)-labelled anti-HLA-G and anti-immunogenic polypeptide antibodies.

Binding of the antibodies is detected by flow cytometry for APC fluorescence or PE fluorescence, with a gate set based on stained untransduced cells. Alternatively, the peptide-HLA-G-GPA fusion protein and the non-human immunogenic polypeptide can be detected by Western blotting following SDS-PAGE separation using anti-HLA-G and anti-immunogenic polypeptide antibodies.

### Example 2. Activation of Immune Tolerance In Vitro by Engineered Enucleated Erythroid Cells Including Peptide-HLA-G-GPA and a Non-Human Immunogenic Polypeptide

Erythroid cells are transduced to include the peptide-HLA-G-GPA fusion protein and the non-human immunogenic polypeptide, for example as described in Example 1.

### Functional Assays

The effects of engineered enucleated erythroid cells including peptide-HLA-G-GPA and the non-human immunogenic polypeptide on T cell suppression are assessed by determining one or more of: (1) inhibition of T cell activity, (2) inhibition of T cell proliferation, and (3) induction of apoptosis of a T cell.

Inhibition of T cell activity is determined, for example, by contacting the engineered enucleated erythroid cells with activated T cells (e.g., CD4⁺ T cells) and performing a cytokine analysis of supernatants with commercially available ELISA kits (R&D SYSTEMS) (e.g., to detecthuman IL-2, IFN-γ, and IL-10 levels. For example, after treatment with the engineered enucleated erythroid cells, detection of IL-2 secretion inhibition, would indicate an anti-proliferative effect.

Inhibition of T cell proliferation is assayed, for example, by labelling T cells with the fluorescent dye 5,6-carboxyfluorescein diacetate succinimidyl ester (CFSE) and contacting the T cells with the engineered enucleated erythroid cells. T cells that proliferate in response to the engineered enucleated erythroid cell will show a reduction in CFSE fluorescence intensity, which is measured by flow cytometry. Alternatively, radioactive thymidine incorporation can be used to assess T cell growth rate in response to the engineered enucleated erythroid cells. Alternatively, inhibition of T cell proliferation is assayed by detecting specific cell proliferation markers such as Ki67 (e.g., using human anti Ki67 antibody, clone AbD02531 (BIORAD).

Induction of T cell apoptosis by the engineered enucleated erythroid cells is assayed using, for example, fluorochrome-conjugated annexin V staining.

To detect and measure immune cell activation following exposure of human immune cells to the engineered enucleated erythroid cells including peptide-HLA-G-GPA fusion protein and non-human immunogenic polypeptide, ex vivo immunoassays with human peripheral blood mononuclear cells (PBMCs) can be used, as described in Salvat et al. (2017) Proc. Nat'l. Acad. Sci. U.S.A. 114(26): E5085-93), the entire contents of which are incorporated herein by reference.

The effects of the engineered enucleated erythroid cells including peptide-HLA-G-GPA fusion protein and non-human immunogenic polypeptide, on the inhibition of B cell proliferation, differentiation, and antibody (Ig) secretion, and on the inhibition of NK cell proliferation and cytotoxicity, is determined as described in Rebmann et al. (2014) J. Immunol Res. 2014: 297073, the entire contents of which are incorporated herein by reference.

1. An engineered enucleated erythroid cell comprising an exogenous human leukocyte antigen-G (HLA-G) polypeptide and an exogenous immunogenic polypeptide, wherein both the exogenous HLA-G polypeptide and the exogenous immunogenic polypeptide are on the cell surface.

2. An engineered enucleated erythroid cell comprising an exogenous human leukocyte antigen-G (HLA-G) polypeptide and an exogenous immunogenic polypeptide, wherein the exogenous HLA-G polypeptide is on the cell surface and the exogenous immunogenic polypeptide is within the cell.

3.-17. (canceled)

18. The engineered enucleated erythroid cell of claim 1, wherein the exogenous HLA-G polypeptide is capable of inducing immune tolerance to the exogenous immunogenic polypeptide upon administration of the cell to a subject.

19.-26. (canceled)

27. The engineered enucleated erythroid cell of claim 1, wherein the exogenous HLA-G polypeptide is bound to an exogenous antigenic polypeptide.

28. (canceled)

29. (canceled)

30. The engineered enucleated erythroid cell of claim 27, wherein the exogenous antigenic polypeptide is covalently bound to the exogenous HLA-G polypeptide.

31. The engineered enucleated erythroid cell of claim 27, wherein the exogenous antigenic polypeptide is non-covalently bound to the exogenous HLA-G polypeptide.

32. The engineered enucleated erythroid cell of claim 1, wherein the exogenous HLA-G polypeptide comprises one or more alpha domains of an HLA-G alpha chain, or a fragment thereof, and a β2M polypeptide, or a fragment thereof.

33. The engineered enucleated erythroid cell of claim 32, wherein the exogenous HLA-G polypeptide is linked to a membrane anchor.

34. The engineered enucleated erythroid cell of claim 32, wherein the exogenous HLA-G polypeptide is a single chain fusion protein comprising an exogenous antigenic polypeptide linked to the exogenous HLA-G polypeptide via a linker.

35. The engineered enucleated erythroid cell of claim 34, wherein the single chain fusion protein further comprises a membrane anchor.

36. (canceled)

37. The engineered enucleated erythroid cell of claim 1, wherein the exogenous immunogenic polypeptide is not bound to the exogenous HLA-G polypeptide.

38. (canceled)

39. The engineered enucleated erythroid cell of claim 1, wherein the engineered enucleated erythroid cell further comprises an exogenous autoantigenic polypeptide.

40. The engineered enucleated erythroid cell of claim 39, wherein the exogenous autoantigenic polypeptide is on the cell surface.

41. (canceled)

42. The engineered enucleated erythroid cell of claim 40, wherein the exogenous autoantigenic polypeptide comprises Formula I in an N-terminal to a C-terminal direction:
X₁-X₂-X₃ (Formula I),
wherein:
X₁ comprises a type II membrane protein or a transmembrane domain thereof;
X₂ comprises a Ii key peptide; and
X₃ comprises an autoantigen.

43. The engineered enucleated erythroid cell of claim 40, wherein the exogenous autoantigenic polypeptide comprises Formula II in an N-terminal to C-terminal direction:
X₁-X₂-X₃-X₄ (Formula II),
wherein:
X₁ comprises a type II membrane protein or a transmembrane domain thereof;
X₂ comprises a linker;
X₃ comprises a Ii key peptide; and
X₄ comprises an autoantigen.

44.-50. (canceled)

51. The engineered enucleated erythroid cell of claim 39, wherein the exogenous autoantigenic polypeptide is within the cell.

52. The engineered enucleated erythroid cell of claim 51, wherein the exogenous autoantigenic polypeptide is on the intracellular side of the plasma membrane.

53. (canceled)

54. The engineered enucleated erythroid cell of claim 52, wherein the exogenous antigenic polypeptide comprises Formula III in an N-terminal to a C-terminal direction:
X₁-X₂-X₃ (Formula III),
wherein:
X₁ comprises a type I membrane protein or a transmembrane domain thereof;
X₂ comprises a Ii key peptide; and
X₃ comprises an autoantigen.

55. The engineered enucleated erythroid cell of claim 52, wherein the exogenous autoantigenic polypeptide comprises Formula IV in an N-terminal to C-terminal direction:
X₁-X₂-X₃-X₄ (Formula IV),
wherein:
X₁ comprises a type I membrane protein or a transmembrane domain thereof;
X₂ comprises a linker;
X₃ comprises a Ii key peptide; and
X₄ comprises an autoantigen.

56.-60. (canceled)

61. The engineered enucleated erythroid cell of claim 52, wherein the exogenous autoantigenic polypeptide comprises Formula VII in an N-terminal to C-terminal direction:
X₁-X₂-X₃-X₄ (Formula VII),
wherein:
X₁ comprises a type I membrane protein or a transmembrane domain thereof;
X₂ comprises a linker;
X₃ comprises a cytoplasmic portion of CD74 or a fragment thereof; and
X₄ comprises an autoantigen.

62.-64. (canceled)

65. The engineered enucleated erythroid cell of claim 52, wherein the exogenous autoantigenic polypeptide comprises Formula VIII in an N-terminal to C-terminal direction:
X₁-X₂-X₃-X₄-X₅ (Formula VIII),
wherein:
X₁ comprises a type I membrane protein or a transmembrane domain thereof;
X₂ comprises a linker;
X₃ comprises a N-terminal cytoplasmic portion of CD74 or a fragment thereof;
X₄ comprises an autoantigen; and
X₅ comprises a C-terminal cytoplasmic portion of CD74.

66.-69. (canceled)

70. The engineered enucleated erythroid cell of claim 52, wherein the exogenous autoantigenic polypeptide comprises Formula XI in an N-terminal to C-terminal direction:
X₁-X₂-X₃-X₄ (Formula XI),
wherein:
X₁ comprises a cytosolic protein or a fragment thereof;
X₂ comprises a linker;
X₃ comprises a cytoplasmic portion of CD74 or a fragment thereof; and
X₄ comprises an autoantigen.

71.-74. (canceled)

75. The engineered enucleated erythroid cell of claim 52, wherein the exogenous autoantigenic polypeptide comprises Formula XII in an N-terminal to C-terminal direction:
X₁-X₂-X₃-X₄-X₅ (Formula XII),
wherein:
X₁ comprises a cytoplasmice protein or a fragment thereof;
X₂ comprises a linker;
X₃ comprises a N-terminal cytoplasmic portion of CD74 or a fragment thereof;
X₄ comprises an autoantigen; and
X₅ comprises a C-terminal cytoplasmic portion of CD74.

76.-81. (canceled)

82. The engineered enucleated erythroid cell of claim 40, wherein the exogenous autoantigenic polypeptide comprises Formula IX in an N-terminal to C-terminal direction:
X₁-X₂-X₃ (Formula IX),
wherein:
X₁ comprises a type II membrane protein or a transmembrane domain thereof;
X₂ comprises a cytoplasmic portion of CD74 or a fragment thereof; and
X₃ comprises an autoantigen.

83. (canceled)

84. (canceled)

85. The engineered enucleated erythroid cell of claim 40, wherein the exogenous autoantigenic polypeptide comprises Formula X in an N-terminal to C-terminal direction:
X₁-X₂-X₃-X₄-X₅ (Formula X),
wherein:
X₁ comprises a type II membrane protein or a transmembrane domain thereof;
X₂ comprises a linker;
X₃ comprises a N-terminal cytoplasmic portion of CD74 or a fragment thereof;
X₄ comprises an autoantigen; and
X₅ comprises a C-terminal cytoplasmic portion of CD74.

86.-89. (canceled)

90. The engineered enucleated erythroid cell of claim 40, wherein the exogenous autoantigenic polypeptide comprises Formula XIII in an N-terminal to C-terminal direction:
X₁-X₂-X₃-X₄ (Formula XIII),
wherein:
X₁ comprises an Ii key peptide;
X₂ comprises an autoantigen;
X₃ comprises a linker; and
X₄ comprises a Type I membrane protein or a transmembrane domain thereof.

91.-97. (canceled)

98. The engineered enucleated erythroid cell of claim 39, wherein the exogenous autoantigenic polypeptide is in the cytosol of the cell.

99. The engineered enucleated erythroid cell of claim 98, wherein the exogenous autoantigenic polypeptide comprises Formula V in an N-terminal to a C-terminal direction:
X₁-X₂-X₃ (Formula V),
wherein:
X₁ comprises a cytosolic polypeptide or a fragment thereof;
X₂ comprises a Ii key peptide; and
X₃ comprises an autoantigen.

100. The engineered enucleated erythroid cell of claim 98, wherein the exogenous autoantigenic polypeptide comprises Formula VI in an N-terminal to C-terminal direction:
X₁-X₂-X₃-X₄ (Formula VI),
wherein:
X₁ comprises a cytosolic polypeptide or a fragment thereof;
X₂ comprises a linker;
X₃ comprises a Ii key peptide; and
X₄ comprises an autoantigen.

101.-108. (canceled)

109. The engineered enucleated erythroid cell of claim 1, wherein the engineered enucleated erythroid cell further comprises at least one exogenous coinhibitory polypeptide.

110.-121. (canceled)

122. An engineered enucleated erythroid cell comprising an exogenous autoantigenic polypeptide and at least one exogenous coinhibitory polypeptide.

123.-203. (canceled)

204. The engineered enucleated erythroid cell of claim 1, wherein the engineered enucleated erythroid cell is a reticulocyte.

205. The engineered enucleated erythroid cell of claim 1, wherein the engineered enucleated erythroid cell is an erythrocyte.

206. The engineered enucleated erythroid cell of claim 1, wherein the engineered enucleated erythroid cell is a human cell.

207. A pharmaceutical composition comprising a plurality of the engineered enucleated erythroid cells of claim 1, and a pharmaceutically acceptable carrier.

208. A method of inducing immune tolerance in a subject to an exogenous immunogenic polypeptide, the method comprising administering to the subject a therapeutically effective amount of the pharmaceutical composition of claim 207, thereby inducing immune tolerance to the exogenous immunogenic polypeptide.

209.-216. (canceled)

217. A method of treating a disease in a subject in need thereof, the method comprising administering to the subject a therapeutically effective amount of the pharmaceutical composition of claim 207, thereby treating the disease in the subject.

218.-234. (canceled)
